SEQUENCE LISTING (1339 sequences)

SEQ ID NO: 1 - DNA, 40 bp, synthetic construct
  source 1..40: mol_type = other DNA
cactcacctccatctccactcctacccatccaactcccac

SEQ ID NO: 2 - DNA, 40 bp, synthetic construct
  source 1..40: mol_type = other DNA
gtgggagttggatgggtaggagtggagatggaggtgagtg

SEQ ID NO: 3 - DNA, 40 bp, synthetic construct
  source 1..40: mol_type = other DNA
cgtttctttgcttgtgtcttgtcttcgtcgtgcttgttcc

SEQ ID NO: 4 - DNA, 40 bp, synthetic construct
  source 1..40: mol_type = other DNA
ggaacaagcacgacgaagacaagacacaagcaaagaaacg

SEQ ID NO: 5 - DNA, 20 bp, synthetic construct
  source 1..20: mol_type = other DNA
acagttattagcccgcattt

SEQ ID NO: 6 - DNA, 20 bp, synthetic construct
  source 1..20: mol_type = other DNA
aaatgcgggctaataactgt

SEQ ID NO: 7 - DNA, 10 bp, synthetic construct
  source 1..10: mol_type = other DNA
ctagatgtat

SEQ ID NO: 8 - DNA, 10 bp, synthetic construct
  source 1..10: mol_type = other DNA
atacatctag

SEQ ID NO: 9 - DNA, 10 bp, synthetic construct
  source 1..10: mol_type = other DNA
taagacagat

SEQ ID NO: 10 - DNA, 10 bp, synthetic construct
  source 1..10: mol_type = other DNA
tactgtctta

SEQ ID NO: 11 - DNA, 33 bp, synthetic construct
  source 1..33: mol_type = other DNA
gtggatgggtaggagtggagatggaggtgagtg

SEQ ID NO: 12 - DNA, 24 bp, synthetic construct
  source 1..24: mol_type = other DNA
tcatttgctaatagtagtagcatt

SEQ ID NO: 13 - DNA, 24 bp, synthetic construct
  source 1..24: mol_type = other DNA
caactaaagtacggtgggatggct

SEQ ID NO: 14 - DNA, 24 bp, synthetic construct
  source 1..24: mol_type = other DNA
cattattagcaaaagaagttttgc

SEQ ID NO: 15 - DNA, 24 bp, synthetic construct
  source 1..24: mol_type = other DNA
accctcattcagggatagcaagcc

SEQ ID NO: 16 - DNA, 24 bp, synthetic construct
  source 1..24: mol_type = other DNA
ttaggattagcggggtggaaccta

SEQ ID NO: 17 - DNA, 24 bp, synthetic construct
  source 1..24: mol_type = other DNA
aggccggaaccagagccaccaccg

SEQ ID NO: 18 - DNA, 24 bp, synthetic construct
  source 1..24: mol_type = other DNA
agaatatcagacgacgacaataaa

SEQ ID NO: 19 - DNA, 24 bp, synthetic construct
  source 1..24: mol_type = other DNA
tcatatgcgttatacaaaggcgtt

SEQ ID NO: 20 - DNA, 24 bp, synthetic construct
  source 1..24: mol_type = other DNA
cgggagaatttaatggaaacagta

SEQ ID NO: 21 - DNA, 24 bp, synthetic construct
  source 1..24: mol_type = other DNA
gcgcgtactttcctcgttagaatc

SEQ ID NO: 22 - DNA, 24 bp, synthetic construct
  source 1..24: mol_type = other DNA
aaagccggcgaacgtgtgccgtaa

SEQ ID NO: 23 - DNA, 24 bp, synthetic construct
  source 1..24: mol_type = other DNA
aattccacgtttgcgtattgggcg

SEQ ID NO: 24 - DNA, 30 bp, synthetic construct
  source 1..30: mol_type = other DNA
ttaagagggtccaatactgcggatagcgag

SEQ ID NO: 25 - DNA, 30 bp, synthetic construct
  source 1..30: mol_type = other DNA
aggcttttcaggtagaaagattcaattacc

SEQ ID NO: 26 - DNA, 30 bp, synthetic construct
  source 1..30: mol_type = other DNA
ttatgcgattgacaagaaccggaggtcaat

SEQ ID NO: 27 - DNA, 30 bp, synthetic construct
  source 1..30: mol_type = other DNA
cataagggacactaaaacactcacattaaa

SEQ ID NO: 28 - DNA, 30 bp, synthetic construct
  source 1..30: mol_type = other DNA
cgggtaaaattcggtcgctgaggaatgaca

SEQ ID NO: 29 - DNA, 30 bp, synthetic construct
  source 1..30: mol_type = other DNA
gtctctgacaccctcagagccacatcaaaa

SEQ ID NO: 30 - DNA, 30 bp, synthetic construct
  source 1..30: mol_type = other DNA
tcaccggaaacgtcaccaatgaattattca

SEQ ID NO: 31 - DNA, 30 bp, synthetic construct
  source 1..30: mol_type = other DNA
ttaaaggtacatataaaagaaacaaacgca

SEQ ID NO: 32 - DNA, 30 bp, synthetic construct
  source 1..30: mol_type = other DNA
ataataactcagagagataacccgaagcgc

SEQ ID NO: 33 - DNA, 30 bp, synthetic construct
  source 1..30: mol_type = other DNA
attagacggagcgtctttccagagctacaa

SEQ ID NO: 34 - DNA, 30 bp, synthetic construct
  source 1..30: mol_type = other DNA
tatataacgtaaatcgtcgctatatttgaa

SEQ ID NO: 35 - DNA, 30 bp, synthetic construct
  source 1..30: mol_type = other DNA
ttacctttacaataacggattcgcaaaatt

SEQ ID NO: 36 - DNA, 30 bp, synthetic construct
  source 1..30: mol_type = other DNA
atttgcaccattttgcggaacaaatttgag

SEQ ID NO: 37 - DNA, 30 bp, synthetic construct
  source 1..30: mol_type = other DNA
gatttagattgctgaacctcaaagtattaa

SEQ ID NO: 38 - DNA, 30 bp, synthetic construct
  source 1..30: mol_type = other DNA
caccgcctgaaagcgtaagaatacattctg

SEQ ID NO: 39 - DNA, 30 bp, synthetic construct
  source 1..30: mol_type = other DNA
tgagtgttcagctgattgcccttgcgcggg

SEQ ID NO: 40 - DNA, 30 bp, synthetic construct
  source 1..30: mol_type = other DNA
gagaggcgacaacatacgagccgctgcagg

SEQ ID NO: 41 - DNA, 30 bp, synthetic construct
  source 1..30: mol_type = other DNA
tcgactctgaagggcgatcggtgcggcctc

SEQ ID NO: 42 - DNA, 30 bp, synthetic construct
  source 1..30: mol_type = other DNA
aggaagatcattaaatgtgagcgtttttaa

SEQ ID NO: 43 - DNA, 30 bp, synthetic construct
  source 1..30: mol_type = other DNA
ccaataggaaactagcatgtcaaggagcaa

SEQ ID NO: 44 - DNA, 32 bp, synthetic construct
  source 1..32: mol_type = other DNA
tagagcttcagaccggaagcaaacctattata

SEQ ID NO: 45 - DNA, 32 bp, synthetic construct
  source 1..32: mol_type = other DNA
gtcagaagattgaatccccctcaacctcgttt

SEQ ID NO: 46 - DNA, 32 bp, synthetic construct
  source 1..32: mol_type = other DNA
aaatattccaaagcggattgcatcgagcttca

SEQ ID NO: 47 - DNA, 32 bp, synthetic construct
  source 1..32: mol_type = other DNA
accagacggaataccacattcaacgagatggt

SEQ ID NO: 48 - DNA, 32 bp, synthetic construct
  source 1..32: mol_type = other DNA
agatttagacgataaaaaccaaaaatcgtcat

SEQ ID NO: 49 - DNA, 32 bp, synthetic construct
  source 1..32: mol_type = other DNA
agtcaggacataggctggctgacctttgaaag

SEQ ID NO: 50 - DNA, 32 bp, synthetic construct
  source 1..32: mol_type = other DNA
ttaatttccaacgtaacaaagctgtccatgtt

SEQ ID NO: 51 - DNA, 32 bp, synthetic construct
  source 1..32: mol_type = other DNA
gagtaatcttttaagaactggctccggaacaa

SEQ ID NO: 52 - DNA, 32 bp, synthetic construct
  source 1..32: mol_type = other DNA
acccaaataactttaatcattgtgatcagttg

SEQ ID NO: 53 - DNA, 32 bp, synthetic construct
  source 1..32: mol_type = other DNA
acttagccattataccaagcgcgagaggacta

SEQ ID NO: 54 - DNA, 32 bp, synthetic construct
  source 1..32: mol_type = other DNA
aaaagaataaccgaactgaccaacttcatcaa

SEQ ID NO: 55 - DNA, 32 bp, synthetic construct
  source 1..32: mol_type = other DNA
ccccagcgggaacgaggcgcagactattcatt

SEQ ID NO: 56 - DNA, 32 bp, synthetic construct
  source 1..32: mol_type = other DNA
aagactttggccgcttttgcgggattaaacag

SEQ ID NO: 57 - DNA, 32 bp, synthetic construct
  source 1..32: mol_type = other DNA
gagttaaattcatgaggaagtttctctttgac

SEQ ID NO: 58 - DNA, 32 bp, synthetic construct
  source 1..32: mol_type = other DNA
cttgatactgaaaatctccaaaaaagcggagt

SEQ ID NO: 59 - DNA, 32 bp, synthetic construct
  source 1..32: mol_type = other DNA
tttcacgtcgatagttgcgccgaccttgcagg

SEQ ID NO: 60 - DNA, 32 bp, synthetic construct
  source 1..32: mol_type = other DNA
ttattctgactggtaataagttttaacaaata

SEQ ID NO: 61 - DNA, 32 bp, synthetic construct
  source 1..32: mol_type = other DNA
aatcctcaaccagaaccaccaccagccccctt

SEQ ID NO: 62 - DNA, 32 bp, synthetic construct
  source 1..32: mol_type = other DNA
gagccgccttaaagccagaatggagatgatac

SEQ ID NO: 63 - DNA, 32 bp, synthetic construct
  source 1..32: mol_type = other DNA
attagcgtccgtaatcagtagcgaattgaggg

SEQ ID NO: 64 - DNA, 32 bp, synthetic construct
  source 1..32: mol_type = other DNA
gccatttgcaaacgtagaaaatacctggcatg

SEQ ID NO: 65 - DNA, 32 bp, synthetic construct
  source 1..32: mol_type = other DNA
agggaaggataagtttattttgtcagccgaac

SEQ ID NO: 66 - DNA, 32 bp, synthetic construct
  source 1..32: mol_type = other DNA
aggtggcagaattatcaccgtcaccattagca

SEQ ID NO: 67 - DNA, 32 bp, synthetic construct
  source 1..32: mol_type = other DNA
aaagttacgcccaataataagagcagccttta

SEQ ID NO: 68 - DNA, 32 bp, synthetic construct
  source 1..32: mol_type = other DNA
cgctaataggaatacccaaaagaaatacataa

SEQ ID NO: 69 - DNA, 32 bp, synthetic construct
  source 1..32: mol_type = other DNA
cagagagaacaaaataaacagccattaaatca

SEQ ID NO: 70 - DNA, 32 bp, synthetic construct
  source 1..32: mol_type = other DNA
agattagtatatagaaggcttatccaagccgt

SEQ ID NO: 71 - DNA, 32 bp, synthetic construct
  source 1..32: mol_type = other DNA
caaatcagtgctattttgcacccagcctaatt

SEQ ID NO: 72 - DNA, 32 bp, synthetic construct
  source 1..32: mol_type = other DNA
aaataagaactttttcaaatatatctgagaga

SEQ ID NO: 73 - DNA, 32 bp, synthetic construct
  source 1..32: mol_type = other DNA
ctacctttagaatccttgaaaacaagaaaaca

SEQ ID NO: 74 - DNA, 32 bp, synthetic construct
  source 1..32: mol_type = other DNA
tttcccttttaacctccggcttagcaaagaac

SEQ ID NO: 75 - DNA, 32 bp, synthetic construct
  source 1..32: mol_type = other DNA
aaattaataccaagttacaaaatcctgaataa

SEQ ID NO: 76 - DNA, 32 bp, synthetic construct
  source 1..32: mol_type = other DNA
ctttgaattacatttaacaatttctaattaat

SEQ ID NO: 77 - DNA, 32 bp, synthetic construct
  source 1..32: mol_type = other DNA
gtagatttgttattaattttaaaaaacaattc

SEQ ID NO: 78 - DNA, 32 bp, synthetic construct
  source 1..32: mol_type = other DNA
tggaagggagcggaattatcatcaactaatag

SEQ ID NO: 79 - DNA, 32 bp, synthetic construct
  source 1..32: mol_type = other DNA
aacattatgtaaaacagaaataaattttacat

SEQ ID NO: 80 - DNA, 32 bp, synthetic construct
  source 1..32: mol_type = other DNA
ccagaaggttagaacctaccatatcctgattg

SEQ ID NO: 81 - DNA, 32 bp, synthetic construct
  source 1..32: mol_type = other DNA
attagagcaatatctggtcagttgcagcagaa

SEQ ID NO: 82 - DNA, 32 bp, synthetic construct
  source 1..32: mol_type = other DNA
gcatcaccagtattagactttacagtttgagt

SEQ ID NO: 83 - DNA, 32 bp, synthetic construct
  source 1..32: mol_type = other DNA
cctcaatccgtcaatagataatacagaaacca

SEQ ID NO: 84 - DNA, 32 bp, synthetic construct
  source 1..32: mol_type = other DNA
gataaaactttttgaatggctattttcaccag

SEQ ID NO: 85 - DNA, 32 bp, synthetic construct
  source 1..32: mol_type = other DNA
agacaataagaggtgaggcggtcatatcaaac

SEQ ID NO: 86 - DNA, 32 bp, synthetic construct
  source 1..32: mol_type = other DNA
tcacacgatgcaacaggaaaaacggaagaact

SEQ ID NO: 87 - DNA, 32 bp, synthetic construct
  source 1..32: mol_type = other DNA
ccagccatccagtaataaaagggacgtggcac

SEQ ID NO: 88 - DNA, 32 bp, synthetic construct
  source 1..32: mol_type = other DNA
agcactaaaaagggcgaaaaaccgaaatccct

SEQ ID NO: 89 - DNA, 32 bp, synthetic construct
  source 1..32: mol_type = other DNA
tataaatcgagagttgcagcaagcgtcgtgcc

SEQ ID NO: 90 - DNA, 32 bp, synthetic construct
  source 1..32: mol_type = other DNA
ggccctgaaaaagaatagcccgagcgtggact

SEQ ID NO: 91 - DNA, 32 bp, synthetic construct
  source 1..32: mol_type = other DNA
agctgcatagcctggggtgcctaagtaaaacg

SEQ ID NO: 92 - DNA, 32 bp, synthetic construct
  source 1..32: mol_type = other DNA
aagtgtaataatgaatcggccaaccaccgcct

SEQ ID NO: 93 - DNA, 32 bp, synthetic construct
  source 1..32: mol_type = other DNA
gaattcgtgccattcgccattcagttccggca

SEQ ID NO: 94 - DNA, 32 bp, synthetic construct
  source 1..32: mol_type = other DNA
acggccagtacgccagctggcgaacatctgcc

SEQ ID NO: 95 - DNA, 32 bp, synthetic construct
  source 1..32: mol_type = other DNA
actgttggagaggatccccgggtaccgctcac

SEQ ID NO: 96 - DNA, 32 bp, synthetic construct
  source 1..32: mol_type = other DNA
ttcgctattgccaagcttgcatgcgaagcata

SEQ ID NO: 97 - DNA, 32 bp, synthetic construct
  source 1..32: mol_type = other DNA
agtttgagattctccgtgggaacaattcgcat

SEQ ID NO: 98 - DNA, 32 bp, synthetic construct
  source 1..32: mol_type = other DNA
ttcatcaacgcactccagccagctgctgcgca

SEQ ID NO: 99 - DNA, 32 bp, synthetic construct
  source 1..32: mol_type = other DNA
cccgtcgggggacgacgacagtatcgggcctc

SEQ ID NO: 100 - DNA, 32 bp, synthetic construct
  source 1..32: mol_type = other DNA
taaatttttgataatcagaaaagcacaaaggc

SEQ ID NO: 101 - DNA, 32 bp, synthetic construct
  source 1..32: mol_type = other DNA
accccggttgttaaatcagctcatagtaacaa

SEQ ID NO: 102 - DNA, 32 bp, synthetic construct
  source 1..32: mol_type = other DNA
tatcaggtaaatcaccatcaatatcaatgcct

SEQ ID NO: 103 - DNA, 32 bp, synthetic construct
  source 1..32: mol_type = other DNA
agacagtccattgcctgagagtcttcatatgt

SEQ ID NO: 104 - DNA, 48 bp, synthetic construct
  source 1..48: mol_type = other DNA
gacggaaaaccatcgatagcagcattgccatcttttcatacaccctca

SEQ ID NO: 105 - DNA, 48 bp, synthetic construct
  source 1..48: mol_type = other DNA
tgccagttataacataaaaacaggacaagaattgagttaacagaagga

SEQ ID NO: 106 - DNA, 78 bp, synthetic construct
  source 1..78: mol_type = other DNA
tttggtggcatcaattctagggcgcgagctgaaaatttcgtttctttgcttgtgtcttgtcttcgtcgtgcttgttcc

SEQ ID NO: 107 - DNA, 78 bp, synthetic construct
  source 1..78: mol_type = other DNA
ttttcccaattctgcgaacccatataacagttgattttcgtttctttgcttgtgtcttgtcttcgtcgtgcttgttcc

SEQ ID NO: 108 - DNA, 78 bp, synthetic construct
  source 1..78: mol_type = other DNA
tttattgctccttttgatattagagagtacctttatttcgtttctttgcttgtgtcttgtcttcgtcgtgcttgttcc

SEQ ID NO: 109 - DNA, 78 bp, synthetic construct
  source 1..78: mol_type = other DNA
tttccataaatcaaaaatccagaaaacgagaatgatttcgtttctttgcttgtgtcttgtcttcgtcgtgcttgttcc

SEQ ID NO: 110 - DNA, 78 bp, synthetic construct
  source 1..78: mol_type = other DNA
tttcgaggcatagtaagagacgccaaaaggaattatttcgtttctttgcttgtgtcttgtcttcgtcgtgcttgttcc

SEQ ID NO: 111 - DNA, 78 bp, synthetic construct
  source 1..78: mol_type = other DNA
tttgaaacaccagaacgagaggcttgccctgacgatttcgtttctttgcttgtgtcttgtcttcgtcgtgcttgttcc

SEQ ID NO: 112 - DNA, 78 bp, synthetic construct
  source 1..78: mol_type = other DNA
tttctgataaattgtgtcgagatttgtatcatcgctttcgtttctttgcttgtgtcttgtcttcgtcgtgcttgttcc

SEQ ID NO: 113 - DNA, 78 bp, synthetic construct
  source 1..78: mol_type = other DNA
tttgaacgagggtagcaacgcgaaagacagcatcgtttcgtttctttgcttgtgtcttgtcttcgtcgtgcttgttcc

SEQ ID NO: 114 - DNA, 78 bp, synthetic construct
  source 1..78: mol_type = other DNA
tttggtttatcagcttgctagcctttaattgtatctttcgtttctttgcttgtgtcttgtcttcgtcgtgcttgttcc

SEQ ID NO: 115 - DNA, 78 bp, synthetic construct
  source 1..78: mol_type = other DNA
tttgggattttgctaaacaaatgaattttctgtattttcgtttctttgcttgtgtcttgtcttcgtcgtgcttgttcc

SEQ ID NO: 116 - DNA, 78 bp, synthetic construct
  source 1..78: mol_type = other DNA
tttacaaactacaacgcctgagtttcgtcaccagttttcgtttctttgcttgtgtcttgtcttcgtcgtgcttgttcc

SEQ ID NO: 117 - DNA, 78 bp, synthetic construct
  source 1..78: mol_type = other DNA
tttagccaccaccctcattgaaccgccaccctcagtttcgtttctttgcttgtgtcttgtcttcgtcgtgcttgttcc

SEQ ID NO: 118 - DNA, 78 bp, synthetic construct
  source 1..78: mol_type = other DNA
tttgagagggttgatataagcggataagtgccgtctttcgtttctttgcttgtgtcttgtcttcgtcgtgcttgttcc

SEQ ID NO: 119 - DNA, 78 bp, synthetic construct
  source 1..78: mol_type = other DNA
tttgtataaacagttaatgttgagtaacagtgccctttcgtttctttgcttgtgtcttgtcttcgtcgtgcttgttcc

SEQ ID NO: 120 - DNA, 78 bp, synthetic construct
  source 1..78: mol_type = other DNA
tttgcaggtcagacgattgttgacaggaggttgagtttcgtttctttgcttgtgtcttgtcttcgtcgtgcttgttcc

SEQ ID NO: 121 - DNA, 78 bp, synthetic construct
  source 1..78: mol_type = other DNA
ttttagcgcgttttcatcgctttagcgtcagactgtttcgtttctttgcttgtgtcttgtcttcgtcgtgcttgttcc

SEQ ID NO: 122 - DNA, 78 bp, synthetic construct
  source 1..78: mol_type = other DNA
tttgcgccaaagacaaaagttcatatggtttaccatttcgtttctttgcttgtgtcttgtcttcgtcgtgcttgttcc

SEQ ID NO: 123 - DNA, 78 bp, synthetic construct
  source 1..78: mol_type = other DNA
tttccgaagccctttttaaagcaatagctatcttatttcgtttctttgcttgtgtcttgtcttcgtcgtgcttgttcc

SEQ ID NO: 124 - DNA, 78 bp, synthetic construct
  source 1..78: mol_type = other DNA
tttttttttgtttaacgtctccaaataagaaacgatttcgtttctttgcttgtgtcttgtcttcgtcgtgcttgttcc

SEQ ID NO: 125 - DNA, 78 bp, synthetic construct
  source 1..78: mol_type = other DNA
tttaacctcccgacttgcggcgaggcgttttagcgtttcgtttctttgcttgtgtcttgtcttcgtcgtgcttgttcc

SEQ ID NO: 126 - DNA, 78 bp, synthetic construct
  source 1..78: mol_type = other DNA
ttttaaaccaagtaccgcattccaagaacgggtattttcgtttctttgcttgtgtcttgtcttcgtcgtgcttgttcc

SEQ ID NO: 127 - DNA, 78 bp, synthetic construct
  source 1..78: mol_type = other DNA
tttagataagtcctgaacacctgtttatcaacaattttcgtttctttgcttgtgtcttgtcttcgtcgtgcttgttcc

SEQ ID NO: 128 - DNA, 78 bp, synthetic construct
  source 1..78: mol_type = other DNA
tttgtaaagtaattctgtcaaagtaccgacaaaagtttcgtttctttgcttgtgtcttgtcttcgtcgtgcttgttcc

SEQ ID NO: 129 - DNA, 78 bp, synthetic construct
  source 1..78: mol_type = other DNA
tttagtagggcttaattgaaaagccaacgctcaactttcgtttctttgcttgtgtcttgtcttcgtcgtgcttgttcc

SEQ ID NO: 130 - DNA, 78 bp, synthetic construct
  source 1..78: mol_type = other DNA
tttaatggtttgaaatacccttctgacctaaattttttcgtttctttgcttgtgtcttgtcttcgtcgtgcttgttcc

SEQ ID NO: 131 - DNA, 78 bp, synthetic construct
  source 1..78: mol_type = other DNA
tttagtcaatagtgaatttttaagacgctgagaagtttcgtttctttgcttgtgtcttgtcttcgtcgtgcttgttcc

SEQ ID NO: 132 - DNA, 78 bp, synthetic construct
  source 1..78: mol_type = other DNA
ttttgagcaaaagaagatgattcatttcaattacctttcgtttctttgcttgtgtcttgtcttcgtcgtgcttgttcc

SEQ ID NO: 133 - DNA, 78 bp, synthetic construct
  source 1..78: mol_type = other DNA
tttcaatataatcctgattgatgatggcaattcattttcgtttctttgcttgtgtcttgtcttcgtcgtgcttgttcc

SEQ ID NO: 134 - DNA, 78 bp, synthetic construct
  source 1..78: mol_type = other DNA
tttgttatctaaaatatctaaaggaattgaggaagtttcgtttctttgcttgtgtcttgtcttcgtcgtgcttgttcc

SEQ ID NO: 135 - DNA, 78 bp, synthetic construct
  source 1..78: mol_type = other DNA
tttacatcgccattaaaaaaactgatagccctaaatttcgtttctttgcttgtgtcttgtcttcgtcgtgcttgttcc

SEQ ID NO: 136 - DNA, 78 bp, synthetic construct
  source 1..78: mol_type = other DNA
ttttcgtctgaaatggattacattttgacgctcaatttcgtttctttgcttgtgtcttgtcttcgtcgtgcttgttcc

SEQ ID NO: 137 - DNA, 78 bp, synthetic construct
  source 1..78: mol_type = other DNA
tttttgattagtaataacattgtagcaatacttcttttcgtttctttgcttgtgtcttgtcttcgtcgtgcttgttcc

SEQ ID NO: 138 - DNA, 78 bp, synthetic construct
  source 1..78: mol_type = other DNA
tttaggaacggtacgccagtaaagggattttagactttcgtttctttgcttgtgtcttgtcttcgtcgtgcttgttcc

SEQ ID NO: 139 - DNA, 78 bp, synthetic construct
  source 1..78: mol_type = other DNA
tttgagcacgtataacgtgctatggttgctttgactttcgtttctttgcttgtgtcttgtcttcgtcgtgcttgttcc

SEQ ID NO: 140 - DNA, 78 bp, synthetic construct
  source 1..78: mol_type = other DNA
tttcgggcgctagggcgctaagaaagcgaaaggagtttcgtttctttgcttgtgtcttgtcttcgtcgtgcttgttcc

SEQ ID NO: 141 - DNA, 78 bp, synthetic construct
  source 1..78: mol_type = other DNA
tttatcacccaaatcaagtgcccactacgtgaacctttcgtttctttgcttgtgtcttgtcttcgtcgtgcttgttcc

SEQ ID NO: 142 - DNA, 78 bp, synthetic construct
  source 1..78: mol_type = other DNA
tttatcctgtttgatggtggccccagcaggcgaaatttcgtttctttgcttgtgtcttgtcttcgtcgtgcttgttcc

SEQ ID NO: 143 - DNA, 78 bp, synthetic construct
  source 1..78: mol_type = other DNA
tttgctcactgcccgctttacattaattgcgttgctttcgtttctttgcttgtgtcttgtcttcgtcgtgcttgttcc

SEQ ID NO: 144 - DNA, 78 bp, synthetic construct
  source 1..78: mol_type = other DNA
tttgtaacgccagggttttaaggcgattaagttggtttcgtttctttgcttgtgtcttgtcttcgtcgtgcttgttcc

SEQ ID NO: 145 - DNA, 78 bp, synthetic construct
  source 1..78: mol_type = other DNA
tttcgttggtgtagatggggtaatgggataggtcatttcgtttctttgcttgtgtcttgtcttcgtcgtgcttgttcc

SEQ ID NO: 146 - DNA, 78 bp, synthetic construct
  source 1..78: mol_type = other DNA
ttttttaaattgtaaacgtattgtataagcaaatatttcgtttctttgcttgtgtcttgtcttcgtcgtgcttgttcc

SEQ ID NO: 147 - DNA, 78 bp, synthetic construct
  source 1..78: mol_type = other DNA
tttgccggagagggtagcttagctgataaattaattttcgtttctttgcttgtgtcttgtcttcgtcgtgcttgttcc

SEQ ID NO: 148 - DNA, 78 bp, synthetic construct
  source 1..78: mol_type = other DNA
tttaaatttttagaaccctttcaacgcaaggataatttcgtttctttgcttgtgtcttgtcttcgtcgtgcttgttcc

SEQ ID NO: 149 - DNA, 78 bp, synthetic construct
  source 1..78: mol_type = other DNA
ttttaagcaataaagcctcaaagaattagcaaaattttcgtttctttgcttgtgtcttgtcttcgtcgtgcttgttcc

SEQ ID NO: 150 - DNA, 25 bp, synthetic construct
  source 1..25: mol_type = other DNA
tgccactactttttttgccaccctc

SEQ ID NO: 151 - DNA, 25 bp, synthetic construct
  source 1..25: mol_type = other DNA
aactgaacattttttttgaataacc

SEQ ID NO: 152 - DNA, 25 bp, synthetic construct
  source 1..25: mol_type = other DNA
gccacgctgtttttttaccagtgag

SEQ ID NO: 153 - DNA, 25 bp, synthetic construct
  source 1..25: mol_type = other DNA
caaaaataattttttttgtttagac

SEQ ID NO: 154 - DNA, 29 bp, synthetic construct
  source 1..29: mol_type = other DNA
gatacatttcgcttttttgaccctgtaat

SEQ ID NO: 155 - DNA, 29 bp, synthetic construct
  source 1..29: mol_type = other DNA
accgtactcaggtttttgatctaaagttt

SEQ ID NO: 156 - DNA, 29 bp, synthetic construct
  source 1..29: mol_type = other DNA
aacatgtaattttttttgaaaccaatcaa

SEQ ID NO: 157 - DNA, 29 bp, synthetic construct
  source 1..29: mol_type = other DNA
gcgtaaccaccatttttgagtaaaagagt

SEQ ID NO: 158 - DNA, 31 bp, synthetic construct
  source 1..31: mol_type = other DNA
cagagggggttttgccttcctgtagccagct

SEQ ID NO: 159 - DNA, 31 bp, synthetic construct
  source 1..31: mol_type = other DNA
gaaccgcctctttacctaaaacgaaagaggc

SEQ ID NO: 160 - DNA, 31 bp, synthetic construct
  source 1..31: mol_type = other DNA
cataaatcaatttagtcagagggtaattgag

SEQ ID NO: 161 - DNA, 31 bp, synthetic construct
  source 1..31: mol_type = other DNA
ccagggtggttttgcaaatgaaaaatctaaa

SEQ ID NO: 162 - DNA, 34 bp, synthetic construct
  source 1..34: mol_type = other DNA
acaaccattttttcatacatggcttttaagcgca

SEQ ID NO: 163 - DNA, 34 bp, synthetic construct
  source 1..34: mol_type = other DNA
ttttatcttttttatccaatcgcaagagttgggt

SEQ ID NO: 164 - DNA, 34 bp, synthetic construct
  source 1..34: mol_type = other DNA
gccaacattttttccactattaaagaaatagggt

SEQ ID NO: 165 - DNA, 34 bp, synthetic construct
  source 1..34: mol_type = other DNA
acaagagtttttttcgcgttttaattcaaaaaga

SEQ ID NO: 166 - DNA, 36 bp, synthetic construct
  source 1..36: mol_type = other DNA
tggatagcaagcccgatttttaatcgtaaacgccat

SEQ ID NO: 167 - DNA, 36 bp, synthetic construct
  source 1..36: mol_type = other DNA
agaaccgcatttaccgttttaccgatatatacgtaa

SEQ ID NO: 168 - DNA, 36 bp, synthetic construct
  source 1..36: mol_type = other DNA
ttgcttcttatatgtattttacgctaacggagaatt

SEQ ID NO: 169 - DNA, 36 bp, synthetic construct
  source 1..36: mol_type = other DNA
acgggcaagttccagttttttctgacctgcaacagt

SEQ ID NO: 170 - DNA, 38 bp, synthetic construct
  source 1..38: mol_type = other DNA
gagaatagaaaggaacaactattttctcaagagaagga

SEQ ID NO: 171 - DNA, 38 bp, synthetic construct
  source 1..38: mol_type = other DNA
ttttattttcatcgtaggaatttttagcctgtttagta

SEQ ID NO: 172 - DNA, 38 bp, synthetic construct
  source 1..38: mol_type = other DNA
caaactatcggccttgctggtttttgagcttgacgggg

SEQ ID NO: 173 - DNA, 38 bp, synthetic construct
  source 1..38: mol_type = other DNA
gagtaatgtgtaggtaaagattttttgttttaaatatg

SEQ ID NO: 174 - DNA, 43 bp, synthetic construct
  source 1..43: mol_type = other DNA
aggacagatgattttttcaccagtagcaccattaccgacttga

SEQ ID NO: 175 - DNA, 43 bp, synthetic construct
  source 1..43: mol_type = other DNA
attaagactcctttttaatatacagtaacagtaccgaaattgc

SEQ ID NO: 176 - DNA, 43 bp, synthetic construct
  source 1..43: mol_type = other DNA
gacaactcgtattttttcctgtgtgaaattgttatccgagctc

SEQ ID NO: 177 - DNA, 43 bp, synthetic construct
  source 1..43: mol_type = other DNA
ccgcttctggttttttcgttaataaaacgaactaaattatacc

SEQ ID NO: 178 - DNA, 45 bp, synthetic construct
  source 1..45: mol_type = other DNA
tgtcgtctcagccctcatatttttttcgccaccctcaggtgtatc

SEQ ID NO: 179 - DNA, 45 bp, synthetic construct
  source 1..45: mol_type = other DNA
taatcggccatcctaattttttttttttttcgagccaacaacgcc

SEQ ID NO: 180 - DNA, 45 bp, synthetic construct
  source 1..45: mol_type = other DNA
ctgtccatttttataatcatttttttcttaatgcgcccacgctgc

SEQ ID NO: 181 - DNA, 45 bp, synthetic construct
  source 1..45: mol_type = other DNA
acttttgcatcggttgtactttttttaacctgtttaggaccatta

SEQ ID NO: 182 - DNA, 51 bp, synthetic construct
  source 1..51: mol_type = other DNA
aagcgaacaattgctgaatataatgctgtatttttttgtgagaaaggccgg

SEQ ID NO: 183 - DNA, 51 bp, synthetic construct
  source 1..51: mol_type = other DNA
aggagtgtaaacatgaaagtattaagaggctttttttgcgaataataattt

SEQ ID NO: 184 - DNA, 51 bp, synthetic construct
  source 1..51: mol_type = other DNA
gcgagaaaataaacaccggaatcataattatttttttcgcccaatagcaag

SEQ ID NO: 185 - DNA, 51 bp, synthetic construct
  source 1..51: mol_type = other DNA
ccaacgtcatcggaaccctaaagggagccctttttttgaacaatattaccg

SEQ ID NO: 186 - DNA, 60 bp, synthetic construct
  source 1..60: mol_type = other DNA
ggaattagagcttttttttcagaccaggcgcgttgggaagattttttttccaggcaaagc

SEQ ID NO: 187 - DNA, 60 bp, synthetic construct
  source 1..60: mol_type = other DNA
aatcatggtcatttttttttttgcccgaactcaggtttaacttttttttcagtatgttag

SEQ ID NO: 188 - DNA, 53 bp, synthetic construct
  source 1..53: mol_type = other DNA
gtcaggaagaggtcatttttgctctggaagtttacagttattagcccgcattt

SEQ ID NO: 189 - DNA, 53 bp, synthetic construct
  source 1..53: mol_type = other DNA
atacatacaacactatcataacatgctttatttacagttattagcccgcattt

SEQ ID NO: 190 - DNA, 53 bp, synthetic construct
  source 1..53: mol_type = other DNA
acaacggaaatccgcgacctgcctcattcatttacagttattagcccgcattt

SEQ ID NO: 191 - DNA, 53 bp, synthetic construct
  source 1..53: mol_type = other DNA
caaaaggttcgaggtgaatttctcgtcacctttacagttattagcccgcattt

SEQ ID NO: 192 - DNA, 53 bp, synthetic construct
  source 1..53: mol_type = other DNA
cagtgccccccctgcctatttctttgctcatttacagttattagcccgcattt

SEQ ID NO: 193 - DNA, 53 bp, synthetic construct
  source 1..53: mol_type = other DNA
agtttgcgcattttcggtcatagagccgcctttacagttattagcccgcattt

SEQ ID NO: 194 - DNA, 53 bp, synthetic construct
  source 1..53: mol_type = other DNA
atgaaatgaaaagtaagcagatacaatcaatttacagttattagcccgcattt

SEQ ID NO: 195 - DNA, 53 bp, synthetic construct
  source 1..53: mol_type = other DNA
taagaacggaggttttgaagcctattattttttacagttattagcccgcattt

SEQ ID NO: 196 - DNA, 53 bp, synthetic construct
  source 1..53: mol_type = other DNA
atttcatgaccgtgtgataaataattcttatttacagttattagcccgcattt

SEQ ID NO: 197 - DNA, 53 bp, synthetic construct
  source 1..53: mol_type = other DNA
gcgaattatgaaacaaacatcatagcgatatttacagttattagcccgcattt

SEQ ID NO: 198 - DNA, 53 bp, synthetic construct
  source 1..53: mol_type = other DNA
acagttgttaggagcactaacatattcctgtttacagttattagcccgcattt

SEQ ID NO: 199 - DNA, 53 bp, synthetic construct
  source 1..53: mol_type = other DNA
aatacctatttacattggcagaagtctttatttacagttattagcccgcattt

SEQ ID NO: 200 - DNA, 53 bp, synthetic construct
  source 1..53: mol_type = other DNA
ggcgatgtttttggggtcgagggcgagaaatttacagttattagcccgcattt

SEQ ID NO: 201 - DNA, 53 bp, synthetic construct
  source 1..53: mol_type = other DNA
ctaactcccagtcgggaaacctggtccacgtttacagttattagcccgcattt

SEQ ID NO: 202 - DNA, 53 bp, synthetic construct
  source 1..53: mol_type = other DNA
attgacccgcatcgtaaccgtgagggggattttacagttattagcccgcattt

SEQ ID NO: 203 - DNA, 53 bp, synthetic construct
  source 1..53: mol_type = other DNA
accgttcatttttgagagatctcccaaaaatttacagttattagcccgcattt

SEQ ID NO: 204 - DNA, 63 bp, synthetic construct
  source 1..63: mol_type = other DNA
ccatgtaccgtaacactgtagcattccacagattccagactttacagttattagcccgcattt

SEQ ID NO: 205 - DNA, 63 bp, synthetic construct
  source 1..63: mol_type = other DNA
agctaatgcagaacgcgagaaaaataatatcctgtctttctttacagttattagcccgcattt

SEQ ID NO: 206 - DNA, 63 bp, synthetic construct
  source 1..63: mol_type = other DNA
ctaaacaggaggccgataatcctgagaagtgtcacgcaaatttacagttattagcccgcattt

SEQ ID NO: 207 - DNA, 63 bp, synthetic construct
  source 1..63: mol_type = other DNA
aatcatacaggcaaggcagagcataaagctaagggagaagtttacagttattagcccgcattt

SEQ ID NO: 208 - DNA, 30 bp, synthetic construct
  source 1..30: mol_type = other DNA
tttcattgagtagatttagtttctatattt

SEQ ID NO: 209 - DNA, 30 bp, synthetic construct
  source 1..30: mol_type = other DNA
aacagttaggtctttaccctgatccaacag

SEQ ID NO: 210 - DNA, 30 bp, synthetic construct
  source 1..30: mol_type = other DNA
gtgaatatagtaaattgggctttaatgcag

SEQ ID NO: 211 - DNA, 30 bp, synthetic construct
  source 1..30: mol_type = other DNA
ctcagcaggctacagaggctttaacaaagt

SEQ ID NO: 212 - DNA, 30 bp, synthetic construct
  source 1..30: mol_type = other DNA
gttagtaactttcaacagtttcaaaggctc

SEQ ID NO: 213 - DNA, 30 bp, synthetic construct
  source 1..30: mol_type = other DNA
gtaccaggtatagcccggaatagaaccgcc

SEQ ID NO: 214 - DNA, 30 bp, synthetic construct
  source 1..30: mol_type = other DNA
gccagcagccttgatattcacaaacggggt

SEQ ID NO: 215 - DNA, 30 bp, synthetic construct
  source 1..30: mol_type = other DNA
tagaaaaggcgacattcaaccgcagaatca

SEQ ID NO: 216 - DNA, 30 bp, synthetic construct
  source 1..30: mol_type = other DNA
atcccaaaaaaatgaaaatagcaagaaaca

SEQ ID NO: 217 - DNA, 30 bp, synthetic construct
  source 1..30: mol_type = other DNA
cttatcactcatcgagaacaagcggtattc

SEQ ID NO: 218 - DNA, 30 bp, synthetic construct
  source 1..30: mol_type = other DNA
ccagtatgaatcgccatatttagtaataag

SEQ ID NO: 219 - DNA, 30 bp, synthetic construct
  source 1..30: mol_type = other DNA
gcttagaatcaaaatcataggttttagtta

SEQ ID NO: 220 - DNA, 30 bp, synthetic construct
  source 1..30: mol_type = other DNA
attatcagtttggattatacttgcgcagag

SEQ ID NO: 221 - DNA, 30 bp, synthetic construct
  source 1..30: mol_type = other DNA
atgcgcgtaccgaacgaaccacgcaaatca

SEQ ID NO: 222 - DNA, 30 bp, synthetic construct
  source 1..30: mol_type = other DNA
ttaaccgtcacttgcctgagtactcatgga

SEQ ID NO: 223 - DNA, 30 bp, synthetic construct
  source 1..30: mol_type = other DNA
ggaagggggcaagtgtagcggtgctacagg

SEQ ID NO: 224 - DNA, 30 bp, synthetic construct
  source 1..30: mol_type = other DNA
ctggtttgttccgaaatcggcatctatcag

SEQ ID NO: 225 - DNA, 30 bp, synthetic construct
  source 1..30: mol_type = other DNA
gtgctgccccagtcacgacgtttgagtgag

SEQ ID NO: 226 - DNA, 30 bp, synthetic construct
  source 1..30: mol_type = other DNA
caggaagtaatattttgttaaaaacggcgg

SEQ ID NO: 227 - DNA, 30 bp, synthetic construct
  source 1..30: mol_type = other DNA
cctttatcatatattttaaatggatattca

SEQ ID NO: 228 - DNA, 75 bp, synthetic construct
  source 1..75: mol_type = other DNA
aaccgagggcaaagacaccacggataaatatttttcactcacctccatctccactcctacccatccaactcccac

SEQ ID NO: 229 - DNA, 27 bp, synthetic construct
  source 1..27: mol_type = other DNA
ttcatttgttctaatagtagtagcatt

SEQ ID NO: 230 - DNA, 27 bp, synthetic construct
  source 1..27: mol_type = other DNA
taccctcattttcagggatagcaagcc

SEQ ID NO: 231 - DNA, 27 bp, synthetic construct
  source 1..27: mol_type = other DNA
tagaatatttcagacgacgacaataaa

SEQ ID NO: 232 - DNA, 27 bp, synthetic construct
  source 1..27: mol_type = other DNA
tgcgcgtattctttcctcgttagaatc

SEQ ID NO: 233 - DNA, 28 bp, synthetic construct
  source 1..28: mol_type = other DNA
tcaactaaagtacggtgttggatggctt

SEQ ID NO: 234 - DNA, 28 bp, synthetic construct
  source 1..28: mol_type = other DNA
tcattattattgcaaaagaagttttgct

SEQ ID NO: 235 - DNA, 28 bp, synthetic construct
  source 1..28: mol_type = other DNA
tttaggattagcggggtttggaacctat

SEQ ID NO: 236 - DNA, 28 bp, synthetic construct
  source 1..28: mol_type = other DNA
taggccggattaccagagccaccaccgt

SEQ ID NO: 237 - DNA, 28 bp, synthetic construct
  source 1..28: mol_type = other DNA
ttcatatgcgttatacattaaggcgttt

SEQ ID NO: 238 - DNA, 28 bp, synthetic construct
  source 1..28: mol_type = other DNA
tcgggagaatttttaatggaaacagtat

SEQ ID NO: 239 - DNA, 28 bp, synthetic construct
  source 1..28: mol_type = other DNA
taaagccggcgaacgtgtttgccgtaat

SEQ ID NO: 240 - DNA, 28 bp, synthetic construct
  source 1..28: mol_type = other DNA
taattccacttgtttgcgtattgggcgt

SEQ ID NO: 241 - DNA, 36 bp, synthetic construct
  source 1..36: mol_type = other DNA
tttaagaggttgtccaatactgcggatttagcgagt

SEQ ID NO: 242 - DNA, 36 bp, synthetic construct
  source 1..36: mol_type = other DNA
taggcttttttcaggtagaaagattcttaattacct

SEQ ID NO: 243 - DNA, 36 bp, synthetic construct
  source 1..36: mol_type = other DNA
tttatgcgattttgacaagaaccggattggtcaatt

SEQ ID NO: 244 - DNA, 36 bp, synthetic construct
  source 1..36: mol_type = other DNA
tcataagggttacactaaaacactcattcattaaat

SEQ ID NO: 245 - DNA, 36 bp, synthetic construct
  source 1..36: mol_type = other DNA
tcgggtaaattattcggtcgctgaggttaatgacat

SEQ ID NO: 246 - DNA, 36 bp, synthetic construct
  source 1..36: mol_type = other DNA
tgtctctgattcaccctcagagccacttatcaaaat

SEQ ID NO: 247 - DNA, 36 bp, synthetic construct
  source 1..36: mol_type = other DNA
ttcaccggattaacgtcaccaatgaattttattcat

SEQ ID NO: 248 - DNA, 36 bp, synthetic construct
  source 1..36: mol_type = other DNA
tttaaaggtttacatataaaagaaacttaaacgcat

SEQ ID NO: 249 - DNA, 36 bp, synthetic construct
  source 1..36: mol_type = other DNA
tataataactttcagagagataacccttgaagcgct

SEQ ID NO: 250 - DNA, 36 bp, synthetic construct
  source 1..36: mol_type = other DNA
tattagacgttgagcgtctttccagattgctacaat

SEQ ID NO: 251 - DNA, 36 bp, synthetic construct
  source 1..36: mol_type = other DNA
ttatataacttgtaaatcgtcgctatttatttgaat

SEQ ID NO: 252 - DNA, 36 bp, synthetic construct
  source 1..36: mol_type = other DNA
tttacctttttacaataacggattcgttcaaaattt

SEQ ID NO: 253 - DNA, 36 bp, synthetic construct
  source 1..36: mol_type = other DNA
tatttgcacttcattttgcggaacaattatttgagt

SEQ ID NO: 254 - DNA, 36 bp, synthetic construct
  source 1..36: mol_type = other DNA
tgatttagattttgctgaacctcaaattgtattaat

SEQ ID NO: 255 - DNA, 36 bp, synthetic construct
  source 1..36: mol_type = other DNA
tcaccgcctttgaaagcgtaagaatattcattctgt

SEQ ID NO: 256 - DNA, 36 bp, synthetic construct
  source 1..36: mol_type = other DNA
ttgagtgttttcagctgattgcccttttgcgcgggt

SEQ ID NO: 257 - DNA, 36 bp, synthetic construct
  source 1..36: mol_type = other DNA
tgagaggcgttacaacatacgagccgttctgcaggt

SEQ ID NO: 258 - DNA, 36 bp, synthetic construct
  source 1..36: mol_type = other DNA
ttcgactctttgaagggcgatcggtgttcggcctct

SEQ ID NO: 259 - DNA, 36 bp, synthetic construct
  source 1..36: mol_type = other DNA
taggaagatttcattaaatgtgagcgtttttttaat

SEQ ID NO: 260 - DNA, 36 bp, synthetic construct
  source 1..36: mol_type = other DNA
tccaataggttaaactagcatgtcaattggagcaat

SEQ ID NO: 261 - DNA, 38 bp, synthetic construct
  source 1..38: mol_type = other DNA
ttagagcttttcagaccggaagcaaacttctattatat

SEQ ID NO: 262 - DNA, 38 bp, synthetic construct
  source 1..38: mol_type = other DNA
tgtcagaagttattgaatccccctcaattcctcgtttt

SEQ ID NO: 263 - DNA, 38 bp, synthetic construct
  source 1..38: mol_type = other DNA
taaatattcttcaaagcggattgcatcttgagcttcat

SEQ ID NO: 264 - DNA, 38 bp, synthetic construct
  source 1..38: mol_type = other DNA
taccagacgttgaataccacattcaacttgagatggtt

SEQ ID NO: 265 - DNA, 38 bp, synthetic construct
  source 1..38: mol_type = other DNA
tagatttagttacgataaaaaccaaaattatcgtcatt

SEQ ID NO: 266 - DNA, 38 bp, synthetic construct
  source 1..38: mol_type = other DNA
tagtcaggattcataggctggctgacctttttgaaagt

SEQ ID NO: 267 - DNA, 38 bp, synthetic construct
  source 1..38: mol_type = other DNA
tttaatttcttcaacgtaacaaagctgtttccatgttt

SEQ ID NO: 268 - DNA, 38 bp, synthetic construct
  source 1..38: mol_type = other DNA
tgagtaatcttttttaagaactggctcttcggaacaat

SEQ ID NO: 269 - DNA, 38 bp, synthetic construct
  source 1..38: mol_type = other DNA
tacccaaatttaactttaatcattgtgttatcagttgt

SEQ ID NO: 270 - DNA, 38 bp, synthetic construct
  source 1..38: mol_type = other DNA
tacttagccttattataccaagcgcgattgaggactat

SEQ ID NO: 271 - DNA, 38 bp, synthetic construct
  source 1..38: mol_type = other DNA
taaaagaatttaaccgaactgaccaacttttcatcaat

SEQ ID NO: 272 - DNA, 38 bp, synthetic construct
  source 1..38: mol_type = other DNA
taagactttttggccgcttttgcgggattttaaacagt

SEQ ID NO: 273 - DNA, 38 bp, synthetic construct
  source 1..38: mol_type = other DNA
tgagttaaattttcatgaggaagtttctttctttgact

SEQ ID NO: 274 - DNA, 38 bp, synthetic construct
  source 1..38: mol_type = other DNA
tcttgatactttgaaaatctccaaaaattagcggagtt

SEQ ID NO: 275 - DNA, 38 bp, synthetic construct
  source 1..38: mol_type = other DNA
ttttcacgtttcgatagttgcgccgacttcttgcaggt

SEQ ID NO: 276 - DNA, 38 bp, synthetic construct
  source 1..38: mol_type = other DNA
tttattctgttactggtaataagttttttaacaaatat

SEQ ID NO: 277 - DNA, 38 bp, synthetic construct
  source 1..38: mol_type = other DNA
taatcctcattaccagaaccaccaccattgcccccttt

SEQ ID NO: 278 - DNA, 38 bp, synthetic construct
  source 1..38: mol_type = other DNA
tgagccgccttttaaagccagaatggattgatgatact

SEQ ID NO: 279 - DNA, 38 bp, synthetic construct
  source 1..38: mol_type = other DNA
tattagcgtttccgtaatcagtagcgattattgagggt

SEQ ID NO: 280 - DNA, 38 bp, synthetic construct
  source 1..38: mol_type = other DNA
tgccatttgttcaaacgtagaaaatacttctggcatgt

SEQ ID NO: 281 - DNA, 38 bp, synthetic construct
  source 1..38: mol_type = other DNA
tagggaaggttataagtttattttgtcttagccgaact

SEQ ID NO: 282 - DNA, 38 bp, synthetic construct
  source 1..38: mol_type = other DNA
taggtggcattgaattatcaccgtcacttcattagcat

SEQ ID NO: 283 - DNA, 38 bp, synthetic construct
  source 1..38: mol_type = other DNA
taaagttacttgcccaataataagagcttagcctttat

SEQ ID NO: 284 - DNA, 38 bp, synthetic construct
  source 1..38: mol_type = other DNA
tcgctaatattggaatacccaaaagaattatacataat

SEQ ID NO: 285 - DNA, 38 bp, synthetic construct
  source 1..38: mol_type = other DNA
tcagagagattacaaaataaacagccattttaaatcat

SEQ ID NO: 286 - DNA, 38 bp, synthetic construct
  source 1..38: mol_type = other DNA
tagattagtttatatagaaggcttatcttcaagccgtt

SEQ ID NO: 287 - DNA, 38 bp, synthetic construct
  source 1..38: mol_type = other DNA
tcaaatcagtttgctattttgcacccattgcctaattt

SEQ ID NO: 288 - DNA, 38 bp, synthetic construct
  source 1..38: mol_type = other DNA
taaataagattactttttcaaatatatttctgagagat

SEQ ID NO: 289 - DNA, 38 bp, synthetic construct
  source 1..38: mol_type = other DNA
tctacctttttagaatccttgaaaacattagaaaacat

SEQ ID NO: 290 - DNA, 38 bp, synthetic construct
  source 1..38: mol_type = other DNA
ttttcccttttttaacctccggcttagttcaaagaact

SEQ ID NO: 291 - DNA, 38 bp, synthetic construct
  source 1..38: mol_type = other DNA
taaattaatttaccaagttacaaaatcttctgaataat

SEQ ID NO: 292 - DNA, 38 bp, synthetic construct
  source 1..38: mol_type = other DNA
tctttgaattttacatttaacaatttctttaattaatt

SEQ ID NO: 293 - DNA, 38 bp, synthetic construct
  source 1..38: mol_type = other DNA
tgtagatttttgttattaattttaaaattaacaattct

SEQ ID NO: 294 - DNA, 38 bp, synthetic construct
  source 1..38: mol_type = other DNA
ttggaagggttagcggaattatcatcattactaatagt

SEQ ID NO: 295 - DNA, 38 bp, synthetic construct
  source 1..38: mol_type = other DNA
taacattatttgtaaaacagaaataaattttttacatt

SEQ ID NO: 296 - DNA, 38 bp, synthetic construct
  source 1..38: mol_type = other DNA
tccagaaggttttagaacctaccatatttcctgattgt

SEQ ID NO: 297 - DNA, 38 bp, synthetic construct
  source 1..38: mol_type = other DNA
tattagagcttaatatctggtcagttgttcagcagaat

SEQ ID NO: 298 - DNA, 38 bp, synthetic construct
  source 1..38: mol_type = other DNA
tgcatcaccttagtattagactttacattgtttgagtt

SEQ ID NO: 299 - DNA, 38 bp, synthetic construct
  source 1..38: mol_type = other DNA
tcctcaatcttcgtcaatagataatacttagaaaccat

SEQ ID NO: 300 - DNA, 38 bp, synthetic construct
  source 1..38: mol_type = other DNA
tgataaaactttttttgaatggctattttttcaccagt

SEQ ID NO: 301 - DNA, 38 bp, synthetic construct
  source 1..38: mol_type = other DNA
tagacaatattagaggtgaggcggtcatttatcaaact

SEQ ID NO: 302 - DNA, 38 bp, synthetic construct
  source 1..38: mol_type = other DNA
ttcacacgatttgcaacaggaaaaacgttgaagaactt

SEQ ID NO: 303 - DNA, 38 bp, synthetic construct
  source 1..38: mol_type = other DNA
tccagccatttccagtaataaaagggattcgtggcact

SEQ ID NO: 304 - DNA, 38 bp, synthetic construct
  source 1..38: mol_type = other DNA
tagcactaattaaagggcgaaaaaccgttaaatccctt

SEQ ID NO: 305 - DNA, 38 bp, synthetic construct
  source 1..38: mol_type = other DNA
ttataaatcttgagagttgcagcaagcttgtcgtgcct

SEQ ID NO: 306 - DNA, 38 bp, synthetic construct
  source 1..38: mol_type = other DNA
tggccctgattaaaagaatagcccgagttcgtggactt

SEQ ID NO: 307 - DNA, 38 bp, synthetic construct
  source 1..38: mol_type = other DNA
tagctgcatttagcctggggtgcctaattgtaaaacgt

SEQ ID NO: 308 - DNA, 38 bp, synthetic construct
  source 1..38: mol_type = other DNA
taagtgtaatttaatgaatcggccaacttcaccgcctt

SEQ ID NO: 309 - DNA, 38 bp, synthetic construct
  source 1..38: mol_type = other DNA
tgaattcgtttgccattcgccattcagttttccggcat

SEQ ID NO: 310 - DNA, 38 bp, synthetic construct
  source 1..38: mol_type = other DNA
tacggccagtttacgccagctggcgaattcatctgcct

SEQ ID NO: 311 - DNA, 38 bp, synthetic construct
  source 1..38: mol_type = other DNA
tactgttggttagaggatccccgggtattccgctcact

SEQ ID NO: 312 - DNA, 38 bp, synthetic construct
  source 1..38: mol_type = other DNA
tttcgctattttgccaagcttgcatgcttgaagcatat

SEQ ID NO: 313 - DNA, 38 bp, synthetic construct
  source 1..38: mol_type = other DNA
tagtttgagttattctccgtgggaacattattcgcatt

SEQ ID NO: 314 - DNA, 38 bp, synthetic construct
  source 1..38: mol_type = other DNA
tttcatcaattcgcactccagccagctttgctgcgcat

SEQ ID NO: 315 - DNA, 38 bp, synthetic construct
  source 1..38: mol_type = other DNA
tcccgtcggttgggacgacgacagtatttcgggcctct

SEQ ID NO: 316 - DNA, 38 bp, synthetic construct
  source 1..38: mol_type = other DNA
ttaaatttttttgataatcagaaaagcttacaaaggct

SEQ ID NO: 317 - DNA, 38 bp, synthetic construct
  source 1..38: mol_type = other DNA
taccccggttttgttaaatcagctcatttagtaacaat

SEQ ID NO: 318 - DNA, 38 bp, synthetic construct
  source 1..38: mol_type = other DNA
ttatcaggtttaaatcaccatcaatatttcaatgcctt

SEQ ID NO: 319 - DNA, 38 bp, synthetic construct
  source 1..38: mol_type = other DNA
tagacagtcttcattgcctgagagtcttttcatatgtt

SEQ ID NO: 320 - DNA, 56 bp, synthetic construct
  source 1..56: mol_type = other DNA
tgacggaaattaccatcgatagcagcattttgccatcttttcatattcaccctcat

SEQ ID NO: 321 - DNA, 56 bp, synthetic construct
  source 1..56: mol_type = other DNA
ttgccagttttataacataaaaacaggttacaagaattgagttaattcagaaggat

SEQ ID NO: 322 - DNA, 80 bp, synthetic construct
  source 1..80: mol_type = other DNA
tttggtggcatcaattctattgggcgcgagctgaaaatttaactactcccactctcaccctcaccctactccaactcaac

SEQ ID NO: 323 - DNA, 80 bp, synthetic construct
  source 1..80: mol_type = other DNA
ttttcccaattctgcgaacttccatataacagttgattttaactactcccactctcaccctcaccctactccaactcaac

SEQ ID NO: 324 - DNA, 80 bp, synthetic construct
  source 1..80: mol_type = other DNA
tttattgctccttttgatattttagagagtacctttatttaactactcccactctcaccctcaccctactccaactcaac

SEQ ID NO: 325 - DNA, 80 bp, synthetic construct
  source 1..80: mol_type = other DNA
tttccataaatcaaaaatcttcagaaaacgagaatgatttaactactcccactctcaccctcaccctactccaactcaac

SEQ ID NO: 326 - DNA, 80 bp, synthetic construct
  source 1..80: mol_type = other DNA
tttcgaggcatagtaagagttacgccaaaaggaattatttaactactcccactctcaccctcaccctactccaactcaac

SEQ ID NO: 327 - DNA, 80 bp, synthetic construct
  source 1..80: mol_type = other DNA
tttgaaacaccagaacgagttaggcttgccctgacgatttaactactcccactctcaccctcaccctactccaactcaac

SEQ ID NO: 328 - DNA, 80 bp, synthetic construct
  source 1..80: mol_type = other DNA
tttctgataaattgtgtcgttagatttgtatcatcgctttaactactcccactctcaccctcaccctactccaactcaac

SEQ ID NO: 329 - DNA, 80 bp, synthetic construct
  source 1..80: mol_type = other DNA
tttgaacgagggtagcaacttgcgaaagacagcatcgtttaactactcccactctcaccctcaccctactccaactcaac

SEQ ID NO: 330 - DNA, 80 bp, synthetic construct
  source 1..80: mol_type = other DNA
tttggtttatcagcttgctttagcctttaattgtatctttaactactcccactctcaccctcaccctactccaactcaac

SEQ ID NO: 331 - DNA, 80 bp, synthetic construct
  source 1..80: mol_type = other DNA
tttgggattttgctaaacattaatgaattttctgtattttaactactcccactctcaccctcaccctactccaactcaac

SEQ ID NO: 332 - DNA, 80 bp, synthetic construct
  source 1..80: mol_type = other DNA
tttacaaactacaacgcctttgagtttcgtcaccagttttaactactcccactctcaccctcaccctactccaactcaac

SEQ ID NO: 333 - DNA, 80 bp, synthetic construct
  source 1..80: mol_type = other DNA
tttagccaccaccctcattttgaaccgccaccctcagtttaactactcccactctcaccctcaccctactccaactcaac

SEQ ID NO: 334 - DNA, 80 bp, synthetic construct
  source 1..80: mol_type = other DNA
tttgagagggttgatataattgcggataagtgccgtctttaactactcccactctcaccctcaccctactccaactcaac

SEQ ID NO: 335 - DNA, 80 bp, synthetic construct
  source 1..80: mol_type = other DNA
tttgtataaacagttaatgttttgagtaacagtgccctttaactactcccactctcaccctcaccctactccaactcaac

SEQ ID NO: 336 - DNA, 80 bp, synthetic construct
  source 1..80: mol_type = other DNA
tttgcaggtcagacgattgttttgacaggaggttgagtttaactactcccactctcaccctcaccctactccaactcaac

SEQ ID NO: 337 - DNA, 80 bp, synthetic construct
  source 1..80: mol_type = other DNA
ttttagcgcgttttcatcgttctttagcgtcagactgtttaactactcccactctcaccctcaccctactccaactcaac

SEQ ID NO: 338 - DNA, 80 bp, synthetic construct
  source 1..80: mol_type = other DNA
tttgcgccaaagacaaaagttttcatatggtttaccatttaactactcccactctcaccctcaccctactccaactcaac

SEQ ID NO: 339 - DNA, 80 bp, synthetic construct
  source 1..80: mol_type = other DNA
tttccgaagccctttttaattagcaatagctatcttatttaactactcccactctcaccctcaccctactccaactcaac

SEQ ID NO: 340 - DNA, 80 bp, synthetic construct
  source 1..80: mol_type = other DNA
tttttttttgtttaacgtctttccaaataagaaacgatttaactactcccactctcaccctcaccctactccaactcaac

SEQ ID NO: 341 - DNA, 80 bp, synthetic construct
  source 1..80: mol_type = other DNA
tttaacctcccgacttgcgttgcgaggcgttttagcgtttaactactcccactctcaccctcaccctactccaactcaac

SEQ ID NO: 342 - DNA, 80 bp, synthetic construct
  source 1..80: mol_type = other DNA
ttttaaaccaagtaccgcattttccaagaacgggtattttaactactcccactctcaccctcaccctactccaactcaac

SEQ ID NO: 343 - DNA, 80 bp, synthetic construct
  source 1..80: mol_type = other DNA
tttagataagtcctgaacattcctgtttatcaacaattttaactactcccactctcaccctcaccctactccaactcaac

SEQ ID NO: 344 - DNA, 80 bp, synthetic construct
  source 1..80: mol_type = other DNA
tttgtaaagtaattctgtcttaaagtaccgacaaaagtttaactactcccactctcaccctcaccctactccaactcaac

SEQ ID NO: 345 - DNA, 80 bp, synthetic construct
  source 1..80: mol_type = other DNA
tttagtagggcttaattgattaaagccaacgctcaactttaactactcccactctcaccctcaccctactccaactcaac

SEQ ID NO: 346 - DNA, 80 bp, synthetic construct
  source 1..80: mol_type = other DNA
tttaatggtttgaaataccttcttctgacctaaattttttaactactcccactctcaccctcaccctactccaactcaac

SEQ ID NO: 347 - DNA, 80 bp, synthetic construct
  source 1..80: mol_type = other DNA
tttagtcaatagtgaatttttttaagacgctgagaagtttaactactcccactctcaccctcaccctactccaactcaac

SEQ ID NO: 348 - DNA, 80 bp, synthetic construct
  source 1..80: mol_type = other DNA
ttttgagcaaaagaagatgttattcatttcaattacctttaactactcccactctcaccctcaccctactccaactcaac

SEQ ID NO: 349 - DNA, 80 bp, synthetic construct
  source 1..80: mol_type = other DNA
tttcaatataatcctgattttgatgatggcaattcattttaactactcccactctcaccctcaccctactccaactcaac

SEQ ID NO: 350 - DNA, 80 bp, synthetic construct
  source 1..80: mol_type = other DNA
tttgttatctaaaatatctttaaaggaattgaggaagtttaactactcccactctcaccctcaccctactccaactcaac

SEQ ID NO: 351 - DNA, 80 bp, synthetic construct
  source 1..80: mol_type = other DNA
tttacatcgccattaaaaattaactgatagccctaaatttaactactcccactctcaccctcaccctactccaactcaac

SEQ ID NO: 352 - DNA, 80 bp, synthetic construct
  source 1..80: mol_type = other DNA
ttttcgtctgaaatggattttacattttgacgctcaatttaactactcccactctcaccctcaccctactccaactcaac

SEQ ID NO: 353 - DNA, 80 bp, synthetic construct
  source 1..80: mol_type = other DNA
tttttgattagtaataacattttgtagcaatacttcttttaactactcccactctcaccctcaccctactccaactcaac

SEQ ID NO: 354 - DNA, 80 bp, synthetic construct
  source 1..80: mol_type = other DNA
tttaggaacggtacgccagtttaaagggattttagactttaactactcccactctcaccctcaccctactccaactcaac

SEQ ID NO: 355 - DNA, 80 bp, synthetic construct
  source 1..80: mol_type = other DNA
tttgagcacgtataacgtgttctatggttgctttgactttaactactcccactctcaccctcaccctactccaactcaac

SEQ ID NO: 356 - DNA, 80 bp, synthetic construct
  source 1..80: mol_type = other DNA
tttcgggcgctagggcgctttaagaaagcgaaaggagtttaactactcccactctcaccctcaccctactccaactcaac

SEQ ID NO: 357 - DNA, 80 bp, synthetic construct
  source 1..80: mol_type = other DNA
tttatcacccaaatcaagtttgcccactacgtgaacctttaactactcccactctcaccctcaccctactccaactcaac

SEQ ID NO: 358 - DNA, 80 bp, synthetic construct
  source 1..80: mol_type = other DNA
tttatcctgtttgatggtgttgccccagcaggcgaaatttaactactcccactctcaccctcaccctactccaactcaac

SEQ ID NO: 359 - DNA, 80 bp, synthetic construct
  source 1..80: mol_type = other DNA
tttgctcactgcccgctttttacattaattgcgttgctttaactactcccactctcaccctcaccctactccaactcaac

SEQ ID NO: 360 - DNA, 80 bp, synthetic construct
  source 1..80: mol_type = other DNA
tttgtaacgccagggttttttaaggcgattaagttggtttaactactcccactctcaccctcaccctactccaactcaac

SEQ ID NO: 361 - DNA, 80 bp, synthetic construct
  source 1..80: mol_type = other DNA
tttcgttggtgtagatgggttgtaatgggataggtcatttaactactcccactctcaccctcaccctactccaactcaac

SEQ ID NO: 362 - DNA, 80 bp, synthetic construct
  source 1..80: mol_type = other DNA
ttttttaaattgtaaacgtttattgtataagcaaatatttaactactcccactctcaccctcaccctactccaactcaac

SEQ ID NO: 363 - DNA, 80 bp, synthetic construct
  source 1..80: mol_type = other DNA
tttgccggagagggtagcttttagctgataaattaattttaactactcccactctcaccctcaccctactccaactcaac

SEQ ID NO: 364 - DNA, 80 bp, synthetic construct
  source 1..80: mol_type = other DNA
tttaaatttttagaaccctttttcaacgcaaggataatttaactactcccactctcaccctcaccctactccaactcaac

SEQ ID NO: 365 - DNA, 80 bp, synthetic construct
  source 1..80: mol_type = other DNA
ttttaagcaataaagcctcttaaagaattagcaaaattttaactactcccactctcaccctcaccctactccaactcaac

SEQ ID NO: 366 - DNA, 27 bp, synthetic construct
  source 1..27: mol_type = other DNA
ttgccactactttttttgccaccctct

SEQ ID NO: 367 - DNA, 27 bp, synthetic construct
  source 1..27: mol_type = other DNA
taactgaacattttttttgaataacct

SEQ ID NO: 368 - DNA, 27 bp, synthetic construct
  source 1..27: mol_type = other DNA
tgccacgctgtttttttaccagtgagt

SEQ ID NO: 369 - DNA, 27 bp, synthetic construct
  source 1..27: mol_type = other DNA
tcaaaaataattttttttgtttagact

SEQ ID NO: 370 - DNA, 31 bp, synthetic construct
  source 1..31: mol_type = other DNA
tgatacatttcgcttttttgaccctgtaatt

SEQ ID NO: 371 - DNA, 31 bp, synthetic construct
  source 1..31: mol_type = other DNA
taccgtactcaggtttttgatctaaagtttt

SEQ ID NO: 372 - DNA, 31 bp, synthetic construct
  source 1..31: mol_type = other DNA
taacatgtaattttttttgaaaccaatcaat

SEQ ID NO: 373 - DNA, 31 bp, synthetic construct
  source 1..31: mol_type = other DNA
tgcgtaaccaccatttttgagtaaaagagtt

SEQ ID NO: 374 - DNA, 33 bp, synthetic construct
  source 1..33: mol_type = other DNA
tcagagggggttttgccttcctgtagccagctt

SEQ ID NO: 375 - DNA, 33 bp, synthetic construct
  source 1..33: mol_type = other DNA
tgaaccgcctctttacctaaaacgaaagaggct

SEQ ID NO: 376 - DNA, 33 bp, synthetic construct
  source 1..33: mol_type = other DNA
tcataaatcaatttagtcagagggtaattgagt

SEQ ID NO: 377 - DNA, 33 bp, synthetic construct
  source 1..33: mol_type = other DNA
tccagggtggttttgcaaatgaaaaatctaaat

SEQ ID NO: 378 - DNA, 38 bp, synthetic construct
  source 1..38: mol_type = other DNA
tacaaccattttttcatacatggcttttttaagcgcat

SEQ ID NO: 379 - DNA, 38 bp, synthetic construct
  source 1..38: mol_type = other DNA
tttttatcttttttatccaatcgcaagattgttgggtt

SEQ ID NO: 380 - DNA, 38 bp, synthetic construct
  source 1..38: mol_type = other DNA
tgccaacattttttccactattaaagaattatagggtt

SEQ ID NO: 381 - DNA, 38 bp, synthetic construct
  source 1..38: mol_type = other DNA
tacaagagtttttttcgcgttttaattcttaaaaagat

SEQ ID NO: 382 - DNA, 40 bp, synthetic construct
  source 1..40: mol_type = other DNA
tgagaatagaaaggaacaactattttctcaagagaaggat

SEQ ID NO: 383 - DNA, 40 bp, synthetic construct
  source 1..40: mol_type = other DNA
tttttattttcatcgtaggaatttttagcctgtttagtat

SEQ ID NO: 384 - DNA, 40 bp, synthetic construct
  source 1..40: mol_type = other DNA
tcaaactatcggccttgctggtttttgagcttgacggggt

SEQ ID NO: 385 - DNA, 40 bp, synthetic construct
  source 1..40: mol_type = other DNA
tgagtaatgtgtaggtaaagattttttgttttaaatatgt

SEQ ID NO: 386 - DNA, 42 bp, synthetic construct
  source 1..42: mol_type = other DNA
ttggatagcttaagcccgatttttaatcgtattaacgccatt

SEQ ID NO: 387 - DNA, 42 bp, synthetic construct
  source 1..42: mol_type = other DNA
tagaaccgcttatttaccgttttaccgatatttatacgtaat

SEQ ID NO: 388 - DNA, 42 bp, synthetic construct
  source 1..42: mol_type = other DNA
tttgcttcttttatatgtattttacgctaacttggagaattt

SEQ ID NO: 389 - DNA, 42 bp, synthetic construct
  source 1..42: mol_type = other DNA
tacgggcaattgttccagttttttctgacctttgcaacagtt

SEQ ID NO: 390 - DNA, 47 bp, synthetic construct
  source 1..47: mol_type = other DNA
taggacagatgattttttcaccagtagcaccattacttcgacttgat

SEQ ID NO: 391 - DNA, 47 bp, synthetic construct
  source 1..47: mol_type = other DNA
tattaagactcctttttaatatacagtaacagtaccttgaaattgct

SEQ ID NO: 392 - DNA, 47 bp, synthetic construct
  source 1..47: mol_type = other DNA
tgacaactcgtattttttcctgtgtgaaattgttatttccgagctct

SEQ ID NO: 393 - DNA, 47 bp, synthetic construct
  source 1..47: mol_type = other DNA
tccgcttctggttttttcgttaataaaacgaactaattattatacct

SEQ ID NO: 394 - DNA, 51 bp, synthetic construct
  source 1..51: mol_type = other DNA
ttgtcgtctttcagccctcatatttttttcgccaccctcattggtgtatct

SEQ ID NO: 395 - DNA, 51 bp, synthetic construct
  source 1..51: mol_type = other DNA
ttaatcggcttcatcctaattttttttttttttcgagccattacaacgcct

SEQ ID NO: 396 - DNA, 51 bp, synthetic construct
  source 1..51: mol_type = other DNA
tctgtccatttttttataatcatttttttcttaatgcgccttcacgctgct

SEQ ID NO: 397 - DNA, 51 bp, synthetic construct
  source 1..51: mol_type = other DNA
tacttttgcttatcggttgtactttttttaacctgtttagttgaccattat

SEQ ID NO: 398 - DNA, 55 bp, synthetic construct
  source 1..55: mol_type = other DNA
taagcgaacttaattgctgaatataatgctgtatttttttgtgagaaaggccggt

SEQ ID NO: 399 - DNA, 55 bp, synthetic construct
  source 1..55: mol_type = other DNA
taggagtgtttaaacatgaaagtattaagaggctttttttgcgaataataatttt

SEQ ID NO: 400 - DNA, 55 bp, synthetic construct
  source 1..55: mol_type = other DNA
tgcgagaaattataaacaccggaatcataattatttttttcgcccaatagcaagt

SEQ ID NO: 401 - DNA, 55 bp, synthetic construct
  source 1..55: mol_type = other DNA
tccaacgtcttatcggaaccctaaagggagccctttttttgaacaatattaccgt

SEQ ID NO: 402 - DNA, 64 bp, synthetic construct
  source 1..64: mol_type = other DNA
tggaattagagcttttttttcagaccaggcgttcgttgggaagattttttttccaggcaaagct

SEQ ID NO: 403 - DNA, 64 bp, synthetic construct
  source 1..64: mol_type = other DNA
taatcatggtcatttttttttttgcccgaactttcaggtttaacttttttttcagtatgttagt

SEQ ID NO: 404 - DNA, 36 bp, synthetic construct
  source 1..36: mol_type = other DNA
ttttcattttgagtagatttagtttttctatatttt

SEQ ID NO: 405 - DNA, 36 bp, synthetic construct
  source 1..36: mol_type = other DNA
taacagttttaggtctttaccctgatttccaacagt

SEQ ID NO: 406 - DNA, 36 bp, synthetic construct
  source 1..36: mol_type = other DNA
tgtgaatatttagtaaattgggctttttaatgcagt

SEQ ID NO: 407 - DNA, 36 bp, synthetic construct
  source 1..36: mol_type = other DNA
tctcagcattggctacagaggctttttaacaaagtt

SEQ ID NO: 408 - DNA, 36 bp, synthetic construct
  source 1..36: mol_type = other DNA
tgttagtattactttcaacagtttcttaaaggctct

SEQ ID NO: 409 - DNA, 36 bp, synthetic construct
  source 1..36: mol_type = other DNA
tgtaccagttgtatagcccggaatattgaaccgcct

SEQ ID NO: 410 - DNA, 36 bp, synthetic construct
  source 1..36: mol_type = other DNA
tgccagcattgccttgatattcacattaacggggtt

SEQ ID NO: 411 - DNA, 36 bp, synthetic construct
  source 1..36: mol_type = other DNA
ttagaaaattggcgacattcaaccgttcagaatcat

SEQ ID NO: 412 - DNA, 36 bp, synthetic construct
  source 1..36: mol_type = other DNA
tatcccaattaaaaatgaaaatagcttaagaaacat

SEQ ID NO: 413 - DNA, 36 bp, synthetic construct
  source 1..36: mol_type = other DNA
tcttatcattctcatcgagaacaagttcggtattct

SEQ ID NO: 414 - DNA, 36 bp, synthetic construct
  source 1..36: mol_type = other DNA
tccagtatttgaatcgccatatttattgtaataagt

SEQ ID NO: 415 - DNA, 36 bp, synthetic construct
  source 1..36: mol_type = other DNA
tgcttagattatcaaaatcataggttttttagttat

SEQ ID NO: 416 - DNA, 36 bp, synthetic construct
  source 1..36: mol_type = other DNA
tattatcattgtttggattatacttttgcgcagagt

SEQ ID NO: 417 - DNA, 36 bp, synthetic construct
  source 1..36: mol_type = other DNA
tatgcgcgtttaccgaacgaaccacttgcaaatcat

SEQ ID NO: 418 - DNA, 36 bp, synthetic construct
  source 1..36: mol_type = other DNA
tttaaccgtttcacttgcctgagtattctcatggat

SEQ ID NO: 419 - DNA, 36 bp, synthetic construct
  source 1..36: mol_type = other DNA
tggaagggttggcaagtgtagcggtttgctacaggt

SEQ ID NO: 420 - DNA, 36 bp, synthetic construct
  source 1..36: mol_type = other DNA
tctggtttttgttccgaaatcggcatttctatcagt

SEQ ID NO: 421 - DNA, 36 bp, synthetic construct
  source 1..36: mol_type = other DNA
tgtgctgcttcccagtcacgacgtttttgagtgagt

SEQ ID NO: 422 - DNA, 36 bp, synthetic construct
  source 1..36: mol_type = other DNA
tcaggaagtttaatattttgttaaattaacggcggt

SEQ ID NO: 423 - DNA, 36 bp, synthetic construct
  source 1..36: mol_type = other DNA
tcctttatttcatatattttaaatgttgatattcat

SEQ ID NO: 424 - DNA, 36 bp, synthetic construct
  source 1..36: mol_type = other DNA
tgtcaggattagaggtcatttttgctttctggaagt

SEQ ID NO: 425 - DNA, 36 bp, synthetic construct
  source 1..36: mol_type = other DNA
tatacatattcaacactatcataacttatgctttat

SEQ ID NO: 426 - DNA, 36 bp, synthetic construct
  source 1..36: mol_type = other DNA
tacaacggttaaatccgcgacctgcttctcattcat

SEQ ID NO: 427 - DNA, 36 bp, synthetic construct
  source 1..36: mol_type = other DNA
tcaaaaggttttcgaggtgaatttctttcgtcacct

SEQ ID NO: 428 - DNA, 36 bp, synthetic construct
  source 1..36: mol_type = other DNA
tatttcatttgaccgtgtgataaatttaattcttat

SEQ ID NO: 429 - DNA, 36 bp, synthetic construct
  source 1..36: mol_type = other DNA
tgcgaattttatgaaacaaacatcatttagcgatat

SEQ ID NO: 430 - DNA, 36 bp, synthetic construct
  source 1..36: mol_type = other DNA
tacagttgttttaggagcactaacatttattcctgt

SEQ ID NO: 431 - DNA, 36 bp, synthetic construct
  source 1..36: mol_type = other DNA
taatacctttatttacattggcagattagtctttat

SEQ ID NO: 432 - DNA, 45 bp, synthetic construct
  source 1..45: mol_type = other DNA
ccatgtaccgtaacactttgtagcattccacagattttccagact

SEQ ID NO: 433 - DNA, 45 bp, synthetic construct
  source 1..45: mol_type = other DNA
ctaaacaggaggccgatttaatcctgagaagtgtttcacgcaaat

SEQ ID NO: 434 - DNA, 36 bp, synthetic construct
  source 1..36: mol_type = other DNA
tcagtgccttccccctgcctatttctttttgctcat

SEQ ID NO: 435 - DNA, 36 bp, synthetic construct
  source 1..36: mol_type = other DNA
tagtttgcttgcattttcggtcatattgagccgcct

SEQ ID NO: 436 - DNA, 36 bp, synthetic construct
  source 1..36: mol_type = other DNA
tatgaaatttgaaaagtaagcagatttacaatcaat

SEQ ID NO: 437 - DNA, 36 bp, synthetic construct
  source 1..36: mol_type = other DNA
ttaagaacttggaggttttgaagcctttattatttt

SEQ ID NO: 438 - DNA, 36 bp, synthetic construct
  source 1..36: mol_type = other DNA
tggcgatgtttttttggggtcgaggttgcgagaaat

SEQ ID NO: 439 - DNA, 36 bp, synthetic construct
  source 1..36: mol_type = other DNA
tctaactcttccagtcgggaaacctttggtccacgt

SEQ ID NO: 440 - DNA, 36 bp, synthetic construct
  source 1..36: mol_type = other DNA
tattgaccttcgcatcgtaaccgtgttagggggatt

SEQ ID NO: 441 - DNA, 36 bp, synthetic construct
  source 1..36: mol_type = other DNA
taccgttcttatttttgagagatctttcccaaaaat

SEQ ID NO: 442 - DNA, 45 bp, synthetic construct
  source 1..45: mol_type = other DNA
agctaatgcagaacgcgttagaaaaataatatcctttgtctttct

SEQ ID NO: 443 - DNA, 45 bp, synthetic construct
  source 1..45: mol_type = other DNA
aatcatacaggcaaggcttagagcataaagctaattgggagaagt

SEQ ID NO: 444 - DNA, 80 bp, synthetic construct
  source 1..80: mol_type = other DNA
tccccagcgttggaacgaggcgcagactttattcatttttcactcacctccatctccactcctacccatccaactcccac

SEQ ID NO: 445 - DNA, 80 bp, synthetic construct
  source 1..80: mol_type = other DNA
taaccgaggttgcaaagacaccacggatttaaatatttttcactcacctccatctccactcctacccatccaactcccac

SEQ ID NO: 446 - DNA, 30 bp, synthetic construct
  source 1..30: mol_type = other DNA
gtcaggaagaggtcatttttgctctggaag

SEQ ID NO: 447 - DNA, 30 bp, synthetic construct
  source 1..30: mol_type = other DNA
atacatacaacactatcataacatgcttta

SEQ ID NO: 448 - DNA, 30 bp, synthetic construct
  source 1..30: mol_type = other DNA
acaacggaaatccgcgacctgcctcattca

SEQ ID NO: 449 - DNA, 30 bp, synthetic construct
  source 1..30: mol_type = other DNA
caaaaggttcgaggtgaatttctcgtcacc

SEQ ID NO: 450 - DNA, 30 bp, synthetic construct
  source 1..30: mol_type = other DNA
cagtgccccccctgcctatttctttgctca

SEQ ID NO: 451 - DNA, 30 bp, synthetic construct
  source 1..30: mol_type = other DNA
agtttgcgcattttcggtcatagagccgcc

SEQ ID NO: 452 - DNA, 30 bp, synthetic construct
  source 1..30: mol_type = other DNA
atgaaatgaaaagtaagcagatacaatcaa

SEQ ID NO: 453 - DNA, 30 bp, synthetic construct
  source 1..30: mol_type = other DNA
taagaacggaggttttgaagcctattattt

SEQ ID NO: 454 - DNA, 30 bp, synthetic construct
  source 1..30: mol_type = other DNA
atttcatgaccgtgtgataaataattctta

SEQ ID NO: 455 - DNA, 30 bp, synthetic construct
  source 1..30: mol_type = other DNA
gcgaattatgaaacaaacatcatagcgata

SEQ ID NO: 456 - DNA, 30 bp, synthetic construct
  source 1..30: mol_type = other DNA
acagttgttaggagcactaacatattcctg

SEQ ID NO: 457 - DNA, 30 bp, synthetic construct
  source 1..30: mol_type = other DNA
aatacctatttacattggcagaagtcttta

SEQ ID NO: 458 - DNA, 30 bp, synthetic construct
  source 1..30: mol_type = other DNA
ggcgatgtttttggggtcgagggcgagaaa

SEQ ID NO: 459 - DNA, 30 bp, synthetic construct
  source 1..30: mol_type = other DNA
ctaactcccagtcgggaaacctggtccacg

SEQ ID NO: 460 - DNA, 30 bp, synthetic construct
  source 1..30: mol_type = other DNA
attgacccgcatcgtaaccgtgagggggat

SEQ ID NO: 461 - DNA, 30 bp, synthetic construct
  source 1..30: mol_type = other DNA
accgttcatttttgagagatctcccaaaaa

SEQ ID NO: 462 - DNA, 40 bp, synthetic construct
  source 1..40: mol_type = other DNA
ccatgtaccgtaacactgtagcattccacagattccagac

SEQ ID NO: 463 - DNA, 40 bp, synthetic construct
  source 1..40: mol_type = other DNA
agctaatgcagaacgcgagaaaaataatatcctgtctttc

SEQ ID NO: 464 - DNA, 40 bp, synthetic construct
  source 1..40: mol_type = other DNA
ctaaacaggaggccgataatcctgagaagtgtcacgcaaa

SEQ ID NO: 465 - DNA, 40 bp, synthetic construct
  source 1..40: mol_type = other DNA
aatcatacaggcaaggcagagcataaagctaagggagaag

SEQ ID NO: 466 - DNA, 30 bp, synthetic construct
  source 1..30: mol_type = other DNA
cgggcgctagggcgctaagaaagcgaaagg

SEQ ID NO: 467 - DNA, 30 bp, synthetic construct
  source 1..30: mol_type = other DNA
atcacccaaatcaagtgcccactacgtgaa

SEQ ID NO: 468 - DNA, 30 bp, synthetic construct
  source 1..30: mol_type = other DNA
atcctgtttgatggtggccccagcaggcga

SEQ ID NO: 469 - DNA, 30 bp, synthetic construct
  source 1..30: mol_type = other DNA
gctcactgcccgctttacattaattgcgtt

SEQ ID NO: 470 - DNA, 30 bp, synthetic construct
  source 1..30: mol_type = other DNA
cgttggtgtagatggggtaatgggataggt

SEQ ID NO: 471 - DNA, 30 bp, synthetic construct
  source 1..30: mol_type = other DNA
tttaaattgtaaacgtattgtataagcaaa

SEQ ID NO: 472 - DNA, 30 bp, synthetic construct
  source 1..30: mol_type = other DNA
gccggagagggtagcttagctgataaatta

SEQ ID NO: 473 - DNA, 30 bp, synthetic construct
  source 1..30: mol_type = other DNA
aaatttttagaaccctttcaacgcaaggat

SEQ ID NO: 474 - DNA, 58 bp, synthetic construct
  source 1..58: mol_type = other DNA
gtgtcgtagacacggtggcatcaattctagggcgcgagctgaaaagtgtcgtagacac

SEQ ID NO: 475 - DNA, 58 bp, synthetic construct
  source 1..58: mol_type = other DNA
gtgtcgtagacactcccaattctgcgaacccatataacagttgatgtgtcgtagacac

SEQ ID NO: 476 - DNA, 58 bp, synthetic construct
  source 1..58: mol_type = other DNA
gtgtcgtagacacattgctccttttgatattagagagtacctttagtgtcgtagacac

SEQ ID NO: 477 - DNA, 58 bp, synthetic construct
  source 1..58: mol_type = other DNA
gtgtcgtagacacccataaatcaaaaatccagaaaacgagaatgagtgtcgtagacac

SEQ ID NO: 478 - DNA, 58 bp, synthetic construct
  source 1..58: mol_type = other DNA
gtgtcgtagacaccgaggcatagtaagagacgccaaaaggaattagtgtcgtagacac

SEQ ID NO: 479 - DNA, 58 bp, synthetic construct
  source 1..58: mol_type = other DNA
gtgtcgtagacacgaaacaccagaacgagaggcttgccctgacgagtgtcgtagacac

SEQ ID NO: 480 - DNA, 58 bp, synthetic construct
  source 1..58: mol_type = other DNA
gtgtcgtagacacctgataaattgtgtcgagatttgtatcatcgcgtgtcgtagacac

SEQ ID NO: 481 - DNA, 58 bp, synthetic construct
  source 1..58: mol_type = other DNA
gtgtcgtagacacgaacgagggtagcaacgcgaaagacagcatcggtgtcgtagacac

SEQ ID NO: 482 - DNA, 58 bp, synthetic construct
  source 1..58: mol_type = other DNA
gtgtcgtagacacggtttatcagcttgctagcctttaattgtatcgtgtcgtagacac

SEQ ID NO: 483 - DNA, 58 bp, synthetic construct
  source 1..58: mol_type = other DNA
gtgtcgtagacacgggattttgctaaacaaatgaattttctgtatgtgtcgtagacac

SEQ ID NO: 484 - DNA, 58 bp, synthetic construct
  source 1..58: mol_type = other DNA
gtgtcgtagacacacaaactacaacgcctgagtttcgtcaccagtgtgtcgtagacac

SEQ ID NO: 485 - DNA, 58 bp, synthetic construct
  source 1..58: mol_type = other DNA
gtgtcgtagacacagccaccaccctcattgaaccgccaccctcaggtgtcgtagacac

SEQ ID NO: 486 - DNA, 58 bp, synthetic construct
  source 1..58: mol_type = other DNA
gtgtcgtagacacgagagggttgatataagcggataagtgccgtcgtgtcgtagacac

SEQ ID NO: 487 - DNA, 58 bp, synthetic construct
  source 1..58: mol_type = other DNA
gtgtcgtagacacgtataaacagttaatgttgagtaacagtgcccgtgtcgtagacac

SEQ ID NO: 488 - DNA, 58 bp, synthetic construct
  source 1..58: mol_type = other DNA
gtgtcgtagacacgcaggtcagacgattgttgacaggaggttgaggtgtcgtagacac

SEQ ID NO: 489 - DNA, 58 bp, synthetic construct
  source 1..58: mol_type = other DNA
gtgtcgtagacactagcgcgttttcatcgctttagcgtcagactggtgtcgtagacac

SEQ ID NO: 490 - DNA, 58 bp, synthetic construct
  source 1..58: mol_type = other DNA
gtgtcgtagacacgcgccaaagacaaaagttcatatggtttaccagtgtcgtagacac

SEQ ID NO: 491 - DNA, 58 bp, synthetic construct
  source 1..58: mol_type = other DNA
gtgtcgtagacacccgaagccctttttaaagcaatagctatcttagtgtcgtagacac

SEQ ID NO: 492 - DNA, 58 bp, synthetic construct
  source 1..58: mol_type = other DNA
gtgtcgtagacacttttttgtttaacgtctccaaataagaaacgagtgtcgtagacac

SEQ ID NO: 493 - DNA, 58 bp, synthetic construct
  source 1..58: mol_type = other DNA
gtgtcgtagacacaacctcccgacttgcggcgaggcgttttagcggtgtcgtagacac

SEQ ID NO: 494 - DNA, 58 bp, synthetic construct
  source 1..58: mol_type = other DNA
gtgtcgtagacactaaaccaagtaccgcattccaagaacgggtatgtgtcgtagacac

SEQ ID NO: 495 - DNA, 58 bp, synthetic construct
  source 1..58: mol_type = other DNA
gtgtcgtagacacagataagtcctgaacacctgtttatcaacaatgtgtcgtagacac

SEQ ID NO: 496 - DNA, 58 bp, synthetic construct
  source 1..58: mol_type = other DNA
gtgtcgtagacacgtaaagtaattctgtcaaagtaccgacaaaaggtgtcgtagacac

SEQ ID NO: 497 - DNA, 58 bp, synthetic construct
  source 1..58: mol_type = other DNA
gtgtcgtagacacagtagggcttaattgaaaagccaacgctcaacgtgtcgtagacac

SEQ ID NO: 498 - DNA, 58 bp, synthetic construct
  source 1..58: mol_type = other DNA
gtgtcgtagacacaatggtttgaaatacccttctgacctaaatttgtgtcgtagacac

SEQ ID NO: 499 - DNA, 58 bp, synthetic construct
  source 1..58: mol_type = other DNA
gtgtcgtagacacagtcaatagtgaatttttaagacgctgagaaggtgtcgtagacac

SEQ ID NO: 500 - DNA, 58 bp, synthetic construct
  source 1..58: mol_type = other DNA
gtgtcgtagacactgagcaaaagaagatgattcatttcaattaccgtgtcgtagacac

SEQ ID NO: 501 - DNA, 58 bp, synthetic construct
  source 1..58: mol_type = other DNA
gtgtcgtagacaccaatataatcctgattgatgatggcaattcatgtgtcgtagacac

SEQ ID NO: 502 - DNA, 58 bp, synthetic construct
  source 1..58: mol_type = other DNA
gtgtcgtagacacgttatctaaaatatctaaaggaattgaggaaggtgtcgtagacac

SEQ ID NO: 503 - DNA, 58 bp, synthetic construct
  source 1..58: mol_type = other DNA
gtgtcgtagacacacatcgccattaaaaaaactgatagccctaaagtgtcgtagacac

SEQ ID NO: 504 - DNA, 58 bp, synthetic construct
  source 1..58: mol_type = other DNA
gtgtcgtagacactcgtctgaaatggattacattttgacgctcaagtgtcgtagacac

SEQ ID NO: 505 - DNA, 58 bp, synthetic construct
  source 1..58: mol_type = other DNA
gtgtcgtagacacttgattagtaataacattgtagcaatacttctgtgtcgtagacac

SEQ ID NO: 506 - DNA, 58 bp, synthetic construct
  source 1..58: mol_type = other DNA
gtgtcgtagacacaggaacggtacgccagtaaagggattttagacgtgtcgtagacac

SEQ ID NO: 507 - DNA, 32 bp, synthetic construct
  source 1..32: mol_type = other DNA
aaccgagggcaaagacaccacggataaatatt

SEQ ID NO: 508 - DNA, 28 bp, synthetic construct
  source 1..28: mol_type = other DNA
catatttaacaacgttaatttcatcttc

SEQ ID NO: 509 - DNA, 28 bp, synthetic construct
  source 1..28: mol_type = other DNA
aatggaaagcgcagtaacagtgcccgta

SEQ ID NO: 510 - DNA, 28 bp, synthetic construct
  source 1..28: mol_type = other DNA
atcagtgaggccacctacagggcgcgta

SEQ ID NO: 511 - DNA, 28 bp, synthetic construct
  source 1..28: mol_type = other DNA
catcaataagcctcgtaggtaaaattaa

SEQ ID NO: 512 - DNA, 31 bp, synthetic construct
  source 1..31: mol_type = other DNA
tttcgggatcgtcaccctcagcagtacaacg

SEQ ID NO: 513 - DNA, 35 bp, synthetic construct
  source 1..35: mol_type = other DNA
agagaatataaagtaccgacagggtattaaaccaa

SEQ ID NO: 514 - DNA, 35 bp, synthetic construct
  source 1..35: mol_type = other DNA
caaccgagtggcaaatagccgtatcagattttttg

SEQ ID NO: 515 - DNA, 35 bp, synthetic construct
  source 1..35: mol_type = other DNA
gaaagcggcgaactagttgaagattagatataatc

SEQ ID NO: 516 - DNA, 35 bp, synthetic construct
  source 1..35: mol_type = other DNA
aggcggataagtgcaccgccaccctcagttagtaa

SEQ ID NO: 517 - DNA, 35 bp, synthetic construct
  source 1..35: mol_type = other DNA
gaaagccggcgaacccaacgtcaaagggtaaatca

SEQ ID NO: 518 - DNA, 35 bp, synthetic construct
  source 1..35: mol_type = other DNA
gcattccacagacatgctttcgaccagggtggttt

SEQ ID NO: 519 - DNA, 35 bp, synthetic construct
  source 1..35: mol_type = other DNA
aattctgccaacagagcccgattagactataaaaa

SEQ ID NO: 520 - DNA, 39 bp, synthetic construct
  source 1..39: mol_type = other DNA
ttgcctgaattaaccgttgtagacgctgcgcgtaacaag

SEQ ID NO: 521 - DNA, 42 bp, synthetic construct
  source 1..42: mol_type = other DNA
ggcattttcgagccagtaataactcatcgagaacgcgagaaa

SEQ ID NO: 522 - DNA, 42 bp, synthetic construct
  source 1..42: mol_type = other DNA
tcgcaagacaaagaacaagcataatttgccttcatttgaatt

SEQ ID NO: 523 - DNA, 42 bp, synthetic construct
  source 1..42: mol_type = other DNA
acatttaacaatagttacaaaaagaaacaataatggaagggt

SEQ ID NO: 524 - DNA, 42 bp, synthetic construct
  source 1..42: mol_type = other DNA
tatacttctgaatgaaatagctgtcacaatatctttaggagc

SEQ ID NO: 525 - DNA, 42 bp, synthetic construct
  source 1..42: mol_type = other DNA
ttacgcataataacactgaacagagaatgaagcctgaacgcg

SEQ ID NO: 526 - DNA, 42 bp, synthetic construct
  source 1..42: mol_type = other DNA
tggtttaccagcgcatcgatagcagcacttagcgtcagactg

SEQ ID NO: 527 - DNA, 42 bp, synthetic construct
  source 1..42: mol_type = other DNA
gttatctaaaatcaatagaaaagtagcgactttgaatggcta

SEQ ID NO: 528 - DNA, 42 bp, synthetic construct
  source 1..42: mol_type = other DNA
ttaaaaataccgaacattctggccaacatggtaatatccaga

SEQ ID NO: 529 - DNA, 42 bp, synthetic construct
  source 1..42: mol_type = other DNA
cgaaccatatcaaatattagatatcatcagattttttcattt

SEQ ID NO: 530 - DNA, 42 bp, synthetic construct
  source 1..42: mol_type = other DNA
gtgaggcggtcagtcattggcagattcagaaaaacgctcatg

SEQ ID NO: 531 - DNA, 42 bp, synthetic construct
  source 1..42: mol_type = other DNA
attaacaagcaaatttgcccgtatcattccttttatgctttg

SEQ ID NO: 532 - DNA, 42 bp, synthetic construct
  source 1..42: mol_type = other DNA
accattaccattagatagcccccttatttcagagccaccacc

SEQ ID NO: 533 - DNA, 42 bp, synthetic construct
  source 1..42: mol_type = other DNA
acagacaatattagaatcaagggaggttgagattagtaataa

SEQ ID NO: 534 - DNA, 42 bp, synthetic construct
  source 1..42: mol_type = other DNA
gagccgccgccagcggccttgatattcaggaacctattattc

SEQ ID NO: 535 - DNA, 42 bp, synthetic construct
  source 1..42: mol_type = other DNA
caatacttctttggcaggtcatgaaagtatagtgtagcggtc

SEQ ID NO: 536 - DNA, 42 bp, synthetic construct
  source 1..42: mol_type = other DNA
agacaggaacggtacgtgctttcctcgtatcggaaccctaaa

SEQ ID NO: 537 - DNA, 42 bp, synthetic construct
  source 1..42: mol_type = other DNA
gcgtattgggcgggtgaattttaaaatacgtcatggtcatag

SEQ ID NO: 538 - DNA, 42 bp, synthetic construct
  source 1..42: mol_type = other DNA
actcatctttgacccgacctgctccatgcaagagtaatcttg

SEQ ID NO: 539 - DNA, 42 bp, synthetic construct
  source 1..42: mol_type = other DNA
tcgaattcgtaataatgccactgaccaactgcgccattcgcc

SEQ ID NO: 540 - DNA, 42 bp, synthetic construct
  source 1..42: mol_type = other DNA
cagtgccaagcttgctcttcgctattactcggcctcaggaag

SEQ ID NO: 541 - DNA, 42 bp, synthetic construct
  source 1..42: mol_type = other DNA
gtgctgccccagtcgggtgccgcgctcaacgctgggtggttc

SEQ ID NO: 542 - DNA, 42 bp, synthetic construct
  source 1..42: mol_type = other DNA
gacagataccgaactacgaagaaacgggcttaaacatcagct

SEQ ID NO: 543 - DNA, 42 bp, synthetic construct
  source 1..42: mol_type = other DNA
aaaccaggcaaattgaaagagaagaactggagccagctttca

SEQ ID NO: 544 - DNA, 42 bp, synthetic construct
  source 1..42: mol_type = other DNA
tggccttcctgtctcattatagcaaaagaacaaacaagagaa

SEQ ID NO: 545 - DNA, 42 bp, synthetic construct
  source 1..42: mol_type = other DNA
gagagtctggaggttttgccataattcgagttatttcaacgc

SEQ ID NO: 546 - DNA, 42 bp, synthetic construct
  source 1..42: mol_type = other DNA
cgggagaagcctcttcaaagcaccattagatacatttcgcaa

SEQ ID NO: 547 - DNA, 42 bp, synthetic construct
  source 1..42: mol_type = other DNA
tacggtggataagatagtcagttcattgagtaagaacaggta

SEQ ID NO: 548 - DNA, 48 bp, synthetic construct
  source 1..48: mol_type = other DNA
aggaattgaggaagttagtctttaatgctaagaatacgtggccatcac

SEQ ID NO: 549 - DNA, 48 bp, synthetic construct
  source 1..48: mol_type = other DNA
ccgagcattcaggctgcgcagcttctggtgccggtcaacattaaatgt

SEQ ID NO: 550 - DNA, 49 bp, synthetic construct
  source 1..49: mol_type = other DNA
atcttaccaaataagaaacgagagataacccacaaagaaaagtaagcag

SEQ ID NO: 551 - DNA, 49 bp, synthetic construct
  source 1..49: mol_type = other DNA
tcaatatatgatgaaacaaactatttgcacgtaaatggcaattcatcaa

SEQ ID NO: 552 - DNA, 49 bp, synthetic construct
  source 1..49: mol_type = other DNA
ctattaagcagaggcgaattacaggtttaacgtcaagaaggagcggaat

SEQ ID NO: 553 - DNA, 49 bp, synthetic construct
  source 1..49: mol_type = other DNA
agattagagcagcctttacagaccctgaacaaagtccgaggaaacgcaa

SEQ ID NO: 554 - DNA, 49 bp, synthetic construct
  source 1..49: mol_type = other DNA
tcaccaattttcatcggcattccgccaccagaaccataaatcctcatta

SEQ ID NO: 555 - DNA, 49 bp, synthetic construct
  source 1..49: mol_type = other DNA
ctaaaacaacccttctgacctaactcaaactatcgaagagtctgtccat

SEQ ID NO: 556 - DNA, 49 bp, synthetic construct
  source 1..49: mol_type = other DNA
aagataacacgaccagtaatataccgccagccattatcctgagaagtgt

SEQ ID NO: 557 - DNA, 49 bp, synthetic construct
  source 1..49: mol_type = other DNA
aaatcacgccatcttttcataccaccctcagaaccatttaccgttccag

SEQ ID NO: 558 - DNA, 49 bp, synthetic construct
  source 1..49: mol_type = other DNA
gtggcgatgcgccgcgagtaagccttgcgagatagatcgccactggtca

SEQ ID NO: 559 - DNA, 49 bp, synthetic construct
  source 1..49: mol_type = other DNA
agccacccagtacctaaacagaagccagctcagagttcggtccaaggcc

SEQ ID NO: 560 - DNA, 49 bp, synthetic construct
  source 1..49: mol_type = other DNA
ttttcaggttttgtcgtctttaaggctccaaaaggccgacaatgacaac

SEQ ID NO: 561 - DNA, 49 bp, synthetic construct
  source 1..49: mol_type = other DNA
aaagaacgatagggttgagtggcaacagctgattgtaatgaatcggcca

SEQ ID NO: 562 - DNA, 49 bp, synthetic construct
  source 1..49: mol_type = other DNA
tcagggccgaaatcggcaaaagcagcaagcggtccctgcccgctttcca

SEQ ID NO: 563 - DNA, 49 bp, synthetic construct
  source 1..49: mol_type = other DNA
ccgccactgggattttgctaaattgcgaataataatcggtcgctgaggc

SEQ ID NO: 564 - DNA, 49 bp, synthetic construct
  source 1..49: mol_type = other DNA
cgtaacgaattgtatcggtttagcttgataccgataggaagtttccatt

SEQ ID NO: 565 - DNA, 49 bp, synthetic construct
  source 1..49: mol_type = other DNA
gcctgtatttcgtcagggcgctggcataagaggctaccgtaacactgag

SEQ ID NO: 566 - DNA, 49 bp, synthetic construct
  source 1..49: mol_type = other DNA
aaagaatccgcctggccctgaaacctgtcgtgccaaacatacgagccgg

SEQ ID NO: 567 - DNA, 49 bp, synthetic construct
  source 1..49: mol_type = other DNA
atgaattacgttgaaaatctcgcccacgcataaccgcaacggctacaga

SEQ ID NO: 568 - DNA, 49 bp, synthetic construct
  source 1..49: mol_type = other DNA
cgaggcgcataggctggctgataatttcaactttaacgttaataaaacg

SEQ ID NO: 569 - DNA, 49 bp, synthetic construct
  source 1..49: mol_type = other DNA
cgatcggtccagccagctttctaacaacccgtcggaaccaataggaacg

SEQ ID NO: 570 - DNA, 49 bp, synthetic construct
  source 1..49: mol_type = other DNA
tgcgggccatgccttccacacgctgcatcccttcaagcccgagtggact

SEQ ID NO: 571 - DNA, 49 bp, synthetic construct
  source 1..49: mol_type = other DNA
gggggattttgaggggacgacaccgtaatgggatattaaaattcgcatt

SEQ ID NO: 572 - DNA, 49 bp, synthetic construct
  source 1..49: mol_type = other DNA
tgtgtcgccggatattcattagagaaacaccagaagaaagattcatcag

SEQ ID NO: 573 - DNA, 49 bp, synthetic construct
  source 1..49: mol_type = other DNA
ccttcatttacttactaaaacggctttgaaccatccaaaaaaccagacg

SEQ ID NO: 574 - DNA, 49 bp, synthetic construct
  source 1..49: mol_type = other DNA
ggacgttgggaagaccaaaatagcgagataatagtaaaatgtaagactt

SEQ ID NO: 575 - DNA, 49 bp, synthetic construct
  source 1..49: mol_type = other DNA
aaataattcgcgtctcgatgaacggtaatcaggtcattgcctaaggata

SEQ ID NO: 576 - DNA, 49 bp, synthetic construct
  source 1..49: mol_type = other DNA
tccatatgtaccttgcatcaaactgcggaccctcgaactaacgatggtt

SEQ ID NO: 577 - DNA, 49 bp, synthetic construct
  source 1..49: mol_type = other DNA
tagtttggaaccagcgcgtttgagggggggcttttccagtcacgatttt

SEQ ID NO: 578 - DNA, 49 bp, synthetic construct
  source 1..49: mol_type = other DNA
gtttagctatgaccttagaacaaggctatcgtaaaccatcaagagcgag

SEQ ID NO: 579 - DNA, 53 bp, synthetic construct
  source 1..53: mol_type = other DNA
cggcaccactgttggaggatcaaattgtacgcgcggagacggttgttccagtt

SEQ ID NO: 580 - DNA, 56 bp, synthetic construct
  source 1..56: mol_type = other DNA
gtaatttaggcagaactttttcaaatattgatgcaaatccaaaccttttttaatgg

SEQ ID NO: 581 - DNA, 56 bp, synthetic construct
  source 1..56: mol_type = other DNA
caatttttagcaagggctgtcagacgacgacaataaacaacaaccaatcaataatc

SEQ ID NO: 582 - DNA, 56 bp, synthetic construct
  source 1..56: mol_type = other DNA
tctgtaacctttttaaatacctcaacagtagggcttaattgaaatttaatggtttg

SEQ ID NO: 583 - DNA, 56 bp, synthetic construct
  source 1..56: mol_type = other DNA
tgaaaacagtcaatacaccggtcatatgcgttatacaaattcttaaataagaataa

SEQ ID NO: 584 - DNA, 56 bp, synthetic construct
  source 1..56: mol_type = other DNA
gccatattatttatgttaagcccaataaatcttaccgaagcccaccacggaataag

SEQ ID NO: 585 - DNA, 56 bp, synthetic construct
  source 1..56: mol_type = other DNA
aaacaaaattaatttagaacctaccatactgattgtttggatactaacaactaata

SEQ ID NO: 586 - DNA, 56 bp, synthetic construct
  source 1..56: mol_type = other DNA
caaagacgcaaagactttttagaattgacccaatccaacgctaacgagcgtctttc

SEQ ID NO: 587 - DNA, 56 bp, synthetic construct
  source 1..56: mol_type = other DNA
attcatatttatttaatagcttaagagcataaacacagagccagccgttgtaccgc

SEQ ID NO: 588 - DNA, 56 bp, synthetic construct
  source 1..56: mol_type = other DNA
gatggccgggagcccgtataacgccagagcaacagccagtcaaacagagatcacct

SEQ ID NO: 589 - DNA, 56 bp, synthetic construct
  source 1..56: mol_type = other DNA
cactacgtgaaccatcacccagcactaatagaatcggattttgaaatacttattta

SEQ ID NO: 590 - DNA, 56 bp, synthetic construct
  source 1..56: mol_type = other DNA
cgccacctatagcctaagttttaagcgtagagccgatcaaaaaattagatcattaa

SEQ ID NO: 591 - DNA, 56 bp, synthetic construct
  source 1..56: mol_type = other DNA
cctcagacgtcgagccttgagtctctgagccacccagcgtttcagtagcgtaaata

SEQ ID NO: 592 - DNA, 56 bp, synthetic construct
  source 1..56: mol_type = other DNA
atctaaaggatagctaggattctatttccaaacaaaccaccatagcgcgtgaaacc

SEQ ID NO: 593 - DNA, 56 bp, synthetic construct
  source 1..56: mol_type = other DNA
gccctcacccatgtgagactctgaaacagacgattattgacatttgcctcgtaatc

SEQ ID NO: 594 - DNA, 56 bp, synthetic construct
  source 1..56: mol_type = other DNA
gagagtttcccttacgaaaaatgacgggctatggtttttataacaatataaaggga

SEQ ID NO: 595 - DNA, 56 bp, synthetic construct
  source 1..56: mol_type = other DNA
gcaccaacctaaaatcaatcataagggagaacggtgtacagatgaattaccttatg

SEQ ID NO: 596 - DNA, 56 bp, synthetic construct
  source 1..56: mol_type = other DNA
cgagtagacaagaaaaatccgcccagcggagggtagatatatttttttcttctgta

SEQ ID NO: 597 - DNA, 56 bp, synthetic construct
  source 1..56: mol_type = other DNA
aaaatctatcattgccaggcgcagacggcgaaagatttcatgagttgcgagccttt

SEQ ID NO: 598 - DNA, 56 bp, synthetic construct
  source 1..56: mol_type = other DNA
tcagaaaaaattttcggattggacagtagccagctcgacggcaagcatagtcggga

SEQ ID NO: 599 - DNA, 56 bp, synthetic construct
  source 1..56: mol_type = other DNA
gggcgcgaatcggtaatgcaagctatttcatatgtattttttattctccatcgcac

SEQ ID NO: 600 - DNA, 56 bp, synthetic construct
  source 1..56: mol_type = other DNA
tagcattaattagctgagaaatattcaagaagatttattttgggtcacgctgccag

SEQ ID NO: 601 - DNA, 59 bp, synthetic construct
  source 1..59: mol_type = other DNA
gcataaagaaattgcgtatattcctgattattgaggatttagaagccctcaatcaatat

SEQ ID NO: 602 - DNA, 59 bp, synthetic construct
  source 1..59: mol_type = other DNA
acaatacagtaacagtattgcggaacaaagatcgtattaaatcctgaaaaatctaaagc

SEQ ID NO: 603 - DNA, 59 bp, synthetic construct
  source 1..59: mol_type = other DNA
aaataattgagcgctaaaacaaagttaccagatacatacataaagttgagggagggaag

SEQ ID NO: 604 - DNA, 60 bp, synthetic construct
  source 1..60: mol_type = other DNA
atacattcagatgaacagaaaaaagaagatgtgagtgaataaccttgctcaattacctga

SEQ ID NO: 605 - DNA, 60 bp, synthetic construct
  source 1..60: mol_type = other DNA
cgacaacaaccaccgatgaataaatcgcttaattttcccttagaatcctaataccaagtt

SEQ ID NO: 606 - DNA, 60 bp, synthetic construct
  source 1..60: mol_type = other DNA
gtagaaaaaggaaacagagggtgaaaatttgctattttgcacccagctatttaacgtcaa

SEQ ID NO: 607 - DNA, 60 bp, synthetic construct
  source 1..60: mol_type = other DNA
gagcgggcgctaccagtacaggaacaagagtcaaagcgacaccacacacgcaagtagaag

SEQ ID NO: 608 - DNA, 60 bp, synthetic construct
  source 1..60: mol_type = other DNA
taactacaacttcttttcaccagtgggagaggcggtttctgtttcctgtgtgcccgggta

SEQ ID NO: 609 - DNA, 60 bp, synthetic construct
  source 1..60: mol_type = other DNA
gtgggaacaaacggtgttaaatcagctcaccccggttgataatgccggagagggtatgcc

SEQ ID NO: 610 - DNA, 60 bp, synthetic construct
  source 1..60: mol_type = other DNA
taaattgggcttgaggaacaacattattgcaacactatcataaatcgtcataaataaagc

SEQ ID NO: 611 - DNA, 60 bp, synthetic construct
  source 1..60: mol_type = other DNA
agatccctgaccccaaatatcgcctgaaacaaagcgaaattgcaggtaaaggaacaactt

SEQ ID NO: 612 - DNA, 60 bp, synthetic construct
  source 1..60: mol_type = other DNA
ttttaatgctgtagctcaacatgttttaaatggcttaggtcttgctttaaccaaaagttg

SEQ ID NO: 613 - DNA, 75 bp, synthetic construct
  source 1..75: mol_type = other DNA
ctcaagagaaggataagcccaataggaatagttagcactcacctccatctccactcctacccatccaactcccac

SEQ ID NO: 614 - DNA, 69 bp, synthetic construct
  source 1..69: mol_type = other DNA
atttgtatccaacgtaacaaagctgctctcgtttctttgcttgtgtcttgtcttcgtcgtgcttgttcc

SEQ ID NO: 615 - DNA, 76 bp, synthetic construct
  source 1..76: mol_type = other DNA
tcccatcgcctgtttatcaacaatagataagtctttcgtttctttgcttgtgtcttgtcttcgtcgtgcttgttcc

SEQ ID NO: 616 - DNA, 78 bp, synthetic construct
  source 1..78: mol_type = other DNA
tttaaaagcctgtttagtaaatcataattactagatttcgtttctttgcttgtgtcttgtcttcgtcgtgcttgttcc

SEQ ID NO: 617 - DNA, 78 bp, synthetic construct
  source 1..78: mol_type = other DNA
tttttaagacgctgagaagatagcgatagcttagatttcgtttctttgcttgtgtcttgtcttcgtcgtgcttgttcc

SEQ ID NO: 618 - DNA, 78 bp, synthetic construct
  source 1..78: mol_type = other DNA
tttaacggattcgcctgatcatcgggagaaacaattttcgtttctttgcttgtgtcttgtcttcgtcgtgcttgttcc

SEQ ID NO: 619 - DNA, 78 bp, synthetic construct
  source 1..78: mol_type = other DNA
tttaaagtttgagtaacataacgttattaattttatttcgtttctttgcttgtgtcttgtcttcgtcgtgcttgttcc

SEQ ID NO: 620 - DNA, 78 bp, synthetic construct
  source 1..78: mol_type = other DNA
tttcacgctgagagccagcccgcctgcaacagtgctttcgtttctttgcttgtgtcttgtcttcgtcgtgcttgttcc

SEQ ID NO: 621 - DNA, 78 bp, synthetic construct
  source 1..78: mol_type = other DNA
tttaatcgtctgaaatggactacattttgacgctctttcgtttctttgcttgtgtcttgtcttcgtcgtgcttgttcc

SEQ ID NO: 622 - DNA, 78 bp, synthetic construct
  source 1..78: mol_type = other DNA
tttaggaggccgattaaagagagcgggagctaaactttcgtttctttgcttgtgtcttgtcttcgtcgtgcttgttcc

SEQ ID NO: 623 - DNA, 78 bp, synthetic construct
  source 1..78: mol_type = other DNA
tttgtcgaggtgccgtaaaaatcaagttttttgggtttcgtttctttgcttgtgtcttgtcttcgtcgtgcttgttcc

SEQ ID NO: 624 - DNA, 78 bp, synthetic construct
  source 1..78: mol_type = other DNA
tttaaaatcctgtttgatgtttgccccagcaggcgtttcgtttctttgcttgtgtcttgtcttcgtcgtgcttgttcc

SEQ ID NO: 625 - DNA, 78 bp, synthetic construct
  source 1..78: mol_type = other DNA
ttttcacattaattgcgtttaatgagtgagctaactttcgtttctttgcttgtgtcttgtcttcgtcgtgcttgttcc

SEQ ID NO: 626 - DNA, 78 bp, synthetic construct
  source 1..78: mol_type = other DNA
tttcatcgtaaccgtgcatttggtgtagatgggcgtttcgtttctttgcttgtgtcttgtcttcgtcgtgcttgttcc

SEQ ID NO: 627 - DNA, 78 bp, synthetic construct
  source 1..78: mol_type = other DNA
tttaaattgtaaacgttaagtataagcaaatattttttcgtttctttgcttgtgtcttgtcttcgtcgtgcttgttcc

SEQ ID NO: 628 - DNA, 78 bp, synthetic construct
  source 1..78: mol_type = other DNA
tttatcaccatcaatatgaggccggagacagtcaatttcgtttctttgcttgtgtcttgtcttcgtcgtgcttgttcc

SEQ ID NO: 629 - DNA, 78 bp, synthetic construct
  source 1..78: mol_type = other DNA
ttttacaggcaaggcaaagaacatccaataaatcatttcgtttctttgcttgtgtcttgtcttcgtcgtgcttgttcc

SEQ ID NO: 630 - DNA, 80 bp, synthetic construct
  source 1..80: mol_type = other DNA
tctgaacaagaaaaataataaggcgttttagcgaacctctcgtttctttgcttgtgtcttgtcttcgtcgtgcttgttcc

SEQ ID NO: 631 - DNA, 80 bp, synthetic construct
  source 1..80: mol_type = other DNA
tccgacttgcgggaggttttaacataaaaacagggaagctcgtttctttgcttgtgtcttgtcttcgtcgtgcttgttcc

SEQ ID NO: 632 - DNA, 80 bp, synthetic construct
  source 1..80: mol_type = other DNA
tgcattagacgggagaattaggaatacccaaaagaactgtcgtttctttgcttgtgtcttgtcttcgtcgtgcttgttcc

SEQ ID NO: 633 - DNA, 80 bp, synthetic construct
  source 1..80: mol_type = other DNA
tgcatgattaagactccttaaggtgaattatcaccgtcatcgtttctttgcttgtgtcttgtcttcgtcgtgcttgttcc

SEQ ID NO: 634 - DNA, 80 bp, synthetic construct
  source 1..80: mol_type = other DNA
tccgacttgagccatttgggtcaccggaaccagagccactcgtttctttgcttgtgtcttgtcttcgtcgtgcttgttcc

SEQ ID NO: 635 - DNA, 80 bp, synthetic construct
  source 1..80: mol_type = other DNA
tcaccggaaccgcctccctccatacatggcttttgatgatcgtttctttgcttgtgtcttgtcttcgtcgtgcttgttcc

SEQ ID NO: 636 - DNA, 80 bp, synthetic construct
  source 1..80: mol_type = other DNA
ttacaggagtgtactggtaacggaataggtgtatcaccgtcgtttctttgcttgtgtcttgtcttcgtcgtgcttgttcc

SEQ ID NO: 637 - DNA, 80 bp, synthetic construct
  source 1..80: mol_type = other DNA
ttactcaggaggtttagtactcaacagtttcagcggagttcgtttctttgcttgtgtcttgtcttcgtcgtgcttgttcc

SEQ ID NO: 638 - DNA, 80 bp, synthetic construct
  source 1..80: mol_type = other DNA
tgagaatagaaaggaacaacgagttaaaggccgcttttgtcgtttctttgcttgtgtcttgtcttcgtcgtgcttgttcc

SEQ ID NO: 639 - DNA, 80 bp, synthetic construct
  source 1..80: mol_type = other DNA
tattcagtgaataaggcttgttaggaataccacattcaatcgtttctttgcttgtgtcttgtcttcgtcgtgcttgttcc

SEQ ID NO: 640 - DNA, 80 bp, synthetic construct
  source 1..80: mol_type = other DNA
tctaatgcagatacataacgacagttcagaaaacgagaatcgtttctttgcttgtgtcttgtcttcgtcgtgcttgttcc

SEQ ID NO: 641 - DNA, 80 bp, synthetic construct
  source 1..80: mol_type = other DNA
ttgaccataaatcaaaaatcagagcttaattgctgaatatcgtttctttgcttgtgtcttgtcttcgtcgtgcttgttcc

SEQ ID NO: 642 - DNA, 51 bp, synthetic construct
  source 1..51: mol_type = other DNA
tcaaaatatcaagaaaacagtacataaatttacagttattagcccgcattt

SEQ ID NO: 643 - DNA, 58 bp, synthetic construct
  source 1..58: mol_type = other DNA
catataaaagaaacaaaagggcgacattggaaacgtttacagttattagcccgcattt

SEQ ID NO: 644 - DNA, 58 bp, synthetic construct
  source 1..58: mol_type = other DNA
gccgtcaatagatagttggcaaatcaacgatagcctttacagttattagcccgcattt

SEQ ID NO: 645 - DNA, 58 bp, synthetic construct
  source 1..58: mol_type = other DNA
ctttacaaacaatttgctgaacctcaaaccagcagtttacagttattagcccgcattt

SEQ ID NO: 646 - DNA, 58 bp, synthetic construct
  source 1..58: mol_type = other DNA
gtatgttagcaaacttgacggaaattatgccagcatttacagttattagcccgcattt

SEQ ID NO: 647 - DNA, 58 bp, synthetic construct
  source 1..58: mol_type = other DNA
ttaatgccccctgcagcggggttttgctaccctcatttacagttattagcccgcattt

SEQ ID NO: 648 - DNA, 58 bp, synthetic construct
  source 1..58: mol_type = other DNA
cccgccgcgcttaagaaaggaagggaagcactatttttacagttattagcccgcattt

SEQ ID NO: 649 - DNA, 58 bp, synthetic construct
  source 1..58: mol_type = other DNA
tgctttgacgagcacccgatttagagctccgtctatttacagttattagcccgcattt

SEQ ID NO: 650 - DNA, 58 bp, synthetic construct
  source 1..58: mol_type = other DNA
taacggggtcagtgagggttgatataagctcagaatttacagttattagcccgcattt

SEQ ID NO: 651 - DNA, 58 bp, synthetic construct
  source 1..58: mol_type = other DNA
aggactaaagacttggcaaaagaatacagccggaatttacagttattagcccgcattt

SEQ ID NO: 652 - DNA, 58 bp, synthetic construct
  source 1..58: mol_type = other DNA
tatccgctcacaatgcaggtcgactctaggaagggtttacagttattagcccgcattt

SEQ ID NO: 653 - DNA, 58 bp, synthetic construct
  source 1..58: mol_type = other DNA
aagtgtaaagcctgacgacgttgtaaaaggcgaaatttacagttattagcccgcattt

SEQ ID NO: 654 - DNA, 58 bp, synthetic construct
  source 1..58: mol_type = other DNA
gacagcatcggaacattataccaagcgcgataaattttacagttattagcccgcattt

SEQ ID NO: 655 - DNA, 58 bp, synthetic construct
  source 1..58: mol_type = other DNA
tttaccagacgacgggatagcgtccaataaagatttttacagttattagcccgcattt

SEQ ID NO: 656 - DNA, 58 bp, synthetic construct
  source 1..58: mol_type = other DNA
actagcatgtcaatttgagagatctacacctcatatttacagttattagcccgcattt

SEQ ID NO: 657 - DNA, 58 bp, synthetic construct
  source 1..58: mol_type = other DNA
agccccaaaaacagccgttctagctgataagattctttacagttattagcccgcattt

SEQ ID NO: 658 - DNA, 58 bp, synthetic construct
  source 1..58: mol_type = other DNA
gaattacgaggcataatccccctcaaattaccctgtttacagttattagcccgcattt

SEQ ID NO: 659 - DNA, 72 bp, synthetic construct
  source 1..72: mol_type = other DNA
aagtaattctgtcctttccttatcattcattaccgcgcccaaatcctgatttacagttattagcccgcattt

SEQ ID NO: 660 - DNA, 72 bp, synthetic construct
  source 1..72: mol_type = other DNA
tataaagccaacgcgaccgtgtgataaaggtctgagagactaatcgtcgtttacagttattagcccgcattt

SEQ ID NO: 661 - DNA, 72 bp, synthetic construct
  source 1..72: mol_type = other DNA
ctaatgcagaacgcctaatttacgagcaatccggtattctaataaatcatttacagttattagcccgcattt

SEQ ID NO: 662 - DNA, 77 bp, synthetic construct
  source 1..77: mol_type = other DNA
caaatcagatatagaaggctttgtagaatgttcagttcgtttctttgcttgtgtcttgtcttcgtcgtgcttgttcc

SEQ ID NO: 663 - DNA, 77 bp, synthetic construct
  source 1..77: mol_type = other DNA
tttattttcatcgtaggaatccaagaacaaaggtattcgtttctttgcttgtgtcttgtcttcgtcgtgcttgttcc

SEQ ID NO: 664 - DNA, 77 bp, synthetic construct
  source 1..77: mol_type = other DNA
tatataactatatgtaaatgcattttagccaacatttcgtttctttgcttgtgtcttgtcttcgtcgtgcttgttcc

SEQ ID NO: 665 - DNA, 77 bp, synthetic construct
  source 1..77: mol_type = other DNA
aacctccggcttaggttgggttgacctagaatcgcttcgtttctttgcttgtgtcttgtcttcgtcgtgcttgttcc

SEQ ID NO: 666 - DNA, 77 bp, synthetic construct
  source 1..77: mol_type = other DNA
agtgaatttatcaaaatcatataaggcgttaccagttcgtttctttgcttgtgtcttgtcttcgtcgtgcttgttcc

SEQ ID NO: 667 - DNA, 77 bp, synthetic construct
  source 1..77: mol_type = other DNA
aagaggagtcaggattagagaaacagttgattcccttcgtttctttgcttgtgtcttgtcttcgtcgtgcttgttcc

SEQ ID NO: 668 - DNA, 77 bp, synthetic construct
  source 1..77: mol_type = other DNA
caaatataccggaagcaaactcgaacgagtagattttcgtttctttgcttgtgtcttgtcttcgtcgtgcttgttcc

SEQ ID NO: 669 - DNA, 77 bp, synthetic construct
  source 1..77: mol_type = other DNA
aaaatttctgtaatacttttgatggtcaataacctttcgtttctttgcttgtgtcttgtcttcgtcgtgcttgttcc

SEQ ID NO: 670 - DNA, 77 bp, synthetic construct
  source 1..77: mol_type = other DNA
tattttatgtaccaaaaacattatattttcatttgttcgtttctttgcttgtgtcttgtcttcgtcgtgcttgttcc

SEQ ID NO: 671 - DNA, 77 bp, synthetic construct
  source 1..77: mol_type = other DNA
aaaagggaaaattaagcaatatctactaatagtagttcgtttctttgcttgtgtcttgtcttcgtcgtgcttgttcc

SEQ ID NO: 672 - DNA, 77 bp, synthetic construct
  source 1..77: mol_type = other DNA
actattaggtcatttttgcggatatgcaactaaagttcgtttctttgcttgtgtcttgtcttcgtcgtgcttgttcc

SEQ ID NO: 673 - DNA, 80 bp, synthetic construct
  source 1..80: mol_type = other DNA
tgagtaatgtagagcataaagctaagctgaaaaggtggttcgtttctttgcttgtgtcttgtcttcgtcgtgcttgttcc

SEQ ID NO: 674 - DNA, 80 bp, synthetic construct
  source 1..80: mol_type = other DNA
aaagcggatttaattgctcctttttctggaagtttcatttcgtttctttgcttgtgtcttgtcttcgtcgtgcttgttcc

SEQ ID NO: 675 - DNA, 30 bp, synthetic construct
  source 1..30: mol_type = other DNA
cagctttcatcaacatgttttaaatatgca

SEQ ID NO: 676 - DNA, 44 bp, synthetic construct
  source 1..44: mol_type = other DNA
tattatagtccaaaagtagtaaaccctgacccgcgacatcgcct

SEQ ID NO: 677 - DNA, 47 bp, synthetic construct
  source 1..47: mol_type = other DNA
actaacatagacttaatcctgaaattatatattccatataacagttg

SEQ ID NO: 678 - DNA, 48 bp, synthetic construct
  source 1..48: mol_type = other DNA
ttaatggtctttttcaaatatagcaacactatcatataatagtaaaat

SEQ ID NO: 679 - DNA, 30 bp, synthetic construct
  source 1..30: mol_type = other DNA
agagtctaaaaatcaggtctttaccctgac

SEQ ID NO: 680 - DNA, 30 bp, synthetic construct
  source 1..30: mol_type = other DNA
acagttcttcgcgtctggccttcctgtagc

SEQ ID NO: 681 - DNA, 44 bp, synthetic construct
  source 1..44: mol_type = other DNA
attcccaattctgcgaacgagtagatttctaaggaatcgtcata

SEQ ID NO: 682 - DNA, 44 bp, synthetic construct
  source 1..44: mol_type = other DNA
gtttagactggatagcgtccaatactgctggtagctatttttga

SEQ ID NO: 683 - DNA, 28 bp, synthetic construct
  source 1..28: mol_type = other DNA
tccctcagagccgcgccggaaacgtcac

SEQ ID NO: 684 - DNA, 28 bp, synthetic construct
  source 1..28: mol_type = other DNA
ttaccttatgcgatagattcatcagttg

SEQ ID NO: 685 - DNA, 28 bp, synthetic construct
  source 1..28: mol_type = other DNA
taccgaacgaaccagaaatggattattt

SEQ ID NO: 686 - DNA, 28 bp, synthetic construct
  source 1..28: mol_type = other DNA
cagtttggatttagaaaggaggccgatt

SEQ ID NO: 687 - DNA, 28 bp, synthetic construct
  source 1..28: mol_type = other DNA
acgtcaaggtgccgctgcgcgcgtgctt

SEQ ID NO: 688 - DNA, 28 bp, synthetic construct
  source 1..28: mol_type = other DNA
aaggataaaaatttttgtaccaaaaaca

SEQ ID NO: 689 - DNA, 32 bp, synthetic construct
  source 1..32: mol_type = other DNA
atcaggtcattgcctggagatctacaaaggct

SEQ ID NO: 690 - DNA, 32 bp, synthetic construct
  source 1..32: mol_type = other DNA
ccctcaaatgctttaaaatattcattgaatcc

SEQ ID NO: 691 - DNA, 32 bp, synthetic construct
  source 1..32: mol_type = other DNA
tggtcaataacctgttagatacatttcgcaaa

SEQ ID NO: 692 - DNA, 35 bp, synthetic construct
  source 1..35: mol_type = other DNA
gaacggtaatcgtatctagctgtaaagattcaaaa

SEQ ID NO: 693 - DNA, 35 bp, synthetic construct
  source 1..35: mol_type = other DNA
cgccaccagaaccaccaccagggggtcagtgcctt

SEQ ID NO: 694 - DNA, 35 bp, synthetic construct
  source 1..35: mol_type = other DNA
ggactaatcggtcgaggctcctgggattccgtaac

SEQ ID NO: 695 - DNA, 35 bp, synthetic construct
  source 1..35: mol_type = other DNA
ataagagaacaacaatcattagaacgcgacgtcaa

SEQ ID NO: 696 - DNA, 35 bp, synthetic construct
  source 1..35: mol_type = other DNA
cgaaagacttcaaaaagaggtcatttttgtgggaa

SEQ ID NO: 697 - DNA, 35 bp, synthetic construct
  source 1..35: mol_type = other DNA
cagaagcaaagcggtataatgctgtagctcaacat

SEQ ID NO: 698 - DNA, 35 bp, synthetic construct
  source 1..35: mol_type = other DNA
aatcagtgaaaaacacattggttaaaaaaacaccg

SEQ ID NO: 699 - DNA, 35 bp, synthetic construct
  source 1..35: mol_type = other DNA
ccgaaatgccaacgattgcgtgtgagcctgcggcg

SEQ ID NO: 700 - DNA, 35 bp, synthetic construct
  source 1..35: mol_type = other DNA
caataaagcagtttgaccatttagctatattttca

SEQ ID NO: 701 - DNA, 36 bp, synthetic construct
  source 1..36: mol_type = other DNA
ggcaaggcaattctactaatagtagtagcattattt

SEQ ID NO: 702 - DNA, 38 bp, synthetic construct
  source 1..38: mol_type = other DNA
tttaccccggttgataatccatgtcaatcatatgtttt

SEQ ID NO: 703 - DNA, 38 bp, synthetic construct
  source 1..38: mol_type = other DNA
tttattaaatttttgttaattttgttaaaattcgcttt

SEQ ID NO: 704 - DNA, 38 bp, synthetic construct
  source 1..38: mol_type = other DNA
tttcaaatcaccatcaataaaaggccggagacagtttt

SEQ ID NO: 705 - DNA, 42 bp, synthetic construct
  source 1..42: mol_type = other DNA
cagagccaccaccctcagagcagtgcccgtaaaatcaccagt

SEQ ID NO: 706 - DNA, 42 bp, synthetic construct
  source 1..42: mol_type = other DNA
attagagccagcataaacagtccaccctcagattaagactcc

SEQ ID NO: 707 - DNA, 42 bp, synthetic construct
  source 1..42: mol_type = other DNA
aagaactggcatgagccaccaaaggaacaaagaataacataa

SEQ ID NO: 708 - DNA, 42 bp, synthetic construct
  source 1..42: mol_type = other DNA
gcctttacagagctaaaggaacaccctcagtatagaaggctt

SEQ ID NO: 709 - DNA, 42 bp, synthetic construct
  source 1..42: mol_type = other DNA
ccgacaagctttcggttttgtgtagcatagggttgattagcg

SEQ ID NO: 710 - DNA, 42 bp, synthetic construct
  source 1..42: mol_type = other DNA
cggaacgagggtagcggagatttgtatcctgctccatgttac

SEQ ID NO: 711 - DNA, 42 bp, synthetic construct
  source 1..42: mol_type = other DNA
aagcaaatcagacagcgaaaggataaattggtaattctgtcc

SEQ ID NO: 712 - DNA, 42 bp, synthetic construct
  source 1..42: mol_type = other DNA
gcctgtttatcaaccatgtaatttaggcgcgttaaataagaa

SEQ ID NO: 713 - DNA, 42 bp, synthetic construct
  source 1..42: mol_type = other DNA
aatagatctcatcgtttgaagtattattgggtaatcagatag

SEQ ID NO: 714 - DNA, 42 bp, synthetic construct
  source 1..42: mol_type = other DNA
atatcccatcctaacagtagggcttaataagcctgtttagta

SEQ ID NO: 715 - DNA, 42 bp, synthetic construct
  source 1..42: mol_type = other DNA
tttacgattccttactacaattctttcctgagttaagcaata

SEQ ID NO: 716 - DNA, 42 bp, synthetic construct
  source 1..42: mol_type = other DNA
tttgacccccagcgtcataagggaaccggatattcattaccc

SEQ ID NO: 717 - DNA, 42 bp, synthetic construct
  source 1..42: mol_type = other DNA
acaaaaggtaaatgtcgaaatgagaaacactcatcttctgac

SEQ ID NO: 718 - DNA, 42 bp, synthetic construct
  source 1..42: mol_type = other DNA
gtgaataaggcttgttgggcttgagatggcagatacataacg

SEQ ID NO: 719 - DNA, 42 bp, synthetic construct
  source 1..42: mol_type = other DNA
ttttagttaattcagaacgaggaattacgaggcatagtaaga

SEQ ID NO: 720 - DNA, 42 bp, synthetic construct
  source 1..42: mol_type = other DNA
tttaacctccggctaagacgctgagaaggaataaccttgctt

SEQ ID NO: 721 - DNA, 42 bp, synthetic construct
  source 1..42: mol_type = other DNA
ttaccagtctaatacatttgaacttctgaataaccattcagg

SEQ ID NO: 722 - DNA, 42 bp, synthetic construct
  source 1..42: mol_type = other DNA
ggatttagaagtatactaatagattagaaggcggtcagtatt

SEQ ID NO: 723 - DNA, 42 bp, synthetic construct
  source 1..42: mol_type = other DNA
gagacgcagaaacacaggcggcctttagacttgtagaacgtc

SEQ ID NO: 724 - DNA, 42 bp, synthetic construct
  source 1..42: mol_type = other DNA
gccgtcaatagaccggaatttgtccgttttaaaacagaggtg

SEQ ID NO: 725 - DNA, 42 bp, synthetic construct
  source 1..42: mol_type = other DNA
aattgaggaaggttgagccagcagcaaattaatgcgcgaact

SEQ ID NO: 726 - DNA, 42 bp, synthetic construct
  source 1..42: mol_type = other DNA
ttgctgagtcagttaagtttgcaccagagaatataataacgg

SEQ ID NO: 727 - DNA, 42 bp, synthetic construct
  source 1..42: mol_type = other DNA
tcgctggaaacgcggtgagaggccatgtctgcgcaggcaaag

SEQ ID NO: 728 - DNA, 42 bp, synthetic construct
  source 1..42: mol_type = other DNA
ccagcagaagatttcgtctcgtatgagccggctcaatcgtct

SEQ ID NO: 729 - DNA, 42 bp, synthetic construct
  source 1..42: mol_type = other DNA
ctacattttgacggtcactgtgggggtttcgccagaatcctg

SEQ ID NO: 730 - DNA, 42 bp, synthetic construct
  source 1..42: mol_type = other DNA
caggaacggtactgccagcacagtcgggaatggcgagaaagg

SEQ ID NO: 731 - DNA, 42 bp, synthetic construct
  source 1..42: mol_type = other DNA
agccggcgaacgacctgtcgtaaaagaatagcccgagatagg

SEQ ID NO: 732 - DNA, 42 bp, synthetic construct
  source 1..42: mol_type = other DNA
ctggtttgtggttttgtaaagaattcgttgtcacttgttcag

SEQ ID NO: 733 - DNA, 42 bp, synthetic construct
  source 1..42: mol_type = other DNA
atcagctcatttttgtggcatcaaagaattagcaaaaatcgg

SEQ ID NO: 734 - DNA, 43 bp, synthetic construct
  source 1..43: mol_type = other DNA
tttccagtcacgacgttgtaaatgccgccagcagttttgtgta

SEQ ID NO: 735 - DNA, 43 bp, synthetic construct
  source 1..43: mol_type = other DNA
tttacatccaataaatcatacattatgactcaacgcgggtgag

SEQ ID NO: 736 - DNA, 48 bp, synthetic construct
  source 1..48: mol_type = other DNA
ccgcgcccaatagcagacgacgacaataaatataaagtaccgctaaat

SEQ ID NO: 737 - DNA, 49 bp, synthetic construct
  source 1..49: mol_type = other DNA
ggagcaaacaagagggagaggcaatgcctgagtaacctcatatatttta

SEQ ID NO: 738 - DNA, 49 bp, synthetic construct
  source 1..49: mol_type = other DNA
taccgcctaggaacccatgtattgctaaacaacttaatctccaaaaaaa

SEQ ID NO: 739 - DNA, 49 bp, synthetic construct
  source 1..49: mol_type = other DNA
aaaatacaaaccgaggaaacggggagaattaactgcgattttttgttta

SEQ ID NO: 740 - DNA, 49 bp, synthetic construct
  source 1..49: mol_type = other DNA
agacaccttaagaaaagtaagtgagcgctaatatcaaaataaacagcca

SEQ ID NO: 741 - DNA, 49 bp, synthetic construct
  source 1..49: mol_type = other DNA
tatagccaaactacaacgcctcgtctttccagacgcggtttatcagctt

SEQ ID NO: 742 - DNA, 49 bp, synthetic construct
  source 1..49: mol_type = other DNA
gaaacaagaacgaggcgcagacgtaacaaagctgcttcaactttaatca

SEQ ID NO: 743 - DNA, 49 bp, synthetic construct
  source 1..49: mol_type = other DNA
ctaatgcttttcgagccagtaaccgaccgtgtgataagacaaagaacgc

SEQ ID NO: 744 - DNA, 49 bp, synthetic construct
  source 1..49: mol_type = other DNA
gaacaagcgccatatttaacacggaatcataattaggttatataactat

SEQ ID NO: 745 - DNA, 49 bp, synthetic construct
  source 1..49: mol_type = other DNA
ctaaaaccaactttgaaagaggagtaatcttgacaactggctcattata

SEQ ID NO: 746 - DNA, 49 bp, synthetic construct
  source 1..49: mol_type = other DNA
tttgcaaataaaaacaatcgcaaataagagaggcaagaacgcgttttta

SEQ ID NO: 747 - DNA, 49 bp, synthetic construct
  source 1..49: mol_type = other DNA
gcggatgggaagccagatttattgtgaaaaatcaacggtcaaattatac

SEQ ID NO: 748 - DNA, 49 bp, synthetic construct
  source 1..49: mol_type = other DNA
gcttaattaacaacccgtcggcagctttccggcactgcgggcctcttcg

SEQ ID NO: 749 - DNA, 49 bp, synthetic construct
  source 1..49: mol_type = other DNA
ctgagcagaggcgaattattcttgcacgtaaaacacaattcatcaatat

SEQ ID NO: 750 - DNA, 49 bp, synthetic construct
  source 1..49: mol_type = other DNA
attaattattcgcctgattgcgtttaacgtcagataggagcggaattat

SEQ ID NO: 751 - DNA, 49 bp, synthetic construct
  source 1..49: mol_type = other DNA
ttgctccaccgtaatgggatagggacgacgacagtgtgctgcaaggcga

SEQ ID NO: 752 - DNA, 49 bp, synthetic construct
  source 1..49: mol_type = other DNA
taaatgtggtgccggaaaccaactgttgggaaggggcgaaacgtacagc

SEQ ID NO: 753 - DNA, 49 bp, synthetic construct
  source 1..49: mol_type = other DNA
caaacggtcaggaagatcgcagccagctggcgaaatggagccgccacgg

SEQ ID NO: 754 - DNA, 49 bp, synthetic construct
  source 1..49: mol_type = other DNA
cgccattcgtggaagggttagaacctaccatatcaattgtttggattat

SEQ ID NO: 755 - DNA, 49 bp, synthetic construct
  source 1..49: mol_type = other DNA
gttaaaccctcataacggaacttgcaggcgctttcttctttgctcgtca

SEQ ID NO: 756 - DNA, 49 bp, synthetic construct
  source 1..49: mol_type = other DNA
cagtgccctaaaacatcgccacagattcaccagtcagccattgcaacag

SEQ ID NO: 757 - DNA, 49 bp, synthetic construct
  source 1..49: mol_type = other DNA
acgctgaatctaaacaactcgatgatgggaaataatcgcgcaaaagaag

SEQ ID NO: 758 - DNA, 49 bp, synthetic construct
  source 1..49: mol_type = other DNA
catcacctttttgaatggctaggccaacagagataactatcggccttgc

SEQ ID NO: 759 - DNA, 49 bp, synthetic construct
  source 1..49: mol_type = other DNA
taaaaaatgctggtctggtcacaaccagcttacggcaaatcgttaacgg

SEQ ID NO: 760 - DNA, 49 bp, synthetic construct
  source 1..49: mol_type = other DNA
gtgccggtgatgaaggaaaaagaacggactattacctccagcattctcc

SEQ ID NO: 761 - DNA, 49 bp, synthetic construct
  source 1..49: mol_type = other DNA
cggctggtaatgggggccgttttcacggtgttcttcgcgtcctgcgctc

SEQ ID NO: 762 - DNA, 49 bp, synthetic construct
  source 1..49: mol_type = other DNA
gctcatggaaatacagaagtgtttttataaagggattttagaaagggaa

SEQ ID NO: 763 - DNA, 49 bp, synthetic construct
  source 1..49: mol_type = other DNA
aaatcctgtttgcgtgagtgagatcccctctgtggtaaacatggggtca

SEQ ID NO: 764 - DNA, 49 bp, synthetic construct
  source 1..49: mol_type = other DNA
ataaatcgccagctgctttccgcgtgcctcatacctgccctggttgcgg

SEQ ID NO: 765 - DNA, 49 bp, synthetic construct
  source 1..49: mol_type = other DNA
aaaataaagaaaacgagaaaagcaaatataggaacgccatcatttgggg

SEQ ID NO: 766 - DNA, 49 bp, synthetic construct
  source 1..49: mol_type = other DNA
tgatattcaaccgtaaactagagaaaagccccaaatgtaaacgttaata

SEQ ID NO: 767 - DNA, 56 bp, synthetic construct
  source 1..56: mol_type = other DNA
agaaccgccaccctagcaccattaccattgagccatttgggattattacgcagtat

SEQ ID NO: 768 - DNA, 56 bp, synthetic construct
  source 1..56: mol_type = other DNA
ctcaggagaaagtacaggagtgaggttgaggcaggtcagacgggcttttgatgata

SEQ ID NO: 769 - DNA, 56 bp, synthetic construct
  source 1..56: mol_type = other DNA
taaaagaatattgacaccgtacggaaccagagccaccaccggaccatcgatagcag

SEQ ID NO: 770 - DNA, 56 bp, synthetic construct
  source 1..56: mol_type = other DNA
acaatcagacaaaacagactgcccccttattagcgtttgccatttgcctttagcgt

SEQ ID NO: 771 - DNA, 56 bp, synthetic construct
  source 1..56: mol_type = other DNA
tttcagggatagcatttcagcggagtgataataattttttcaaggccgcttttgcg

SEQ ID NO: 772 - DNA, 56 bp, synthetic construct
  source 1..56: mol_type = other DNA
aacggaatacccaaaaacagggaagcgcaaatgaaaatagcaatccggtattctaa

SEQ ID NO: 773 - DNA, 56 bp, synthetic construct
  source 1..56: mol_type = other DNA
caacggcgagttaacgttgaatcaacagagcccaaaccctcagaaccgccaccctc

SEQ ID NO: 774 - DNA, 56 bp, synthetic construct
  source 1..56: mol_type = other DNA
acagcatggatcgtttgcgaagaatagaccctcatagaaccgtaatgccgagtaac

SEQ ID NO: 775 - DNA, 56 bp, synthetic construct
  source 1..56: mol_type = other DNA
tgctgatgcaaatcccaaaatagagaatccttgaaatgtaaataaacacacgccaa

SEQ ID NO: 776 - DNA, 56 bp, synthetic construct
  source 1..56: mol_type = other DNA
actaaagtacggtgtctggaagtttcttgagggggaccctcggagaaaattgaaat

SEQ ID NO: 777 - DNA, 56 bp, synthetic construct
  source 1..56: mol_type = other DNA
acatttactgtaaattagatttaggttgctagaaatgagaataaaaataggtatta

SEQ ID NO: 778 - DNA, 56 bp, synthetic construct
  source 1..56: mol_type = other DNA
acaatttcatttgaattaccttgtgagtagtcaatctacctttcatatgcgctcaa

SEQ ID NO: 779 - DNA, 56 bp, synthetic construct
  source 1..56: mol_type = other DNA
gagagtatcaaagctaacggaccagtcatcatcaagacagatggcaaaaaaaatac

SEQ ID NO: 780 - DNA, 56 bp, synthetic construct
  source 1..56: mol_type = other DNA
ttttgattatcgcgggtagaatttaagaagaaccgaactgacactcatcggaagtt

SEQ ID NO: 781 - DNA, 56 bp, synthetic construct
  source 1..56: mol_type = other DNA
gagcgagtgctgaaattgcataactaatgtttaattcattcattagccgagtacaa

SEQ ID NO: 782 - DNA, 56 bp, synthetic construct
  source 1..56: mol_type = other DNA
accaagttacaaaaagaaattgcgtagattcctgattatcagtattaaatcctttg

SEQ ID NO: 783 - DNA, 56 bp, synthetic construct
  source 1..56: mol_type = other DNA
ttttcagtttgaatatcaagataattttcccttcgagaggctatgatgaaacaaac

SEQ ID NO: 784 - DNA, 56 bp, synthetic construct
  source 1..56: mol_type = other DNA
atagactttctccgattgccgttccggccagcctccggccagatccgccgggcgcg

SEQ ID NO: 785 - DNA, 56 bp, synthetic construct
  source 1..56: mol_type = other DNA
ctggaggagcgtggagccgcagcggatctcagagggggggatatcggcccggattg

SEQ ID NO: 786 - DNA, 56 bp, synthetic construct
  source 1..56: mol_type = other DNA
taaaggtgcactcaagcacatgatgctgtggtgaaacaatcgcgatcggcgcttct

SEQ ID NO: 787 - DNA, 56 bp, synthetic construct
  source 1..56: mol_type = other DNA
aattaactggtaatacattctttagtcttgaaaaatgaaaggcccgaaccatcata

SEQ ID NO: 788 - DNA, 56 bp, synthetic construct
  source 1..56: mol_type = other DNA
tattaaaggaacccctggcaaagagcggaaagagttaccgccacacgacgatagcc

SEQ ID NO: 789 - DNA, 56 bp, synthetic construct
  source 1..56: mol_type = other DNA
tctatcaaatcaaggccgcgctgctttgttctttgaactcaagaaccctgacaata

SEQ ID NO: 790 - DNA, 56 bp, synthetic construct
  source 1..56: mol_type = other DNA
cagagcataaagctaattaagcgcgagctgaaaagtaaccaatttaaataacagga

SEQ ID NO: 791 - DNA, 56 bp, synthetic construct
  source 1..56: mol_type = other DNA
ttagaactgtgtaggataaattaatgccaatcgatagattgtattgaccataaatc

SEQ ID NO: 792 - DNA, 59 bp, synthetic construct
  source 1..59: mol_type = other DNA
tactgaacaaagtcagatatcccaatccaaagacttgcgggaggtagaacaagcaagcc

SEQ ID NO: 793 - DNA, 59 bp, synthetic construct
  source 1..59: mol_type = other DNA
gaataacccacaagaatagagcctaatttgctattttgcacccagtcattccaagaacg

SEQ ID NO: 794 - DNA, 59 bp, synthetic construct
  source 1..59: mol_type = other DNA
tcaatgaattttctgtaaaaaggagcctttacataaccgatatatagactttttcatga

SEQ ID NO: 795 - DNA, 60 bp, synthetic construct
  source 1..60: mol_type = other DNA
acctccctaagaaaaacaccccagaaggatacataaaggtggcaacataccgaacaaagt

SEQ ID NO: 796 - DNA, 60 bp, synthetic construct
  source 1..60: mol_type = other DNA
tagttgccagttacagagagagccctttacggaataagtttattttgtcgctatcttacc

SEQ ID NO: 797 - DNA, 60 bp, synthetic construct
  source 1..60: mol_type = other DNA
gcccacgattgtatttagtaaccagtaccggaataggtgtatcaccgtaactgagtttcg

SEQ ID NO: 798 - DNA, 60 bp, synthetic construct
  source 1..60: mol_type = other DNA
cagtaataaaagggatccagaacaatatctgtccatcacgcatcctcgttagaatcgtgt

SEQ ID NO: 799 - DNA, 60 bp, synthetic construct
  source 1..60: mol_type = other DNA
tgtccagcatcagccccttacactggtggcgcgcctgtgcacgggtaccgagctcgcctg

SEQ ID NO: 800 - DNA, 60 bp, synthetic construct
  source 1..60: mol_type = other DNA
cagatcccacggcagcaaataaaaagctcattacgacggttaagttgtttgagggtcacg

SEQ ID NO: 801 - DNA, 60 bp, synthetic construct
  source 1..60: mol_type = other DNA
tttcaccgcctggccctgagagagttgcagagacggatacgagctgtttcgcatcagcat

SEQ ID NO: 802 - DNA, 78 bp, synthetic construct
  source 1..78: mol_type = other DNA
tttgggagaagcctttattcctgtaatacttttgctttcactcacctccatctccactcctacccatccaactcccac

SEQ ID NO: 803 - DNA, 69 bp, synthetic construct
  source 1..69: mol_type = other DNA
catcgacccgcaagaatgccaacggctttcgtttctttgcttgtgtcttgtcttcgtcgtgcttgttcc

SEQ ID NO: 804 - DNA, 76 bp, synthetic construct
  source 1..76: mol_type = other DNA
atttacccaaataaatcctcattaaagccagaatttcgtttctttgcttgtgtcttgtcttcgtcgtgcttgttcc

SEQ ID NO: 805 - DNA, 78 bp, synthetic construct
  source 1..78: mol_type = other DNA
tttgcattttcggtcatagtagcgcgttttcatcgtttcgtttctttgcttgtgtcttgtcttcgtcgtgcttgttcc

SEQ ID NO: 806 - DNA, 78 bp, synthetic construct
  source 1..78: mol_type = other DNA
tttgtttaccagcgccaaaatagaaaattcatatgtttcgtttctttgcttgtgtcttgtcttcgtcgtgcttgttcc

SEQ ID NO: 807 - DNA, 78 bp, synthetic construct
  source 1..78: mol_type = other DNA
tttcaagaaacaatgaaatagcccaataataagagtttcgtttctttgcttgtgtcttgtcttcgtcgtgcttgttcc

SEQ ID NO: 808 - DNA, 78 bp, synthetic construct
  source 1..78: mol_type = other DNA
tttccaacgctaacgagcgtttatcctgaatcttatttcgtttctttgcttgtgtcttgtcttcgtcgtgcttgttcc

SEQ ID NO: 809 - DNA, 78 bp, synthetic construct
  source 1..78: mol_type = other DNA
tttcaataatcggctgtctgcatgtagaaaccaattttcgtttctttgcttgtgtcttgtcttcgtcgtgcttgttcc

SEQ ID NO: 810 - DNA, 78 bp, synthetic construct
  source 1..78: mol_type = other DNA
tttaccagtataaagccaacgttatacaaattctttttcgtttctttgcttgtgtcttgtcttcgtcgtgcttgttcc

SEQ ID NO: 811 - DNA, 78 bp, synthetic construct
  source 1..78: mol_type = other DNA
ttttcataggtctgagagaagtgaatttatcaaaatttcgtttctttgcttgtgtcttgtcttcgtcgtgcttgttcc

SEQ ID NO: 812 - DNA, 78 bp, synthetic construct
  source 1..78: mol_type = other DNA
ttttacataaatcaatatatttttaatggaaacagtttcgtttctttgcttgtgtcttgtcttcgtcgtgcttgttcc

SEQ ID NO: 813 - DNA, 78 bp, synthetic construct
  source 1..78: mol_type = other DNA
ttttacatcgggagaaacacagtaacagtaccttttttcgtttctttgcttgtgtcttgtcttcgtcgtgcttgttcc

SEQ ID NO: 814 - DNA, 78 bp, synthetic construct
  source 1..78: mol_type = other DNA
ttttgcggaacaaagaaacagtaacattatcattttttcgtttctttgcttgtgtcttgtcttcgtcgtgcttgttcc

SEQ ID NO: 815 - DNA, 78 bp, synthetic construct
  source 1..78: mol_type = other DNA
tttcctcaatcaatatctgacctcaaatatcaaactttcgtttctttgcttgtgtcttgtcttcgtcgtgcttgttcc

SEQ ID NO: 816 - DNA, 78 bp, synthetic construct
  source 1..78: mol_type = other DNA
tttaagaatacgtggcacatctgacctgaaagcgttttcgtttctttgcttgtgtcttgtcttcgtcgtgcttgttcc

SEQ ID NO: 817 - DNA, 78 bp, synthetic construct
  source 1..78: mol_type = other DNA
tttcttgcctgagtagaagattagtaataacatcatttcgtttctttgcttgtgtcttgtcttcgtcgtgcttgttcc

SEQ ID NO: 818 - DNA, 78 bp, synthetic construct
  source 1..78: mol_type = other DNA
tttgggcgcgtactatggtttaatgcgccgctacatttcgtttctttgcttgtgtcttgtcttcgtcgtgcttgttcc

SEQ ID NO: 819 - DNA, 78 bp, synthetic construct
  source 1..78: mol_type = other DNA
tttcgtgaaccatcacccagggcgatggcccactatttcgtttctttgcttgtgtcttgtcttcgtcgtgcttgttcc

SEQ ID NO: 820 - DNA, 80 bp, synthetic construct
  source 1..80: mol_type = other DNA
ttggaaagcgcagtctctgagggttttgctcagtaccagtcgtttctttgcttgtgtcttgtcttcgtcgtgcttgttcc

SEQ ID NO: 821 - DNA, 80 bp, synthetic construct
  source 1..80: mol_type = other DNA
tgcggataagtgccgtcgagtccacagacagccctcatatcgtttctttgcttgtgtcttgtcttcgtcgtgcttgttcc

SEQ ID NO: 822 - DNA, 80 bp, synthetic construct
  source 1..80: mol_type = other DNA
tgttagcgtaacgatctaaaaggtgaatttcttaaacagtcgtttctttgcttgtgtcttgtcttcgtcgtgcttgttcc

SEQ ID NO: 823 - DNA, 80 bp, synthetic construct
  source 1..80: mol_type = other DNA
tcttgataccgatagttgcggtaatgccactacgaaggctcgtttctttgcttgtgtcttgtcttcgtcgtgcttgttcc

SEQ ID NO: 824 - DNA, 80 bp, synthetic construct
  source 1..80: mol_type = other DNA
taccaacctaaaacgaaagagaacggtgtacagaccaggtcgtttctttgcttgtgtcttgtcttcgtcgtgcttgttcc

SEQ ID NO: 825 - DNA, 80 bp, synthetic construct
  source 1..80: mol_type = other DNA
tcgcataggctggctgacctggacgttgggaagaaaaattcgtttctttgcttgtgtcttgtcttcgtcgtgcttgttcc

SEQ ID NO: 826 - DNA, 80 bp, synthetic construct
  source 1..80: mol_type = other DNA
tctacgttaataaaacgaacgaaccagaccggaagcaaatcgtttctttgcttgtgtcttgtcttcgtcgtgcttgttcc

SEQ ID NO: 827 - DNA, 80 bp, synthetic construct
  source 1..80: mol_type = other DNA
tctccaacaggtcaggattattggtgtagatgggcgcattcgtttctttgcttgtgtcttgtcttcgtcgtgcttgttcc

SEQ ID NO: 828 - DNA, 80 bp, synthetic construct
  source 1..80: mol_type = other DNA
tcgtaaccgtgcatctgccagggtaacgccagggttttctcgtttctttgcttgtgtcttgtcttcgtcgtgcttgttcc

SEQ ID NO: 829 - DNA, 80 bp, synthetic construct
  source 1..80: mol_type = other DNA
tagcaccgtcggtggtgccatgccgggttacctgcagcctcgtttctttgcttgtgtcttgtcttcgtcgtgcttgttcc

SEQ ID NO: 830 - DNA, 80 bp, synthetic construct
  source 1..80: mol_type = other DNA
tagcggtgccggtgccccctctgtgtgaaattgttatcctcgtttctttgcttgtgtcttgtcttcgtcgtgcttgttcc

SEQ ID NO: 831 - DNA, 80 bp, synthetic construct
  source 1..80: mol_type = other DNA
tgctcacaattccacacaacgcaacagctgattgccctttcgtttctttgcttgtgtcttgtcttcgtcgtgcttgttcc

SEQ ID NO: 832 - DNA, 51 bp, synthetic construct
  source 1..51: mol_type = other DNA
attagaccaataatgttagcaaacgtagtttacagttattagcccgcattt

SEQ ID NO: 833 - DNA, 58 bp, synthetic construct
  source 1..58: mol_type = other DNA
ctgaggcttgcaggtacagaggctttgacaagcgctttacagttattagcccgcattt

SEQ ID NO: 834 - DNA, 58 bp, synthetic construct
  source 1..58: mol_type = other DNA
aggcgttttagcgattttcatcgtaggatgttcagtttacagttattagcccgcattt

SEQ ID NO: 835 - DNA, 58 bp, synthetic construct
  source 1..58: mol_type = other DNA
ccttaaatcaagataaccaagtaccgcaaagtccttttacagttattagcccgcattt

SEQ ID NO: 836 - DNA, 58 bp, synthetic construct
  source 1..58: mol_type = other DNA
tgacaacaaccatctccattaaacgggtgaatacatttacagttattagcccgcattt

SEQ ID NO: 837 - DNA, 58 bp, synthetic construct
  source 1..58: mol_type = other DNA
ggaataccacattccaaaaagattaagagcttagatttacagttattagcccgcattt

SEQ ID NO: 838 - DNA, 58 bp, synthetic construct
  source 1..58: mol_type = other DNA
tttaccagacgacgaagaagttttgccacaattactttacagttattagcccgcattt

SEQ ID NO: 839 - DNA, 58 bp, synthetic construct
  source 1..58: mol_type = other DNA
aacatagcgatagctcgtcgctattaataaacaaatttacagttattagcccgcattt

SEQ ID NO: 840 - DNA, 58 bp, synthetic construct
  source 1..58: mol_type = other DNA
acaacattattacattttaattcgagctcctttaatttacagttattagcccgcattt

SEQ ID NO: 841 - DNA, 58 bp, synthetic construct
  source 1..58: mol_type = other DNA
taacctcaccggaagggatagctctcacgggtaaatttacagttattagcccgcattt

SEQ ID NO: 842 - DNA, 58 bp, synthetic construct
  source 1..58: mol_type = other DNA
tacaaacaattcgaatatctttaggagccctgcaatttacagttattagcccgcattt

SEQ ID NO: 843 - DNA, 58 bp, synthetic construct
  source 1..58: mol_type = other DNA
gttattaattttaaggcaaatcaacagttctaaagtttacagttattagcccgcattt

SEQ ID NO: 844 - DNA, 58 bp, synthetic construct
  source 1..58: mol_type = other DNA
ccagtgccaagcttaaacttaaatttctaatcccgtttacagttattagcccgcattt

SEQ ID NO: 845 - DNA, 58 bp, synthetic construct
  source 1..58: mol_type = other DNA
tgctgcggccagaatcctcacagttgaggctaacttttacagttattagcccgcattt

SEQ ID NO: 846 - DNA, 58 bp, synthetic construct
  source 1..58: mol_type = other DNA
gaggccaccgagtagagctaaacaggagcgggcgctttacagttattagcccgcattt

SEQ ID NO: 847 - DNA, 58 bp, synthetic construct
  source 1..58: mol_type = other DNA
cgttgtagcaatacacgagcacgtataataaccactttacagttattagcccgcattt

SEQ ID NO: 848 - DNA, 58 bp, synthetic construct
  source 1..58: mol_type = other DNA
acgatccagcgcagaatcatggtcatagccggaagtttacagttattagcccgcattt

SEQ ID NO: 849 - DNA, 72 bp, synthetic construct
  source 1..72: mol_type = other DNA
gccagcattgacaggtactggtaataagttattctgaaacatggtttagtttacagttattagcccgcattt

SEQ ID NO: 850 - DNA, 72 bp, synthetic construct
  source 1..72: mol_type = other DNA
ataatcaaaatcacatcagtagcgacaggggagggaaggtaaaacgcaatttacagttattagcccgcattt

SEQ ID NO: 851 - DNA, 72 bp, synthetic construct
  source 1..72: mol_type = other DNA
ttgatattcacaaagttccagtaagcgtagagaaggattaggatataagtttacagttattagcccgcattt

SEQ ID NO: 852 - DNA, 77 bp, synthetic construct
  source 1..77: mol_type = other DNA
ttaagaggctgagactcctcacatacatattggccttcgtttctttgcttgtgtcttgtcttcgtcgtgcttgttcc

SEQ ID NO: 853 - DNA, 77 bp, synthetic construct
  source 1..77: mol_type = other DNA
ccctgcctatttcggaacctattttaacagccgccttcgtttctttgcttgtgtcttgtcttcgtcgtgcttgttcc

SEQ ID NO: 854 - DNA, 77 bp, synthetic construct
  source 1..77: mol_type = other DNA
gaattatcaccgtcaccgacttagcaagcaccctcttcgtttctttgcttgtgtcttgtcttcgtcgtgcttgttcc

SEQ ID NO: 855 - DNA, 77 bp, synthetic construct
  source 1..77: mol_type = other DNA
cggaaattattcattaaaggtcaatgaaaaccgccttcgtttctttgcttgtgtcttgtcttcgtcgtgcttgttcc

SEQ ID NO: 856 - DNA, 77 bp, synthetic construct
  source 1..77: mol_type = other DNA
gggcgacattcaaccgattgaaatcaagtcttttcttcgtttctttgcttgtgtcttgtcttcgtcgtgcttgttcc

SEQ ID NO: 857 - DNA, 77 bp, synthetic construct
  source 1..77: mol_type = other DNA
cacattacgcggggagaggcggtttgatggtggttttcgtttctttgcttgtgtcttgtcttcgtcgtgcttgttcc

SEQ ID NO: 858 - DNA, 77 bp, synthetic construct
  source 1..77: mol_type = other DNA
actgcccgcattaatgaatcgcggcaaaatcccttttcgtttctttgcttgtgtcttgtcttcgtcgtgcttgttcc

SEQ ID NO: 859 - DNA, 77 bp, synthetic construct
  source 1..77: mol_type = other DNA
gaaagcgagcttgacggggaagttgagtgttgttcttcgtttctttgcttgtgtcttgtcttcgtcgtgcttgttcc

SEQ ID NO: 860 - DNA, 77 bp, synthetic construct
  source 1..77: mol_type = other DNA
tagggcgtaaagggagcccccgaacaagagtccacttcgtttctttgcttgtgtcttgtcttcgtcgtgcttgttcc

SEQ ID NO: 861 - DNA, 77 bp, synthetic construct
  source 1..77: mol_type = other DNA
cacacccttttttggggtcgaagggcgaaaaaccgttcgtttctttgcttgtgtcttgtcttcgtcgtgcttgttcc

SEQ ID NO: 862 - DNA, 77 bp, synthetic construct
  source 1..77: mol_type = other DNA
cataaagttcttttcaccagtgcaagcggtccacgttcgtttctttgcttgtgtcttgtcttcgtcgtgcttgttcc

SEQ ID NO: 863 - DNA, 80 bp, synthetic construct
  source 1..80: mol_type = other DNA
agcggtcacgtaaagcactaaatcgaacgtggactccattcgtttctttgcttgtgtcttgtcttcgtcgtgcttgttcc

SEQ ID NO: 864 - DNA, 80 bp, synthetic construct
  source 1..80: mol_type = other DNA
gggtgcctaatattgggcgccagggccccagcaggcgattcgtttctttgcttgtgtcttgtcttcgtcgtgcttgttcc

SEQ ID NO: 865 - DNA, 18 bp, synthetic construct
  source 1..18: mol_type = other DNA
taatcagtacattttcgg

SEQ ID NO: 866 - DNA, 18 bp, synthetic construct
  source 1..18: mol_type = other DNA
ggaaattatgccagcaaa

SEQ ID NO: 867 - DNA, 20 bp, synthetic construct
  source 1..20: mol_type = other DNA
aaaaatagaagccttgagag

SEQ ID NO: 868 - DNA, 21 bp, synthetic construct
  source 1..21: mol_type = other DNA
tttttcaggaacgagccgctt

SEQ ID NO: 869 - DNA, 21 bp, synthetic construct
  source 1..21: mol_type = other DNA
gcgcataactgaccctttcaa

SEQ ID NO: 870 - DNA, 21 bp, synthetic construct
  source 1..21: mol_type = other DNA
cagttacgaatcttcttcaag

SEQ ID NO: 871 - DNA, 22 bp, synthetic construct
  source 1..22: mol_type = other DNA
agagagaaacgatttgaaacgg

SEQ ID NO: 872 - DNA, 22 bp, synthetic construct
  source 1..22: mol_type = other DNA
agagccttagccaggaacaaac

SEQ ID NO: 873 - DNA, 22 bp, synthetic construct
  source 1..22: mol_type = other DNA
tattctacaatcatgcaaatat

SEQ ID NO: 874 - DNA, 22 bp, synthetic construct
  source 1..22: mol_type = other DNA
ttgcaacctcatggagtaataa

SEQ ID NO: 875 - DNA, 26 bp, synthetic construct
  source 1..26: mol_type = other DNA
ccaccctcattttcaactttgaataa

SEQ ID NO: 876 - DNA, 26 bp, synthetic construct
  source 1..26: mol_type = other DNA
acggaacaacattatatttaccctgt

SEQ ID NO: 877 - DNA, 26 bp, synthetic construct
  source 1..26: mol_type = other DNA
aattacgaggcaaagctaattaagca

SEQ ID NO: 878 - DNA, 26 bp, synthetic construct
  source 1..26: mol_type = other DNA
aagttttgccagatcataccatttgg

SEQ ID NO: 879 - DNA, 26 bp, synthetic construct
  source 1..26: mol_type = other DNA
caaatgctttaaaaatggtagtttga

SEQ ID NO: 880 - DNA, 26 bp, synthetic construct
  source 1..26: mol_type = other DNA
gcattccacagaccaaatcagaccag

SEQ ID NO: 881 - DNA, 26 bp, synthetic construct
  source 1..26: mol_type = other DNA
caatatgatattcgagccgtccgctc

SEQ ID NO: 882 - DNA, 26 bp, synthetic construct
  source 1..26: mol_type = other DNA
ctgagagtctggggtcataccaagct

SEQ ID NO: 883 - DNA, 26 bp, synthetic construct
  source 1..26: mol_type = other DNA
gccccaaaaacacagtcacaagttgg

SEQ ID NO: 884 - DNA, 26 bp, synthetic construct
  source 1..26: mol_type = other DNA
gctcattttttatggcgaacgggcct

SEQ ID NO: 885 - DNA, 26 bp, synthetic construct
  source 1..26: mol_type = other DNA
gtaacaacccgtgctgcgcaggcaaa

SEQ ID NO: 886 - DNA, 26 bp, synthetic construct
  source 1..26: mol_type = other DNA
tttgctaaacaaaactttggacctgc

SEQ ID NO: 887 - DNA, 26 bp, synthetic construct
  source 1..26: mol_type = other DNA
tcgtgccagctgtacatttattaccg

SEQ ID NO: 888 - DNA, 26 bp, synthetic construct
  source 1..26: mol_type = other DNA
gacgggcaacagtatcggctttgatt

SEQ ID NO: 889 - DNA, 26 bp, synthetic construct
  source 1..26: mol_type = other DNA
ctgtttgatggttcacgcagtgtttt

SEQ ID NO: 890 - DNA, 26 bp, synthetic construct
  source 1..26: mol_type = other DNA
aagagtccactaagacaggagaatca

SEQ ID NO: 891 - DNA, 26 bp, synthetic construct
  source 1..26: mol_type = other DNA
ataattttttcatcatcgctaaaaca

SEQ ID NO: 892 - DNA, 26 bp, synthetic construct
  source 1..26: mol_type = other DNA
aaatcaagtttttgacgagcgccgcg

SEQ ID NO: 893 - DNA, 26 bp, synthetic construct
  source 1..26: mol_type = other DNA
ttcttaaacagcaacctaataaagac

SEQ ID NO: 894 - DNA, 27 bp, synthetic construct
  source 1..27: mol_type = other DNA
agagccacaccatcaacagatcgagcc

SEQ ID NO: 895 - DNA, 27 bp, synthetic construct
  source 1..27: mol_type = other DNA
aaatcaccggaacccgccactcagagc

SEQ ID NO: 896 - DNA, 27 bp, synthetic construct
  source 1..27: mol_type = other DNA
tttatcccaatcgcgctaactgaacaa

SEQ ID NO: 897 - DNA, 27 bp, synthetic construct
  source 1..27: mol_type = other DNA
ccagctacaattaagaaacttgagtta

SEQ ID NO: 898 - DNA, 27 bp, synthetic construct
  source 1..27: mol_type = other DNA
tgcgggaggtttgcagataccgaagcc

SEQ ID NO: 899 - DNA, 27 bp, synthetic construct
  source 1..27: mol_type = other DNA
tcattaccgcgcatacccaaccgagga

SEQ ID NO: 900 - DNA, 27 bp, synthetic construct
  source 1..27: mol_type = other DNA
ccgcactcatcggtagaaacttattac

SEQ ID NO: 901 - DNA, 27 bp, synthetic construct
  source 1..27: mol_type = other DNA
ggacgattggtgttccgtggctttcat

SEQ ID NO: 902 - DNA, 27 bp, synthetic construct
  source 1..27: mol_type = other DNA
gatgcaaatccaaattctttgtttagt

SEQ ID NO: 903 - DNA, 27 bp, synthetic construct
  source 1..27: mol_type = other DNA
gagctctcgactcgtccacgcaaaatc

SEQ ID NO: 904 - DNA, 27 bp, synthetic construct
  source 1..27: mol_type = other DNA
tattcatttcaaacaagaatttatcaa

SEQ ID NO: 905 - DNA, 27 bp, synthetic construct
  source 1..27: mol_type = other DNA
ttgaggaaggtttatcattttaatttt

SEQ ID NO: 906 - DNA, 27 bp, synthetic construct
  source 1..27: mol_type = other DNA
cttgctgaaccttgattataggagcgg

SEQ ID NO: 907 - DNA, 27 bp, synthetic construct
  source 1..27: mol_type = other DNA
tcagtattaacactgaataaatcctga

SEQ ID NO: 908 - DNA, 27 bp, synthetic construct
  source 1..27: mol_type = other DNA
tagccctaaaacaaataaatcaaaatt

SEQ ID NO: 909 - DNA, 27 bp, synthetic construct
  source 1..27: mol_type = other DNA
ctgaaagcgtaagtaccttaacgtcag

SEQ ID NO: 910 - DNA, 28 bp, synthetic construct
  source 1..28: mol_type = other DNA
tcatcgggcgacagatcgataagcacca

SEQ ID NO: 911 - DNA, 28 bp, synthetic construct
  source 1..28: mol_type = other DNA
gcgtcacgctgaaattaacatcttccgg

SEQ ID NO: 912 - DNA, 28 bp, synthetic construct
  source 1..28: mol_type = other DNA
gtagcaaggtggctgttaaacatcaaaa

SEQ ID NO: 913 - DNA, 28 bp, synthetic construct
  source 1..28: mol_type = other DNA
tgtaaagacaaacttcgtagaatgggta

SEQ ID NO: 914 - DNA, 28 bp, synthetic construct
  source 1..28: mol_type = other DNA
ataattcttcctgaatttgctgtctgga

SEQ ID NO: 915 - DNA, 28 bp, synthetic construct
  source 1..28: mol_type = other DNA
gacaaaggggttataatcataagcttag

SEQ ID NO: 916 - DNA, 28 bp, synthetic construct
  source 1..28: mol_type = other DNA
ccttttacatcctttagaaggctgcagg

SEQ ID NO: 917 - DNA, 28 bp, synthetic construct
  source 1..28: mol_type = other DNA
ccttttttaatgcaacatgattctgtcc

SEQ ID NO: 918 - DNA, 28 bp, synthetic construct
  source 1..28: mol_type = other DNA
ggcgatcggaaagaaatcaatacttgca

SEQ ID NO: 919 - DNA, 28 bp, synthetic construct
  source 1..28: mol_type = other DNA
aaaagcataacagtacagttaggttgtc

SEQ ID NO: 920 - DNA, 28 bp, synthetic construct
  source 1..28: mol_type = other DNA
caaacccagcaaatcaacagtcagaaga

SEQ ID NO: 921 - DNA, 28 bp, synthetic construct
  source 1..28: mol_type = other DNA
gccagctcaatcaatggccgcaggaatc

SEQ ID NO: 922 - DNA, 28 bp, synthetic construct
  source 1..28: mol_type = other DNA
gaatggtaccgaacgtgagtttgccaac

SEQ ID NO: 923 - DNA, 29 bp, synthetic construct
  source 1..29: mol_type = other DNA
ggtcatttttttaaaattccatattttcc

SEQ ID NO: 924 - DNA, 29 bp, synthetic construct
  source 1..29: mol_type = other DNA
tcaatagtgaattatttaaagtagggctt

SEQ ID NO: 925 - DNA, 29 bp, synthetic construct
  source 1..29: mol_type = other DNA
ccttgcttctgtagaatatgaggcatttt

SEQ ID NO: 926 - DNA, 29 bp, synthetic construct
  source 1..29: mol_type = other DNA
gcggggagcgtattaattaatcgggtacc

SEQ ID NO: 927 - DNA, 29 bp, synthetic construct
  source 1..29: mol_type = other DNA
gccctgagcaagcgtagaggaaattaatt

SEQ ID NO: 928 - DNA, 29 bp, synthetic construct
  source 1..29: mol_type = other DNA
ccttataatagccctagcgatggtctgag

SEQ ID NO: 929 - DNA, 30 bp, synthetic construct
  source 1..30: mol_type = other DNA
accgcctccctcagagcagagccccttatt

SEQ ID NO: 930 - DNA, 30 bp, synthetic construct
  source 1..30: mol_type = other DNA
atcgcactctcaaatattttcgcatttaac

SEQ ID NO: 931 - DNA, 31 bp, synthetic construct
  source 1..31: mol_type = other DNA
agggagttaaaggggtagcaacggctacaga

SEQ ID NO: 932 - DNA, 31 bp, synthetic construct
  source 1..31: mol_type = other DNA
ccaacaggtcagtggcttagagcttaattgc

SEQ ID NO: 933 - DNA, 31 bp, synthetic construct
  source 1..31: mol_type = other DNA
gaaaatagcagcgcgcattagacgggagaat

SEQ ID NO: 934 - DNA, 31 bp, synthetic construct
  source 1..31: mol_type = other DNA
gtgcatctgccactttccggcaccgcttctg

SEQ ID NO: 935 - DNA, 31 bp, synthetic construct
  source 1..31: mol_type = other DNA
ttgaaataccgaaacaccggaatcataatta

SEQ ID NO: 936 - DNA, 31 bp, synthetic construct
  source 1..31: mol_type = other DNA
agactactccggccagggcgccgtaaagcac

SEQ ID NO: 937 - DNA, 31 bp, synthetic construct
  source 1..31: mol_type = other DNA
gcgaacgtggcgtagcggtcacgctgcgcgt

SEQ ID NO: 938 - DNA, 32 bp, synthetic construct
  source 1..32: mol_type = other DNA
agtttctatgcaaggtcacgcgacagtggaag

SEQ ID NO: 939 - DNA, 32 bp, synthetic construct
  source 1..32: mol_type = other DNA
aaaagtattcgacaactcgtattaaatccttt

SEQ ID NO: 940 - DNA, 34 bp, synthetic construct
  source 1..34: mol_type = other DNA
gagccatttgggttaccatctgcctagtcagtgc

SEQ ID NO: 941 - DNA, 34 bp, synthetic construct
  source 1..34: mol_type = other DNA
gattgagggaggaggtgaaagcggggactcctca

SEQ ID NO: 942 - DNA, 34 bp, synthetic construct
  source 1..34: mol_type = other DNA
ttaaataatatttatcaattaccaacgctaacga

SEQ ID NO: 943 - DNA, 34 bp, synthetic construct
  source 1..34: mol_type = other DNA
atcataaagaataccgtgtgataaatatcatctt

SEQ ID NO: 944 - DNA, 34 bp, synthetic construct
  source 1..34: mol_type = other DNA
ttccctgaaaacagagatagctccaacgtcaaag

SEQ ID NO: 945 - DNA, 35 bp, synthetic construct
  source 1..35: mol_type = other DNA
ggtcagaccaccccctcagaaccgccaggtaatca

SEQ ID NO: 946 - DNA, 35 bp, synthetic construct
  source 1..35: mol_type = other DNA
ctgtagcgcgtttagcgttttgatattttgaggca

SEQ ID NO: 947 - DNA, 35 bp, synthetic construct
  source 1..35: mol_type = other DNA
caccaatgaaaccaatcaagtcatacatctgaatt

SEQ ID NO: 948 - DNA, 35 bp, synthetic construct
  source 1..35: mol_type = other DNA
cgatgaacggtaaagcatgtagaacgctaatagta

SEQ ID NO: 949 - DNA, 35 bp, synthetic construct
  source 1..35: mol_type = other DNA
gaaaacttattttaagcccccgaaagcggcgctag

SEQ ID NO: 950 - DNA, 35 bp, synthetic construct
  source 1..35: mol_type = other DNA
gaattcgcatcaagaaaacaagggcgccccgcctg

SEQ ID NO: 951 - DNA, 35 bp, synthetic construct
  source 1..35: mol_type = other DNA
cttgaggtaagcgtttgcctttagcgcagaaacgt

SEQ ID NO: 952 - DNA, 35 bp, synthetic construct
  source 1..35: mol_type = other DNA
taccgtttggcctgccatcttttcataaagtcaga

SEQ ID NO: 953 - DNA, 36 bp, synthetic construct
  source 1..36: mol_type = other DNA
gagaggtaggattttatcaccgtcatagattcaacc

SEQ ID NO: 954 - DNA, 36 bp, synthetic construct
  source 1..36: mol_type = other DNA
taatgccgctattttttatttatcagatatagaagg

SEQ ID NO: 955 - DNA, 36 bp, synthetic construct
  source 1..36: mol_type = other DNA
ggagaaacaatcaataatcggttatcatgcaaggat

SEQ ID NO: 956 - DNA, 36 bp, synthetic construct
  source 1..36: mol_type = other DNA
agagaaatgcccctagcaaggccggacagccgactt

SEQ ID NO: 957 - DNA, 36 bp, synthetic construct
  source 1..36: mol_type = other DNA
ggaacgcattcgcatgtataaatgtaccaagagaat

SEQ ID NO: 958 - DNA, 36 bp, synthetic construct
  source 1..36: mol_type = other DNA
ggcggatgggatactaaagttattttggctccacat

SEQ ID NO: 959 - DNA, 37 bp, synthetic construct
  source 1..37: mol_type = other DNA
cgagatcgccattatcaaataactatatgtaaatgct

SEQ ID NO: 960 - DNA, 37 bp, synthetic construct
  source 1..37: mol_type = other DNA
aattttatacaatcgcaactgacctaaatttaatggt

SEQ ID NO: 961 - DNA, 38 bp, synthetic construct
  source 1..38: mol_type = other DNA
aaaatagagcaacataatgcaggtagaaagattcatca

SEQ ID NO: 962 - DNA, 38 bp, synthetic construct
  source 1..38: mol_type = other DNA
ttgcatcaaaaattgattctgtagctcgcatcgtaacc

SEQ ID NO: 963 - DNA, 38 bp, synthetic construct
  source 1..38: mol_type = other DNA
agacgtaataagaaatcgtattaagacgctgagaagag

SEQ ID NO: 964 - DNA, 38 bp, synthetic construct
  source 1..38: mol_type = other DNA
taaatcgggacaaaaaccgtctatttaggttaacgcga

SEQ ID NO: 965 - DNA, 39 bp, synthetic construct
  source 1..39: mol_type = other DNA
aaccgaggctggctgaccttaaaaaccaaatgtttagac

SEQ ID NO: 966 - DNA, 39 bp, synthetic construct
  source 1..39: mol_type = other DNA
ggatatcctgacgagaaacaattcaacctatcataaccc

SEQ ID NO: 967 - DNA, 39 bp, synthetic construct
  source 1..39: mol_type = other DNA
gcagtataacggaccaatagtttagcgaacctcccgact

SEQ ID NO: 968 - DNA, 39 bp, synthetic construct
  source 1..39: mol_type = other DNA
aacgcaaaagtaatgaagccattagttgctattttgcac

SEQ ID NO: 969 - DNA, 39 bp, synthetic construct
  source 1..39: mol_type = other DNA
cttttttaagagcttatcctaaaataaacagccatatta

SEQ ID NO: 970 - DNA, 39 bp, synthetic construct
  source 1..39: mol_type = other DNA
agcccataattgacaaataatttgtttaacgtcaaaaat

SEQ ID NO: 971 - DNA, 39 bp, synthetic construct
  source 1..39: mol_type = other DNA
caatagcaataaagaaacagaatatatgtgagtgaataa

SEQ ID NO: 972 - DNA, 39 bp, synthetic construct
  source 1..39: mol_type = other DNA
atgaataaaacagatcgccataaaacagaggtgaggcgg

SEQ ID NO: 973 - DNA, 39 bp, synthetic construct
  source 1..39: mol_type = other DNA
atttgctatacttccgcctggaaaaatctaaagcatcac

SEQ ID NO: 974 - DNA, 39 bp, synthetic construct
  source 1..39: mol_type = other DNA
ttgtttatattcccaaatataatcaacagttgaaaggaa

SEQ ID NO: 975 - DNA, 39 bp, synthetic construct
  source 1..39: mol_type = other DNA
aattatgtaacatatctaaaagagccgtcaatagataat

SEQ ID NO: 976 - DNA, 40 bp, synthetic construct
  source 1..40: mol_type = other DNA
actacgattgacccccagcgataccctgccgaaagacttc

SEQ ID NO: 977 - DNA, 40 bp, synthetic construct
  source 1..40: mol_type = other DNA
ctcatctaggcaccttgatacttcggtcgctgaggcttgc

SEQ ID NO: 978 - DNA, 40 bp, synthetic construct
  source 1..40: mol_type = other DNA
aacggagtacttagccggaaccggaatcgagaatgaccat

SEQ ID NO: 979 - DNA, 40 bp, synthetic construct
  source 1..40: mol_type = other DNA
tccatgtatttgtacgttgaagcttgctttcgaggtgaat

SEQ ID NO: 980 - DNA, 40 bp, synthetic construct
  source 1..40: mol_type = other DNA
ggcttgctcattaccagcccttgaattttctgtatgggat

SEQ ID NO: 981 - DNA, 40 bp, synthetic construct
  source 1..40: mol_type = other DNA
caccagttagcaagcctcagaaccgccaccctcagagcca

SEQ ID NO: 982 - DNA, 40 bp, synthetic construct
  source 1..40: mol_type = other DNA
ccattagataacaggattaagaccagaccggaagcaaact

SEQ ID NO: 983 - DNA, 40 bp, synthetic construct
  source 1..40: mol_type = other DNA
ggcgcgaatttcgcacagttctagtcagaagcaaagcgga

SEQ ID NO: 984 - DNA, 40 bp, synthetic construct
  source 1..40: mol_type = other DNA
ataaagcccaataaagggggtatattcattgaatccccct

SEQ ID NO: 985 - DNA, 40 bp, synthetic construct
  source 1..40: mol_type = other DNA
aatacttgagcatatagtaagcgagaggcttttgcaaaag

SEQ ID NO: 986 - DNA, 40 bp, synthetic construct
  source 1..40: mol_type = other DNA
gtaacgccgccagcaccaatacaacattaaatgtgagcga

SEQ ID NO: 987 - DNA, 40 bp, synthetic construct
  source 1..40: mol_type = other DNA
tgcatgcgttttccggaagatttaaatttttgttaaatca

SEQ ID NO: 988 - DNA, 40 bp, synthetic construct
  source 1..40: mol_type = other DNA
acaattctaatcatagcaaacccggttgataatcagaaaa

SEQ ID NO: 989 - DNA, 40 bp, synthetic construct
  source 1..40: mol_type = other DNA
tgattgcagtaacagaatacgtctttaatgcgcgaactga

SEQ ID NO: 990 - DNA, 40 bp, synthetic construct
  source 1..40: mol_type = other DNA
aaacattaaccctaaaggggttaattagcctcaatcgggc

SEQ ID NO: 991 - DNA, 40 bp, synthetic construct
  source 1..40: mol_type = other DNA
gagcggggttgcttttggggtagcttgacggggaaagccg

SEQ ID NO: 992 - DNA, 40 bp, synthetic construct
  source 1..40: mol_type = other DNA
tataatcggattttttaaagaactacgtgaaccatcaccc

SEQ ID NO: 993 - DNA, 40 bp, synthetic construct
  source 1..40: mol_type = other DNA
tttatcaaatctccaggaacaactaaaggaattgcgaata

SEQ ID NO: 994 - DNA, 40 bp, synthetic construct
  source 1..40: mol_type = other DNA
agtaatactgtccaggttccgtgttgttccagtttggaac

SEQ ID NO: 995 - DNA, 40 bp, synthetic construct
  source 1..40: mol_type = other DNA
ccagccactcaaacctgattggccccagcaggcgaaaatc

SEQ ID NO: 996 - DNA, 41 bp, synthetic construct
  source 1..41: mol_type = other DNA
aaaaaaggcgttttaattcttaaacgccagcagccctcctc

SEQ ID NO: 997 - DNA, 42 bp, synthetic construct
  source 1..42: mol_type = other DNA
acagcatctgaggaagtttccagagcttctttaattataaga

SEQ ID NO: 998 - DNA, 43 bp, synthetic construct
  source 1..43: mol_type = other DNA
ttaaaacgaggcgtcaagcaacaccgttgcaagactccaagaa

SEQ ID NO: 999 - DNA, 44 bp, synthetic construct
  source 1..44: mol_type = other DNA
aattagatcattaagaaggtaccagcgcatcaatagaaaattca

SEQ ID NO: 1000 - DNA, 44 bp, synthetic construct
  source 1..44: mol_type = other DNA
aaatcaaaaaacgtccaatactggaggcgcgcgaaacaaagtac

SEQ ID NO: 1001 - DNA, 44 bp, synthetic construct
  source 1..44: mol_type = other DNA
aaatatcggataaatcaggtcttttataccaaatacgtaatgcc

SEQ ID NO: 1002 - DNA, 44 bp, synthetic construct
  source 1..44: mol_type = other DNA
ataccgttgaattgcagaagatgatgaaacaaacacaagctaac

SEQ ID NO: 1003 - DNA, 45 bp, synthetic construct
  source 1..45: mol_type = other DNA
tcgtttaaatgtaggaataccacccagaacatcttgacaagaacc

SEQ ID NO: 1004 - DNA, 45 bp, synthetic construct
  source 1..45: mol_type = other DNA
tggataggaatccagacgacgatcatcaagggtcaatcataaggg

SEQ ID NO: 1005 - DNA, 46 bp, synthetic construct
  source 1..46: mol_type = other DNA
gttgagattttaagacaccaacgtttcaggcgagtaaattgggctt

SEQ ID NO: 1006 - DNA, 47 bp, synthetic construct
  source 1..47: mol_type = other DNA
acatttgaggatttagaagtattagaatagattatatcttgttggca

SEQ ID NO: 1007 - DNA, 47 bp, synthetic construct
  source 1..47: mol_type = other DNA
ttaaaaactattagtggcacagccaacagagatagaacccttctgac

SEQ ID NO: 1008 - DNA, 48 bp, synthetic construct
  source 1..48: mol_type = other DNA
aggtggcaacatataaaagtgtcacacaaagactatagccgtgccgtc

SEQ ID NO: 1009 - DNA, 48 bp, synthetic construct
  source 1..48: mol_type = other DNA
cttacattgcgggttttagaaaagggtgatttccctgtcgagataaat

SEQ ID NO: 1010 - DNA, 48 bp, synthetic construct
  source 1..48: mol_type = other DNA
ctttccagatgagtgcaacatacaaccgtaaggctatcaggtcattgc

SEQ ID NO: 1011 - DNA, 49 bp, synthetic construct
  source 1..49: mol_type = other DNA
cggagacagagattcaaccctcatattacagatacataacgccaaaagg

SEQ ID NO: 1012 - DNA, 49 bp, synthetic construct
  source 1..49: mol_type = other DNA
cgctatttcaaatctacatcctacatttagcaaaaagttacaaaatcgc

SEQ ID NO: 1013 - DNA, 50 bp, synthetic construct
  source 1..50: mol_type = other DNA
tctacgttaaagaactgtttaatttcagggaacaaactacaacgcctgta

SEQ ID NO: 1014 - DNA, 50 bp, synthetic construct
  source 1..50: mol_type = other DNA
cttcgctcattcagcggattcagatgggcaacatgttgcggagattagag

SEQ ID NO: 1015 - DNA, 55 bp, synthetic construct
  source 1..55: mol_type = other DNA
atcaccgtactcaggaggtttagtacatataagaaaagggcgacagtgaataagt

SEQ ID NO: 1016 - DNA, 59 bp, synthetic construct
  source 1..59: mol_type = other DNA
gtctgaaatggattatttacattggcaaatacccattaattttttcttttcaccagtga

SEQ ID NO: 1017 - DNA, 18 bp, synthetic construct
  source 1..18: mol_type = other DNA
atcaccagtgcagcaccg

SEQ ID NO: 1018 - DNA, 21 bp, synthetic construct
  source 1..21: mol_type = other DNA
ttgcgggcgatatacgatagt

SEQ ID NO: 1019 - DNA, 21 bp, synthetic construct
  source 1..21: mol_type = other DNA
cagtttctagtaaacatagtt

SEQ ID NO: 1020 - DNA, 21 bp, synthetic construct
  source 1..21: mol_type = other DNA
tcacattcgctcacccaacgc

SEQ ID NO: 1021 - DNA, 23 bp, synthetic construct
  source 1..23: mol_type = other DNA
cttaatggcaagtgagaaaggaa

SEQ ID NO: 1022 - DNA, 24 bp, synthetic construct
  source 1..24: mol_type = other DNA
ccaggcggataacggaataggtgt

SEQ ID NO: 1023 - DNA, 24 bp, synthetic construct
  source 1..24: mol_type = other DNA
tgaatataatgcccaattctgcga

SEQ ID NO: 1024 - DNA, 24 bp, synthetic construct
  source 1..24: mol_type = other DNA
gtaatttaggcaaaagtaccgaca

SEQ ID NO: 1025 - DNA, 24 bp, synthetic construct
  source 1..24: mol_type = other DNA
gtgccggaaaccaactgttgggaa

SEQ ID NO: 1026 - DNA, 28 bp, synthetic construct
  source 1..28: mol_type = other DNA
cgccacccaggaggcacaaaccgcagtc

SEQ ID NO: 1027 - DNA, 28 bp, synthetic construct
  source 1..28: mol_type = other DNA
tggtcataggagcacgcgcgacaggtaa

SEQ ID NO: 1028 - DNA, 28 bp, synthetic construct
  source 1..28: mol_type = other DNA
caccaggccacgctggagtaccaccaga

SEQ ID NO: 1029 - DNA, 31 bp, synthetic construct
  source 1..31: mol_type = other DNA
attaccttatgcgattttataaaacgaacta

SEQ ID NO: 1030 - DNA, 31 bp, synthetic construct
  source 1..31: mol_type = other DNA
ctgagtaatgtgtaggtaatcaaatcaccat

SEQ ID NO: 1031 - DNA, 31 bp, synthetic construct
  source 1..31: mol_type = other DNA
gtaaagcctggggtgcctatcgggaaacctg

SEQ ID NO: 1032 - DNA, 31 bp, synthetic construct
  source 1..31: mol_type = other DNA
aaacaataacggattcgccgcagaggcgaat

SEQ ID NO: 1033 - DNA, 35 bp, synthetic construct
  source 1..35: mol_type = other DNA
aagggacattctggacaatattaaacgaggaattt

SEQ ID NO: 1034 - DNA, 40 bp, synthetic construct
  source 1..40: mol_type = other DNA
tggcttttaacgggtttcggaggctgagttttgctcagta

SEQ ID NO: 1035 - DNA, 40 bp, synthetic construct
  source 1..40: mol_type = other DNA
gggaagatttagcgaggtgatggcccacgtggaggttgag

SEQ ID NO: 1036 - DNA, 42 bp, synthetic construct
  source 1..42: mol_type = other DNA
gttaaattgcgtaattgaccggactggccgcgtgaccacgtt

SEQ ID NO: 1037 - DNA, 46 bp, synthetic construct
  source 1..46: mol_type = other DNA
ttatttaaacgcaagcaaacagaacaatttattttcatcgtaggaa

SEQ ID NO: 1038 - DNA, 46 bp, synthetic construct
  source 1..46: mol_type = other DNA
cgggtatagaaacgtcctgattacctgaacaatttcatttgaatta

SEQ ID NO: 1039 - DNA, 48 bp, synthetic construct
  source 1..48: mol_type = other DNA
agtaccaaagcgaaggaagcactattaagaaaacgtcataaaatagta

SEQ ID NO: 1040 - DNA, 24 bp, synthetic construct
  source 1..24: mol_type = other DNA
ggctttgaggacaacgaaagaggc

SEQ ID NO: 1041 - DNA, 24 bp, synthetic construct
  source 1..24: mol_type = other DNA
aaaagaatacacctgataaattgt

SEQ ID NO: 1042 - DNA, 24 bp, synthetic construct
  source 1..24: mol_type = other DNA
gtcgaaatccgcaaagaggacaga

SEQ ID NO: 1043 - DNA, 24 bp, synthetic construct
  source 1..24: mol_type = other DNA
tgaacggtgtacaacgtaacaaag

SEQ ID NO: 1044 - DNA, 24 bp, synthetic construct
  source 1..24: mol_type = other DNA
ctgctcattcagtaatcattgtga

SEQ ID NO: 1045 - DNA, 24 bp, synthetic construct
  source 1..24: mol_type = other DNA
tgattaagactcatacatacataa

SEQ ID NO: 1046 - DNA, 24 bp, synthetic construct
  source 1..24: mol_type = other DNA
taccagaaggaaaaagaactggca

SEQ ID NO: 1047 - DNA, 24 bp, synthetic construct
  source 1..24: mol_type = other DNA
atagctatcttagccgaacaaagt

SEQ ID NO: 1048 - DNA, 24 bp, synthetic construct
  source 1..24: mol_type = other DNA
aacccacaagaaaatgaaatagca

SEQ ID NO: 1049 - DNA, 24 bp, synthetic construct
  source 1..24: mol_type = other DNA
taactgaacacctatcagagagat

SEQ ID NO: 1050 - DNA, 24 bp, synthetic construct
  source 1..24: mol_type = other DNA
acgagtagatttcaataacctgtt

SEQ ID NO: 1051 - DNA, 24 bp, synthetic construct
  source 1..24: mol_type = other DNA
gaattagcaaaaatcggttgtacc

SEQ ID NO: 1052 - DNA, 24 bp, synthetic construct
  source 1..24: mol_type = other DNA
aaaaacattatgtaaatgcaatgc

SEQ ID NO: 1053 - DNA, 24 bp, synthetic construct
  source 1..24: mol_type = other DNA
aaaggtaaagtattcagctaatgc

SEQ ID NO: 1054 - DNA, 24 bp, synthetic construct
  source 1..24: mol_type = other DNA
ccaacgctcaaccaacgccaacat

SEQ ID NO: 1055 - DNA, 24 bp, synthetic construct
  source 1..24: mol_type = other DNA
gggcgatcggtgagggggatgtgc

SEQ ID NO: 1056 - DNA, 24 bp, synthetic construct
  source 1..24: mol_type = other DNA
cgacggccagtggctgtttcctgt

SEQ ID NO: 1057 - DNA, 24 bp, synthetic construct
  source 1..24: mol_type = other DNA
gtgaaattgttagaagcataaagt

SEQ ID NO: 1058 - DNA, 24 bp, synthetic construct
  source 1..24: mol_type = other DNA
attttcaggtttttacatcgggag

SEQ ID NO: 1059 - DNA, 24 bp, synthetic construct
  source 1..24: mol_type = other DNA
gaacctaccatagaaattgcgtag

SEQ ID NO: 1060 - DNA, 24 bp, synthetic construct
  source 1..24: mol_type = other DNA
attcatcaatatatggaagggtta

SEQ ID NO: 1061 - DNA, 24 bp, synthetic construct
  source 1..24: mol_type = other DNA
gaaaccaccagacagatgatggca

SEQ ID NO: 1062 - DNA, 24 bp, synthetic construct
  source 1..24: mol_type = other DNA
gcccgaacgttattgcggaacaaa

SEQ ID NO: 1063 - DNA, 24 bp, synthetic construct
  source 1..24: mol_type = other DNA
aaccaccacacccacgtataacgt

SEQ ID NO: 1064 - DNA, 24 bp, synthetic construct
  source 1..24: mol_type = other DNA
gctttcctcgttaacggtacgcca

SEQ ID NO: 1065 - DNA, 24 bp, synthetic construct
  source 1..24: mol_type = other DNA
gaatcctgagaaaattaaccgttg

SEQ ID NO: 1066 - DNA, 24 bp, synthetic construct
  source 1..24: mol_type = other DNA
tagcaatacttccttgctggtaat

SEQ ID NO: 1067 - DNA, 24 bp, synthetic construct
  source 1..24: mol_type = other DNA
atccagaacaattgacgctcaatc

SEQ ID NO: 1068 - DNA, 31 bp, synthetic construct
  source 1..31: mol_type = other DNA
atcctaatttacgagcatgttaaaccaagta

SEQ ID NO: 1069 - DNA, 32 bp, synthetic construct
  source 1..32: mol_type = other DNA
ccgccgccagcattgaagaaccaccaccagag

SEQ ID NO: 1070 - DNA, 32 bp, synthetic construct
  source 1..32: mol_type = other DNA
catgaaagtattaagaacctattattctgaaa

SEQ ID NO: 1071 - DNA, 32 bp, synthetic construct
  source 1..32: mol_type = other DNA
tactggtaataagttttgatgatacaggagtg

SEQ ID NO: 1072 - DNA, 32 bp, synthetic construct
  source 1..32: mol_type = other DNA
aagccagaatggaaagaaataaatcctcatta

SEQ ID NO: 1073 - DNA, 18 bp, synthetic construct
  source 1..18: mol_type = other DNA
tcatagcccaccaccgga

SEQ ID NO: 1074 - DNA, 18 bp, synthetic construct
  source 1..18: mol_type = other DNA
tatggtttaaatattgac

SEQ ID NO: 1075 - DNA, 19 bp, synthetic construct
  source 1..19: mol_type = other DNA
agtcagcagggaactttac

SEQ ID NO: 1076 - DNA, 19 bp, synthetic construct
  source 1..19: mol_type = other DNA
atctacatctagctaaggc

SEQ ID NO: 1077 - DNA, 21 bp, synthetic construct
  source 1..21: mol_type = other DNA
ggcgctgcgccgcttactatg

SEQ ID NO: 1078 - DNA, 21 bp, synthetic construct
  source 1..21: mol_type = other DNA
agctaaaattaaagagtgagg

SEQ ID NO: 1079 - DNA, 21 bp, synthetic construct
  source 1..21: mol_type = other DNA
aaagagtacatcacagaagaa

SEQ ID NO: 1080 - DNA, 22 bp, synthetic construct
  source 1..22: mol_type = other DNA
gcgccatcagccaggtttgagg

SEQ ID NO: 1081 - DNA, 22 bp, synthetic construct
  source 1..22: mol_type = other DNA
caactactagcggaaaggttac

SEQ ID NO: 1082 - DNA, 24 bp, synthetic construct
  source 1..24: mol_type = other DNA
tagctatattttaggcaaggcaaa

SEQ ID NO: 1083 - DNA, 24 bp, synthetic construct
  source 1..24: mol_type = other DNA
agaacgcgcctgaaataatatccc

SEQ ID NO: 1084 - DNA, 24 bp, synthetic construct
  source 1..24: mol_type = other DNA
ctagaaaaagccaccagtataaag

SEQ ID NO: 1085 - DNA, 24 bp, synthetic construct
  source 1..24: mol_type = other DNA
tgcaaggcgattgacgttgtaaaa

SEQ ID NO: 1086 - DNA, 26 bp, synthetic construct
  source 1..26: mol_type = other DNA
gagaggtatggtttgaggcactttga

SEQ ID NO: 1087 - DNA, 28 bp, synthetic construct
  source 1..28: mol_type = other DNA
taggaacccacactgagtttcgtagcgt

SEQ ID NO: 1088 - DNA, 28 bp, synthetic construct
  source 1..28: mol_type = other DNA
aacgatctagtctttccagacgtagcgg

SEQ ID NO: 1089 - DNA, 28 bp, synthetic construct
  source 1..28: mol_type = other DNA
agtgtataagcccgagaatagaaaaaaa

SEQ ID NO: 1090 - DNA, 28 bp, synthetic construct
  source 1..28: mol_type = other DNA
aaaggctcctttaattgtatcggtgcgc

SEQ ID NO: 1091 - DNA, 32 bp, synthetic construct
  source 1..32: mol_type = other DNA
gagatgggctcattataccaggggaagaaaaa

SEQ ID NO: 1092 - DNA, 36 bp, synthetic construct
  source 1..36: mol_type = other DNA
cgacaatgacgcccacgcataacatcgtcacgaaag

SEQ ID NO: 1093 - DNA, 41 bp, synthetic construct
  source 1..41: mol_type = other DNA
aaatcggctggtttcccttcaagggtgggaatcggtgcccg

SEQ ID NO: 1094 - DNA, 47 bp, synthetic construct
  source 1..47: mol_type = other DNA
aagttatgtccgtaatgtatgcgccaccctcagaaccgccaccccaa

SEQ ID NO: 1095 - DNA, 21 bp, synthetic construct
  source 1..21: mol_type = other DNA
taaccctatacactaaaacac

SEQ ID NO: 1096 - DNA, 21 bp, synthetic construct
  source 1..21: mol_type = other DNA
ttaaacaaatctccaaaaaaa

SEQ ID NO: 1097 - DNA, 21 bp, synthetic construct
  source 1..21: mol_type = other DNA
gcggccatgccccctgcctat

SEQ ID NO: 1098 - DNA, 21 bp, synthetic construct
  source 1..21: mol_type = other DNA
gtagcatttgagccatttggg

SEQ ID NO: 1099 - DNA, 84 bp, synthetic construct
  source 1..84: mol_type = other DNA
atttgtagcgcataaagataagagccagtttcactcacctccatctccactcctacccatccaactcccactttctagatgtat

SEQ ID NO: 1100 - DNA, 84 bp, synthetic construct
  source 1..84: mol_type = other DNA
attgcgtatattcctaccgaatctaaagtttcactcacctccatctccactcctacccatccaactcccactttctagatgtat

SEQ ID NO: 1101 - DNA, 84 bp, synthetic construct
  source 1..84: mol_type = other DNA
taccatactgattgttaatgcatcaatatttcactcacctccatctccactcctacccatccaactcccactttctagatgtat

SEQ ID NO: 1102 - DNA, 28 bp, synthetic construct
  source 1..28: mol_type = other DNA
aggcaaagcaaggcaacagccatattat

SEQ ID NO: 1103 - DNA, 32 bp, synthetic construct
  source 1..32: mol_type = other DNA
atttacattgggtgaggcggtgtacagaccag

SEQ ID NO: 1104 - DNA, 32 bp, synthetic construct
  source 1..32: mol_type = other DNA
cgaacgtggcgttttagacctcagcagcgaaa

SEQ ID NO: 1105 - DNA, 33 bp, synthetic construct
  source 1..33: mol_type = other DNA
ttgagggcaccgactaacatctcaattctacta

SEQ ID NO: 1106 - DNA, 91 bp, synthetic construct
  source 1..91: mol_type = other DNA
acgcaatgtcaaatcaccatcagccccagttaaaatttcactcacctccatctccactcctacccatccaactcccactttctagatgtat

SEQ ID NO: 1107 - DNA, 35 bp, synthetic construct
  source 1..35: mol_type = other DNA
aagcagaaaattaatgccggaactagcataaccaa

SEQ ID NO: 1108 - DNA, 91 bp, synthetic construct
  source 1..91: mol_type = other DNA
aatatgatattcaaccgttctaccccggttgttaatttcactcacctccatctccactcctacccatccaactcccactttctagatgtat

SEQ ID NO: 1109 - DNA, 91 bp, synthetic construct
  source 1..91: mol_type = other DNA
gagggtagctatttttgagagtcgatgaaaaataatttcactcacctccatctccactcctacccatccaactcccactttctagatgtat

SEQ ID NO: 1110 - DNA, 35 bp, synthetic construct
  source 1..35: mol_type = other DNA
atcgtcgaaagaagagagcggaaagagtctgtcca

SEQ ID NO: 1111 - DNA, 35 bp, synthetic construct
  source 1..35: mol_type = other DNA
aatcttgtgaattattttaagaactggctcattat

SEQ ID NO: 1112 - DNA, 35 bp, synthetic construct
  source 1..35: mol_type = other DNA
aagaaacacaaacaactaacaactaatagattaga

SEQ ID NO: 1113 - DNA, 35 bp, synthetic construct
  source 1..35: mol_type = other DNA
acattatattaaatatctaaaatatcttaccctca

SEQ ID NO: 1114 - DNA, 35 bp, synthetic construct
  source 1..35: mol_type = other DNA
aattaaccgttgtaatccagaagtaacagtacctt

SEQ ID NO: 1115 - DNA, 35 bp, synthetic construct
  source 1..35: mol_type = other DNA
cgggcgctagggcgtagaatcatgatgaaacaaac

SEQ ID NO: 1116 - DNA, 35 bp, synthetic construct
  source 1..35: mol_type = other DNA
agtccactattaaaaatcaagaacatagcgatagc

SEQ ID NO: 1117 - DNA, 35 bp, synthetic construct
  source 1..35: mol_type = other DNA
gagccggagcctcccagacgaaggtttcacgcaac

SEQ ID NO: 1118 - DNA, 35 bp, synthetic construct
  source 1..35: mol_type = other DNA
ttaatgaatcggccgcggtcctaaatgctgatgca

SEQ ID NO: 1119 - DNA, 35 bp, synthetic construct
  source 1..35: mol_type = other DNA
taagaggtcattttagaccggaggtgtatcaccgt

SEQ ID NO: 1120 - DNA, 35 bp, synthetic construct
  source 1..35: mol_type = other DNA
tcacagttgaggattccacacctagaaaaagcctg

SEQ ID NO: 1121 - DNA, 35 bp, synthetic construct
  source 1..35: mol_type = other DNA
ctggtaatgggtaatccagcgaggcagaggcattt

SEQ ID NO: 1122 - DNA, 35 bp, synthetic construct
  source 1..35: mol_type = other DNA
ttacactggtgtgtttacctgaccgacaaaaggta

SEQ ID NO: 1123 - DNA, 35 bp, synthetic construct
  source 1..35: mol_type = other DNA
ccattagatacattgaagtttttgaggcaggtcag

SEQ ID NO: 1124 - DNA, 35 bp, synthetic construct
  source 1..35: mol_type = other DNA
ctccggccagagcaggtggtgaaaccaatcaataa

SEQ ID NO: 1125 - DNA, 35 bp, synthetic construct
  source 1..35: mol_type = other DNA
gataaccgacggccctcaggagtaaccgatatttt

SEQ ID NO: 1126 - DNA, 35 bp, synthetic construct
  source 1..35: mol_type = other DNA
acgtacagcgccattacatcgtatagaaggcttat

SEQ ID NO: 1127 - DNA, 35 bp, synthetic construct
  source 1..35: mol_type = other DNA
tagactttctccgtttaaattagcgaacctcccga

SEQ ID NO: 1128 - DNA, 91 bp, synthetic construct
  source 1..91: mol_type = other DNA
ggtgaagacgccaggcgcaacgtaacaactggccttttcactcacctccatctccactcctacccatccaactcccactttctagatgtat

SEQ ID NO: 1129 - DNA, 36 bp, synthetic construct
  source 1..36: mol_type = other DNA
cactgcccgctttccgatggtgagcgtaacgatcta

SEQ ID NO: 1130 - DNA, 44 bp, synthetic construct
  source 1..44: mol_type = other DNA
  modified_base 1: mod_base = OTHER; note = Biotin modified nucleotide
tttggggtttccggaataagcaaacgagcttcaaagcgaacgct

SEQ ID NO: 1131 - DNA, 98 bp, synthetic construct
  source 1..98: mol_type = other DNA
gaatacccaaagacgccagtttgaggaaatatttaaattgtatttcactcacctccatctccactcctacccatccaactcccactttctagatgtat

SEQ ID NO: 1132 - DNA, 42 bp, synthetic construct
  source 1..42: mol_type = other DNA
cgaggaattattttgcgcatcagatcgcactccagcgacgtt

SEQ ID NO: 1133 - DNA, 95 bp, synthetic construct
  source 1..95: mol_type = other DNA
ttcaaaagggtgagaaaggccgtataagcaaataaaaattttcactcacctccatctccactcctacccatccaactcccactttctagatgtat

SEQ ID NO: 1134 - DNA, 42 bp, synthetic construct
  source 1..42: mol_type = other DNA
tggtttacagtagcgtaaaactcaccggaaacaatcgtaaaa

SEQ ID NO: 1135 - DNA, 45 bp, synthetic construct
  source 1..45: mol_type = other DNA
  modified_base 1: mod_base = OTHER; note = Biotin modified nucleotide
tttgatagcacgtttgcagtgatgaaggggcaaatggtcaataac

SEQ ID NO: 1136 - DNA, 42 bp, synthetic construct
  source 1..42: mol_type = other DNA
aacgtcacaaaatcaaagccgtccggcaaacgcggcagcatc

SEQ ID NO: 1137 - DNA, 42 bp, synthetic construct
  source 1..42: mol_type = other DNA
accgcctaaacaaaagcggggcgggtcactgttgcgcctgtg

SEQ ID NO: 1138 - DNA, 45 bp, synthetic construct
  source 1..45: mol_type = other DNA
  modified_base 1: mod_base = OTHER; note = Biotin modified nucleotide
tttaccgttccagttaagaatgcggcgggcggatggcttagagct

SEQ ID NO: 1139 - DNA, 42 bp, synthetic construct
  source 1..42: mol_type = other DNA
gaaagcgttcggaacactctgtctgccagcacgcggggtgcc

SEQ ID NO: 1140 - DNA, 42 bp, synthetic construct
  source 1..42: mol_type = other DNA
attaagacaccctctaatgagaaacctgtcgtgcccagcagg

SEQ ID NO: 1141 - DNA, 42 bp, synthetic construct
  source 1..42: mol_type = other DNA
gtgcctttttgatggcattgaccaccctgcattttgaatcaa

SEQ ID NO: 1142 - DNA, 42 bp, synthetic construct
  source 1..42: mol_type = other DNA
accctcaaagttttcgaaaattagcccgagataggggaaccc

SEQ ID NO: 1143 - DNA, 42 bp, synthetic construct
  source 1..42: mol_type = other DNA
tgaatttattgtattaaagggaagggaagaaagcgacaggag

SEQ ID NO: 1144 - DNA, 42 bp, synthetic construct
  source 1..42: mol_type = other DNA
tgaatttgacagcagccgattaatcagtgaggccagctcatg

SEQ ID NO: 1145 - DNA, 42 bp, synthetic construct
  source 1..42: mol_type = other DNA
tttttcagagtgagacgcctgacccatggtatagctgctcag

SEQ ID NO: 1146 - DNA, 42 bp, synthetic construct
  source 1..42: mol_type = other DNA
cagaggctataccagaaatacaccagtcacacgacccagcag

SEQ ID NO: 1147 - DNA, 98 bp, synthetic construct
  source 1..98: mol_type = other DNA
ttcattataatttcaccagtcaggacgtagcaccgcctgcaatttcactcacctccatctccactcctacccatccaactcccactttctagatgtat

SEQ ID NO: 1148 - DNA, 42 bp, synthetic construct
  source 1..42: mol_type = other DNA
ccttaacatttgaggatttaggccgtcaatagataattgcga

SEQ ID NO: 1149 - DNA, 42 bp, synthetic construct
  source 1..42: mol_type = other DNA
gtgttgacgctcaatcgtctgacagggccagaatcctgagaa

SEQ ID NO: 1150 - DNA, 42 bp, synthetic construct
  source 1..42: mol_type = other DNA
tttttataaagggaagaaaggagcccccaaaagaacctgttt

SEQ ID NO: 1151 - DNA, 42 bp, synthetic construct
  source 1..42: mol_type = other DNA
gatttagagcttgacggggctaagcaaaatcccttataaatc

SEQ ID NO: 1152 - DNA, 42 bp, synthetic construct
  source 1..42: mol_type = other DNA
actcacattaattgcgttgcctgccgttttcacggtcatacc

SEQ ID NO: 1153 - DNA, 42 bp, synthetic construct
  source 1..42: mol_type = other DNA
agctgcaaagcctgtgcctgtactgcgccctgcggaggtgtc

SEQ ID NO: 1154 - DNA, 42 bp, synthetic construct
  source 1..42: mol_type = other DNA
aggcgctttcgcactcaattgtctaaagttaaacgatgctga

SEQ ID NO: 1155 - DNA, 42 bp, synthetic construct
  source 1..42: mol_type = other DNA
agtgccaagctttcagaggtataggacgacgacagtatcggc

SEQ ID NO: 1156 - DNA, 44 bp, synthetic construct
  source 1..44: mol_type = other DNA
atagtggagccgccacgggaacgggcctttcatcttttcataat

SEQ ID NO: 1157 - DNA, 45 bp, synthetic construct
  source 1..45: mol_type = other DNA
ttcagcgcgttgaagttcagagaatccccctcaaatgaaagccgg

SEQ ID NO: 1158 - DNA, 45 bp, synthetic construct
  source 1..45: mol_type = other DNA
cattaaacaaaagacgtttacgtaagagcaacactataatggatt

SEQ ID NO: 1159 - DNA, 49 bp, synthetic construct
  source 1..49: mol_type = other DNA
acaaagtccctgaaaggtcactccggcaccgcttcacgccagggttttc

SEQ ID NO: 1160 - DNA, 49 bp, synthetic construct
  source 1..49: mol_type = other DNA
tcttaccagataacgattctctcgccattcaggctctggcgaaaggggg

SEQ ID NO: 1161 - DNA, 49 bp, synthetic construct
  source 1..49: mol_type = other DNA
gaagcgccaaaatagattaagagtcccggaatttggccagcagttgggc

SEQ ID NO: 1162 - DNA, 49 bp, synthetic construct
  source 1..49: mol_type = other DNA
gagcgtcaatcagaacataaatttcgtctcgtcgccagcttacggctgg

SEQ ID NO: 1163 - DNA, 49 bp, synthetic construct
  source 1..49: mol_type = other DNA
gcacccagcgtttttctgctcataacggaacgtgcaatgccaacggcag

SEQ ID NO: 1164 - DNA, 49 bp, synthetic construct
  source 1..49: mol_type = other DNA
gtaggaacatgtagccatccctttgctcgtcataaggtgccccctgcat

SEQ ID NO: 1165 - DNA, 49 bp, synthetic construct
  source 1..49: mol_type = other DNA
aagaaaagtaatttcagtgtctcttcgcgtccgtgaagcataaagtgta

SEQ ID NO: 1166 - DNA, 49 bp, synthetic construct
  source 1..49: mol_type = other DNA
tgcagaaataaagtcagccagtaccgagctcgaataaattgttatccgc

SEQ ID NO: 1167 - DNA, 49 bp, synthetic construct
  source 1..49: mol_type = other DNA
ttgagaaataattaaacatacggggagaggcggttgccctgagagagtt

SEQ ID NO: 1168 - DNA, 49 bp, synthetic construct
  source 1..49: mol_type = other DNA
taaggcgctatatgacgctgggttgttccagtttgggtgccgtaaagca

SEQ ID NO: 1169 - DNA, 49 bp, synthetic construct
  source 1..49: mol_type = other DNA
tgacctaacgcgagcccttcagactccaacgtcaacactacgtgaacca

SEQ ID NO: 1170 - DNA, 49 bp, synthetic construct
  source 1..49: mol_type = other DNA
ttttaacccttgaattttttggtgtagcggtcacgcgtataacgtgctt

SEQ ID NO: 1171 - DNA, 49 bp, synthetic construct
  source 1..49: mol_type = other DNA
acataaaacatttatgctttgttctttgattagtaactatcggccttgc

SEQ ID NO: 1172 - DNA, 49 bp, synthetic construct
  source 1..49: mol_type = other DNA
attgtgtgatgaacggtcagtattaaatttaggaataccacaagattca

SEQ ID NO: 1173 - DNA, 49 bp, synthetic construct
  source 1..49: mol_type = other DNA
tgctcatccgaacttgttactaaagaggcgggtaacagggagaaccatc

SEQ ID NO: 1174 - DNA, 49 bp, synthetic construct
  source 1..49: mol_type = other DNA
acaaagctaaattgaaaaatctacgttaggtagaattcaactaggcata

SEQ ID NO: 1175 - DNA, 49 bp, synthetic construct
  source 1..49: mol_type = other DNA
gaaaaacccgagtagagctaaaaaggagctaaatcgttgagttttgccc

SEQ ID NO: 1176 - DNA, 49 bp, synthetic construct
  source 1..49: mol_type = other DNA
agccattgcaacagaaaagggacattctttaaaaatgattatcagatga

SEQ ID NO: 1177 - DNA, 49 bp, synthetic construct
  source 1..49: mol_type = other DNA
tccgtttaaaatcccggcgaaccagtcaccagcttgttggtgtagatgg

SEQ ID NO: 1178 - DNA, 49 bp, synthetic construct
  source 1..49: mol_type = other DNA
tggcagcggttgtggtttaccttgggtatggtgccgaccgtacattttt

SEQ ID NO: 1179 - DNA, 56 bp, synthetic construct
  source 1..56: mol_type = other DNA
tcaccgtagggaagataaagggactccttgtgtaggtaaagatagaaccatttcaa

SEQ ID NO: 1180 - DNA, 56 bp, synthetic construct
  source 1..56: mol_type = other DNA
agagccacaggaggcattccaactaaagtacggtgtcccgccgggcgcggttgcgg

SEQ ID NO: 1181 - DNA, 56 bp, synthetic construct
  source 1..56: mol_type = other DNA
attttcacatagttgttccgaaatcgagcggattgcatcaaattatagtcagaagc

SEQ ID NO: 1182 - DNA, 56 bp, synthetic construct
  source 1..56: mol_type = other DNA
taccgattcgtcaccaggaacggtactaatagtaaaatgtttgttttgccagaggg

SEQ ID NO: 1183 - DNA, 56 bp, synthetic construct
  source 1..56: mol_type = other DNA
gaggcgaaatatacacaatatagagatagaaccctgatagccctaaaacacctcaa

SEQ ID NO: 1184 - DNA, 56 bp, synthetic construct
  source 1..56: mol_type = other DNA
gcgaacttctgacctggtaatgcaatacacgagcactgcgcgtcacccagaacgtg

SEQ ID NO: 1185 - DNA, 56 bp, synthetic construct
  source 1..56: mol_type = other DNA
taccgcctcacgcatcctcgtctggcaagggtcgagaacaaggcagcaaaacgcgc

SEQ ID NO: 1186 - DNA, 56 bp, synthetic construct
  source 1..56: mol_type = other DNA
aagattatttaattctccaaccttttgataattgcatatgcatataacagttgatt

SEQ ID NO: 1187 - DNA, 56 bp, synthetic construct
  source 1..56: mol_type = other DNA
ccgcctgtgcgtattcacaatccccgggcggtgccacatccccaccgtccatcctc

SEQ ID NO: 1188 - DNA, 56 bp, synthetic construct
  source 1..56: mol_type = other DNA
agtcgggtgagctagggggtttggtgcttatgagctcattgcttgccgtcacaggc

SEQ ID NO: 1189 - DNA, 56 bp, synthetic construct
  source 1..56: mol_type = other DNA
atttgcctgagagaatgtgctgcgccatcgtgggagccatcaacggtaatcgtaaa

SEQ ID NO: 1190 - DNA, 27 bp, synthetic construct
  source 1..27: mol_type = other DNA
tttaaacgtagaaaagaccctgtattt

SEQ ID NO: 1191 - DNA, 27 bp, synthetic construct
  source 1..27: mol_type = other DNA
tttattcattaaaggggcaaggcattt

SEQ ID NO: 1192 - DNA, 27 bp, synthetic construct
  source 1..27: mol_type = other DNA
tttctgtagcgcgtttttcatttgttt

SEQ ID NO: 1193 - DNA, 27 bp, synthetic construct
  source 1..27: mol_type = other DNA
tttaccaccagagcccccaattctttt

SEQ ID NO: 1194 - DNA, 27 bp, synthetic construct
  source 1..27: mol_type = other DNA
tttataagttttaacaatgctgtattt

SEQ ID NO: 1195 - DNA, 27 bp, synthetic construct
  source 1..27: mol_type = other DNA
tttgtcgagagggttgattagagattt

SEQ ID NO: 1196 - DNA, 27 bp, synthetic construct
  source 1..27: mol_type = other DNA
tttgtcaccagtacagcccgaaagttt

SEQ ID NO: 1197 - DNA, 27 bp, synthetic construct
  source 1..27: mol_type = other DNA
tttaggaattgcgaaataaatcaattt

SEQ ID NO: 1198 - DNA, 27 bp, synthetic construct
  source 1..27: mol_type = other DNA
tttattcggtcgctgccaatactgttt

SEQ ID NO: 1199 - DNA, 27 bp, synthetic construct
  source 1..27: mol_type = other DNA
tttaaggcaccaaccaaccaaaatttt

SEQ ID NO: 1200 - DNA, 27 bp, synthetic construct
  source 1..27: mol_type = other DNA
tttacggtcaatcatatacataacttt

SEQ ID NO: 1201 - DNA, 27 bp, synthetic construct
  source 1..27: mol_type = other DNA
tttctgacgagaaacgaactaacgttt

SEQ ID NO: 1202 - DNA, 27 bp, synthetic construct
  source 1..27: mol_type = other DNA
tttctggtctggtcaacgggtattttt

SEQ ID NO: 1203 - DNA, 27 bp, synthetic construct
  source 1..27: mol_type = other DNA
tttagagacgcagaagaggttttgttt

SEQ ID NO: 1204 - DNA, 27 bp, synthetic construct
  source 1..27: mol_type = other DNA
ttttgcgggcctctttttgtttaattt

SEQ ID NO: 1205 - DNA, 27 bp, synthetic construct
  source 1..27: mol_type = other DNA
tttcaacattaaatgcaataataattt

SEQ ID NO: 1206 - DNA, 29 bp, synthetic construct
  source 1..29: mol_type = other DNA
aatagcaaaggctatcaggtcattgcttt

SEQ ID NO: 1207 - DNA, 29 bp, synthetic construct
  source 1..29: mol_type = other DNA
gccgccaatacaggagtgtactggtattt

SEQ ID NO: 1208 - DNA, 30 bp, synthetic construct
  source 1..30: mol_type = other DNA
ttttttagttaatttcgttatacaaatttt

SEQ ID NO: 1209 - DNA, 30 bp, synthetic construct
  source 1..30: mol_type = other DNA
tttcttttttaatggtgagaagagtcattt

SEQ ID NO: 1210 - DNA, 30 bp, synthetic construct
  source 1..30: mol_type = other DNA
ttttaatggaagggtacaataacggatttt

SEQ ID NO: 1211 - DNA, 33 bp, synthetic construct
  source 1..33: mol_type = other DNA
tttgcgaacgagtagatttagtttgactgttta

SEQ ID NO: 1212 - DNA, 38 bp, synthetic construct
  source 1..38: mol_type = other DNA
tttacgacgacaataaacaaagtaattctgtccagttt

SEQ ID NO: 1213 - DNA, 38 bp, synthetic construct
  source 1..38: mol_type = other DNA
ttttatttttgaatggctatacgtggcacagacaattt

SEQ ID NO: 1214 - DNA, 38 bp, synthetic construct
  source 1..38: mol_type = other DNA
tttgagtagaagaactcaaataacatcacttgcctttt

SEQ ID NO: 1215 - DNA, 38 bp, synthetic construct
  source 1..38: mol_type = other DNA
tttcgctacagggcgcgtagccgcgcttaatgcgcttt

SEQ ID NO: 1216 - DNA, 38 bp, synthetic construct
  source 1..38: mol_type = other DNA
ttttatcagggcgatggccagggcgaaaaaccgtcttt

SEQ ID NO: 1217 - DNA, 38 bp, synthetic construct
  source 1..38: mol_type = other DNA
tttgtgagacgggcaacaggtttttcttttcaccattt

SEQ ID NO: 1218 - DNA, 38 bp, synthetic construct
  source 1..38: mol_type = other DNA
tttagctgtttcctgtgtgtcgtaatcatggtcatttt

SEQ ID NO: 1219 - DNA, 38 bp, synthetic construct
  source 1..38: mol_type = other DNA
tttggcatcagatgccgggtcagcaaatcgttaacttt

SEQ ID NO: 1220 - DNA, 39 bp, synthetic construct
  source 1..39: mol_type = other DNA
tttttatgcctgagtaatattacgcagtatgttagcttt

SEQ ID NO: 1221 - DNA, 39 bp, synthetic construct
  source 1..39: mol_type = other DNA
tttttctgagagtctggtcctgtagccagctttcatttt

SEQ ID NO: 1222 - DNA, 40 bp, synthetic construct
  source 1..40: mol_type = other DNA
tttttgttgaaaggaattgagagttggcaaatcaacattt

SEQ ID NO: 1223 - DNA, 40 bp, synthetic construct
  source 1..40: mol_type = other DNA
tttcggaatcgtcataaatattcattaaacgagctgacta

SEQ ID NO: 1224 - DNA, 42 bp, synthetic construct
  source 1..42: mol_type = other DNA
tttttgaacaacattattacaataaaacaccagaacgagtag

SEQ ID NO: 1225 - DNA, 43 bp, synthetic construct
  source 1..43: mol_type = other DNA
tgaaagcgtaagaattagtcttttggattatacttctgaattt

SEQ ID NO: 1226 - DNA, 43 bp, synthetic construct
  source 1..43: mol_type = other DNA
taaccaccacacccctatggtacaatttcatttgaattacttt

SEQ ID NO: 1227 - DNA, 43 bp, synthetic construct
  source 1..43: mol_type = other DNA
tgggcgccagggtgctgattgaaaactttttcaaatatatttt

SEQ ID NO: 1228 - DNA, 54 bp, synthetic construct
  source 1..54: mol_type = other DNA
tttaaaccaagtaccgcactccaagagcagcaaccgcaagcggacttatcaaac

SEQ ID NO: 1229 - DNA, 55 bp, synthetic construct
  source 1..55: mol_type = other DNA
tttgagcaagaaacaatgattaagcctgagcgatgttgggaagggcgatcggttt

SEQ ID NO: 1230 - DNA, 55 bp, synthetic construct
  source 1..55: mol_type = other DNA
tttcgtcaaaaatgaaaatacgatttcgctattggatagctctcacggaaaattt

SEQ ID NO: 1231 - DNA, 55 bp, synthetic construct
  source 1..55: mol_type = other DNA
tttaagccttaaatcaagacttgcggacagcgggtagaacgtcagcgtggtgttt

SEQ ID NO: 1232 - DNA, 55 bp, synthetic construct
  source 1..55: mol_type = other DNA
tttgccaaaaggaattacgaatgcagaagggaatcagtgaataaggcttgccttt

SEQ ID NO: 1233 - DNA, 55 bp, synthetic construct
  source 1..55: mol_type = other DNA
tttagcgagaggcttttgccgataaataaaacgtagccggaacgaggcgcagttt

SEQ ID NO: 1234 - DNA, 55 bp, synthetic construct
  source 1..55: mol_type = other DNA
tttaaatcaggtctttaccaatgacctaataatgcccacgcataaccgatatttt

SEQ ID NO: 1235 - DNA, 55 bp, synthetic construct
  source 1..55: mol_type = other DNA
tttacttcaaatatcgcgtagaggaaaactacaaatagaaaggaacaactaattt

SEQ ID NO: 1236 - DNA, 55 bp, synthetic construct
  source 1..55: mol_type = other DNA
tttgtacctttaattgctcaggtcaggatataataccgtaacactgagtttcttt

SEQ ID NO: 1237 - DNA, 55 bp, synthetic construct
  source 1..55: mol_type = other DNA
tttgctcaacatgttttaatgaatatggggtcataccaggcggataagtgccttt

SEQ ID NO: 1238 - DNA, 55 bp, synthetic construct
  source 1..55: mol_type = other DNA
tttgggcgcgagctgaaaagctatatttcatcgcagagccgccaccagaaccttt

SEQ ID NO: 1239 - DNA, 55 bp, synthetic construct
  source 1..55: mol_type = other DNA
tttaagaattagcaaaatttcatacatgaattagtttgcctttagcgtcagattt

SEQ ID NO: 1240 - DNA, 55 bp, synthetic construct
  source 1..55: mol_type = other DNA
tttatacttttgcgggagaacattattacatacgtaaatattgacggaaattttt

SEQ ID NO: 1241 - DNA, 57 bp, synthetic construct
  source 1..57: mol_type = other DNA
cagacgaaaaagaaagactggatagcgtaggcttgaatacgtaatgccactacgttt

SEQ ID NO: 1242 - DNA, 57 bp, synthetic construct
  source 1..57: mol_type = other DNA
ggtggcacaataaaaagcaataccaaaaagcctttctcatatattttaaatgcattt

SEQ ID NO: 1243 - DNA, 65 bp, synthetic construct
  source 1..65: mol_type = other DNA
ttttcttaccagtataaagccatttcgtttctttgcttgtgtcttgtcttcgtcgtgcttgttcc

SEQ ID NO: 1244 - DNA, 65 bp, synthetic construct
  source 1..65: mol_type = other DNA
tttatagtgaatttatcaaaattttcgtttctttgcttgtgtcttgtcttcgtcgtgcttgttcc

SEQ ID NO: 1245 - DNA, 65 bp, synthetic construct
  source 1..65: mol_type = other DNA
ttttcgcctgattgctttgaattttcgtttctttgcttgtgtcttgtcttcgtcgtgcttgttcc

SEQ ID NO: 1246 - DNA, 65 bp, synthetic construct
  source 1..65: mol_type = other DNA
tttaattttaaaagtttgagtatttcgtttctttgcttgtgtcttgtcttcgtcgtgcttgttcc

SEQ ID NO: 1247 - DNA, 71 bp, synthetic construct
  source 1..71: mol_type = other DNA
cgacattagaaacgcaaaagaactggcatttcgtttctttgcttgtgtcttgtcttcgtcgtgcttgttcc

SEQ ID NO: 1248 - DNA, 71 bp, synthetic construct
  source 1..71: mol_type = other DNA
aacataaatcagaggaagccctttttaatttcgtttctttgcttgtgtcttgtcttcgtcgtgcttgttcc

SEQ ID NO: 1249 - DNA, 71 bp, synthetic construct
  source 1..71: mol_type = other DNA
gtcacaatcaatcataccagaaggaaactttcgtttctttgcttgtgtcttgtcttcgtcgtgcttgttcc

SEQ ID NO: 1250 - DNA, 71 bp, synthetic construct
  source 1..71: mol_type = other DNA
ccaatgaaaatcacccagcgccaaagactttcgtttctttgcttgtgtcttgtcttcgtcgtgcttgttcc

SEQ ID NO: 1251 - DNA, 71 bp, synthetic construct
  source 1..71: mol_type = other DNA
ttaccaaccagttaattagacgggagaatttcgtttctttgcttgtgtcttgtcttcgtcgtgcttgttcc

SEQ ID NO: 1252 - DNA, 71 bp, synthetic construct
  source 1..71: mol_type = other DNA
ttagttgataagaaagcagcctttacagtttcgtttctttgcttgtgtcttgtcttcgtcgtgcttgttcc

SEQ ID NO: 1253 - DNA, 71 bp, synthetic construct
  source 1..71: mol_type = other DNA
gaaccgcttattaggcaccgtaatcagttttcgtttctttgcttgtgtcttgtcttcgtcgtgcttgttcc

SEQ ID NO: 1254 - DNA, 71 bp, synthetic construct
  source 1..71: mol_type = other DNA
tttttatacgcgaggctacaattttatctttcgtttctttgcttgtgtcttgtcttcgtcgtgcttgttcc

SEQ ID NO: 1255 - DNA, 71 bp, synthetic construct
  source 1..71: mol_type = other DNA
accggaaccagacattagcaaggccggatttcgtttctttgcttgtgtcttgtcttcgtcgtgcttgttcc

SEQ ID NO: 1256 - DNA, 71 bp, synthetic construct
  source 1..71: mol_type = other DNA
cagtctctattcacccctcagagccgcctttcgtttctttgcttgtgtcttgtcttcgtcgtgcttgttcc

SEQ ID NO: 1257 - DNA, 71 bp, synthetic construct
  source 1..71: mol_type = other DNA
gataagtttacgagtcattaccgcgccctttcgtttctttgcttgtgtcttgtcttcgtcgtgcttgttcc

SEQ ID NO: 1258 - DNA, 71 bp, synthetic construct
  source 1..71: mol_type = other DNA
acatgttttatcattcatcgagaacaagtttcgtttctttgcttgtgtcttgtcttcgtcgtgcttgttcc

SEQ ID NO: 1259 - DNA, 71 bp, synthetic construct
  source 1..71: mol_type = other DNA
ggattaggtataaacagtaagcgtcatatttcgtttctttgcttgtgtcttgtcttcgtcgtgcttgttcc

SEQ ID NO: 1260 - DNA, 71 bp, synthetic construct
  source 1..71: mol_type = other DNA
acagtagagagaatcgcgcctgtttatctttcgtttctttgcttgtgtcttgtcttcgtcgtgcttgttcc

SEQ ID NO: 1261 - DNA, 71 bp, synthetic construct
  source 1..71: mol_type = other DNA
cctattattctgatataaagccagaatgtttcgtttctttgcttgtgtcttgtcttcgtcgtgcttgttcc

SEQ ID NO: 1262 - DNA, 71 bp, synthetic construct
  source 1..71: mol_type = other DNA
gagccacgtaccgcggctgagactcctctttcgtttctttgcttgtgtcttgtcttcgtcgtgcttgttcc

SEQ ID NO: 1263 - DNA, 71 bp, synthetic construct
  source 1..71: mol_type = other DNA
gaccgtgcggaatctcgccatatttaactttcgtttctttgcttgtgtcttgtcttcgtcgtgcttgttcc

SEQ ID NO: 1264 - DNA, 71 bp, synthetic construct
  source 1..71: mol_type = other DNA
caactttcagccctgggatagcaagccctttcgtttctttgcttgtgtcttgtcttcgtcgtgcttgttcc

SEQ ID NO: 1265 - DNA, 71 bp, synthetic construct
  source 1..71: mol_type = other DNA
ctgagagacaaagaaatttaatggtttgtttcgtttctttgcttgtgtcttgtcttcgtcgtgcttgttcc

SEQ ID NO: 1266 - DNA, 71 bp, synthetic construct
  source 1..71: mol_type = other DNA
gtcgtctttccaaattctcagaaccgcctttcgtttctttgcttgtgtcttgtcttcgtcgtgcttgttcc

SEQ ID NO: 1267 - DNA, 71 bp, synthetic construct
  source 1..71: mol_type = other DNA
cttaaacgcctttatctgtatgggattttttcgtttctttgcttgtgtcttgtcttcgtcgtgcttgttcc

SEQ ID NO: 1268 - DNA, 71 bp, synthetic construct
  source 1..71: mol_type = other DNA
ccttgctttagaatctccggcttaggtttttcgtttctttgcttgtgtcttgtcttcgtcgtgcttgttcc

SEQ ID NO: 1269 - DNA, 71 bp, synthetic construct
  source 1..71: mol_type = other DNA
catgaggtgcgggaagttgcgccgacaatttcgtttctttgcttgtgtcttgtcttcgtcgtgcttgttcc

SEQ ID NO: 1270 - DNA, 71 bp, synthetic construct
  source 1..71: mol_type = other DNA
tacaaaaattaatttcaatatatgtgagtttcgtttctttgcttgtgtcttgtcttcgtcgtgcttgttcc

SEQ ID NO: 1271 - DNA, 71 bp, synthetic construct
  source 1..71: mol_type = other DNA
tcggaacgagggcactttgctttcgaggtttcgtttctttgcttgtgtcttgtcttcgtcgtgcttgttcc

SEQ ID NO: 1272 - DNA, 71 bp, synthetic construct
  source 1..71: mol_type = other DNA
tcatcgccagcgattttgaggactaaagtttcgtttctttgcttgtgtcttgtcttcgtcgtgcttgttcc

SEQ ID NO: 1273 - DNA, 71 bp, synthetic construct
  source 1..71: mol_type = other DNA
acagaaatcagatgattattcatttcaatttcgtttctttgcttgtgtcttgtcttcgtcgtgcttgttcc

SEQ ID NO: 1274 - DNA, 71 bp, synthetic construct
  source 1..71: mol_type = other DNA
cccaaatgaggacacgaaatccgcgacctttcgtttctttgcttgtgtcttgtcttcgtcgtgcttgttcc

SEQ ID NO: 1275 - DNA, 71 bp, synthetic construct
  source 1..71: mol_type = other DNA
cattttgtataatctcaaaattatttgctttcgtttctttgcttgtgtcttgtcttcgtcgtgcttgttcc

SEQ ID NO: 1276 - DNA, 71 bp, synthetic construct
  source 1..71: mol_type = other DNA
ggctggctgacctcagagtacaacggagtttcgtttctttgcttgtgtcttgtcttcgtcgtgcttgttcc

SEQ ID NO: 1277 - DNA, 71 bp, synthetic construct
  source 1..71: mol_type = other DNA
aactttaatcattgacaagaaccggatatttcgtttctttgcttgtgtcttgtcttcgtcgtgcttgttcc

SEQ ID NO: 1278 - DNA, 71 bp, synthetic construct
  source 1..71: mol_type = other DNA
aagtattagactttcaccagaaggagcgtttcgtttctttgcttgtgtcttgtcttcgtcgtgcttgttcc

SEQ ID NO: 1279 - DNA, 21 bp, synthetic construct
  source 1..21: mol_type = other DNA
ttaactgaaagaaaattcata

SEQ ID NO: 1280 - DNA, 21 bp, synthetic construct
  source 1..21: mol_type = other DNA
aatagcaaggccaccaccgga

SEQ ID NO: 1281 - DNA, 21 bp, synthetic construct
  source 1..21: mol_type = other DNA
aacgccaacaaacatgaaagt

SEQ ID NO: 1282 - DNA, 21 bp, synthetic construct
  source 1..21: mol_type = other DNA
gggttatatgacgttagtaaa

SEQ ID NO: 1283 - DNA, 21 bp, synthetic construct
  source 1..21: mol_type = other DNA
ttacctgagtagcaacggcta

SEQ ID NO: 1284 - DNA, 21 bp, synthetic construct
  source 1..21: mol_type = other DNA
gaattatcattcatcaagagt

SEQ ID NO: 1285 - DNA, 28 bp, synthetic construct
  source 1..28: mol_type = other DNA
tgattaatggcaacatataaacaaccga

SEQ ID NO: 1286 - DNA, 28 bp, synthetic construct
  source 1..28: mol_type = other DNA
gaaaagtaattgagcgctaataaacagg

SEQ ID NO: 1287 - DNA, 28 bp, synthetic construct
  source 1..28: mol_type = other DNA
aaaagggaattagagccagcaaaccatc

SEQ ID NO: 1288 - DNA, 28 bp, synthetic construct
  source 1..28: mol_type = other DNA
agagaatttatcccaatccaactatttt

SEQ ID NO: 1289 - DNA, 28 bp, synthetic construct
  source 1..28: mol_type = other DNA
agcgacacggtcatagccccccaccctc

SEQ ID NO: 1290 - DNA, 28 bp, synthetic construct
  source 1..28: mol_type = other DNA
ctgaatcccggtattctaagatttcatc

SEQ ID NO: 1291 - DNA, 28 bp, synthetic construct
  source 1..28: mol_type = other DNA
accctcaacgattggccttgatgaattt

SEQ ID NO: 1292 - DNA, 28 bp, synthetic construct
  source 1..28: mol_type = other DNA
caagccgtcggctgtctttcccagctaa

SEQ ID NO: 1293 - DNA, 28 bp, synthetic construct
  source 1..28: mol_type = other DNA
catggctgagtaacagtgcccgattagc

SEQ ID NO: 1294 - DNA, 28 bp, synthetic construct
  source 1..28: mol_type = other DNA
aacaatatcgagccagtaataggcttaa

SEQ ID NO: 1295 - DNA, 28 bp, synthetic construct
  source 1..28: mol_type = other DNA
aagagaaactcaggaggtttacaccctc

SEQ ID NO: 1296 - DNA, 28 bp, synthetic construct
  source 1..28: mol_type = other DNA
acgctcatttagtatcatatgcatcttc

SEQ ID NO: 1297 - DNA, 28 bp, synthetic construct
  source 1..28: mol_type = other DNA
aataggatagcattccacagacaacagt

SEQ ID NO: 1298 - DNA, 28 bp, synthetic construct
  source 1..28: mol_type = other DNA
aaataccaatccaatcgcaagactacct

SEQ ID NO: 1299 - DNA, 28 bp, synthetic construct
  source 1..28: mol_type = other DNA
tgctaaaaggctccaaaaggaagcttga

SEQ ID NO: 1300 - DNA, 28 bp, synthetic construct
  source 1..28: mol_type = other DNA
cataggtttagattaagacgcaaacagt

SEQ ID NO: 1301 - DNA, 28 bp, synthetic construct
  source 1..28: mol_type = other DNA
tgacaacttaaaggccgctttaagtttc

SEQ ID NO: 1302 - DNA, 28 bp, synthetic construct
  source 1..28: mol_type = other DNA
tgaataaatcaagaaaacaaatcgcgca

SEQ ID NO: 1303 - DNA, 28 bp, synthetic construct
  source 1..28: mol_type = other DNA
acttttttcatctttgaccccctgataa

SEQ ID NO: 1304 - DNA, 28 bp, synthetic construct
  source 1..28: mol_type = other DNA
accaagtttacatcgggagaatagaacc

SEQ ID NO: 1305 - DNA, 28 bp, synthetic construct
  source 1..28: mol_type = other DNA
tgctccagaccaactttgaaacaacgta

SEQ ID NO: 1306 - DNA, 28 bp, synthetic construct
  source 1..28: mol_type = other DNA
acgtaaatggcaattcatcaacggaaca

SEQ ID NO: 1307 - DNA, 28 bp, synthetic construct
  source 1..28: mol_type = other DNA
aaaacaggaagattggagacaaataacg

SEQ ID NO: 1308 - DNA, 28 bp, synthetic construct
  source 1..28: mol_type = other DNA
tgtcaatcatatgtagctgattagccga

SEQ ID NO: 1309 - DNA, 28 bp, synthetic construct
  source 1..28: mol_type = other DNA
agcaaacaagagaaatctacaatagcta

SEQ ID NO: 1310 - DNA, 35 bp, synthetic construct
  source 1..35: mol_type = other DNA
accattaccatttccagagcctaatttgcgctaac

SEQ ID NO: 1311 - DNA, 35 bp, synthetic construct
  source 1..35: mol_type = other DNA
taaatcctcattaatatcccatcctaatcctgaac

SEQ ID NO: 1312 - DNA, 35 bp, synthetic construct
  source 1..35: mol_type = other DNA
agaaccgccaccaaataagaataaacactgataaa

SEQ ID NO: 1313 - DNA, 35 bp, synthetic construct
  source 1..35: mol_type = other DNA
cggtttatcagcattaattaattttccctctgtaa

SEQ ID NO: 1314 - DNA, 35 bp, synthetic construct
  source 1..35: mol_type = other DNA
agcgcgaaacaaattttcaggtttaacgtaaagaa

SEQ ID NO: 1315 - DNA, 31 bp, synthetic construct
  source 1..31: mol_type = other DNA
  modified_base 1: mod_base = OTHER; note = Biotin modified nucleotide
ttttcagttgtgggaagggcttgagatggtt

SEQ ID NO: 1316 - DNA, 31 bp, synthetic construct
  source 1..31: mol_type = other DNA
  modified_base 1: mod_base = OTHER; note = Biotin modified nucleotide
tttcagcaaatgaaaaacgaaccacagtaat

SEQ ID NO: 1317 - DNA, 31 bp, synthetic construct
  source 1..31: mol_type = other DNA
  modified_base 1: mod_base = OTHER; note = Biotin modified nucleotide
tttatatcaataggagcattcgacaactcgt

SEQ ID NO: 1318 - DNA, 31 bp, synthetic construct
  source 1..31: mol_type = other DNA
  modified_base 1: mod_base = OTHER; note = Biotin modified nucleotide
tttttcgcattaaatttttgataatcagaaa

SEQ ID NO: 1319 - DNA, 31 bp, synthetic construct
  source 1..31: mol_type = other DNA
  modified_base 1: mod_base = OTHER; note = Biotin modified nucleotide
ttttaggaacacaaacggcggattggaaacc

SEQ ID NO: 1320 - DNA, 38 bp, synthetic construct
  source 1..38: mol_type = other DNA
  modified_base 1: mod_base = OTHER; note = Biotin modified nucleotide
tttcgcaagggctaaatcggttgtaaagcctcagagca

SEQ ID NO: 1321 - DNA, 31 bp, synthetic construct
  source 1..31: mol_type = other DNA
  modified_base 1: mod_base = OTHER; note = Biotin modified nucleotide
tttcatcaccttgctgaatcgccaggccaac

SEQ ID NO: 1322 - DNA, 31 bp, synthetic construct
  source 1..31: mol_type = other DNA
  modified_base 1: mod_base = OTHER; note = Biotin modified nucleotide
tttaacgttatgcatctaccacggaataagt

SEQ ID NO: 1323 - DNA, 31 bp, synthetic construct
  source 1..31: mol_type = other DNA
  modified_base 1: mod_base = OTHER; note = Biotin modified nucleotide
tttatcagctatgggatcaaagtcagagggt

SEQ ID NO: 1324 - DNA, 31 bp, synthetic construct
  source 1..31: mol_type = other DNA
  modified_base 1: mod_base = OTHER; note = Biotin modified nucleotide
tttttcgcgtcccgtcgccacaagaattgag

SEQ ID NO: 1325 - DNA, 38 bp, synthetic construct
  source 1..38: mol_type = other DNA
  modified_base 1: mod_base = OTHER; note = Biotin modified nucleotide
tttcagtgccacgctgaaacagagcagattcctacatt

SEQ ID NO: 1326 - DNA, 39 bp, synthetic construct
  source 1..39: mol_type = other DNA
  modified_base 1: mod_base = OTHER; note = Biotin modified nucleotide
ttttctggtcgaaggttcctttgcccgaacgttattttt

SEQ ID NO: 1327 - DNA, 31 bp, synthetic construct; identical to SEQ ID NO: 1315
  source 1..31: mol_type = other DNA

SEQ ID NO: 1328 - DNA, 31 bp, synthetic construct; identical to SEQ ID NO: 1316
  source 1..31: mol_type = other DNA

SEQ ID NO: 1329 - DNA, 31 bp, synthetic construct; identical to SEQ ID NO: 1317
  source 1..31: mol_type = other DNA

SEQ ID NO: 1330 - DNA, 31 bp, synthetic construct; identical to SEQ ID NO: 1318
  source 1..31: mol_type = other DNA

SEQ ID NO: 1331 - DNA, 31 bp, synthetic construct; identical to SEQ ID NO: 1319
  source 1..31: mol_type = other DNA

SEQ ID NO: 1332 - DNA, 38 bp, synthetic construct; identical to SEQ ID NO: 1320
  source 1..38: mol_type = other DNA

SEQ ID NO: 1333 - DNA, 31 bp, synthetic construct; identical to SEQ ID NO: 1321
  source 1..31: mol_type = other DNA

SEQ ID NO: 1334 - DNA, 31 bp, synthetic construct; identical to SEQ ID NO: 1322
  source 1..31: mol_type = other DNA

SEQ ID NO: 1335 - DNA, 31 bp, synthetic construct; identical to SEQ ID NO: 1323
  source 1..31: mol_type = other DNA

SEQ ID NO: 1336 - DNA, 31 bp, synthetic construct; identical to SEQ ID NO: 1324
  source 1..31: mol_type = other DNA

SEQ ID NO: 1337 - DNA, 38 bp, synthetic construct; identical to SEQ ID NO: 1325
  source 1..38: mol_type = other DNA

SEQ ID NO: 1338 - DNA, 39 bp, synthetic construct; identical to SEQ ID NO: 1326
  source 1..39: mol_type = other DNA

SEQ ID NO: 1339 - DNA, 19060 bp, synthetic construct
  source 1..19060: mol_type = other DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcgcttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgcaaggagggcgtttccgttcttcttcgtcataacttaatgtttttatttaaaataccctctgaaaagaaaggaaacgacaggtgctgaaagcgaggctttttggcctctgtcgtttcctttctctgtttttgtccgtggaatgaacaatggaagtcaacaaaaagcagctggctgacattttcggtgcgagtatccgtaccattcagaactggcaggaacagggaatgcccgttctgcgaggcggtggcaagggtaatgaggtgctttatgactctgccgccgtcataaaatggtatgccgaaagggatgctgaaattgagaacgaaaagctgcgccgggaggttgaagaactgcggcaggccagcgaggcagatctccagccaggaactattgagtacgaacgccatcgacttacgcgtgcgcaggccgacgcacaggaactgaagaatgccagagactccgctgaagtggtggaaaccgcattctgtactttcgtgctgtcgcggatcgcaggtgaaattgccagtattctcgacgggctccccctgtcggtgcagcggcgttttccggaactggaaaaccgacatgttgatttcctgaaacgggatatcatcaaagccatgaacaaagcagccgcgctggatgaactgataccggggttgctgagtgaatatatcgaacagtcaggttaacaggctgcggcattttgtccgcgccgggcttcgctcactgttcaggccggagccacagaccgccgttgaatgggcggatgctaattactatctcccgaaagaatccgcataccaggaagggcgctgggaaacactgccctttcagcgggccatcatgaatgcgatgggcagcgactacatccgtgaggtgaatgtggtgaagtctgcccgtgtcggttattccaaaatgctgctgggtgtttatgcctactttatagagcataagcagcgcaacacccttatctggttgccgacggatggtgatgccgagaactttatgaaaacccacgttgagccgactattcgtgatattccgtcgctgctggcgctggccccgtggtatggcaaaaagcaccgggataacacgctcaccatgaagcgtttcactaatgggcgtggcttctggtgcctgggcggtaaagcggcaaaaaactaccgtgaaaagtcggtggatgtggcgggttatgatgaacttgctgcttttgatgatgatattgaacaggaaggctctccgacgttcctgggtgacaagcgtattgaaggctcggtctggccaaagtccatccgtggctccacgccaaaagtgagaggcacctgtcagattgagcgtgcagccagtgaatccccgcattttatgcgttttcatgttgcctgcccgcattgcggggaggagcagtatcttaaatttggcgacaaagagacgccgtttggcctcaaatggacgccggatgacccctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggcgggattgacccgaccattgtgtatgaacgctcgaaaaaacatgggctgttccgggtgatccccattaaaggggcatccgtctacggaaagccggtggccagcatgccacgtaagcgaaacaaaaacggggtttaccttaccgaaatcggtacggataccgcgaaagagcagatttataaccgcttcacactgacgccggaaggggatgaaccgcttcccggtgccgttcacttcccgaataacccggatatttttgatctgaccgaagcgcagcagctgactgctgaagagcaggtcgaaaaatgggtggatggcaggaaaaaaatactgtgggacagcaaaaagcgacgcaatgaggcactcgactgcttcgtttatgcgctggcggcgctgcgcatcagtatttcccgctggcagctggatctcagtgcgctgctggcgagcctgcaggaagaggatggtgcagcaaccaacaagaaaacactggcagattacgcccgtgccttatccggagaggatgaatgacgcgacaggaagaacttgccgctgcccgtgcggcactgcatgacctgatgacaggtaaacgggtggcaacagtacagaaagacggacgaagggtggagtttacggccacttccgtgtctgacctgaaaaaatatattgcagagctggaagtgcagaccggcatgacacagcgacgcaggggacctgcaggattttatgtatgaaaacgcccaccattcccacccttctggggccggacggcatgacatcgctgcgcgaatatgccggttatcacggcggtggcagcggatttggagggcagttgcggtcgtggaacccaccgagtgaaagtgtggatgcagccctgttgcccaactttacccgtggcaatgcccgcgcagacgatctggtacgcaataacggctatgccgccaacgccatccagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcctcgttcattcacgtttttgaacccgtggaggacgggcagactcgcggtgcaaatgtgttttacagcgtgatggagcagatgaagatgctcgacacgctgcagaacacgcagctgcagagcgccattgtgaaggcgatgtatgccgccaccattgagagtgagctggatacgcagtcagcgatggattttattctgggcgcgaacagtcaggagcagcgggaaaggctgaccggctggattggtgaaattgccgcgtattacgccgcagcgccggtccggctgggaggcgcaaaagtaccgcacctgatgccgggtgactcactgaacctgcagacggctcaggatacggataacggctactccgtgtttgagcagtcactgctgcggtatatcgctgccgggctgggtgtctcgtatgagcagctttcccggaattacgcccagatgagctactccacggcacgggccagtgcgaacgagtcgtgggcgtactttatggggcggcgaaaattcgtcgcatcccgtcaggcgagccagatgtttctgtgctggctggaagaggccatcgttcgccgcgtggtgacgttaccttcaaaagcgcgcttcagttttcaggaagcccgcagtgcctgggggaactgcgactggataggctccggtcgtatggccatcgatggtctgaaagaagttcaggaagcggtgatgctgatagaagccggactgagtacctacgagaaagagtgcgcaaaacgcggtgacgactatcaggaaatttttgcccagcaggtccgtgaaacgatggagcgccgtgcagccggtcttaaaccgcccgcctgggcggctgcagcatttgaatccgggctgcgacaatcaacagaggaggagaagagtgacagcagagctgcgtaatctcccgcatattgccagcatggcctttaatgagccgctgatgcttgaacccgcctatgcgcgggttttcttttgtgcgcttgcaggccagcttgggatcagcagcctgacggatgcggtgtccggcgacagcctgactgcccaggaggcactcgcgacgctggcattatccggtgatgatgacggaccacgacaggcccgcagttatcaggtcatgaacggcatcgccgtgctgccggtgtccggcacgctggtcagccggacgcgggcgctgcagccgtactcggggatgaccggttacaacggcattatcgcccgtctgcaacaggctgccagcgatccgatggtggacggcattctgctcgatatggacacgcccggcgggatggtggcgggggcatttgactgcgctgacatcatcgcccgtgtgcgtgacataaaaccggtatgggcgcttgccaacgacatgaactgcagtgcaggtcagttgcttgccagtgccgcctcccggcgtctggtcacgcagaccgcccggacaggctccatcggcgtcatgatggctcacagtaattacggtgctgcgctggagaaacagggtgtggaaatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaatttacgccgggatatgtcaagccgaagcatgaagtgaatccgcagatgaccctgcgtcgcctgccggatgaagatccgcagaatctggcggacccggcttaccgccgccgtcgcatcatcatgcagaacatgcgtgacgaagagctggccattgctcaggtcgaagagatgcaggcagtttctgccgtgcttaagggcaaatacaccatgaccggtgaagccttcgatccggttgaggtggatatgggccgcagtgaggagaataacatcacgcagtccggcggcacggagtggagcaagcgtgacaagtccacgtatgacccgaccgacgatatcgaagcctacgcgctgaacgccagcggtgtggtgaatatcatcgtgttcgatccgaaaggctgggcgctgttccgttccttcaaagccgtcaaggagaagctggatacccgtcgtggctctaattccgagctggagacagcggtgaaagacctgggcaaagcggtgtcctataaggggatgtatggcgatgtggccatcgtcgtgtattccggacagtacgtggaaaacggcgtcaaaaagaacttcctgccggacaacacgatggtgctggggaacactcaggcacgcggtctgcgcacctatggctgcattcaggatgcggacgcacagcgcgaaggcattaacgcctctgcccgttacccgaaaaactgggtgaccaccggcgatccggcgcgtgagttcaccatgattcagtcagcaccgctgatgctgctggctgaccctgatgagttcgtgtccgtacaactggcgtaatcatggcccttcggggccattgtttctctgtggaggagtccatgacgaaagatgaactgattgcccgtctccgctcgctgggtgaacaactgaaccgtgatgtcagcctgacggggacgaaagaagaactggcgctccgtgtggcagagctgaaagaggagcttgatgacacggatgaaactgccggtcaggacacccctctcagccgggaaaatgtgctgaccggacatgaaaatgaggtgggatcagcgcagccggataccgtgattctggatacgtctgaactggtcacggtcgtggcactggtgaagctgcatactgatgcacttcacgccacgcgggatgaacctgtggcatttgtgctgccgggaacggcgtttcgtgtctctgccggtgtggcagccgaaatgacagagcgcggcctggccagaatgcaataacgggaggcgctgtggctgatttcgataacctgttcgatgctgccattgcccgcgccgatgaaacgatacgcgggtacatgggaacgtcagccaccattacatccggtgagcagtcaggtgcggtgatacgtggtgtttttgatgaccctgaaaatatcagctatgccggacagggcgtgcgcgttgaaggctccagcccgtccctgtttgtccggactgatgaggtgcggcagctgcggcgtggagacacgctgaccatcggtgaggaaaatttctgggtagatcgggtttcgccggatgatggcggaagttgtcatctctggcttggacggggcgtaccgcctgccgttaaccgtcgccgctgaaagggggatgtatggccataaaaggtcttgagcaggccgttgaaaacctcagccgtatcagcaaaacggcggtgcctggtgccgccgcaatggccattaaccgcgttgcttcatccgcgatatcgcagtcggcgtcacaggttgcccgtgagacaaaggtacgccggaaactggtaaaggaaagggccaggctgaaaagggccacggtcaaaaatccgcaggccagaatcaaagttaaccggggggatttgcccgtaatcaagctgggtaatgcgcgggttgtcctttcgcgccgcaggcgtcgtaaaaaggggcagcgttcatccctgaaaggtggcggcagcgtgcttgtggtgggtaaccgtcgtattcccggcgcgtttattcagcaactgaaaaatggccggtggcatgtcatgcagcgtgtggctgggaaaaaccgttaccccattgatgtggtgaaaatcccgatggcggtgccgctgaccacggcgtttaaacaaaatattgagcggatacggcgtgaacgtcttccgaaagagctgggctatgcgctgcagcatcaactgaggatggtaataaagcgatgaaacatactgaactccgtgcagccgtactggatgcactggagaagcatgacaccggggcgacgttttttgatggtcgccccgctgtttttgatgaggcggattttccggcagttgccgtttatctcaccggcgctgaatacacgggcgaagagctggacagcgatacctggcaggcggagctgcatatcgaagttttcctgcctgctcaggtgccggattcagagctggatgcgtggatggagtcccggatttatccggtgatgagcgatatcccggcactgtcagatttgatcaccagtatggtggccagcggctatgactaccggcgcgacgatgatgcgggcttgtggagttcagccgatctgacttatgtcattacctatgaaatgtgaggacgctatgcctgtaccaaatcctacaatgccggtgaaaggtgccgggaccaccctgtgggtttataaggggagcggtgacccttacgcgaatccgctttcagacgttgactggtcgcgtctggcaaaagttaaagacctgacgcccggcgaactgaccgctgagtcctatgacgacagctatctcgatgatgaagatgcagactggactgcgaccgggcaggggcagaaatctgccggagataccagcttcacgctggcgtggatgcccggagagcaggggcagcaggcgctgctggcgtggtttaatgaaggcgatacccgtgcctataaaatccgcttcccgaacggcacggtcgatgtgttccgtggctgggtcagcagtatcggtaaggcggtgacggcgaaggaagtgatcacccgcacggtgaaagtcaccaatgtgggacgtccgtcgatggcagaagatcgcagcacggtaacagcggcaaccggcatgaccgtgacgcctgccagcacctcggtggtgaaagggcagagcaccacgctgaccgtggccttccagccggagggcgtaaccgacaagagctttcgtgcggtgtctgcggataaaacaaaagccaccgtgtcggtcagtggtatgaccatcaccgtgaacggcgttgctgcaggcaaggtcaacattccggttgtatccggtaatggtgagtttgctgcggttgcagaaattaccgtcaccgccagttaatccggagagtcagcgatgttcctgaaaaccgaatcatttgaacataacggtgtgaccgtcacgctttctgaactgtcagccctgcagcgcattgagcatctcgccctgatgaaacggcaggcagaacaggcggagtcagacagcaaccggaagtttactgtggaagacgccatcagaaccggcgcgtttctggtggcgatgtccctgtggcataaccatccgcagaagacgcagatgccgtccatgaatgaagccgttaaacagattgagcaggaagtgcttaccacctggcccacggaggcaatttctcatgctgaaaacgtggtgtaccggctgtctggtatgtatgagtttgtggtgaataatgcccctgaacagacagaggacgccgggcccgcagagcctgtttctgcgggaaagtgttcgacggtgagctgagttttgccctgaaactggcgcgtgagatggggcgacccgactggcgtgccatgcttgccgggatgtcatccacggagtatgccgactggcaccgcttttacagtacccattattttcatgatgttctgctggatatgcacttttccgggctgacgtacaccgtgctcagcctgtttttcagcgatccggatatgcatccgctggatttcagtctgctgaaccggcgcgaggctgacgaagagcctgaagatgatgtgctgatgcagaaagcggcagggcttgccggaggtgtccgctttggcccggacgggaatgaagttatccccgcttccccggatgtggcggacatgacggaggatgacgtaatgctgatgacagtatcagaagggatcgcaggaggagtccggtatggctgaaccggtaggcgatctggtcgttgatttgagtctggatgcggccagatttgacgagcagatggccagagtcaggcgtcatttttctggtacggaaagtgatgcgaaaaaaacagcggcagtcgttgaacagtcgctgagccgacaggcgctggctgcacagaaagcggggatttccgtcgggcagtataaagccgccatgcgtatgctgcctgcacagttcaccgacgtggccacgcagcttgcaggcgggcaaagtccgtggctgatcctgctgcaacagggggggcaggtgaaggactccttcggcgggatgatccccatgttcagggggcttgccggtgcgatcaccctgccgatggtgggggccacctcgctggcggtggcgaccggtgcgctggcgtatgcctggtatcagggcaactcaaccctgtccgatttcaacaaaacgctggtcctttccggcaatcaggcgggactgacggcagatcgtatgctggtcctgtccagagccgggcaggcggcagggctgacgtttaaccagaccagcgagtcactcagcgcactggttaaggcgggggtaagcggtgaggctcagattgcgtccatcagccagagtgtggcgcgtttctcctctgcatccggcgtggaggtggacaaggtcgctgaagccttcgggaagctgaccacagacccgacgtcggggctgacggcgatggctcgccagttccataacgtgtcggcggagcagattgcgtatgttgctcagttgcagcgttccggcgatgaagccggggcattgcaggcggcgaacgaggccgcaacgaaagggtttgatgaccagacccgccgcctgaaagagaacatgggcacgctggagacctgggcagacaggactgcgcgggcattcaaatccatgtgggatgcggtgctggatattggtcgtcctgataccgcgcaggagatgctgattaaggcagaggctgcgtataagaaagcagacgacatctggaatctgcgcaaggatgattattttgttaacgatgaagcgcgggcgcgttactgggatgatcgtgaaaaggcccgtcttgcgcttgaagccgcccgaaagaaggctgagcagcagactcaacaggacaaaaatgcgcagcagcagagcgataccgaagcgtcacggctgaaatataccgaagaggcgcagaaggcttacgaacggctgcagacgccgctggagaaatataccgcccgtcaggaagaactgaacaaggcactgaaagacgggaaaatcctgcaggcggattacaacacgctgatggcggcggcgaaaaaggattatgaagcgacgctgaaaaagccgaaacagtccagcgtgaaggtgtctgcgggcgatcgtcaggaagacagtgctcatgctgccctgctgacgcttcaggcagaactccggacgctggagaagcatgccggagcaaatgagaaaatcagccagcagcgccgggatttgtggaaggcggagagtcagttcgcggtactggaggaggcggcgcaacgtcgccagctgtctgcacaggagaaatccctgctggcgcataaagatgagacgctggagtacaaacgccagctggctgcacttggcgacaaggttacgtatcaggagcgcctgaacgcgctggcgcagcaggcggataaattcgcacagcagcaacgggcaaaacgggccgccattgatgcgaaaagccgggggctgactgaccggcaggcagaacgggaagccacggaacagcgcctgaaggaacagtatggcgataatccgctggcgctgaataacgtcatgtcagagcagaaaaagacctgggcggctgaagaccagcttcgcgggaactggatggcaggcctgaagtccggctggagtgagtgggaagagagcgccacggacagtatgtcgcaggtaaaaagtgcagccacgcagacctttgatggtattgcacagaatatggcggcgatgctgaccggcagtgagcagaactggcgcagcttcacccgttccgtgctgtccatgatgacagaaattctgcttaagcaggcaatggtggggattgtcgggagtatcggcagcgccattggcggggctgttggtggcggcgcatccgcgtcaggcggtacagccattcaggccgctgcggcgaaattccattttgcaaccggaggatttacgggaaccggcggcaaatatgagccagcggggattgttcaccgtggtgagtttgtcttcacgaaggaggcaaccagccggattggcgtggggaatctttaccggctgatgcgcggctatgccaccggcggttatgtcggtacaccgggcagcatggcagacagccggtcgcaggcgtccgggacgtttgagcagaataaccatgtggtgattaacaacgacggcacgaacgggcagataggtccggctgctctgaaggcggtgtatgacatggcccgcaagggtgcccgtgatgaaattcagacacagatgcgtgatggtggcctgttctccggaggtggacgatgaagaccttccgctggaaagtgaaacccggtatggatgtggcttcggtcccttctgtaagaaaggtgcgctttggtgatggctattctcagcgagcgcctgccgggctgaatgccaacctgaaaacgtacagcgtgacgctttctgtcccccgtgaggaggccacggtactggagtcgtttctggaagagcacgggggctggaaatcctttctgtggacgccgccttatgagtggcggcagataaaggtgacctgcgcaaaatggtcgtcgcgggtcagtatgctgcgtgttgagttcagcgcagagtttgaacaggtggtgaactgatgcaggatatccggcaggaaacactgaatgaatgcacccgtgcggagcagtcggccagcgtggtgctctgggaaatcgacctgacagaggtcggtggagaacgttattttttctgtaatgagcagaacgaaaaaggtgagccggtcacctggcaggggcgacagtatcagccgtatcccattcaggggagcggttttgaactgaatggcaaaggcaccagtacgcgccccacgctgacggtttctaacctgtacggtatggtcaccgggatggcggaagatatgcagagtctggtcggcggaacggtggtccggcgtaaggtttacgcccgttttctggatgcggtgaacttcgtcaacggaaacagttacgccgatccggagcaggaggtgatcagccgctggcgcattgagcagtgcagcgaactgagcgcggtgagtgcctcctttgtactgtccacgccgacggaaacggatggcgctgtttttccgggacgtatcatgctggccaacacctgcacctggacctatcgcggtgacgagtgcggttatagcggtccggctgtcgcggatgaatatgaccagccaacgtccgatatcacgaaggataaatgcagcaaatgcctgagcggttgtaagttccgcaataacgtcggcaactttggcggcttcctttccattaacaaactttcgcagtaaatcccatgacacagacagaatcagcgattctggcgcacgcccggcgatgtgcgccagcggagtcgtgcggcttcgtggtaagcacgccggagggggaaagatatttcccctgcgtgaatatctccggtgagccggaggctatttccgtatgtcgccggaagactggctgcaggcagaaatgcagggtgagattgtggcgctggtccacagccaccccggtggtctgccctggctgagtgaggccgaccggcggctgcaggtgcagagtgatttgccgtggtggctggtctgccgggggacgattcataagttccgctgtgtgccgcatctcaccgggcggcgctttgagcacggtgtgacggactgttacacactgttccgggatgcttatcatctggcggggattgagatgccggactttcatcgtgaggatgactggtggcgtaacggccagaatctctatctggataatctggaggcgacggggctgtatcaggtgccgttgtcagcggcacagccgggcgatgtgctgctgtgctgttttggttcatcagtgccgaatcacgccgcaatttactgcggcgacggcgagctgctgcaccatattcctgaacaactgagcaaacgagagaggtacaccgacaaatggcagcgacgcacacactccctctggcgtcaccgggcatggcgcgcatctgcctttacggggatttacaacgatttggtcgccgcatcgaccttcgtgtgaaaacgggggctgaagccatccgggcactggccacacagctcccggcgtttcgtcagaaactgagcgacggctggtatcaggtacggattgccgggcgggacgtcagcacgtccgggttaacggcgcagttacatgagactctgcctgatggcgctgtaattcatattgttcccagagtcgccggggccaagtcaggtggcgtattccagattgtcctgggggctgccgccattgccggatcattctttaccgccggagccacccttgcagcatggggggcagccattggggccggtggtatgaccggcatcctgttttctctcggtgccagtatggtgctcggtggtgtggcgcagatgctggcaccgaaagccagaactccccgtatacagacaacggataacggtaagcagaacacctatttctcctcactggataacatggttgcccagggcaatgttctgcctgttctgtacggggaaatgcgcgtggggtcacgcgtggtttctcaggagatcagcacggcagacgaaggggacggtggtcaggttgtggtgattggtcgctgatgcaaaatgttttatgtgaaaccgcctgcgggcggttttgtcatttatggagcgtgaggaatgggtaaaggaagcagtaaggggcataccccgcgcgaagcgaaggacaacctgaagtccacgcagttgctgagtgtgatcgatgccatcagcgaagggccgattgaaggtccggtggatggcttaaaaagcgtgctgctgaacagtacgccggtgctggacactgaggggaataccaacatatccggtgtcacggtggtgttccgggctggtgagcaggagcagactccgccggagggatttgaatcctccggctccgagacggtgctgggtacggaagtgaaatatgacacgccgatcacccgcaccattacgtctgcaaacatcgaccgtctgcgctttaccttcggtgtacaggcactggtggaaaccacctcaaagggtgacaggaatccgtcggaagtccgcctgctggttcagatacaacgtaacggtggctgggtgacggaaaaagacatcaccattaagggcaaaaccacctcgcagtatctggcctcggtggtgatgggtaacctgccgccgcgcccgtttaatatccggatgcgcaggatgacgccggacagcaccacagaccagctgcagaacaaaacgctctggtcgtcatacactgaaatcatcgatgtgaaacagtgctacccgaacacggcactggtcggcgtgcaggtggactcggagcagttcggcagccagcaggtgagccgtaattatcatctgcgcgggcgtattctgcaggtgccgtcgaactataacccgcagacgcggcaatacagcggtatctgggacggaacgtttaaaccggcatacagcaacaacatggcctggtgtctgtgggatatgctgacccatccgcgctacggcatggggaaacgtcttggtgcggcggatgtggataaatgggcgctgtatgtcatcggccagtactgcgaccagtcagtgccggacggctttggcggcacggagccgcgcatcacctgtaatgcgtacctgaccacacagcgtaaggcgtgggatgtgctcagcgatttctgctcggcgatgcgctgtatgccggtatggaacgggcagacgctgacgttcgtgcaggaccgaccgtcggataagacgtggacctataaccgcagtaatgtggtgatgccggatgatggcgcgccgttccgctacagcttcagcgccctgaaggaccgccataatgccgttgaggtgaactggattgacctcttaagctagctaacagtcctatgaatcaactacttagatggtattagtgacctgtaacagagcattagcgcaaggtgatttttgtcttcttgcgctaattttttgtcatcaaacctgtcgcactccttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtg

(19) United States
(12) Patent Application Publication
(10) Pub. No.: US 2024/0417720 A1
(43) Pub. Date: Dec. 19, 2024

(54) MAKING AND USING STRUCTURED NUCLEIC ACID PARTICLES

(71) Applicant: Nautilus Subsidiary, Inc., Seattle, WA (US)

(72) Inventors: Michael Augusto DARCY, Fremont, CA (US); Hongji QIAN, Sunnyvale, CA (US); Tural AKSEL, Redwood City, CA (US); Sanjib GUHA, Redwood City, CA (US); Chol YUN, San Diego, CA (US); Ilaria SANDEI, Redwood City, CA (US); Kota KANESHIGE, Sunnyvale, CA (US); Jonathan LEANO, Foster City, CA (US); Hugo Iván Arrieta OLIVA, Redwood City, CA (US); Maximilian PRESS, Seattle, WA (US)

(21) Appl. No.: 18/744,286

(22) Filed: Jun. 14, 2024

Related U.S. Application Data
(60) Provisional application No. 63/555,763, filed on Feb. 20, 2024.
(60) Provisional application No. 63/508,220, filed on Jun. 14, 2023.

(51) Int. Cl. C12N 15/11

(52) U.S. Cl. CPC C12N 15/11; C12N 2310/3513; C12N 2310/3517; C12N 2310/3519; C12N 2310/51

(57) ABSTRACT

The present disclosure provides a structured nucleic acid particle comprising a scaffold strand and a plurality of staple strands hybridized to the scaffold, forming a structured nucleic acid particle having a first planar structure coupled to a first protruding structure extending from a face of the first planar structure. Also, the present disclosure provides a structured nucleic acid particle comprising a scaffold strand and a plurality of staple strands hybridized to the scaffold, forming a structured nucleic acid particle having a plurality of planar faces. In some instances, the structured nucleic acid particle has six planar faces.

### CROSS-REFERENCE TO RELATED APPLICATIONS

This application claims priority to U.S. Provisional Application No. 63/508,220 filed on Jun. 14, 2023 and U.S. Provisional Application No. 63/555,763 filed on Feb. 20, 2024, each of which are incorporated herein by reference.

### REFERENCE SEQUENCE LISTING

This application contains a Sequence Listing, which is being submitted electronically in XML file format in accordance with WIPO Standard ST.26 and is hereby incorporated by reference in its entirety. The XML copy, created on Jun. 13, 2024, is named “50109_4016_US_SL.xml” and is 1,242,413 bytes in size.

### BACKGROUND

The ordering of molecules at the nanoscale is a critical problem for numerous technological applications, including analytical and bioanalytical methods, catalysis and biocatalysis. Of particular interest are methods of arranging molecules into arrays on surfaces where the length scales of surface features or surface irregularities often approach the length scale of molecules that are to be arranged at the surface or interface. For example, analytical techniques that resolve single molecules are of interest for numerous biological applications, including genomics, transcriptomics, and proteomics.

It is preferable for many analytical techniques to form arrays that are substantially uniform, both in terms of having an analyte at substantially all sites of the array, and in terms of each of the sites having no more than a single analyte. As the number of sites in arrays approach scales capable of accommodating whole genomes, transcriptomes or proteomes, directed deposition of known analytes at known sites becomes prohibitively impractical. Random deposition of analytes can be used, but uniformity and high occupancy are in tension in accordance with Poisson statistics. As occupancy increases, uniformity decreases and vice versa. What is needed are arrays and methods for their manufacture that provide increased uniformity and occupancy of analytes therein. The present disclosure satisfies this need and others as well.

### SUMMARY

The present disclosure provides a structured nucleic acid particle, including a single stranded nucleic acid scaffold; and a plurality of single stranded nucleic acid staple strands hybridized to the scaffold; wherein the plurality of staple strands hybridized to the scaffold form the scaffold into a structured nucleic acid particle having a first planar structure coupled to a first protruding structure extending from a face of the first planar structure. Optionally, the structured nucleic acid particle includes a plurality of handles selected from the group consisting of one or more analyte handles, one or more dockers, one or more label handles, one or more attachment handles, and a combination thereof. Alternatively or additionally, an analyte is attached to the first protruding structure at a face opposite from the first planar structure. In a further option, the first protruding structure ranges from about 10 nm-40 nm, about 15 nm-40 nm or about 15 nm-35 nm in length. In another option, the structured nucleic acid particle further comprises a second planar structure extending in parallel from the first planar structure. In a yet further option, the structured nucleic acid particle is a nucleic acid origami comprising helices arranged in a hexagonal lattice and optionally, the single stranded nucleic acid scaffold of the nucleic acid origami ranges from about 7 kb-22 kb in length.

The present disclosure provides a structured nucleic acid particle, including a single stranded nucleic acid scaffold; and a plurality of single stranded nucleic acid staple strands hybridized to the scaffold; wherein the plurality of staple strands when hybridized to the scaffold nucleic acid form the scaffold into a structured nucleic acid particle having a plurality of planar faces. In some instances, a structured nucleic acid particle has at least 6 planar faces. Optionally, the structured nucleic acid particle includes a plurality of affinity reagents attached to at least one planar face of the plurality of planar faces, a plurality of tethers attached to at least one planar face of the plurality of planar faces, a plurality of labels attached to at least one planar face of the plurality of planar faces, or any combination thereof. Alternatively or additionally, the structured nucleic acid particle is a nucleic acid origami comprising at least 70 helices arranged in a hexagonal lattice, and optionally the single stranded nucleic acid scaffold of the nucleic acid origami ranges from about 7 kb-22 kb in length. In a further option, the structured nucleic acid particle comprises an isotropic shape, and additionally or alternatively, the isotropic shape is a cube or cuboidal shape. In a yet further option, the structured nucleic acid particle comprises an anisotropic shape.

The present disclosure provides a structured nucleic acid particle, including (a) a scaffold strand having a nucleotide sequence; (b) a staple strand having a nucleotide sequence hybridized to the scaffold strand; and (c) a handle strand having a nucleotide sequence hybridized to the scaffold strand or to the staple strand. Optionally, the structured nucleic acid particle includes a plurality of different staple strands having different nucleotide sequences hybridized to the scaffold strand. Alternatively or additionally, each of the staple strands is hybridized to two non-contiguous regions in the nucleotide sequence of the scaffold strand.

The present disclosure provides a structured nucleic acid particle, including (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand, wherein the one or more first handles comprise a first nucleotide sequence; (d) one or more second handles hybridized to the scaffold strand or to a staple strand, wherein the one or more second handles comprise a second nucleotide sequence; and (e) one or more third handles hybridized to the scaffold strand or to a staple strand, wherein the one or more third handles comprise a third nucleotide sequence. Optionally, the one or more first handles can be configured to attach one or more analytes to the structured nucleic acid particle, the one or more second handles can be configured to attach one or more labels to the structured nucleic acid particle, and/or the one or more third handles can be configured to attach the structured nucleic acid particle to a solid support (e.g., a surface of an array or particle).

The present disclosure provides a method of making a structured nucleic acid particle. The method can include steps of (a) providing a nucleic acid composition including (i) a scaffold strand hybridized to a plurality of different staple strands, and (ii) one or more first handle strands hybridized to the scaffold strand or to a staple strand; (b) hybridizing a first complementary oligonucleotide to at least one of the first handle strands, wherein the first complementary oligonucleotide includes a nucleotide sequence that is complementary to a nucleotide sequence of the at least one first handle strand. Optionally, the nucleic acid composition further includes (iii) one or more second handle strands hybridized to the scaffold strand or to a staple strand; and the method further includes a step of (c) hybridizing a second complementary oligonucleotide to at least one of the second handle strands, wherein the second complementary oligonucleotide includes a nucleotide sequence that is complementary to a nucleotide sequence of the at least one second handle strand. In a further option, the nucleic acid composition further includes (iv) one or more third handle strands hybridized to the scaffold strand or to a staple strand; and the method further includes a step of (d) hybridizing a third complementary oligonucleotide to at least one of the third handle strands, wherein the third complementary oligonucleotide includes a nucleotide sequence that is complementary to a nucleotide sequence of the at least one third handle strand.

Also provided herein is a structured nucleic acid particle, including (a) a scaffold strand having a nucleotide sequence; (b) a plurality of different staple strands having different nucleotide sequences hybridized to the scaffold strand, wherein each of the staple strands is hybridized to two non-contiguous regions in the nucleotide sequence of the scaffold strand; and (c) a handle strand having a nucleotide sequence hybridized to the scaffold strand or to a staple strand, wherein the handle strand is attached to the scaffold strand or to the staple strand by a covalent cross-link.

Further provided is a structured nucleic acid particle including (a) a nucleic acid scaffold hybridized to a plurality of nucleic acid staples to form a nucleic acid origami having an isotropic shape; and (b) an affinity moiety attached to the nucleic acid origami.

The present disclosure provides a structured nucleic acid particle that is adjustable to at least two different conformational states. The structured nucleic acid particle can be attached to an analyte, wherein, in a first of the at least two conformations, the analyte is accessible for binding to an affinity reagent that recognizes the analyte, and wherein in a second of the at least two conformations the analytes is inhibited from binding to the affinity reagent. Optionally, the structured nucleic acid particle is configured to toggle between at least two states, for example, being capable of converting from the first conformation to the second conformation, and also being capable of converting from the second conformation to the first conformation. Accordingly, the structured nucleic acid particle, when in the first conformation, can be bound to the affinity reagent via the analyte. The structured nucleic acid particle can be unbound to (or dissociated from) the affinity reagent in the second conformation, even when the affinity reagent is in diffusional contact with the structured nucleic acid particle.

The present disclosure provides a method of reversibly binding an affinity reagent to an analyte. The method can include steps of (a) binding an analyte to an affinity reagent, wherein the analyte is attached to a structured nucleic acid particle during the binding, wherein the structured nucleic acid is in a first conformation; and (b) converting the structured nucleic acid particle to a second conformation, wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation; and (d) binding a second affinity reagent to the analyte.

The present disclosure provides a method for purifying a structured nucleic acid particle having nucleic acid origami. The method can include steps of (a) providing a mixture comprising a nucleic acid origami and excess reactants for assembly or modification of the nucleic acid origami; and (b) separating the structured nucleic acid particles from the excess reactants. Exemplary excess reactants include, but are not limited to, nucleic acid scaffolds, nucleic acid staples, nucleic acid handles, labels, affinity reagents (e.g., antibodies or nucleic acid aptamers), analytes of interest (e.g., proteins), tethers or dockers.

Optionally, a method for purifying a structured nucleic acid particle having nucleic acid origami can include steps of (a) forming a mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; and (b) separating the structured nucleic acid particles from nucleic acid scaffolds and nucleic acid staples of the mixture.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1A shows a series of reactions for attaching a structured nucleic acid particle (square with solid fill) to a protein (globule), label (seven-point star), and solid support (square with hatched lines) via hybridization of nucleic acid moieties.

FIG. 1B shows a structured nucleic acid particle (square with solid fill) having an analyte handle (dashed line), eight label handles (dotted lines) and nine attachment handles (solid lines).

FIG. 1C shows a diagram of a structured nucleic acid particle (square with solid fill) attached to a single protein analyte (globule) via a single nucleic acid hybrid (dashed double line), eight labels (seven-point stars) via respective nucleic acid hybrids (dotted double lines) and to a solid support (square with hatched lines) via nine nucleic acid hybrids (solid double lines).

FIG. 2A shows a diagram of a reversible reaction in which a structured nucleic acid particle is attached to a protein by a nucleic acid linker strand that can be toggled between an extended conformation (left side of the reaction) and a hairpin conformation (right side of the reaction) by respective presence and absence of an oligonucleotide that is complementary to the linker strand.

FIG. 2B shows a diagram of a reversible reaction in which a structured nucleic acid particle is attached to a protein by a nucleic acid linker strand that can be toggled between a proud conformation (left side of the reaction) and a flush conformation (right side of the reaction) by respective presence and absence of an oligonucleotide that is complementary to the linker strand.

FIG. 2C shows a diagram of a reversible reaction in which a structured nucleic acid particle is attached to a protein by a nucleic acid linker strand that can be toggled between a proud conformation (left side of the reaction) and a flush conformation (right side of the reaction) by respective presence and absence of an oligonucleotide that is complementary to a nucleic acid strand on the surface of the structured nucleic acid particle, the linker strand being complementary to the strand on the surface of the structured nucleic acid particle.

FIG. 2D shows a diagram of a reversible reaction in which a structured nucleic acid particle is attached to a protein by a nucleic acid linker strand that can be toggled between a proud conformation (left side of the reaction) and a flush conformation (right side of the reaction) by respective absence and presence of an oligonucleotide splint that is complementary to the linker strand and to a nucleic acid strand on the surface of the structured nucleic acid particle.

FIG. 3 shows a diagram of a reversible reaction in which a protein is attached to a surface of a structured nucleic acid particle that can be toggled between an open conformation (planar tile on the left side of the reaction) and a closed conformation (cylinder on the right side of the reaction).

FIG. 4A shows an image of a gel loaded as indicated above each lane, wherein “Ladder” is a 1 Kb Plus DNA ladder, “Scaffold” is the Tile2 scaffold, “Tile2” is the folded Tile2 origami including scaffold and staples. Lanes 5 through 18 are labeled with the UV irradiation duration and where relevant the duration of incubation at 60° C.

FIG. 4B shows an image of a gel loaded as indicated above each lane, wherein “Ladder” is a 1 Kb Plus DNA ladder, “Scaffold” is the dTile scaffold, “dTile” is the folded dTile origami including scaffold and staples. Lanes 5 through 18 are labeled with the UV irradiation duration and where relevant the duration of incubation at 60° C.

FIG. 4C shows an image of a gel loaded as indicated above each lane, wherein “Ladder” is a 1 Kb Plus DNA ladder, “Scaffold” is the Tile2 scaffold, “Tile” is the folded Tile2 origami including scaffold and staples. Lanes 5 through 18 are labeled with the duration of UV irradiation in the presence of 8-MOP and where relevant the duration of incubation at 60° C.

FIG. 5A shows an image of a gel loaded as indicated above each lane, wherein “L” is a 1 Kb Plus DNA ladder, “S” is the Tile2 scaffold, “non-cross-linked” is the folded dTile origami including scaffold and staples which was not irradiated with UV light. The labels above lanes 4 and 5 indicate the temperature at which the non-cross-linked Tile2 scaffold was incubated.

FIG. 5B shows an image of a gel loaded as indicated above each lane, wherein “Ladder” is a 1 Kb Plus DNA ladder, “Scaffold” is the Tile2 scaffold, “dTile_310nm” is the folded dTile origami including scaffold and staples which was irradiated with 310 nm UV light. The labels above lanes 4 through 11 indicate the temperature at which the cross-linked dTile was incubated after cross-linking, “Tile_365nm/8-MOP” indicates the Tile2 origami irradiated with 365 nm UV in the presence of 8-MOP and the labels above lanes 13 through 20 indicate the temperature at which the cross-linked Tile2 was incubated after cross-linking.

FIG. 6 shows an image of a gel loaded as indicated above each lane, wherein “Ladder” is a 1 Kb Plus DNA ladder, “Scaffold” is the Tile2 scaffold, “non-cross-linked” is the folded Tile2 origami including scaffold and staples, “310 nm”, “365n/8-MOP” is cross-linked Tile2, “1×FOB-10 rt, 1 h” indicates incubation for 1 hour at room temperature in 1× folding buffer including 10 mM Mg⁺², “1×FOB-10 rt, overnight” indicates incubation overnight at room temperature in 1× folding buffer including 10 mM Mg⁺², “1×FOB-0 rt, 1 h” indicates incubation for 1 hour at room temperature in 1× folding buffer lacking Mg⁺², “1×FOB-0 rt, overnight” indicates incubation overnight at room temperature in 1× folding buffer lacking Mg⁺².

FIG. 7 shows an image of a gel loaded as indicated above each lane, wherein “L” is a 1 Kb Plus DNA ladder, “S” is the Tile2 scaffold, “FOB” indicates incubation overnight at room temperature in 1× folding buffer including 10 mM Mg⁺², “4M Urea” indicates incubation overnight at room temperature in 1× folding buffer including 10 mM Mg⁺² and 4M urea, “8M urea” indicates incubation overnight at room temperature in 1× folding buffer including 10 mM Mg⁺² and 8M urea, “non-cross-linked” is non-cross-linked Tile2, “dTile_310nm” is cross-linked dTile, and “Tile_365nm/8-MOP” is cross-linked Tile 2.

FIG. 8 shows an image of a gel loaded as indicated above each lane, wherein “Ladder” is a 1 Kb Plus DNA ladder; “Scaffold” is the Tile2 scaffold; “FOB10” indicates incubation overnight at room temperature in 1× folding buffer including 10 mM Mg⁺²; “2% SDS”, “6% SDS” and “10% SDS” indicate incubation overnight at room temperature in 1× folding buffer including 10 mM Mg⁺² and one of 2%, 6% or 10% SDS, respectively; “non-cross-linked” is non-cross-linked Tile2; “dTile_310nm” is cross-linked dTile; and “Tile_365nm/8-MOP” is cross-linked Tile 2.

FIG. 9 is a plot of binding rates for an array of T181 peptides in the presence of DNA origami tiles each having a plurality of handles each attached to a fluorophore and having a plurality of handles each attached to an anti-T181 peptide antibody.

FIG. 10A provides diagrammatic representations of a square lattice of nucleic acid helices.

FIG. 10B provides diagrammatic representations of a hexagonal lattice of nucleic acid helices.

FIG. 11 provides a profile view of a nucleic acid origami having a planar region (10) and a post (11).

FIG. 12A shows a diagram demonstrating modes of flexibility or rigidity for a post relative to an origami surface to which it is attached.

FIG. 12B is a perspective view of a cross-section of a post, wherein the cross-section is parallel to the surface plane of the origami domain to which it is attached.

FIG. 12C shows the use of nucleic acid trusses to support a post attached to an origami domain.

FIG. 13 shows two configurations for a post extending from an origami surface.

FIG. 14, in the upper view, shows a cross-section of a ten-helix bundle for a post, and, in the lower view shows a profile of the helices in the post.

FIG. 15A shows dimensions for an isotropic DNA origami structure having a cube shape.

FIG. 15B shows a schematic of helix packing for a DNA origami structure having the cube shape and dimensions of FIG. 15A.

FIG. 15C shows an exemplary helix from the origami structure of FIG. 15B.

FIG. 16 shows a numbering scheme for the helices of the cube-shaped DNA origami of FIG. 15B.

FIGS. 17A, 17B and 17C show photographs of agarose gels used to evaluate yields for origami structures folded in different conditions.

FIG. 18 shows a photograph of an agarose gel used to separate origami structures from excess staples.

FIGS. 19A-19B depict a structured nucleic acid particle. FIG. 19A shows a profile view of a three-dimensional model for a structured nucleic acid particle. FIG. 19B shows a perspective view of the structured nucleic acid particle of FIG. 19A.

FIGS. 20A-20B depict DNA origami having pegboard structures.

FIG. 21 shows a DNA origami structure having a cuboid (cuboidal) or brick shape. Plane A is opposite to Plane B. Plane C is opposite to Plane E. Plane D is opposite to Plane F. A schematic of helix packing for a DNA origami structure having a cuboid or brick shape is shown. An exemplary helix from the origami structure is also shown.

FIG. 22 shows an exemplary DNA origami structure having a cuboid or brick shape with tethers and staple strands on Plane A and label oligonucleotides on Plane B. Plane A is opposite to Plane B.

FIG. 23 shows another exemplary DNA origami structure having a cuboid or brick shape with tethers and staple strands on Planes C, D, E, and F and label oligonucleotides on Plane B. Plane B is opposite to Plane A. Plane D is opposite to Plane E. Plane F is opposite to Plane E.

FIG. 24 shows an AFM image of exemplary DNA origami structures having parallelepipedon structures and post structures.

### DETAILED DESCRIPTION

The present disclosure provides structured nucleic acid particles that can be engineered in a variety of configurations to facilitate analytical and preparative manipulations for molecules of interest. The structured nucleic acid particles can be engineered to attach various moieties in a variety of spatial locations in or on the particle. For example, structured nucleic acid particles having origami structures can be folded to provide nucleic acid strands with unique handle sequences at particular locations relative to each other. Complementary strands can be synthesized to include moieties of interest such as labels, molecular targets (e.g., biologically active molecules) for analytical assays, affinity reagents, unique molecular identifiers (e.g., unique nucleotide sequences or amino acid sequences) or the like. Similarly, complementary strands can be attached to materials such as solid supports, sites of microarrays, other structured nucleic acid particles, or particles containing materials other than nucleic acids. The complementary strands having attached moieties can be contacted with the structured nucleic acid particles under conditions that are amenable to nucleic acid hybridization resulting in spontaneous placement of the moieties at the engineered locations of the handle strands.

A structured nucleic acid particle can be further engineered to attach a variable number of moieties of variable types. In a particular configuration, a structured nucleic acid particle can be configured to attach a single moiety of a given type. For example, a structured nucleic acid particle can be engineered to have a unique handle sequence at a desired location and the particle can be hybridized to a complementary oligonucleotide to which is attached an analyte of interest. As such, a structured nucleic acid particle can be configured to have one and only one moiety of a selected type, such as one and only one analyte of interest. Alternatively or additionally, a structured nucleic acid particle can be configured to have at least 2, 5, 10, 25, 50 or more moieties of a selected type. For example, a structured nucleic acid particle can include multiple strands having the same sequence, wherein the strands are located at predefined locations, and the particle can be incubated with a plurality of oligonucleotides that are complementary to the sequence and attached to a moiety of interest. As a further example, a disc-shaped or tile-shaped structured nucleic acid particle can be engineered to have a plurality of strands on the perimeter of the disc or tile, wherein the strands have the same nucleotide sequence, and a plurality of oligonucleotides having a complementary sequence and attached luminophore can spontaneously hybridize with the particle to attach a plurality of luminophores on the perimeter. Indeed, the ability to engineer structured nucleic acid particles to have a desired number and spatial organization of multiple different moieties can provide powerful tools. Continuing with the example of particles having multiple luminophores on the perimeter, the particles can further include an analyte of interest located on a face of the disc or tile, thereby maintaining adequate separation between the luminophores and analyte to prevent unwanted quenching of the luminophores by the analyte. Separation of moieties can also be achieved by attaching the moieties to different sides or facets of an isotropic origami structure.

Structured nucleic acid particles, such as those having origami structures, are stable in a variety of environments due to extensive Watson-Crick bonding between component strands. However, in some cases it may be desirable to use a structured nucleic acid particle in a relatively harsh environment that compromises the structural integrity of the particle. For example, an analytical method may include a step of non-covalently binding an affinity reagent to a protein which is attached to a structured nucleic acid particle, and a step that uses relatively harsh conditions to remove the affinity reagent from the protein. As set forth herein, a structured nucleic acid particle can be cross-linked to introduce artificial bonds between strands or between regions of a strand, thereby increasing the stability of the particle in harsh environments such as those having chemical denaturants, high temperatures, or low concentrations of divalent cations.

Also provided herein are structured nucleic acid particles that are capable of conformational changes that alter the accessibility of an attached analyte to an affinity reagent that recognizes the analyte. For example, a protein can be attached to a structured nucleic acid particle via a linker or post that can toggle between a proud conformation that facilitates binding of an antibody that recognizes an epitope in the protein and a flush conformation that inhibits binding of the antibody to the protein. In another example, a surface of a structured nucleic acid particle to which an analyte is attached can toggle between an open configuration, in which the analyte is accessible to an affinity reagent, and a closed conformation in which at least a region of the structured nucleic acid particle inhibits the affinity reagent from contacting the analyte. The inhibition can be due to steric blocking, charge repulsion, polarity repulsion or the like.

Terms used herein will be understood to take on their ordinary meaning in the relevant art unless specified otherwise. Several terms used herein and their meanings are set forth below.

As used herein, the term “affinity reagent” or “affinity agent” refers to a molecule or other substance that is capable of specifically or reproducibly binding to an analyte (e.g., polypeptide or fragment thereof) or moiety (e.g., post-translational modification of a polypeptide). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to polypeptides include, but are not limited to, antibodies or functional fragments thereof (e.g., Fab′ fragments, F(ab′)₂ fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof. An affinity reagent or affinity agent can be referred to as an affinity moiety when attached to a retaining component or when complexed with a plurality of other affinity moieties to form a probe.

As used herein, the term “array” refers to a population of analytes (e.g., proteins) that are associated with unique identifiers such that the analytes can be distinguished from each other. A unique identifier can be a solid support (e.g., particle or bead), structured nucleic acid particle (SNAP), retaining component, site (e.g., spatial address) on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent or non-covalent bonds. An array can include different analytes that are each attached to different unique identifiers. An array can include different unique identifiers that are attached to the same or similar analytes. An array can include separate solid supports, or separate sites on the same solid support, that each bear a different analyte, wherein the different analytes can be identified according to the locations of the solid supports or sites. Analytes that can be included in an array can be, for example, nucleic acids such as structured nucleic acid particles, polypeptides, enzymes, affinity reagents, ligands, or receptors.

As used herein, the term “artificial” when used in reference to a substance (e.g., nucleotide or amino acid), means that the substance is made by human activity rather than occurring naturally. For example, a nucleic acid that is made by human activity or has a non-naturally occurring sequence of nucleotides is referred to as an “artificial nucleic acid.” The term “artificial” can be used to refer to a moiety of a molecule, such that an artificial moiety is a moiety that is made by human activity and/or added to a molecule by human activity. For example, an artificial moiety can be present on a nucleotide of a nucleic acid or on an amino acid of a protein.

As used herein, the term “attached” refers to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding profile” refers to a plurality of binding outcomes for a protein or other analyte. The binding outcomes can be obtained from independent binding observations, for example, independent binding outcomes can be acquired using different affinity reagents, respectively. A binding profile can include empirical measurement outcomes, candidate measurement outcomes or both. A binding profile can exclude empirical measurement outcomes or candidate measurement outcomes.

As used herein, the term “bioorthogonal reaction” refers to a chemical reaction that can occur within a biological system (in vitro and/or in vivo) without interfering with some or all native biological processes, functions, or activities of the biological system. A bioorthogonal reaction may be further characterized as being inert to components of a biological system other than those targeted by the bioorthogonal reaction. A bioorthogonal reaction may include a click reaction. Bioorthogonal or click reactions may include Staudinger ligation, copper-free click reactions, nitrone dipole cycloaddition, norbornene cycloaddition, oxanobornadiene cycloaddition, tetrazine ligation, [4+1] cycloaddition, tetrazole photoclick reactions, or quadricyclane ligation. A bioorthogonal reaction may utilize an enzymatic approach, such as attachment between a first molecule and a second molecule by an enzyme such as a sortase, a ligase, or a subtiligase. A bioorthogonal reaction may utilize an irreversible peptide capture system, such as SpyCatcher/SpyTag, SnoopCatcher/SnoopTag, or SdyCatcher/SdyTag. A “bioorthogonal reactant” is a molecule that is converted to a different product in a bioorthogonal reaction.

As used herein, the term “click reaction” refers to a single-step, thermodynamically-favorable conjugation reactions utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −50 kJ/mol, −100 kJ/mol, −200 kJ/mol, −300 kJ/mol, −400 kJ/mol, or less than −500 kJ/mol. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1]cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference. A “click reactant” is a molecule that is converted to a different product in a click reaction.

The term “comprising” is intended herein to be open-ended, including not only the recited elements, but further encompassing any additional elements.

As used herein, the term “conformation,” when used in reference to a molecule or particle, refers to the shape or proportionate dimensions of the molecule or particle. At the molecular level conformation can be characterized by the spatial arrangement of a molecule that results from the rotation of its atoms about their bonds. The conformation of a macromolecule, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A molecule or particle having a given composition may take on more than one conformation with or without changes to its composition. For example, a protein having a given amino acid sequence (i.e. protein primary structure) may take on different conformations at the secondary, tertiary or quaternary level, and a nucleic acid having a given nucleotide sequence (i.e. nucleic acid primary structure) may take on different conformations at the secondary, tertiary or quaternary level.

As used herein, the term “docker” refers to a molecule or moiety that is configured to interact with a tether or that is interacting with a tether. A docker can be a moiety of a substance, object, molecule, solid support, address or site of an array, particle, or bead. A docker can include a polymer, nucleic acid strand, nucleic acid duplex, nucleotide sequence, protein, affinity reagent, epitope, paratope, receptor, ligand or the like. A docker can interact with a tether via covalent or non-covalent bonding.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to a molecule or part of a molecule, which is recognized by or binds specifically to an affinity reagent. Epitopes may include amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, miniprotein or other probe. An epitope can optionally bind an antibody to elicit an immune response. However, an epitope need not necessarily participate in, nor be capable of, eliciting an immune response.

As used herein, the term “exogenous,” when used in reference to a moiety of a molecule, means a moiety that is not present in a natural analog of the molecule. For example, an exogenous label of an amino acid is a label that is not present on a naturally occurring amino acid. Similarly, an exogenous label that is present on an antibody is not found on the antibody in its native milieu.

As used herein, the term “fluid-phase,” when used in reference to a molecule, means the molecule is in a state wherein it is mobile in a fluid, for example, being capable of diffusing through the fluid. A fluid-phase molecule is not in a state of immobilization on a solid-phase support.

As used herein, the term “handle,” refers to a molecule, moiety, structure or other group on one structural entity, that provides a means for attachment, coupling, affinity, or other association between that structural entity and another molecular or structural entity. For example, in some cases as described herein, a handle may be configured to interact with one or more of a scaffold strand, a staple strand, a plurality of staple strands, an analyte, and a plurality of analytes. A handle can be a moiety of a substance, object, molecule, solid support, address or site of an array, particle, or bead. A handle can include a polymer, nucleic acid strand, nucleic acid duplex, nucleotide sequence, protein, affinity reagent, epitope, paratope, receptor, ligand, reactive chemical moiety, or the like. A handle can be a double stranded nucleic acid molecule or a double stranded nucleic acid molecule. In some configurations, a handle can include a handle strand, an oligonucleotide that hybridizes with a handle strand, a staple strand, or a region of a scaffold strand. In some embodiments, a rigid handle is a post which is also referred to as a protruding structure.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g., for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “measurement outcome” refers to information resulting from observation or examination of a process. For example, the measurement outcome for contacting an affinity reagent with an analyte, such as a protein, can be referred to as a “binding outcome.” A measurement outcome can be positive or negative. For example, observation of binding is a positive binding outcome and observation (or perception) of non-binding is a negative binding outcome. A measurement outcome can be a null outcome in the event an apparent positive or negative outcome does not result from a given measurement.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include polyhedrons having 6, 7, 8, 9, 10 or more sides; cuboids such as cubes or rectangular cuboids; nanotubes; nanowires; cages; tiles; disks; nanospheres; blocks; bricks; and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “paratope” refers to a molecule or part of an affinity reagent, which recognizes or binds specifically to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

As used herein, the term “protruding structure” or “post,” as used interchangeably, refers to a relatively rigid molecular structure that in many cases may include at least one end, a handle. In the context of an origami based post, such a post may include multiple helices that form a lattice (e.g., square lattice or hexagonal lattice). In some embodiments, a post includes 1, 2, 3, 4, 5, 6, 7, 8, 9, 10, 11, 12, 13, 14, 15, 16, 17, 18, 19, 20 or more helices. The helices can run along the length of the post (i.e. the length running from a surface (planar surface) of the structured nucleic acid particle to a point at which an analyte of interest is attached). In some instances, helices can run orthogonal to the length of the post, for example stacked like logs in the wall of a log cabin. A post can include a handle strand, an oligonucleotide that hybridizes with a handle strand, a staple strand, or a region of a scaffold strand. In some configurations, a post can include multiple regions of a scaffold strand. As such a scaffold strand can have helices running along a first plane (e.g., an xy plane in a Cartesian system) and can also include one or more helices running non-parallel to the first plane (e.g., running orthogonal along the z axis of the Cartesian system).

As used herein, the term “protein” refers to a molecule including two or more amino acids joined by a peptide bond. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

As used herein, the term “site,” when used in reference to an array, means a location in an array occupied by, or configured to be occupied by, a particular molecule or analyte such as a protein, nucleic acid, structured nucleic acid particle or reactive moiety. A site can contain only a single molecule, or it can contain a population of several molecules of the same species (i.e. an ensemble of the molecules). Alternatively, a site can include a population of molecules that are different species. Sites of an array are typically discrete. The discrete sites can be contiguous, or they can have interstitial spaces between each other. An array useful herein can have, for example, sites that are separated by less than 100 microns, 10 microns, 1 micron, 0.5 micron, 0.1 micron, 0.01 micron or less. Alternatively or additionally, an array can have sites that are separated by at least 0.01 micron, 0.1 micron, 0.5 micron, 1 micron, 10 microns, 100 microns or more. The sites can each have an area of less than 1 square millimeter, 500 square microns, 100 square microns, 25 square microns, 1 square micron or less. An array can include at least about 1×10⁴, 1×10⁵, 1×10⁶, 1×10⁷, 1×10⁸, 1×10⁹, 1×10¹⁰, 1×10¹¹, 1×10¹², or more sites, some or all of which are occupied by analytes or molecules.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass, modified or functionalized glass, plastics (including acrylics, polystyrene and copolymers of styrene and other materials, polypropylene, polyethylene, polybutylene, polyurethanes, Teflon™ cyclic olefins, polyimides etc.), nylon, ceramics, resins, Zeonor™, silica or silica-based materials including silicon and modified silicon, carbon, metals, inorganic glasses, optical fiber bundles, and polymers.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure (i.e. the helical twist or direction of the polynucleotide strand) of a SNAP can be configured to be more dense than a nucleic acid molecule of similar length in a random coil or other non-structured state. A SNAP can optionally be modified to permit attachment of additional molecules to the SNAP. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “tether” refers to a molecule or moiety that is configured to interact with a docker or that is interacting with a docker. A tether can be a moiety of a substance, object, molecule, solid support, address or site of an array, particle, or bead. A tether can include a polymer, nucleic acid strand, nucleic acid duplex, nucleotide sequence, protein, affinity reagent, epitope, paratope, receptor, ligand or the like. A tether can interact with a docker via covalent or non-covalent bonding.

As used herein, the term “unique identifier” refers to a moiety, object or substance that is associated with an analyte and that is distinct from other identifiers, throughout one or more steps of a process. The moiety, object or substance can be, for example, a solid support such as a particle or bead; a location on a solid support; a site in an array; a tag; a label such as a luminophore; a molecular barcode such as a nucleic acid having a unique nucleotide sequence or a polypeptide having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

As used herein, the term “vessel” refers to an enclosure that contains a substance. The enclosure can be permanent or temporary with respect to the timeframe of a method set forth herein or with respect to one or more steps of a method set forth herein. Exemplary vessels include, but are not limited to, a well (e.g., in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

The present disclosure provides a structured nucleic acid particle, including (a) a scaffold strand having a nucleotide sequence; and (b) a staple strand having a nucleotide sequence hybridized to the scaffold strand. The present disclosure also provides a structured nucleic acid particle, including (a) a scaffold strand having a nucleotide sequence; (b) a staple strand having a nucleotide sequence hybridized to the scaffold strand; and (c) a handle strand having a nucleotide sequence hybridized to the scaffold strand or to the staple strand. Optionally, the structured nucleic acid particle includes a plurality of different staple strands having different nucleotide sequences hybridized to the scaffold strand. Alternatively or additionally, each of the staple strands is hybridized to two non-contiguous regions in the nucleotide sequence of the scaffold strand.

Structured nucleic acid particles can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Further examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference.

In some cases, the overall shape of a nucleic acid origami can be anisotropic. For example, a disk- or tile-shaped origami can include a side, planar face (e.g., face), or facet having a relatively large surface area compared to one or more other sides of the structure that are orthogonal to the side, planar face (e.g., face), or facet. The side, planar face (e.g., face), or facet can provide a surface that interacts with a solid support, for example, at an array feature. As such, the shape of the side, planar face (e.g., face), or facet can provide a footprint for the origami structure. In some cases, the footprint can be complementary to the shape of a surface feature such as an address or site of an array. The relative sizes and shapes for the nucleic acid origami footprint and the surface feature can be configured to accommodate a single origami structure while sterically hindering any additional origami structures from simultaneously occupying the surface feature. An analyte (e.g., protein) can be attached to a side, planar face (e.g., face), or facet of a disk-shaped or tile-shaped origami. The attachment can optionally occur via a linker, handle or post (protruding structure). The side, face, or facet of the origami to which the analyte is attached can be opposite a side, face, or facet of the origami that attaches to a surface feature. As such, the surface area of the origami face can provide spatial isolation of the analyte from other analytes that are attached to other addresses or sites in an array. Other anisotropic shapes for nucleic acid origami include, but are not limited to, cylinders, cones, ovoids, rectangular cuboids, or tubules.

In other cases, the overall shape of a nucleic acid origami can be isotropic. For example, an isotropic nucleic acid origami can have a polyhedron shape in which all sides or facets of the polyhedron have substantially the same surface area and shape. In some cases, an isotropic polyhedron can have sides, faces, or facets with differing surface areas and/or shapes. An example is a Goldberg polyhedron having a combination of hexagons and pentagons such as an overall shape that is recognizable in a soccer ball or buckyball. Isotropic polyhedrons can have 6, 8, 10, 12 or more sides. As the number of sides of an isotropic polyhedron increases the overall shape approaches a sphere shape, which is also isotropic.

Isotropic nucleic acid origami can be particularly useful for use in fluid phase. For example, the radius of gyration for an isotropic origami can be smaller than that of an anisotropic origami having the same mass. The isotropic shape can increase rate of diffusion compared to anisotropic shapes of the same mass. Moreover, the fluid dynamics of an isotropic origami can be more predictable than an anisotropic origami having the same mass. An isotropic nucleic acid origami can be attached to one or more affinity moieties (e.g., antibodies or aptamers), thereby providing a probe. The affinity moieties can be present on one or more sides of the nucleic acid origami. For example, affinity moieties can be present on all sides of an isotropic nucleic acid structure. This can provide a higher probability of the probe binding to an immobilized analyte that is recognized by the affinity moieties. An exemplary nucleic acid origami having an isotropic shape and options for attaching affinity moieties and other moieties are described in Example VI.

Accordingly, the present disclosure provides a structured nucleic acid particle including (a) a nucleic acid scaffold hybridized to a plurality of nucleic acid staples to form a nucleic acid origami having an isotropic shape; and (b) an affinity moiety attached to the nucleic acid origami. Optionally, the isotropic shape is a cube. The cube can include sides having dimensions of at least 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, or longer. Alternatively or additionally, the cube can include sides having dimensions of at most 32 nm, 31 nm, 30 nm, 29 nm, 28 nm, 27 nm, 26 nm, 25 nm, 24 nm, 23 nm, 22 nm, 21 nm, 20 nm, 19 nm, 18 nm, 17 nm, 16 nm, 15 nm, 14 nm, 13 nm, 12 nm or less. More generally, an isotropic nucleic acid origami can have a minimum length of at least 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, or longer. Alternatively or additionally, an isotropic nucleic acid origami can have a maximum length of at most 32 nm, 31 nm, 30 nm, 29 nm, 28 nm, 27 nm, 26 nm, 25 nm, 24 nm, 23 nm, 22 nm, 21 nm, 20 nm, 19 nm, 18 nm, 17 nm, 16 nm, 15 nm, 14 nm, 13 nm, 12 nm or less. In some embodiments, the dimensions of the cube are from about 15-30 nm on a side, and particularly approximately 24 nm on a side+/−5 nm. In some embodiments, the dimensions of the isotropic shape are from about 10-35 nm on a side, and particularly approximately 15-30 nm on a side+/−5 nm.

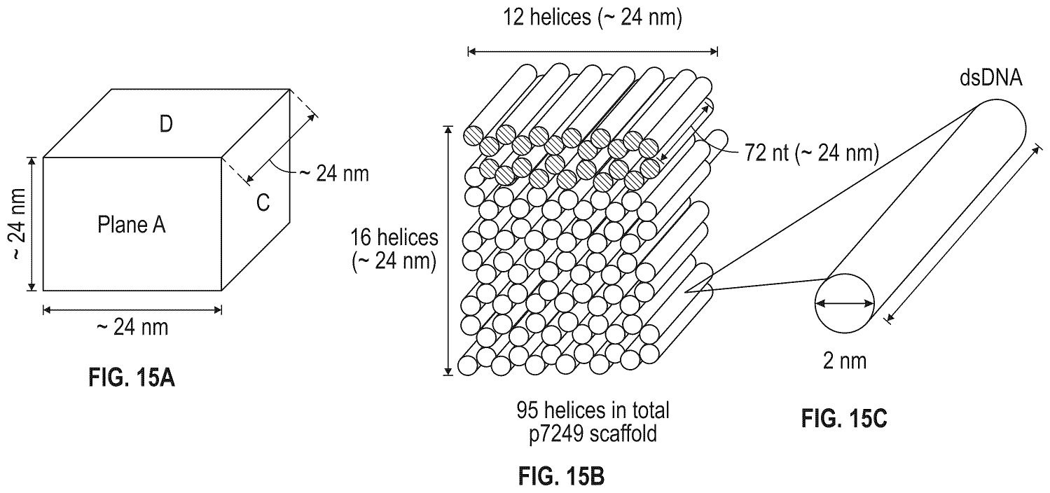

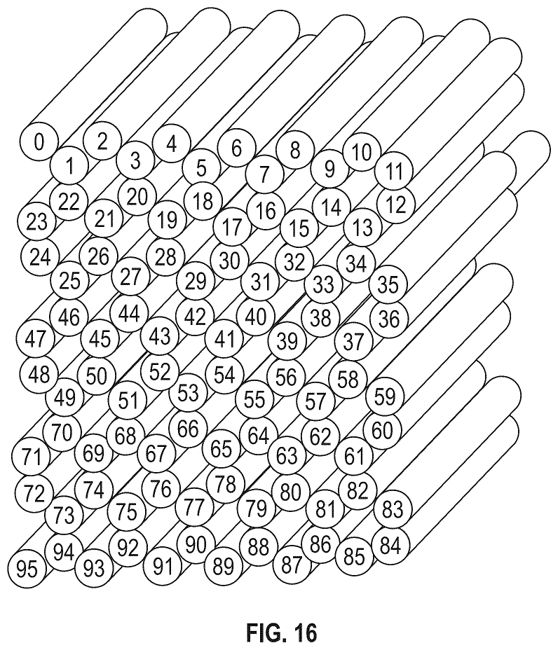

An isotropic nucleic acid origami can include a plurality of helices that run parallel to each other, the helices being packed to form a hexagonal lattice. See, for example, FIGS. 15 and 16 in which 96 helices are hexagonally packed in a cube having dimensions of 24 nm×24 nm×24 nm. In some embodiments, an isotropic nucleic acid origami includes 20, 25, 30, 35, 40, 45, 50, 55, 60, 61, 62, 63, 64, 65, 66, 67, 68, 69, 70, 71, 72, 73, 74, 75, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more helices packed to form a specified architecture. In some instances, an isotropic nucleic acid origami includes 20, 25, 30, 35, 40, 45, 50, 55, 60, 65, 70, 75, 80, 85, 90, 95, 100, 105, 110 or more helices packed to form a hexagonal lattice. In many embodiments, any nucleic acid origami described herein such as, but not limited to, an anisotropic nucleic acid origami and a nucleic acid origami containing a protruding structure includes 20, 25, 30, 35, 40, 45, 50, 55, 60, 61, 62, 63, 64, 65, 66, 67, 68, 69, 70, 71, 72, 73, 74, 75, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more helices. In many embodiments, any nucleic acid origami described herein includes 20, 25, 30, 35, 40, 45, 50, 55, 60, 61, 62, 63, 64, 65, 66, 67, 68, 69, 70, 71, 72, 73, 74, 75, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 or more helices packed to form a hexagonal lattice.

An isotropic nucleic acid origami can display affinity moieties, analytes of interest, labels, tethers, dockers, handles, posts or other moieties set forth herein on at least one side, face, or facet. Optionally, moieties set forth herein can be displayed on a plurality of sides, face, or facets, up to and including all sides, face, or facets, of an isotropic nucleic acid origami. One or more of the sides, face, or facets can include an arrangement of moieties that is the same as or similar to an arrangement set forth herein for other origami shapes. For example, a side, face, or facet of an isotropic nucleic acid origami can display at least one affinity moiety and can optionally also display at least one tether. In another example, a side, face, or facet of an isotropic nucleic acid origami can display at least one analyte of interest (e.g., protein) and can optionally also display at least one docker. In some cases, it may be beneficial to display different moieties on different sides, faces, or facets of an isotropic nucleic acid origami. For example, an isotropic nucleic acid origami can display labels on at least one side, face, or facet and can also display affinity moieties on at least one other side or faces. It will be understood that moieties can be displayed on a side or facet of an isotropic nucleic acid origami due to being attached to a region of the nucleic acid structure that effectively occurs on the side, face, or facet.

In some configurations a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; US Pat. App. Pub. No. 2022/0162684 A1, or U.S. patent application Ser. No. 18/058,000, each of which is incorporated herein by reference.

A structured nucleic acid particle may have any of a variety of sizes and shapes to accommodate use in a desired application. As set forth above, a structured nucleic acid particle can have a regular or symmetric shape (e.g., an isotropic shape) or, alternatively, a structured nucleic acid particle can have an irregular or asymmetric shape (e.g., an anisotropic shape). The shape can be rigid or pliable. The size or shape of a structured nucleic acid particle can be characterized with respect to length, area, or volume. The length, area or volume can be characterized in terms of a minimum, maximum, or average for a population. For example, a structured nucleic acid particle can have a minimum, maximum or average length of at least about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285 nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385 nm, 390 nm, 395 nm, 400 nm, 410 nm, 420 nm, 430 nm, 440 nm, 450 nm, 460 nm, 470 nm, 480 nm, 490 nm, 500 nm, 600 nm, 700 nm, 800 nm, 900 nm, 1 μm, 2 um, 3 μm, 4 um, 5 μm or more. Alternatively or additionally, a structured nucleic acid particle can have a minimum, maximum or average length of no more than about 5 μm, 4 um, 3 μm, 2 um, 1 μm, 900 nm, 800 nm, 700 nm, 600 nm, 500 nm, 490 nm, 480 nm, 470 nm, 460 nm, 450 nm, 440 nm, 430 nm, 420 nm, 410 nm, 400 nm, 395 nm, 390 nm, 385 nm. 380 nm, 375 nm, 370 nm, 365 nm, 360 nm, 355 nm, 350 nm, 345 nm, 340 nm, 335 nm, 330 nm, 325 nm, 320 nm, 315 nm, 310 nm, 305 nm, 300 nm, 295 nm, 290 nm, 285 nm. 280 nm, 275 nm, 270 nm, 265 nm, 260 nm, 255 nm, 250 nm, 245 nm, 240 nm, 235 nm, 230 nm, 225 nm, 220 nm, 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, 10 nm or less. Optionally, a structured nucleic acid particle can have a minimum, maximum or average volume of at least about 1 um³, 10 um³, 100 um³, 1 mm³ or more. Alternatively or additionally, a structured nucleic acid particle can have a minimum, maximum or average volume of no more than about 1 mm³, 100 um³, 10 um³, 1 um³ or less.

As described herein, a structured nucleic acid particle may have any of a variety of sizes and shapes including, but not limited to, a cube, a brick, a parallelepipedon structure set forth in Examples VI, IX, and XII.

A structured nucleic acid particle can be characterized with respect to its footprint (e.g., occupied area on a surface). The footprint may have a regular shape or an approximately regular shape, such as triangular, square, rectangular, circular, ovoid, or polygonal shape. Optionally, the minimum, maximum or average area for a structured nucleic acid particle footprint can be at least about 10 nm², 100 nm², 1 um², 10 um², 100 um², 1 mm² or more. Alternatively or additionally, the minimum, maximum or average area for a structured nucleic acid particle footprint can be at most about 1 mm², 100 um², 10 um², 1 um², 100 nm², 10 nm², or less.

The footprint of a structured nucleic acid particle can be composed of an origami structure including a plurality of scaffold strands. For example, the origami structure can include at least 2, 3, 4 or more scaffold strands. Alternatively, the footprint of a structured nucleic acid particle can be composed of an origami structure including a single scaffold strand, albeit hybridized to a plurality of oligonucleotides. Thus, the footprint of a structured nucleic acid particle can be composed of one and only one scaffold strand. The size of the scaffold (i.e. nucleotide sequence length of the scaffold) used to form a nucleic acid origami can be increased to increase the size of the footprint of the origami. For example, a scaffold can have a length of at least 7.5 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb or more. Alternatively or additionally, a scaffold strand can have a length of at most 30 kb, 29 kb, 28 kb, 27 kb, 26 kb, 25 kb, 24 kb, 23 kb, 22 kb, 21 kb, 20 kb, 19 kb, 18 kb, 17 kb, 16 kb, 15 kb, 14 kb, 13 kb, 12 kb, 11 kb, 10 kb, 9 kb, 8 kb, 7.5 kb or less. In some embodiments, the single stranded nucleic acid scaffold ranges in length from about 7 kb-22 kb, about 8 kb-22 kb, about 9 kb-22 kb, about 10 kb-22 kb, about 11 kb-22 kb, about 12 kb-22 kb, about 13 kb-22 kb, about 14 kb-22 kb, about 7 kb-20 kb, about 7 kb-19 kb, about 7 kb-18 kb, about 8 kb-22 kb, about 8 kb-21 kb, about 8 kb-20 kb, about 8 kb-19 kb, or the like. In some embodiments, the single stranded nucleic acid scaffold has a nucleotide sequence set forth in SEQ ID NO: 1339.

A structured nucleic acid particle that is made or used in a method set forth herein can be in a fluid phase state (e.g., suspended in a fluid) or in a solid phase state (e.g., immobilized on a solid support or other non-fluid such as a gel or solid support material). For example, a population of structured nucleic acid particles can be colloidal for some, or all steps of a method set forth herein. Alternatively, a population of structured nucleic acid particles can be immobilized in, or on a solid support for some, or all steps of a method set forth herein.

In some configurations, a nucleic acid origami may include a scaffold strand and a plurality of staple strands. The scaffold can be configured as a single, continuous strand of nucleic acid, and the staples can be formed by nucleic acid strands that hybridize, in whole or in part, with the scaffold strand. A structured nucleic acid particle (e.g., having origami or nanoball structures) may include regions of single-stranded nucleic acid, regions of double-stranded nucleic acid, or combinations thereof. A structured nucleic acid particle (e.g., having origami or nanoball structures) can include an artificial cross-link between two or more nucleic acid strands therein.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e., having a 3′ end and 5′ end) or circular (i.e., closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include a plurality of oligonucleotides that are hybridized to a scaffold strand. A first region of an oligonucleotide sequence can be hybridized to a scaffold strand while a second region is not hybridized to the scaffold strand. One or both of the regions can be located at or near the 5′ end of the oligonucleotide, at or near the 3′ end of the oligonucleotide, or in a region of the oligonucleotide that is between the end regions. The second region of the oligonucleotide can be in a single stranded state or, alternatively, can participate in a hairpin or other self-annealed structure in the oligonucleotide. Optionally, the second region of the oligonucleotide can include an attachment moiety that is configured to form a covalent or non-covalent bond with a reactive moiety, such as an amino acid of a polypeptide, a reactive moiety attached to the surface of a solid support or a reactive moiety of a label. As set forth in further detail herein, an attachment moiety of a particle can bond with a reactive moiety of a protein. In some cases, the second region of an oligonucleotide component of a nucleic acid origami can hybridize to a complementary oligonucleotide to form a double-stranded region. The first and second regions of the oligonucleotide can be adjacent to each other in the nucleotide sequence of the oligonucleotide or separated by a spacer region in the sequence. The spacer region can be single stranded, for example, to provide relative flexibility. Alternatively, the spacer region can be double stranded or at least partially double stranded, for example, to provide relative rigidity.

An oligonucleotide can include two sequence regions that are hybridized to a scaffold strand, for example, to function as a “staple” that constrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand. One or more regions of an oligonucleotide that hybridize to a scaffold strand can be located at or near the 5′ end of the oligonucleotide, at or near the 3′ end of the oligonucleotide, or in a region of the oligonucleotide that is between the end regions. Oligonucleotides can be configured to hybridize with a nucleic acid scaffold, another oligonucleotide, a staple oligonucleotide, or a combination thereof. The oligonucleotides can be linear (i.e. having a 3′ end and a 5′ end) or closed (i.e. circular, lacking both 3′ and 5′ ends).

An oligonucleotide that is included in a structured nucleic acid particle can have any of a variety of lengths. An oligonucleotide may have a length of at least about 10, 25, 50, 100, 250, 500, or more nucleotides. Alternatively or additionally, an oligonucleotide may have a length of no more than about 500, 250, 100, 50, 25, 10, or fewer nucleotides. Preferably, oligonucleotides have a length of about 100 nucleotides or less. In some embodiments, oligonucleotide that is included in a structured nucleic acid particle is no more than 100, 99, 98, 97, 98, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10 or fewer nucleotides. An oligonucleotide in a nucleic acid origami may hybridize with another oligonucleotide or a scaffold strand forming a particular number of base pairs. An oligonucleotide may form a hybridization region of at least about 5, 6, 7, 8, 9, 10, 15, 20, 25, 50 or more consecutive or total base pairs. Alternatively or additionally, an oligonucleotide may form a hybridization region of no more than about 50, 25, 20, 15, 10, 9, 8, 7, 6, 5, or fewer consecutive or total base pairs.

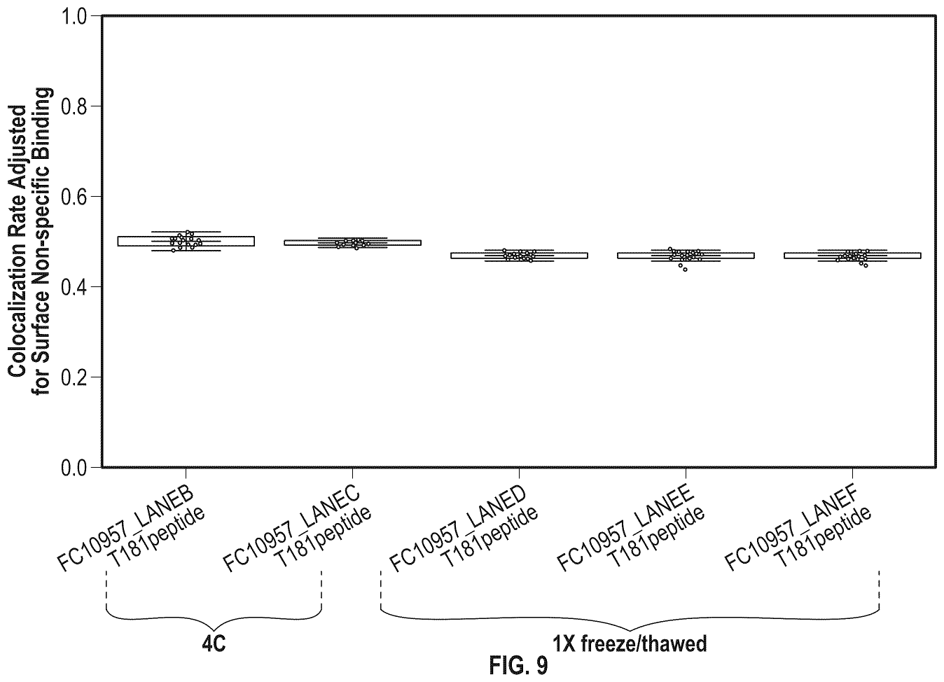

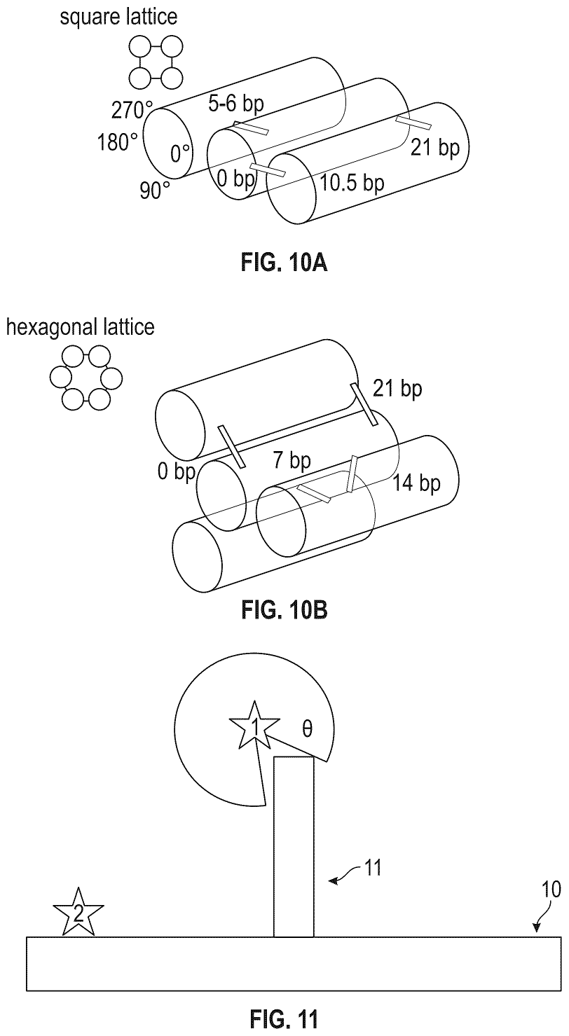

The relative orientations of nucleic acid helices in a nucleic acid origami can be described in terms of lattice topology. For purposes of illustrating the lattice topology, the helices can be approximated as B-form DNA which has period around the helical axis of 10.5 base pairs (bp) per revolution (360 degrees), or 21 bp per two revolutions (720 degrees). FIG. 10A provides diagrammatic representations of a square lattice. As illustrated by the four helices shown end-on in the upper left of FIG. 10A, a given helix in an optimal square lattice will have closest contact with neighboring helices every quarter revolution (90 degrees). However, 90 degrees occurs every 2.625 base pairs in B-form DNA, which is not a whole number. Thus, to avoid unnecessary strain on the lattice, crossovers between a given helix and its neighbors, for example, via staples or cross-links, need not occur every 90 degrees. Optionally, a helix can form crossovers with a particular neighbor helix every two revolutions, a distance which equates to a whole number of base pairs (i.e., 21 base pairs). As illustrated by the perspective view in the lower right of FIG. 10A, the middle helix has crossover points with a first neighbor helix at a first position (0 bp) and at a second position (21 bp), the two positions being separated by two revolutions along the middle helix. The middle helix does not have a crossover every revolution as illustrated by the absence of a crossover with the first neighbor at 10.5 bp. Optionally, a helix that is positioned between two neighbor helices can form a crossover with the first neighbors that is separated by 5 to 6 bp from the crossover with the second neighbor. As illustrated by the perspective view in the lower right of FIG. 10A, the middle helix has a crossover point with the first neighbor helix at a first position (0 bp) and with the second neighbor at a second position (5-6 bp), the two positions being separated by a half revolutions along the middle helix. Examples of nucleic acid origami structures having square lattices include the origami tile set forth in Example I, and the dTile and Tile2 structures set forth in Example II.

FIG. 10B provides diagrammatic representations of a hexagonal lattice of helices. As illustrated by the six helices shown end-on in the upper left of FIG. 10B, a given helix in an optimal hexagonal lattice will have closest contact with neighboring helices every 120 degrees. This equates to a whole number increment of 7 bp along the given helix. As illustrated by the perspective view in the lower right of FIG. 10B, the middle helix has crossover points with a first neighbor helix at a first position (0 bp) and at a second position (21 bp), the two positions being separated by two revolutions along the middle helix. The middle helix also has crossovers with two other neighboring helices. Specifically, the middle helix has crossovers with the first helix at the 0 bp position, then at the 7 bp position with a second helix and at the 14 bp position with a third helix. The larger density of crossovers for the hexagonal lattice, as compared to the square lattice, can provide for a more stable structure in the case of the hexagonal lattice. Exemplary designs for nucleic acid origami having hexagonal lattices are set forth in Examples V, VI, and XII and shown in the figures.

Topologies that can be useful in the methods or compositions set forth herein include a hexagonal (also defined as “honeycomb”) lattice or square (also defined as “rhombic”) lattice. In some embodiments, nucleic acid helices are disposed as a hexagonal lattice to form a single-layer sheet including a single-layer sheet containing a protruding structure (a post). For instance, each double helix is arranged in a set number of turns long and arranged horizontally in a ‘zig-zag’pattern. In some embodiments, nucleic acid helices are disposed as a hexagonal lattice to form a multilayer structure to from a three-dimensional structure such as, but not limited to, a cube, cuboid, or brick structure. Alternatively, nucleic acid helices can be disposed as a square lattice to form a single-layer sheet. In some embodiments, nucleic acid helices can be disposed as a square lattice to form a multilayer structure to from a three-dimensional structure. Exemplary nucleic acid origami designs for hexagonal lattices and square lattices are set forth in Wickham et al., Nature Comm. 11:5768 (2020), which is incorporated herein by reference.

Any of the nucleic acid origami structures described herein can include sites for attaching a reactive moiety, analyte of interest (e.g., protein), label, docker, tether, affinity moiety, linker, solid support or other moiety. Described solely as an example, each site can be spaced approximately 8 nm apart from six nearest neighbors, projecting in different directions. In another descriptive example, a single-layer origami tile can include sites that project from one face, arranged in a square lattice with roughly 5 nm spacing. In some designs, the sites in the rectangular tile project from only one face of the tile.

A reactive moiety, analyte of interest (e.g., protein), label, docker, tether, affinity moiety, linker, solid support or other moiety can be attached to nucleic acid origami via a scaffold component or oligonucleotide component of the origami structure. For example, the scaffold or oligonucleotide can include one or more nucleotide analog(s) that attach covalently or non-covalently to a reactive moiety, protein, label, linker, solid support or other moiety. A nucleic acid origami can include one or more oligonucleotide components each having a reactive moiety, analyte of interest (e.g., protein), label, docker, tether, affinity moiety, linker, solid support or other moiety. A nucleic acid origami, or other particle set forth herein, can include at least 1, 2, 5, 10, 20, 30, 40, 50, 75, 100 or more moieties of a type set forth herein. Alternatively or additionally, a nucleic acid origami, or other particle set forth herein, can include at most 100, 75, 50, 40, 30, 20, 10, 5, 2, or 1 moieties of a type set forth herein.

A structured nucleic acid particle (e.g., nucleic acid origami, or nucleic acid nanoball) may be designed and formed by appropriate techniques including, for example, those set forth in further detail below or those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org). Further examples of nucleic acid origami design are set forth in Examples IV, V and VI herein.

The present disclosure provides a method for making a structured nucleic acid particle that includes a nucleic acid origami component. The method can include steps of (a) forming a mixture including a plurality of nucleic acid scaffolds and nucleic acid staples, thereby producing structured nucleic acid particles, the structured nucleic acid particles each including a nucleic acid scaffold and a plurality of nucleic acid staples folded into a nucleic acid origami; and (b) separating the structured nucleic acid particles from nucleic acid scaffolds and nucleic acid staples of the mixture.

A nucleic acid origami can be formed between a scaffold nucleic acid and a plurality of staple nucleic acids. The nucleic acids can be combined to form a mixture and the nucleic acids can be denatured, for example, due to elevated temperature or presence of chemical denaturants. The nucleic acids can spontaneously fold into a nucleic acid origami structure by reducing the temperature or removing chemical denaturants. Exemplary conditions for folding nucleic acids into origami structures are set forth in Rothemund, Nature 440:297-302 (2006); U.S. Pat. Nos. 8,501,923, 9,340,416 or 11,203,612; US Pat. App. Pub. No. 2022/0162684 A1; or U.S. patent application Ser. No. 17/692,035 or 18/448,000, each of which is incorporated herein by reference. Further methods are set forth in the Examples section herein.

In some configurations, a method for assembling a nucleic acid origami can be carried out in fluid phase. For example, scaffold nucleic acids, staple nucleic acids and any other components that are to be incorporated into a nucleic acid origami can be contacted with each other in solution. Once nucleic acid origami structures have been formed, the origami structures can be separated from unbound nucleic acid scaffolds and unbound nucleic acid staples. Unbound scaffolds and staples can be separated from origami using chromatography such as reverse phase chromatography, size exclusion chromatography, ion exchange chromatography (e.g., using insoluble supports derivatized with diethylaminoethyl or other anion exchange media), affinity chromatography or the like. Unbound scaffolds and staples can be separated from origami using dialysis. Dialysis membranes can be selected to have a molecular weight cutoff that allows unbound scaffolds and/or staples to pass through the membrane while origami structures are retained. Separation can also be achieved by filtration, for example, using molecular weight cutoff filters that pass scaffolds, staples and other relatively small mixture components into the filtrate while the retentate contains the origami structures. Useful filtration apparatus include, but are not limited to, stirred filter devices, such as an Amicon^{M} stirred cell, or tangential flow filtration devices.

In other configurations, a nucleic acid origami can be assembled on a solid-phase support such as a bead, particle or planar surface. For example, an immobilized nucleic acid scaffold can be contacted with a plurality of fluid-phase staples to produce an immobilized nucleic acid origami. Immobilization can occur using any of a variety of attachment chemistries known in the art or set forth herein, for example, in the context of attaching a protein to a structured nucleic acid particle. Immobilization of nucleic acid origami on solid support provides for convenient separation of the origami from unreacted components or other fluid phase components by physically separating the fluid from the solid support. Any of a variety of methods can be used including, but not limited to filtration, sedimentation, centrifugation, magnetic attraction of paramagnetic or magnetic beads, electrical attraction of charged beads, fluid decanting or fluid aspiration.

Optionally, attachment of a nucleic acid origami or component thereof (e.g., a scaffold nucleic acid or handle nucleic acid) to a solid support can be mediated via hybridization of a solid support-attached nucleic acid strand to a complementary strand of the origami or component thereof. In some cases, it may be beneficial to incorporate a non-natural nucleotide into one or both strands of the hybrid to increase the stability of the hybrid. For example, duplex structures having locked nucleic acids can provide a more stable structure than those having naturally occurring nucleotides. This can be beneficial for maintaining attachment of the origami, or component thereof, during temperature changes that occur during origami assembly. Example VII sets forth methods for assembling nucleic acid origami using scaffold nucleic acids attached to solid supports via duplexes having locked nucleic acids.

Structured nucleic acid particles having nucleic acid origami can be attached to affinity moieties or labels to form probes. For example, affinity moieties or labels can include a nucleic acid component having a nucleotide sequence that hybridizes to a complementary sequence in an origami structure. The probes can be separated from one or more unreacted components of the attachment process such as origami particles, affinity moiety precursors (e.g., affinity reagents) or labels. It will be understood that affinity moieties or labels need not be attached to nucleic acid origami via hybridization of nucleic acid sequences. For example, affinity moieties or labels can be attached via covalent reaction between a chemically derivatized origami and a chemically derivatized affinity reagent or label. Useful chemistries for attaching functionalized nucleic acids, affinity moieties, labels or other moieties to structured nucleic acid particles, such as nucleic acid origami, include any of a variety of those set forth herein or known in the art. Useful chemistries are also set forth in U.S. Pat. No. 11,203,612 and US Pat. App. Pub. No. 2022/0162684 A1, each of which is incorporated herein by reference.

Once probes have been formed, they can be separated from unreacted components, such as unreacted structured nucleic acid particles, dockers, tethers, labels and/or affinity reagents. Separation can employ chromatography, dialysis, filtration or other techniques set forth herein in the context of separating nucleic acid origami from excess nucleic acid components used to produce the origami. For example, separation can be achieved using a filter or dialysis membrane having a molecular weight cutoff that allows passage of affinity reagents and/or structured nucleic acid particles while retaining the probes. Separation can also be achieved, for example, by precipitation of affinity reagents using conditions wherein the probes remain in solution. For example, antibodies and other protein-based affinity reagents can be precipitated by salting out (e.g., using ammonium sulfate), by non-ionic hydrophilic polymers (e.g., dextrans or polyethylene glycols), or other reagents that can preferentially precipitate proteins rather than nucleic acids. Alternatively, probes can be selectively precipitated using conditions wherein affinity reagents are soluble. For example, structured nucleic acids can be precipitated by ethanol or isopropanol while retaining protein-based affinity reagents in solution. Other separation techniques include, for example, chromatography (e.g., anion exchange or size exclusion), gel electrophoresis, liquid-liquid extraction or solid-phase extraction.

A nucleic acid origami can be separated from any of a variety of reagents or reaction mixtures set forth herein for modifying an origami. The separation can be performed whether the origami is assembled prior to or during reaction with reagents other than staples and scaffolds. For example, the separation methods and apparatus set forth herein can be used to separate nucleic acid origami from unreacted labels, affinity reagents, analytes of interest (e.g., target proteins), nucleic acid handles, tethers, dockers or other components set forth herein in the context of assembling or modifying nucleic acid origami.

Accordingly, the present disclosure provides a method for purifying a structured nucleic acid particle having nucleic acid origami. The method can include steps of (a) providing a mixture comprising a nucleic acid origami and excess reactants for assembly or modification of the nucleic acid origami; and (b) separating the structured nucleic acid particles from the excess reactants. Exemplary excess reactants include, but are not limited to, nucleic acid scaffolds, nucleic acid staples, nucleic acid handles, labels, affinity reagents (e.g., antibodies or nucleic acid aptamers), analytes of interest (e.g., proteins), tethers or dockers.

Another type of structured nucleic acid particle is a nucleic acid nanoball. Nucleic acid nanoballs may be fabricated by a method such as rolling circle amplification using a circular template to generate a nucleic acid amplicon consisting of a concatemer of template complements. The amplicon can be further modified to include cross-links, for example, in the form of staples that hybridize to different regions of the amplicon. Exemplary methods for making nucleic acid nanoballs are described, for example, in U.S. Pat. No. 8,445,194, which is incorporated herein by reference.

A structured nucleic acid particle can be attached to any of a variety of analytes of interest. An analyte of interest can be attached to a surface of a structured nucleic acid particle. For example, a nucleic acid origami can be folded to form a surface with an extended area such as a planar surface (e.g., on a tile) or a curved surface (e.g., on a barrel or cylinder). Optionally, an analyte of interest can be attached to a handle on an origami structure that protrudes away from a facet or side of the origami structure. Exemplary handles include those having sequences set forth in further detail below. A handle can be configured to be double stranded or single stranded. Single stranded handles are generally more flexible than double stranded handles. A handle can optionally include a more substantial structure to, for example, to provide increased rigidity or improved accessibility of attached analytes to other reagents such as affinity reagents. An example of a rigid handle is a post. A post can include multiple helices that form a lattice (e.g., square lattice or hexagonal lattice). The helices can run along the length of the post (i.e. the length running from a surface of the structured nucleic acid particle to a point at which an analyte of interest is attached), for example, as occurs in the CBPost origami structure set forth in Example V. Optionally, the helices can run orthogonal to the length of the post, for example stacked like logs in the wall of a log cabin. A handle or post can include a handle strand, an oligonucleotide that hybridizes with a handle strand, a staple strand, or a region of a scaffold strand. In some configurations, a post can include multiple regions of a scaffold strand. As such a scaffold strand can have helices running along a first plane (e.g., an xy plane in a Cartesian system) and can also include one or more helices running non-parallel to the first plane (e.g., running orthogonal along the z axis of the Cartesian system). Further examples of post structures and configurations are set forth in Example IV.

Exemplary analytes that can be used in a method or composition set forth herein include, but are not limited to, a tissue, cell, organelle, virus, nucleic acid (e.g., DNA or RNA), carbohydrate (e.g., monosaccharide, oligosaccharide or polysaccharide), vitamin, enzyme cofactor, hormone, small molecule such as a candidate therapeutic agent, metabolite, nucleotide, nucleoside, amino acid, sugar, lipid, or the like. Proteins are particularly well suited for use in a composition or method set forth herein. Sources for proteins and techniques for manipulating proteins are exemplified below and can be extended to other biological analytes using modifications known or determinable by those skilled in the art.

A protein can be derived from a natural or synthetic source. Exemplary sources include, but are not limited to a biological tissue, fluid, cell or subcellular compartment (e.g., organelle). For example, a sample can be derived from a tissue biopsy, biological fluid (e.g., blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or protein synthesis reaction. Any sample where a protein is a native or expected constituent can be used. For example, sources for gastric enzymes may include cells from digestive organs, a sample from a gastric duct, or a fluid sample from a digestive organ (e.g., bile). In a second example, a primary source for a cancer biomarker protein may be a tumor biopsy sample. Other sources include environmental samples or forensic samples.

Exemplary organisms from which a protein can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. A protein can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A protein can be derived from a homogeneous culture or population of the above organisms or alternatively from a collection of several different organisms, for example, in a community or ecosystem.

In some cases, a protein can be derived from an organism that is collected from a host organism. A protein may be derived from a parasitic, pathogenic, symbiotic, or latent organism collected from a host organism. A protein can be derived from an organism, tissue, cell or biological fluid that is known or suspected of being associated with a disease state or disorder (e.g., an oncogenic virus). Alternatively, a protein can be derived from an organism, tissue, cell or biological fluid that is known or suspected of not being associated with a particular disease state or disorder. For example, one or more proteins isolated from such a source can be used as a control for comparison to results acquired from a source that is known or suspected of being associated with the particular disease state or disorder. A sample may include a microbiome. A sample may include a plurality of proteins contributed by microbiome constituents. In some cases, one or more proteins used in a method, composition or apparatus set forth herein may be obtained from a single organism (e.g., an individual human), single cell, single organelle, or single protein-containing particle (e.g., a viral particle).

In some cases, one or more proteins can be obtained from a single cell, protein-containing particle (e.g., a viral particle), or a fragment thereof. A single cell, protein-containing particle, or fragment thereof may be collected by any known method in the art, such as fluorescence assisted cell sorting, magnetic-assisted cell sorting, and buoyancy-assisted cell sorting. In some cases, a single cell, protein-containing particle, or fragment thereof may be collected by an emulsion technique such as liposome or micellar capture.

A protein can optionally be separated or isolated from other components of the source for the protein(s). For example, one or more proteins can be separated or isolated from lipids, nucleic acids, hormones, enzyme cofactors, vitamins, metabolites, microtubules, organelles (e.g., nucleus, mitochondria, chloroplast, endoplasmic reticulum, vesicle, cytoskeleton, vacuole, lysosome, cell membrane, cytosol or Golgi apparatus), other proteins or the like. Protein separation can be carried out using methods known in the art such as centrifugation (e.g., to separate membrane fractions from soluble fractions), density gradient centrifugation (e.g., to separate different types of organelles), precipitation, affinity capture, adsorption, liquid-liquid extraction, solid-phase extraction, chromatography (e.g., affinity chromatography, ion exchange chromatography, reverse phase chromatography, size exclusion chromatography, electrophoresis (e.g., polyacrylamide gel electrophoresis) or the like. Particularly useful protein separation methods are set forth in Scopes, Protein Purification Principles and Practice, Springer; 3rd edition (1993).

A protein can be in a native conformation or denatured conformation. For example, a protein can be in a native conformation, whereby it is capable of performing native function(s) such as catalysis of its natural substrate(s) or binding to its natural substrate(s). Alternatively, a protein can be in a denatured conformation whereby it is incapable of performing certain native function(s) such as catalysis of its natural substrate(s) or binding to its natural substrate(s). A protein can be in a native conformation for some manipulations set forth herein and in a denatured conformation for other manipulations set forth herein. A protein may be denatured at any stage during manipulation, including for example, upon removal from a native milieu or at a later stage of processing such as a stage where the protein is separated from other cellular components, fractionated from other proteins, functionalized to include a reactive moiety, attached to a particle or solid support, contacted with an affinity reagent, detected, digested to produce fragments, or other process. Any of a variety of denaturants can be used such as heat (e.g., temperatures greater than about 40° C., 60° C., 80° C. or higher), excessive pH (e.g., pH lower than 4.0, 3.0 or 2.0; or pH greater than 10.0, 11.0 or 12.0); chaotropic agents (e.g., urea, guanidinium chloride, or sodium dodecyl sulfate), organic solvent (e.g., chloroform or ethanol), physical agitation (e.g., sonication) or radiation. A denatured protein may be refolded, for example, reverting to a native state for one or more steps of a process set forth herein.

A method of the present disclosure can include a step of attaching a protein to a structured nucleic acid particle. A protein that is to be attached to a structured nucleic acid particle can include at least one amino acid that is reactive with an attachment moiety on the particle. Optionally, attachment can exploit a reactive moiety that is present in a natural amino acid. For example, attachment can occur between an attachment moiety on a structured nucleic acid particle and (A) an amine that is present at the amino terminus of a protein or in the side chain of a lysine, histidine or arginine side chain; (B) a sulfur that is present in the side chain of a cysteine or methionine; (C) a carboxylate that is present at the carboxy terminus of a protein or in the side chain of an aspartic acid or glutamic acid; (D) an oxygen that is present in the side chain of a serine, threonine or tyrosine; or (E) an amide that is present in the side chain of a glutamine or asparagine.

Optionally, a protein can be modified to incorporate an exogenous moiety that is reactive with an attachment moiety on a structured nucleic acid particle. For example, one or more amino acids of a protein can be modified to include an exogenous moiety that forms a covalent bond with a chemically reactive attachment moiety on a structured nucleic acid particle. Optionally, one or more amino acids of a protein can be modified to include an exogenous moiety that participates in a binding reaction to form a non-covalent bond with an attachment moiety on a structured nucleic acid particle. An amino acid can be functionalized with an exogenous moiety by exploiting reactivities of amines, sulfurs, carboxylates, oxygens, amides or other reactive moieties found in native amino acids. Exemplary reactive moieties (e.g., native or exogenous to proteins) and attachment moieties with which they react are set forth below or in WO 2019/195633 A1; US Pat. App. Pub. Nos. 2021/0101930 A1, 2022/0162684 A1 or 2022/0227890 A1; or U.S. Pat. Nos. 11,203,612 or 11,505,796, each of which is incorporated herein by reference.

A protein and an attachment moiety with which the protein will react can include components of a SpyTag/SpyCatcher system (See, Zakeri et al. Proceedings Nat'l Acad. Sciences USA. 109 (12): E690-7 (2012)). In this system, a 13 amino acid tag protein (Spy Tag) forms a first coupling handle, with a 12.3 kDa protein (Spy-Catcher) forming the partner to the first coupling handle. Optionally, the Spy Catcher can be attached to a protein. The Spy Catcher can irreversibly bond to a Spy Tag on a particle through an isopeptide bond. As will be appreciated, either the Spy Tag or the Spy Catcher can be on a structured nucleic acid particle and a protein can be functionalized with the other partner.

In some configurations, an attachment moiety on a structured nucleic acid particle can be reactive in a click reaction. Attachment can be accomplished in part by chemical reaction of a click moiety with a reactive moiety on a protein. The chemical conjugation may proceed via an amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddtion reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof.

Moieties that participate in cycloaddition reactions may be utilized to attach a protein, or fragment thereof, to a structured nucleic acid particle. In cycloaddition reactions, two or more unsaturated moieties form a cyclic product with a reduction in the degree of unsaturation, these reaction partners are typically absent from natural systems, and so the use of cycloadditions for conjugation utilizes the introduction of unnatural functionality within a coupling partner.

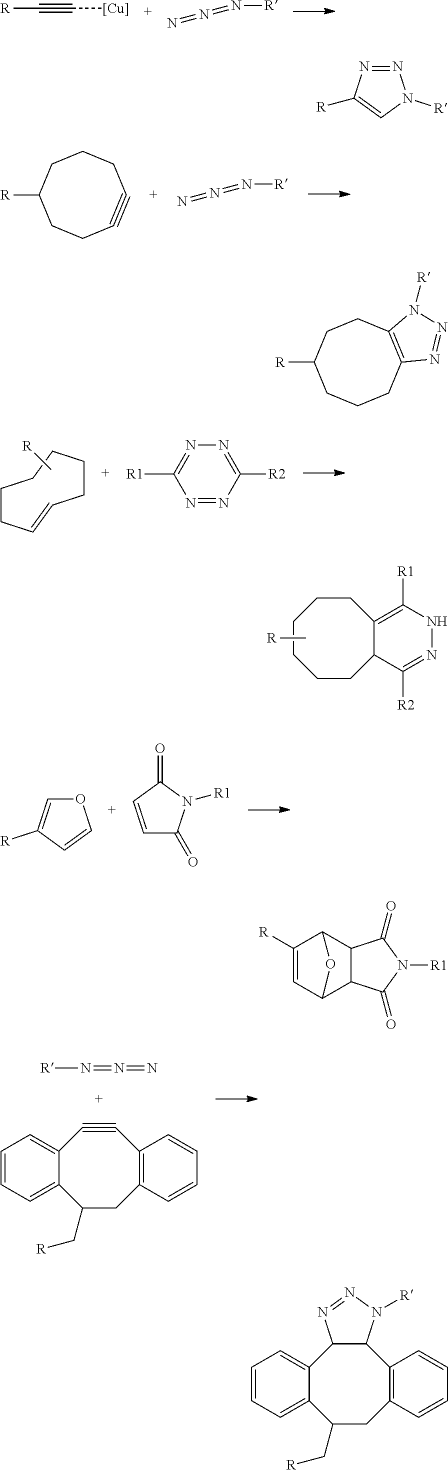

In some cases, moieties that participate in Copper-Catalyzed Azide-Alkyne Cycloadditions (CuAAC) may be utilized to attach a protein to a structured nucleic acid particle. Optionally, moieties that participate in Strain-Promoted Azide-Alkyne Cycloadditions (SPAAC) may be utilized. One of an azide or alkyne can be connected to a protein and reacted with a structured nucleic acid particle connected to the other. A CuAAC or SPAAC reaction can be performed to produce a triazole attachment of a protein to a structured nucleic acid particle.

Moieties that participate in inverse-electron demand Diels-Alder (IEDDA) reactions may be utilized to attach a protein to a structured nucleic acid particle. One of a 1,2,4,5-tetrazine moiety, strained alkene moiety or strained alkyne can be connected to an antibody conjugate and, optionally, subjected to an IEDDA reaction. Exemplary moieties include, but are not limited to, trans-cyclooctenes, functionalized norbornene derivatives, triazines, or spirohexene. In some cases, a maleimide or furan can be used as an attachment moiety and, optionally, used in a hetero-Diels-Alder cycloaddition between a maleimide and furan. In some cases, a Diels-Alder reaction can achieve covalent coupling of a diene moiety with an alkene moiety to form a six-membered ring complex for attachment.

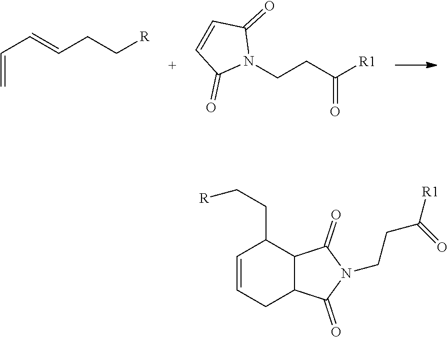

A protein can be attached to a structured nucleic acid particle via binding moieties having affinity for each other. For example, a protein can include a first binding moiety that binds to a second binding moiety on a structured nucleic acid particle. A first binding moiety may bind with a second binding moiety in a non-covalent manner. Some binding moieties can also be chemically reactive or catalytic (e.g., kinases, proteases, etc.). A binding moiety can be chemically non-reactive and non-catalytic, thereby not permanently altering the chemical structure of another binding moiety to which it binds. Exemplary pairs of binding moieties include, but are not limited to, an antibody, such as a full-length antibody or functional fragment thereof which bind to epitopes. Other useful binding moiety pairs include, for example, (strept)avidin (or analogs thereof) and biotin (or analogs thereof), complementary nucleic acids, nucleic acid aptamers and their ligands, lectins and carbohydrates or the like.

The present disclosure provides a structured nucleic acid particle, including (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand, wherein the one or more first handles comprise a first nucleotide sequence; (d) one or more second handles hybridized to the scaffold strand or to a staple strand, wherein the one or more second handles comprise a second nucleotide sequence; and (e) one or more third handles hybridized to the scaffold strand or to a staple strand, wherein the one or more third handles comprise a third nucleotide sequence. In some configurations the structured nucleic acid particle can further include (f) one or more fourth handles hybridized to the scaffold strand or to a staple strand, wherein the one or more fourth handles comprise a fourth nucleotide sequence. Optionally, the one or more first handles can be configured to attach one or more analytes to the structured nucleic acid particle, the one or more second handles can be configured to attach one or more labels to the structured nucleic acid particle, the one or more third handles can be configured to attach the structured nucleic acid particle to a solid support (e.g., a surface of an array or particle), and/or the one or more fourth handles can be configured to attach an assay reagent, such as an affinity reagent or to attach a second structured nucleic acid particle.

The first, second, third and fourth nucleotide sequences in the above structured nucleic acid particle can differ from each other, thereby providing specificity for attachment of complementary oligonucleotides functionalized with the moieties exemplified above or other moieties of interest. For example, the first nucleotide sequence can be complementary to a first oligonucleotide that is optionally attached to an analyte of interest, the second nucleotide sequence can be complementary to a second oligonucleotide that is optionally attached to a label, the third nucleotide sequence can be complementary to a third oligonucleotide that is optionally attached to a solid support (e.g., a surface of an array or particle) and/or the fourth nucleotide sequence can be complementary to a fourth oligonucleotide that is optionally attached to an affinity reagent or structured nucleic acid particle (e.g., a structured nucleic acid particle attached to at least one affinity moiety and to at least one label moiety). The first, second, third and fourth nucleotide sequences can also be designed for orthogonal annealing when in the presence of each other. As such, the first handle can anneal to the first oligonucleotide without substantially annealing to the second oligonucleotide, third oligonucleotide, fourth oligonucleotide, or to any of the four types of handles. The first oligonucleotide can anneal to the first handle without substantially annealing to the second handle, third handle, fourth handle, or any of the four types of oligonucleotides. Similarly, the second handle can anneal to the second oligonucleotide without substantially annealing to the first oligonucleotide, third oligonucleotide, fourth oligonucleotide or any of the four types of oligonucleotides. The second oligonucleotide can anneal to the second handle without substantially annealing to the first handle, third handle, fourth handle, or any of the four types of oligonucleotides. Moreover, the third handle can anneal to the third oligonucleotide without substantially annealing to the first oligonucleotide, second oligonucleotide, fourth oligonucleotide, or any of the four types of handles. The third oligonucleotide can anneal to the third handle without substantially annealing to the first handle, second handle, fourth handle, or any of the four types of oligonucleotides.

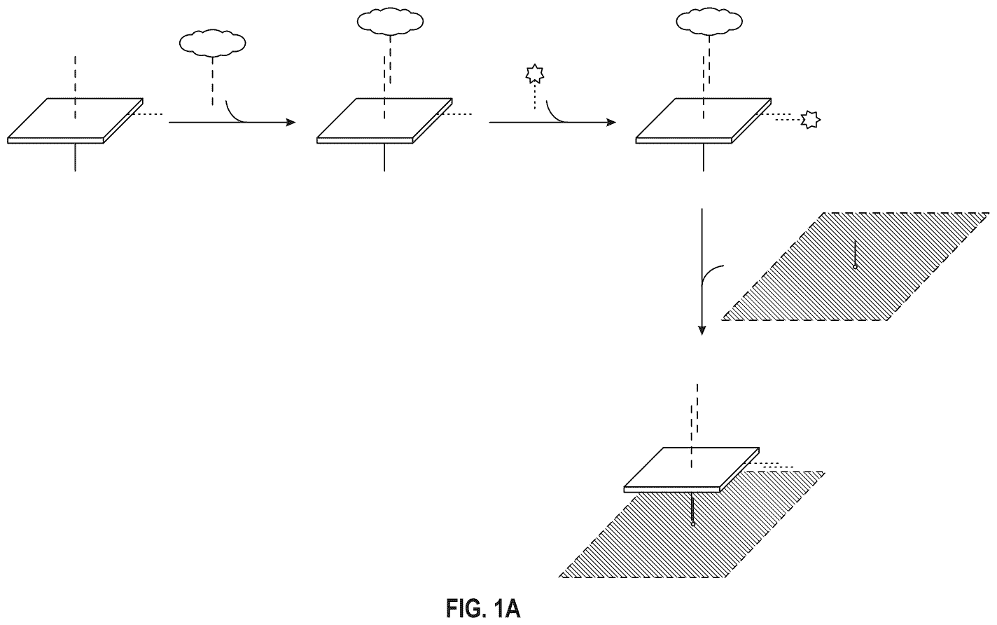

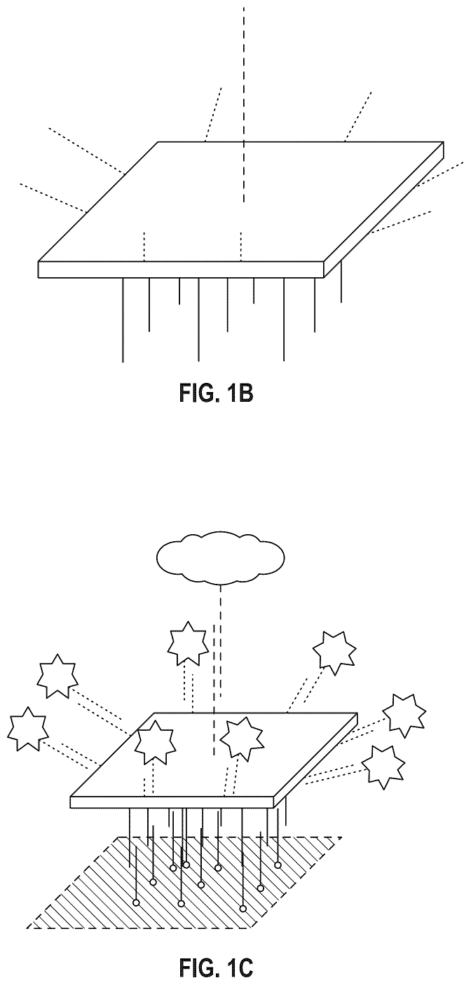

FIG. 1A shows a reaction series for making a structured nucleic acid particle (square tile) attached to an analyte handle (dashed line), a label handle (dotted line) and attachment handle (solid line). In the first step of the reaction series, a protein (globule) having an attached oligonucleotide moiety (dashed line) is attached to the structured nucleic acid particle via hybridization of the oligonucleotide moiety to the analyte handle. Due to the orthogonal nature of the handle sequences, the oligonucleotide moiety of the protein does not hybridize to other sequences in the structured nucleic acid particle such as the label handle and attachment handle. Proceeding to the second step, a label (seven-point star) having an attached oligonucleotide moiety (dotted line) is attached to the structured nucleic acid particle via hybridization of the oligonucleotide moiety to the label handle. The oligonucleotide moiety of the label does not hybridize to other sequences in the structured nucleic acid particle such as the analyte handle and attachment handle. In the third step, the structured nucleic acid particle is attached to a solid support (plane with hatched lines and dotted border) via hybridization of the attachment handle to a complementary oligonucleotide (solid line) on the solid support. The oligonucleotide moiety on the solid support does not hybridize to other sequences in the structured nucleic acid particle such as the analyte handle and label handle. The order of steps shown in the figure is exemplary. Attachment can occur serially in a different order. Moreover, two of the attachment steps, or all three attachment steps, can occur simultaneously. The principles exemplified by FIG. 1A for three handles and three complementary oligonucleotides, can be extended to an assay reagent handle and its complementary oligonucleotide.

A structured nucleic acid particle can include a plurality of handles of a particular type. Handles of a given type can have the same nucleotide sequence as each other or they can have at least a region of sequence in common. FIG. 1B shows a diagram of a structured nucleic acid particle (square tile) having eight label handles (dotted lines), nine attachment handles (solid lines), and a single analyte handle (dashed line). FIG. 1C shows a diagram of the structured nucleic acid particle attached to a single protein analyte (globule) via a single nucleic acid hybrid (dashed double line), eight labels (seven-point stars) via respective nucleic acid hybrids (dotted double lines) and to a solid support via nine nucleic acid hybrids (solid double lines). The number of handles of each type is exemplary and can be extended to four or more handles. A structured nucleic acid particle can include at least 1, 2, 3, 4, 5, 10, 15, 20, 25, 30, 40, 50 or more handles of a particular type. Alternatively or additionally, a structured nucleic acid particle can include at most 50, 40, 30, 25, 20, 15, 10, 5, 4, 3, 2 or 1 handles of a particular type. The number of complementary oligonucleotide types can be in one or more of these ranges as well.

Handles can be positioned in any of a variety of relative orientations on a structured nucleic acid particle. For example, one or more attachment handles can be present on a first side of a nucleic acid origami particle and one or more analyte handles can be placed on a second side of the nucleic acid origami particle. The first and second side can face in different directions, for example, in opposite directions as exemplified in FIGS. 1A, 1B and 1C. Orienting the analyte handle(s) in a different direction from the attachment handle(s) can allow the analyte to be positioned away from a surface to which the nucleic acid origami is immobilized, thereby facilitating access of an analyte on the analyte handle to reagents in a fluid that interacts with the surface immobilized nucleic acid origami particle. One or more labels can be oriented away from an analyte by virtue of the label handle(s) being attached to a different side of the nucleic acid origami particle than a side to which the analyte handle is attached. This relative orientation can prevent quenching of signal from the label that may occur if the analyte contacts the label or is otherwise too proximal. Similarly, this relative orientation can prevent damage to the analyte by the label(s), for example, due to radicals or other reactive species released by a label (e.g., a luminescent label) when excited. Alternatively or additionally to the relative orientation of label(s) to analytes, one or more labels can be oriented away from a surface by virtue of the label handle(s) being attached to a different side of the nucleic acid origami particle than a side to which one or more attachment handle(s) is/are attached. This orientation can inhibit the surface from quenching label(s) and can increase observability of label(s) due to appropriate positioning of a detector relative to other components in a detected sample.

As an alternative or addition to positioning handles at particular relative orientations, a structured nucleic acid particle can be configured to position handles at particular distances from each other. For example, a first handle can be attached to a structured nucleic acid particle at a position that is at least 10 nm, 20 nm, 30 nm, 40 nm, 50 nm or further from the attachment point for the nearest other handle. As such, a label handle can be separated from a nearest neighbor attachment handle, analyte handle or other label handle; an analyte handle can be separated from a nearest neighbor attachment handle, label handle or other analyte handle; or an attachment handle can be separated from a nearest neighbor label handle, analyte handle or other attachment handle. By appropriate choice of attachment point for handles and length of the handles, a structured nucleic acid particle can maintain attached moieties at particular distances from each other. For example, two nearest moieties can be maintained at a separation distance of at least about 10 nm, 20 nm, 30 nm, 40 nm, 50 nm or further from each other. As such, a label can be separated from a nearest neighbor attachment moiety, analyte or other label; an analyte can be separated from a nearest neighbor attachment moiety, label or other analyte; or an attachment moiety can be separated from a nearest neighbor label, analyte or other attachment moiety.

In particular configurations, more than one first handle (e.g., an analyte handle) in a structured nucleic acid particle can each have a common sequence, such as a sequence set forth in Example I or listed in Table 1. Optionally, more than one second handle (e.g., a label handle) in the above structured nucleic acid particle can each have a common sequence, such as a sequence listed in Table 1. Optionally, more than one third handle (e.g., an attachment handle) in the above structured nucleic acid particle can each have a common sequence, such as a sequence listed in Table 1. Optionally, more than one fourth handle (e.g., a reagent handle or particle handle) in the above structured nucleic acid particle can each have a common sequence, such as a sequence listed in Table 1.

In some configurations, a structured nucleic acid particle can include (a) a scaffold strand having a nucleotide sequence; (b) a plurality of different staple strands having different nucleotide sequences, respectively, wherein the staple strands are hybridized to the scaffold strand; (c) a first handle hybridized to the scaffold strand or to a staple strand, wherein the first handle includes the nucleotide sequence of APT 40.2 (SEQ ID NO:1), TCOv40.2 (SEQ ID NO:2), DYE40.22.9 (SEQ ID NO:3), DYE647v40.22.9 (SEQ ID NO:4), JF20.22.9 (SEQ ID NO:5), JFDBCOv20.22.9 (SEQ ID NO:6), Tether1 (SEQ ID NO:7), Docker1 (SEQ ID NO:8), Tether2 (SEQ ID NO:9) or Docker2(SEQ ID NO:10). Optionally, the structured nucleic acid particle can further include (d) a second handle hybridized to the scaffold strand or to a staple strand, wherein the second handle includes the nucleotide sequence of APT 40.2 (SEQ ID NO:1), TCOv40.2 (SEQ ID NO:2), DYE40.22.9 (SEQ ID NO:3), DYE647v40.22.9 (SEQ ID NO:4), JF20.22.9 (SEQ ID NO:5), JFDBCOv20.22.9 (SEQ ID NO:6), Tether1 (SEQ ID NO:7), Docker1 (SEQ ID NO:8), Tether2 (SEQ ID NO:9) or Docker2 (SEQ ID NO:10), wherein nucleotide sequence of the second handle is different from the nucleotide sequence of the first handle strand. Further optionally, the structured nucleic acid particle can further include (e) a third handle hybridized to the scaffold strand or to a staple strand, wherein the third handle includes the nucleotide sequence of APT 40.2 (SEQ ID NO:1), TCOv40.2 (SEQ ID NO:2), DYE40.22.9 (SEQ ID NO:3), DYE647v40.22.9 (SEQ ID NO:4), JF20.22.9 (SEQ ID NO:5), JFDBCOv20.22.9 (SEQ ID NO:6), Tether1 (SEQ ID NO:7), Docker1 (SEQ ID NO:8), Tether2 (SEQ ID NO:9) or Docker2 (SEQ ID NO:10), wherein the nucleotide sequence of the third handle is different from the nucleotide sequences of the first and second handle strands. Further optionally, the structured nucleic acid particle can further include (f) a fourth handle hybridized to the scaffold strand or to a staple strand, wherein the fourth handle includes the nucleotide sequence of APT 40.2 (SEQ ID NO:1), TCOv40.2 (SEQ ID NO:2), DYE40.22.9 (SEQ ID NO:3), DYE647v40.22.9 (SEQ ID NO:4), JF20.22.9 (SEQ ID NO:5), JFDBCOv20.22.9 (SEQ ID NO:6), Tether1 (SEQ ID NO:7), Docker1 (SEQ ID NO:8), Tether2 (SEQ ID NO:9) or Docker2 (SEQ ID NO:10), wherein the nucleotide sequence of the fourth handle is different from the nucleotide sequences of the first, second and third handle strands.

In some configurations, a structured nucleic acid particle can include (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand, wherein each of the one or more first handles have the nucleotide sequence of APT 40.2 (SEQ ID NO:1) hybridized to the nucleotide sequence of TCOv40.2 (SEQ ID NO:2); (d) one or more second handles hybridized to the scaffold strand or to a staple strand; and (e) one or more third handles hybridized to the scaffold strand or to a staple strand. Optionally, the nucleotide sequence of APT 40.2 (SEQ ID NO:1) is present in a nucleic acid strand that is hybridized to the scaffold strand or to a staple strand of the plurality of different staple strands, and the nucleotide sequence of TCOv40.2 (SEQ ID NO:2) is present in a nucleic acid strand functionalized with an analyte of interest. Alternatively, the nucleotide sequence of TCOv40.2 (SEQ ID NO:2) is present in a nucleic acid strand that is hybridized to the scaffold strand or to a staple strand of the plurality of different staple strands, and the nucleotide sequence of APT 40.2 (SEQ ID NO:1) is present in a nucleic acid strand functionalized with an analyte of interest.

In some configurations, a structured nucleic acid particle can include (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand; (d) one or more second handles hybridized to the scaffold strand or to a staple strand, wherein each of the one or more second handles has the nucleotide sequence of DYE40.22.9 (SEQ ID NO:3) hybridized to the nucleotide sequence of DYE647v40.22.9 (SEQ ID NO:4); and (e) one or more third handles hybridized to the scaffold strand or to a staple strand. Optionally, the nucleotide sequence of DYE40.22.9 (SEQ ID NO:3) is present in a nucleic acid strand that is hybridized to the scaffold strand or to a staple strand of the plurality of different staple strands, and the nucleotide sequence of DYE647v40.22.9 (SEQ ID NO:4) is present in a nucleic acid strand functionalized with a label. Optionally, the nucleotide sequence of DYE647v40.22.9 (SEQ ID NO:4) is present in a nucleic acid strand that is hybridized to the scaffold strand or to a staple strand of the plurality of different staple strands, and the nucleotide sequence of DYE40.22.9 (SEQ ID NO:3) is present in a nucleic acid strand functionalized with a label.

In some configurations, a structured nucleic acid particle can include (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand; (d) one or more second handles hybridized to the scaffold strand or to a staple strand; and (e) one or more third handles hybridized to the scaffold strand or to a staple strand, wherein each of the one or more third handles has the nucleotide sequence of JF20.22.9 (SEQ ID NO:5) hybridized to the nucleotide sequence of JFDBCOv20.22.9 (SEQ ID NO:6). Optionally, the nucleotide sequence of JF20.22.9 (SEQ ID NO:5) is present in a nucleic acid strand that is hybridized to the scaffold strand or to a staple strand of the plurality of different staple strands, and the nucleotide sequence of JFDBCOv20.22.9 (SEQ ID NO:6) is present in a nucleic acid strand functionalized with a solid support. Optionally, the nucleotide sequence of JFDBCOv20.22.9 (SEQ ID NO:6) is present in a nucleic acid strand that is hybridized to the scaffold strand or to a staple strand of the plurality of different staple strands, and the nucleotide sequence of JF20.22.9 (SEQ ID NO:5) is present in a nucleic acid strand functionalized with a solid support.

In some configurations, a structured nucleic acid particle can include (a) a scaffold strand having a nucleotide sequence; (b) a plurality of different staple strands having different nucleotide sequences, respectively, wherein the staple strands are hybridized to the scaffold strand; (c) one or more first handles hybridized to the scaffold strand or to a staple strand; (d) one or more second handles hybridized to the scaffold strand or to a staple strand; (e) one or more third handles hybridized to the scaffold strand or to a staple strand; and (f) one or more fourth handles hybridized to the scaffold strand or to a staple strand, wherein each of the one or more fourth handles has the nucleotide sequence of Tether1 (SEQ ID NO:7) or Tether2 (SEQ ID NO:9) hybridized to the nucleotide sequence of Docker1 (SEQ ID NO:8) or Docker2 (SEQ ID NO:10), respectively. Optionally, the nucleotide sequence of Tether1 (SEQ ID NO:7) or Tether2 (SEQ ID NO:9) is present in a nucleic acid strand that is hybridized to the scaffold strand or to a staple strand of the plurality of different staple strands, and the nucleotide sequence of Docker1 (SEQ ID NO:8) or Docker2 (SEQ ID NO:10) is present in a nucleic acid strand functionalized with a second structured nucleic acid particle. Thus, a tether and docker pair can mediate interaction between two structured nucleic acid particles.

Dockers and tethers can be useful for increasing avidity of a binding interaction between an analyte and an affinity reagent. In particular embodiments, the analyte can be attached to a structured nucleic acid particle that includes at least one docker and the affinity reagent can be attached to at least one tether, wherein the docker has a nucleotide sequence that is complementary to a nucleotide sequence of the tether. Optionally, the affinity reagent includes an affinity moiety that is attached to a retaining component (such as a second structured nucleic acid) and the retaining component is attached to the at least one tether. Avidity of a binding interaction between an affinity reagent and analyte can include interaction between a paratope of the affinity reagent and an epitope of the analyte, and can further include interaction between the docker and tether.

A docker can be associated with an analyte via covalent and/or non-covalent attachment of the docker to a structured nucleic acid particle to which the analyte is also attached. Similarly, a tether can be associated with an affinity reagent via covalent and/or non-covalent attachment of the docker to a retaining component (such as a second structured nucleic acid) to which the affinity reagent is also attached. Exemplary attachment chemistries include those set forth herein in the context of attaching analytes and affinity reagents to retaining components, addresses or sites of an array, solid supports, labels, etc. In some configurations, a docker or tether can be attached to a retaining component (e.g., structured nucleic acid particle) via a handle.

A variety of different types of dockers and tethers can be employed to increase avidity of binding between an analyte and affinity reagent. The type of docker and tether that is to be used in combination with a particular analyte and affinity reagent pair can be selected based on known or expected affinity of the affinity reagent for the analyte. For example, a method that employs a first affinity reagent having relatively strong affinity for a particular analyte can utilize a docker and tether pair having relatively weak affinity, whereas a method that employs a second affinity reagent having weaker affinity for the analyte can utilize a docker and tether pair having higher affinity compared to the pair used for the first affinity reagent. Accordingly, the probability of forming a complex and duration of the complex can be tuned by appropriate choice of docker type and tether type.

A docker can be any molecule or moiety that is capable of binding to a tether and a tether can be any molecule or moiety that is capable of binding to a docker. A particularly useful docker or tether is a nucleic acid strand having a nucleotide sequence that complements nucleotide sequences of a tether or docker, respectively. A nucleic acid strand that is used as a docker or tether can include a sequence of at least 3, 4, 5, 6, 7, 8, 9, 10, 12, 14, 16, 18, 20, 25 or more nucleotides. Alternatively or additionally, a nucleic acid strand that is used as a docker or tether can include a sequence of at most 25, 20, 18, 16, 14, 12, 10, 9, 8, 7, 6, 5, 4, 3 or fewer nucleotides. Other useful dockers or tethers include, for example, a receptor that recognizes a ligand, a ligand that recognizes a receptor, an affinity reagent that recognizes an analyte, an analyte that recognizes an affinity reagent, a paratope that recognizes an epitope, an epitope that recognizes a paratope, or a reactive moiety that forms a covalent bond with another reactive moiety. Exemplary dockers or tethers include, but are not limited to, an antibody, Fab′ fragment, F(ab′)2 fragment, single-chain variable fragments, di-scFv, tri-scFv, microantibody, nucleic acid aptamer, affibody, affilin, affimer, affitin, alphabody, anticalin, avimer, miniprotein, DARPin, monobody, nanoCLAMP, lectin, carbohydrate, SpyCatcher or SpyTag. In some configurations, a docker or tether can be a protein that recognizes a nucleic acid sequence such as a DNA binding protein or RNA binding protein. Exemplary nucleic acid-binding proteins, which can be used as dockers or tethers, and the nucleic acid moieties to which they bind, which can be used as tethers or dockers, respectively, include a Toll-Like Receptor (TLR) which binds to DNA having a CpG moiety, transcription factor which binds to a specific nucleic acid sequence, or histone protein(s) which binds to DNA. Further examples are provided in the Eukaryotic nucleic acid binding protein database (ENPD). See Leung et al. Nucleic Acids Res. 47 (Database issue): D322-D329 (2019), which is incorporated herein by reference.

A further variable that can be employed to tune binding between an analyte and affinity reagent is the number of dockers associated with the analyte and/or the number of tethers associated with the affinity reagent. For example, a method that employs a first affinity reagent having relatively strong affinity for an analyte can utilize a relatively low number of docker-tether pairs, whereas a method that employs a second affinity reagent having weaker affinity for the analyte can utilize a greater number of docker-tether pairs compared to the number(s) used for the first affinity reagent.

An analyte can be associated with a single docker or, alternatively, with a plurality of dockers. For example, an analyte can be associated with at least 2, 3, 4, 5, 6, 7, 8, 9, 10, 15, 20, 25, 50 or more dockers. Alternatively or additionally, an analyte can be associated with at most 50, 25, 20, 15, 10, 9, 8, 7, 6, 5, 4, 3, 2 or fewer dockers. The dockers can be substantially identical to each other, thereby recognizing the same tethers. Alternatively, a plurality of dockers can include dockers that differ from each other. In some cases, the different dockers will recognize different tethers. It is also possible for the different dockers to recognize the same tethers. In some configurations, an analyte and the docker with which it is associated will have binding characteristics that are orthogonal to each other. As such, a paratope of an affinity reagent that recognizes or binds to the analyte will not recognize or bind to the docker, and a tether that recognizes or binds to the docker will not recognize or bind to the analyte.

An affinity reagent can be associated with a plurality of tethers. For example, an affinity reagent can be associated with at least 2, 3, 4, 5, 6, 7, 8, 9, 10, 15, 20, 25, 50 or more tethers. Alternatively or additionally, an affinity reagent can be associated with at most 50, 25, 20, 15, 10, 9, 8, 7, 6, 5, 4, 3, 2 or fewer tethers. The tethers can be substantially identical to each other, thereby recognizing the same dockers. Alternatively, a plurality of tethers can include tethers that differ from each other. In some cases, the different tethers will recognize different dockers. It is also possible for the different tethers to recognize the same dockers. In some configurations, an affinity reagent and the tether with which it is associated will have orthogonal binding recognition. As such, an analyte that recognizes or binds to a paratope of the affinity reagent will not recognize or bind to the tether, and a docker that recognizes or binds to the tether will not recognize or bind to the paratope.

A binding event can be tuned via a combination of the number and type of docker-tether pairs used. This can be illustrated in the context of nucleic acid dockers and tethers having complementary nucleotide sequences. For example, the maintenance of a complex between an analyte and affinity reagent can be increased by increasing the number of dockers and tethers present in the complex and also by increasing the avidity of each docker for its complementary tether. The avidity of binding between a nucleic acid docker and tether can be increased, for example, by increasing the length of the complementary sequences, increasing the GC content of the complementary sequences, or otherwise increasing the melting temperature (Tₘ) of the duplex formed by the complementary sequences. The length of the complementary sequences can be at least 3, 4, 5, 6, 7, 8, 9, 10, 12, 14, 16, 18, 20, 25 or more nucleotides. Alternatively or additionally, the length of the complementary sequences can be at most 25, 20, 18, 16, 14, 12, 10, 9, 8, 7, 6, 5, 4, 3 or fewer nucleotides. The GC content of the complementary sequences can be at least 25%, 40%, 50%, 60%, 75%, or higher. Alternatively or additionally, the GC content of the complementary sequences can be at most 75%, 60%, 50%, 40%, 25% or lower.

In particular configurations, a single analyte handle (i.e. one and only one analyte handle) is present in a structured nucleic acid particle. Optionally, the analyte handle is attached to a post. A structured nucleic acid particle having only a single analyte can be useful for separating individual analytes within a plurality of analytes. For example, individual proteins from a proteome or other complex protein sample can be attached to respective structured nucleic acids in an array. As such, the proteins can be individually resolved, for example, in a binding assay or sequencing assay. Any of a variety of other analytes can be individually attached to a structured nucleic acid particle including, but not limited to, a tissue, cell, organelle, virus, nucleic acid (e.g., DNA or RNA), carbohydrate (e.g., monosaccharide, oligosaccharide or polysaccharide), vitamin, enzyme cofactor, hormone, small molecule such as a candidate therapeutic agent, metabolite, nucleotide, nucleoside, amino acid, sugar, lipid, or the like. These and other analytes known in the art can be used in combination with compositions and methods set forth herein. It will be understood that a structured nucleic acid particle can, in some configurations, include a plurality of analyte handles each attached to an analyte or reactive moiety set forth herein.

A structured nucleic acid particle can include a single label handle or label (i.e. one and only one label handle, or one and only one label). Alternatively, a structured nucleic acid particle can include a plurality of label handles and/or labels. For example, the plurality can include at least 2, 3, 4, 5, 6, 7, 8, 9, 10, 12, 15, 20, 25, 50 or more labels and/or label handles. Alternatively or additionally, a plurality can include at most 50, 25, 20, 15, 12, 10, 9, 8, 7, 6, 5, 4, 3, or 2 labels and/or label handles. A plurality of labels present on a structured nucleic acid particle can be of a single type (i.e. having the same structure as each other) or of different types. Any of a variety of labels set forth herein or known in the art can be attached to a label handle. Labels can produce signals that are detected in a method set forth herein. For example, optical emission from luminescent labels can be detected. Increasing the number of labels of the same type can allow for increased signal to noise. In some cases, different labels can produce overlapping signals. For example, a first label handle can be attached to a first luminescent label and a second label handle can be attached to a second luminescent label, wherein the first luminescent label has a different structure from the second luminescent label, and wherein the first luminescent label and the second luminescent label emit luminescence at an overlapping region of the electromagnetic spectrum. The first and second labels may also produce signals in other regions of the spectrum that do not overlap. As such, the labels can be detected in the overlapping region of the spectrum to achieve signal amplification, or the labels can be detected in non-overlapping regions of the spectrum to allow the labels to be distinguished from each other. In some configurations, two or more different labels that are present in a structured nucleic acid need not produce overlapping signals when detected in a method set forth herein. This can be due to the two or more different labels being incapable of producing overlapping signals or due to use of a detector that does not distinguish signals from the different labels.

A structured nucleic acid particle can include a single attachment handle and/or bond to a solid support (i.e. one and only one label handle, or one and only one bond). Alternatively, a structured nucleic acid particle can include a plurality of attachment handles and/or bonds to solid support. For example, the plurality can include at least 2, 3, 4, 5, 6, 7, 8, 9, 10, 12, 15, 20, 25, 50 or more attachments handles or bonds. Alternatively or additionally, a plurality can include at most 50, 25, 20, 15, 12, 10, 9, 8, 7, 6, 5, 4, 3, or 2 attachment handles or bonds. Increasing the number of attachment handles on a structured nucleic acid particle can strengthen the bond between of the structured nucleic acid particle and a solid support. If a weaker bond strength is desired, fewer attachment handles can be used.

A structured nucleic acid particle can include a single assay reagent handle and/or bond to an assay reagent (i.e. one and only one assay reagent handle, or one and only one bond). The assay reagent handle can be used for interaction with a structured nucleic acid particle, such as a structured nucleic acid particle that is attached to an affinity moiety. Alternatively, a structured nucleic acid particle can include a plurality of assay reagent handles, bonds to assay reagent(s) and/or bonds to second nucleic acid particles. For example, the plurality can include at least 2, 3, 4, 5, 6, 7, 8, 9, 10, 12, 15, 20, 25, 50 or more assay reagent handles or bonds. Alternatively or additionally, a plurality can include at most 50, 25, 20, 15, 12, 10, 9, 8, 7, 6, 5, 4, 3, or 2 assay reagent handles or bonds. Increasing the number of assay reagent handles on a structured nucleic acid particle can strengthen the bond between of the structured nucleic acid particle and an assay reagent or second structured nucleic acid particle. If a weaker bond strength is desired, fewer assay reagent handles can be used.

Optionally, a handle nucleic acid can be attached to a reactive moiety, such as a bioorthogonal reactant, click reactant, receptor (e.g., avidin, streptavidin or lectin), ligand (e.g., biotin or analog thereof or carbohydrate), SpyCatcher™ or SpyTag™. The reactive moiety can be configured to react with a reactive moiety on an analyte, label, solid support or other substance such that it becomes attached to the handle as a reaction product.

The present disclosure provides a method of making a structured nucleic acid particle. The method can include steps of (a) providing a nucleic acid composition including (i) a scaffold strand hybridized to a plurality of different staple strands, and (ii) one or more first handle strands hybridized to the scaffold strand or to a staple strand; (b) hybridizing a first complementary oligonucleotide to at least one of the first handle strands, wherein the first complementary oligonucleotide includes a nucleotide sequence that is complementary to a nucleotide sequence of the at least one first handle strand. Optionally, the nucleic acid composition further includes (iii) one or more second handle strands hybridized to the scaffold strand or to a staple strand; and the method further includes a step of (c) hybridizing a second complementary oligonucleotide to at least one of the second handle strands, wherein the second complementary oligonucleotide includes a nucleotide sequence that is complementary to a nucleotide sequence of the at least one second handle strand. In a further option, the nucleic acid composition further includes (iv) one or more third handle strands hybridized to the scaffold strand or to a staple strand; and the method further includes a step of (d) hybridizing a third complementary oligonucleotide to at least one of the third handle strands, wherein the third complementary oligonucleotide includes a nucleotide sequence that is complementary to a nucleotide sequence of the at least one third handle strand. In yet a further option, the nucleic acid composition further includes (v) one or more fourth handle strands hybridized to the scaffold strand or to a staple strand; and the method further includes a step of (e) hybridizing a fourth complementary oligonucleotide to at least one of the fourth handle strands, wherein the fourth complementary oligonucleotide includes a nucleotide sequence that is complementary to a nucleotide sequence of the at least one fourth handle strand.

A complementary oligonucleotide can be attached to a non-nucleic acid moiety, whereby hybridization of the complementary oligonucleotide to a handle strand of a structured nucleic acid particle results in attachment of the non-nucleic acid moiety to the to the structured nucleic acid particle. A complementary strand can be attached to any of a variety of non-nucleic acid moieties including, but not limited to, an analyte of interest, label, solid support, assay reagent (e.g., affinity reagent) or other molecule set forth herein. Different types of moieties can be directed to particular handle strands by virtue of Watson-Crick base-pairing specificity. For example, an analyte of interest can be directed to an analyte handle by virtue of being attached to an oligonucleotide having a nucleotide sequence that is complementary to a sequence of the analyte handle strand. Furthermore, a label can be directed to a label handle strand by virtue of being attached to an oligonucleotide having a nucleotide sequence that is complementary to a sequence of the label handle strand. Further still, a structured nucleic acid particle can be attached to a solid support by virtue of hybridizing to an immobilized oligonucleotide having a nucleotide sequence that is complementary to a sequence of the label handle strand. Exemplary nucleotide sequences that can be used for handles and complementary oligonucleotides in a method set forth herein are provided in Table 1.

A handle or complementary oligonucleotide set forth herein, including but not limited to those having sequences listed in Table 1, can optionally include an artificial cross-linking moiety. A structured nucleic acid particle (e.g., having a nucleic acid origami structure) can be stabilized by one or more artificial cross-links. An artificial cross-link can (1) attach a handle to a complementary oligonucleotide, (2) attach a handle to a scaffold, (3) attach a handle to a staple, (4) attach a staple to a scaffold; (5) attach a first staple to a second staple, or (6) attach a first region of a scaffold to a second region of the scaffold. One, some or all of the foregoing pairs can be attached via an artificial cross-link in a structured nucleic acid particle set forth herein.

A handle, such as a post, can be attached to a moiety of interest (e.g., an analyte, label, affinity moiety, particle or solid support) via an artificial polymer. A polymer can provide advantages of being inert to reagents that interact with the moiety of interest or providing a desired level of flexibility or rigidity to the handle. A particularly useful polymer is polyethylene glycol (PEG) including, but not limited to, those having an n of 1, 2, 3, 4, 5, 6, 7, 8, 9, 10 or longer. Alternatively or additionally, PEG can have an n of at most 10, 9, 8, 7, 6, 5, 4 3, 2 or 1.

As set forth previously herein, a plurality of complementary oligonucleotides can share a common nucleotide sequence that hybridizes to a particular type of label strand in a structured nucleic acid particle. A method of the present disclosure can be configured to hybridize one or more analyte handle strands of a structured nucleic acid particle to one or more analyte oligonucleotides, wherein the analyte oligonucleotides are attached to an analyte of interest. The nucleotide sequences that participate in hybridization can be the same for all analyte handle strands and all analyte oligonucleotides, respectively. Additionally, a method of the present disclosure can be configured to hybridize one or more label handle strands of a structured nucleic acid particle to one or more label oligonucleotides, wherein the label oligonucleotides are attached to a label. The nucleotide sequences that participate in hybridization of the label handle strands to the label oligonucleotides can be the same. However, the nucleotide sequences that participate in hybridization of the label handle strands to the label oligonucleotides can differ substantially from the nucleotide sequences that participate in hybridization of the analyte handle strands to the analyte oligonucleotides. The nucleotide sequences that participate in hybridization of the analyte oligonucleotides to the analyte handle strands can be designed to not substantially cross-hybridize with nucleotide sequences that participate in hybridization of the label oligonucleotides to the label handle strands. Conversely, the nucleotide sequences that participate in hybridization of the label oligonucleotides to the label handle strands can be designed to not substantially cross-hybridize with nucleotide sequences that participate in hybridization of the analyte oligonucleotides to the analyte handle strands. As such, both pairs of sequences can be present in a common hybridization reaction without resulting in unwanted formation of mis-hybridized species.

A method of the present disclosure can be configured to hybridize one or more attachment handle strands of a structured nucleic acid particle to one or more attachment oligonucleotides, wherein the attachment oligonucleotides are attached to a solid support (e.g., particle or site of an array). The nucleotide sequences that participate in hybridization of the attachment handle strands to the attachment oligonucleotides can be the same. However, the nucleotide sequences that participate in hybridization of the attachment handle strands to the attachment oligonucleotides can differ substantially from the nucleotide sequences that participate in hybridization of the analyte handle strands to the analyte oligonucleotides. The nucleotide sequences that participate in hybridization of the attachment handle strands to the attachment oligonucleotides can also differ substantially from the nucleotide sequences that participate in hybridization of the analyte handle strands to the analyte oligonucleotides. The nucleotide sequences that participate in hybridization of the attachment oligonucleotides to the attachment handle strands can be designed to not substantially cross-hybridize with nucleotide sequences that participate in hybridization of the analyte oligonucleotides to the analyte handle strands. Moreover, the nucleotide sequences that participate in hybridization of the attachment oligonucleotides to the attachment handle strands can be designed to not substantially cross-hybridize with nucleotide sequences that participate in hybridization of the label oligonucleotides to the label handle strands. As such, all three pairs of sequences can be present in a common hybridization reaction without resulting in unwanted formation of mis-hybridized species.

Also provided herein is a structured nucleic acid particle, including (a) a scaffold strand having a nucleotide sequence; (b) a plurality of different staple strands having different nucleotide sequences hybridized to the scaffold strand, wherein each of the staple strands is hybridized to two non-contiguous regions in the nucleotide sequence of the scaffold strand; and (c) a handle strand having a nucleotide sequence hybridized to the scaffold strand or to a staple strand, wherein the handle strand is attached to the scaffold strand or to the staple strand by an artificial cross-link.

The applications for which structured nucleic acid particles are used, and the chemical environments where they are deployed can be expanded by stabilizing the structure of the particle. Watson-Crick base pairing and other chemical forces allow structured nucleic acid particles to maintain structural integrity in a fairly wide range of physiological conditions. However, conditions outside of the normal physiological range can be useful when assaying analytes that are attached to structured nucleic acid particles as set forth herein. For example, a protein that is attached to a structured nucleic acid particle can be identified via a binding assay in which a series of different affinity reagents are bound to the protein and then dissociated from the protein. Although the binding event typically occurs under physiological conditions, dissociation is favored by harsher conditions. Moreover, in some assays it is beneficial for the protein to be denatured, a state that is facilitated by conditions that are harsher than physiological conditions. Conditions employed to denature proteins and dissociate affinity reagents from proteins can increase the risk of disrupting the non-covalent forces that maintain the structural integrity of structured nucleic acid particles. This risk can be reduced or mitigated by modifying structured nucleic acid particles to incorporate artificial moieties such as artificial nucleotide analogs that interact more strongly than naturally occurring nucleotides or covalent cross-links between nucleic acid components that would otherwise be maintained by non-covalent forces. Exemplary artificial nucleotides and cross-links for stabilized structured nucleic acid particles are set forth below along with methods of creating the stabilized particles.

A particularly useful cross-link is a cyclobutane pyrimidine dimer such as a cyclobutane thymidine dimer, cyclobutane cytidine dimer, or cyclobutane cytidine-thymidine dimer. A cyclobutane pyrimidine dimer can be formed by UV irradiation of a structured nucleic acid particle having proximal pyrimidines. Conditions for UV cross-linking are set forth in further detail in Example II herein or in Gerling et al., Sci. Adv. 4: eaau1157 (2018), which is incorporated herein by reference. Locations for cross-links in a structured nucleic acid particle can be determined by evaluating the folded structure to identify proximal pyrimidines. Software developed for folding nucleic acid origami structures, such as programs set forth herein, can be used. Cross-links can be engineered into a structured nucleic acid particle by placing pyrimidines (e.g., thymidines, cytidines or analogs thereof) at locations in the structure that are adjacent to each other. The pyrimidines can be placed using molecular biology techniques for site directed mutagenesis (e.g., to modify scaffolds that are derived from biological systems) or using techniques for oligonucleotide synthesis (e.g., to modify staples or handles that are synthetically derived). Particularly useful locations for pyrimidines that participate in cross-links include, but are not limited to, termini of adjacent strands, strand breaks at crossover sites, or loops within a strand.

A cyclobutane pyrimidine dimer can cross-link different strands in a structured nucleic acid particle. For example, a first region of a staple strand can be cross-linked to a second region of the scaffold strand via a cyclobutane pyrimidine dimer, a staple strand can be cross-linked to a scaffold strand via a cyclobutane pyrimidine dimer, a first staple strand can be cross-linked to a second staple strand via a cyclobutane pyrimidine dimer, a staple strand can be cross-linked to a handle strand via a cyclobutane pyrimidine dimer, a handle strand can be cross-linked to a scaffold strand via a cyclobutane pyrimidine dimer, or a first handle strand can be cross-linked to a second handle strand via a cyclobutane pyrimidine dimer. In some configurations, a cyclobutane pyrimidine dimer can cross-link a nucleic acid strand at regions that are distant from each other in the linear sequence of the strand but proximal in the three-dimensional conformation of the nucleic acid strand. For example, a cyclobutane pyrimidine dimer can cross-link a pair of pyrimidines that are separated by a gap in the nucleotide sequence of a scaffold strand, staple strand or handle strand.

A structured nucleic acid particle can be cross-linked using introduced cross-linking reagents. Intercalators can be useful as cross-linkers. Exemplary cross-linkers include psoralens which can be useful for photo-cross-linking a structured nucleic acid particle. Psoralens are tricyclic furocoumarins that can intercalate nucleic acids and when irradiated with long wave UV (e.g., 315-400 nm) result in cross-linking pyrimidines. Transitional metals can be useful for cross-linking structured nucleic acid particle. Examples include, but are not limited to, metals in the platinum group of the periodic table (e.g., ruthenium, rhodium, palladium, osmium, iridium, and platinum). Pt(II) includes two liable ligands that react with nucleotides to form inter and intra-cross-linking duplex DNA An exemplary Pt(II) reagent is cisplatin (cis-diamminedichloroplatinum(II)). The extent of the inter-strand cross-linking can be influenced by the size and nature of the non-liable ligands. Copper is a metal ion and complex that can be used to cross-link structured nucleic acid particles through the bases of the nucleic acid. Zinc ions and complexes can also interact with nucleotides to form cross-links in double stranded regions of structured nucleic acid particles. Other nucleic acid cross-linking reagents include, for example, nitrogen mustards, Chloro ethyl nitroso urea (CENU), carmustine (BCNU), Mitomycin C, nitrous acid, or bifunctional aldehydes.

Artificial nucleotide analogs can be used for cross-linking. For example, nucleic acids can include nucleotides having moieties that react in a click reaction or bioorthogonal reaction. The nucleotides can be introduced at locations in a structured nucleic acid particle that are adjacent in its three-dimensional structure. Modified nucleotides can be introduced during oligonucleotide synthesis or by enzymatic extension of the 3′ end of a nucleic acid. A structured nucleic acid particle can be configured to include locked nucleic acid (LNA). Non-covalent binding interactions between LNA and DNA are stronger than those between two strands of DNA. Peptide nucleic acid (PNA) can also be used to stabilize structured nucleic acid particles. Since PNA employs an amide bond linkage between nucleotides, stability can be gained by increasing the distance between the charges of the phosphodiester backbone. This can make structured nucleic acid particles more resistant to variation in concentration in Group II ions.

Another useful nucleotide analog is an abasic nucleotide. An abasic nucleotide can be introduced into a nucleic acid strand of a structured nucleic acid particle to form a crosslink with a complementary nucleic acid strand. The aldehyde moiety of a DNA abasic nucleotide can generate an interstrand cross-link via imine formation with the exocyclic N²-amino group of a guanine residue on the opposing strand in 5′-d(CAp) sequences (where Ap is an abasic site). See Dutta et al., J. Am. Chem. Soc. 129:1852-1853 (2007), which is incorporated herein by reference. Abasic nucleotides in nucleic acid strands are stable in 0.2M triethylammonium acetate buffer (pH6) at 5° C. or less. The crosslinking reaction can be induced by reducing pH or addition of sodium borohydride. A useful abasic nucleotide is the Abasic II Phosphoramidite (5-O-Dimethoxytrityl-1-O-tert-butyldimethylsilyl-2-deoxyribose-3-[(2-cyanoethyl)-(N,N-diisopropyl)]-phosphoramidite) available from Glen Research (Sterling, VA).

Artificial nucleotides and nucleic acid cross-links, such as those exemplified above, can be used to strengthen attachment between a handle strand of a structured nucleic acid particle and an oligonucleotide that hybridizes to the handle strand. For example, a nucleotide sequence of an attachment handle can be cross-linked to a complementary sequence of an attachment oligonucleotide, a nucleotide sequence of an analyte handle can be cross-linked to a complementary sequence of an analyte oligonucleotide, and/or a nucleotide sequence of a label handle can be cross-linked to a complementary sequence of a label oligonucleotide. As such, the present disclosure provides a method of hybridizing a handle sequence of a structured nucleic acid particle to a complementary oligonucleotide and cross-linking the hybridized species to form a covalent linkage. Similarly, an attachment handle and/or attachment oligonucleotide can include artificial nucleotides; an analyte handle and/or analyte oligonucleotide can include artificial nucleotides, or a label handle and/or label oligonucleotide can include artificial nucleotides. As such, the present disclosure provides a method of hybridizing a handle sequence of a structured nucleic acid particle to a complementary oligonucleotide and stabilizing the hybridized species via interactions between artificial nucleotides or interactions between artificial nucleotides and naturally occurring nucleotides. A solid support can be covalently attached to a structured nucleic acid particle via an inter-strand cross-link set forth herein, an analyte of interest can be covalently attached to a structured nucleic acid particle via an inter-strand cross-link set forth herein, and or a label can be covalently attached to a structured nucleic acid particle via an inter-strand cross-link set forth herein. Exemplary methods for photo-cross-linking structured nucleic acid particles are set forth in Example II herein.

Another approach for stabilizing structured nucleic acid particles is to coat the particle with an inorganic or organic substance. Coatings can increase stability by increasing resistance to enzymatic digestion (e.g., inhibition of nucleases) or by reducing sensitivity to low ion concentrations. For example, oligolysine-based coatings can be used, for example, as set forth in Ponnuswamy et al. Nature Communications volume 8, Article number: 15654 (2017), which is incorporated herein by reference. A structured nucleic acid particle can be coated with calcium phosphate, for example, as set forth in Wu et al., ACS Nano 15:1555-1565 (2021), which is incorporated herein by reference.

Stabilization strategies, such as those set forth herein, can be employed for differential processing of structured nucleic acid particles. For configurations in which structured nucleic acid particles are attached to different molecules (e.g., analytes, affinity moieties, etc.), differential stability of the particles can be exploited to achieve differential processing of those molecules.

In a first example, structured nucleic acid particles that are attached to analytes of interest can have increased stability compared to structured nucleic acid particles that are attached to affinity moieties. Turning to the more specific example of a binding assay configured to detect analytes, a first set of structured nucleic acid particles can be attached to the analytes and a second set of structured nucleic acid particles can be attached to affinity moieties that recognize the analytes, wherein the first set of structured nucleic acid particles have higher stability than the second set of structured nucleic acid particles. The assay can include a binding step in which particle-attached affinity moieties bind to particle-attached analytes, followed by a dissociation step in which the particle-attached affinity moieties are removed from the particle-attached analytes. The dissociation step can use conditions that denature the second subset of structured nucleic acid particles but do not substantially compromise the structural integrity of the first subset structured nucleic acid particles at least in part due to stabilization of the first subset. Accordingly, the analyte-attached particles can be subjected to subsequent processing, for example, subsequent binding of affinity reagents to the attached analytes.

In a second example, a first subset of structured nucleic acid particles that are attached to a first subset of analytes can have increased stability compared to a second subset of structured nucleic acid particles that are attached to a second subset of analytes. The two subsets of analytes can, for example, be derived from different biological samples or from different aliquots of a biological sample. The two subsets of particle attached analytes can be mixed in a pool, for example, for use in a multiplexed detection assay. The second subset of analytes can be separated from the first subset of analytes, or otherwise rendered undetectable in the assay, by a process that includes denaturing the second subset of structured nucleic acid particles under conditions that do not substantially compromise the structural integrity of the first subset structured nucleic acid particles.

Structured nucleic acid particles can be differentially denatured using any of a variety of conditions appropriate for the type of stabilization employed. For example, interactions between naturally occurring nucleotides in structured nucleic acid particles are sensitive to the presence of divalent metal ions, and these particles can be denatured by removal or reduction in the concentration of the divalent metal ions. In contrast, structured nucleic acid particles that are stabilized by covalent cross-links, or artificial nucleotides such as LNA or PNA, are stable in solutions having little to no divalent metal ions. As such, divalent metal ions can be removed of their concentration reduced to differentially denature non-stabilized particles in the presence of stabilized particles. Other conditions that can be applied to achieve differential denaturation include, for example, increasing temperature; increasing pH (e.g., by adding NaOH); adding chemical denaturants such as dimethylsulfoxide (DMSO), formamide or alcohol (e.g., ethanol or isopropanol); increasing ionic strength (e.g., increasing concentration of salts such as NaCl or KCl); and/or physical perturbations such as sonication. The present disclosure provides a structured nucleic acid particle that is adjustable to at least two different conformational states. The structured nucleic acid particle can be attached to an analyte, wherein, in a first of the at least two conformations, the analyte is accessible for binding to an affinity reagent that recognizes the analyte, and wherein in a second of the at least two conformations the analytes is inhibited from binding to the affinity reagent. Optionally, the structured nucleic acid particle is configured to toggle between at least two states, for example, being capable of converting from the first conformation to the second conformation, and also being capable of converting from the second conformation to the first conformation. Accordingly, the structured nucleic acid particle, when in the first conformation, can be bound to the affinity reagent via the analyte. The structured nucleic acid particle can be unbound to (or dissociated from) the affinity reagent in the second conformation, even when the affinity reagent is in diffusional contact with the structured nucleic acid particle.

The present disclosure provides a method of reversibly binding an affinity reagent to an analyte. The method can include steps of (a) binding an analyte to an affinity reagent, wherein the analyte is attached to a structured nucleic acid particle during the binding, wherein the structured nucleic acid is in a first conformation; and (b) converting the structured nucleic acid particle to a second conformation, wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation; and (d) binding a second affinity reagent to the analyte.

In some configurations, a structured nucleic acid particle that is adjustable to multiple conformations can be attached to an analyte via a linker, wherein the analyte is more distant from a surface of the structured nucleic acid particle in a first conformation and less distant from the surface in a second conformation. The proximity of the analyte to the surface can inhibit binding of the analyte to an affinity reagent that is otherwise capable of recognizing and binding to the analyte. For example, the affinity reagent can be inhibited from binding the analyte in conditions that would otherwise favor binding such as conditions in which the affinity reagent or analyte is present at a concentration that is greater than or equal to the equilibrium binding constant (Kₐ). Inhibition can arise due to steric interference, for example, between the structured nucleic acid particle and the affinity reagent. Alternatively or additionally, inhibition can arise due to charge repulsion between the structured nucleic acid particle and affinity reagent, incompatible polarity between the structured nucleic acid particle and affinity reagent, or other interactions that result in repulsion between the structured nucleic acid particle and affinity reagent.

A linker can be composed of any material or substance that is capable of attaching a nucleic acid to another analyte. Polymers can be particularly useful. Linear polymers are particularly useful but branched polymers can be used in some cases. A linker can include a synthetic polymer and/or biological polymer. Exemplary components of synthetic polymers include, but are not limited to, polyethylene, polyethylene glycol, polypropylene, polystyrene, polyvinyl chloride, polyacrylamide, hydrocarbons, polyester, or artificial nucleic acids such as peptide nucleic acid (PNA), locked nucleic acid (LNA) or Hachimoji DNA. Exemplary biological polymers include, but are not limited to, nucleic acids such as DNA or RNA, proteins, polysaccharides, lipids or the like. In some cases, a linker can include a handle or can be attached to a handle. Moreover, a handle can include a linker or be attached to a linker. It will be understood that a linker can have a composition set forth herein in the context of handles. Conversely, a handle can have a composition set forth herein in the context of linkers.

A linker can include a first moiety that connects to a structured nucleic acid particle and a second moiety that connects to an analyte. The length of a linker between the first moiety and the second moiety can be selected to suit a particular use. The length of the linker will be understood to be measured along a path that passes through the linker in the shortest possible distance from the first moiety to the second moiety. As such, the length is typically the same whether the linker is straight or curved. This is the case whether the shortest distance between any particular region of the structured nucleic acid particle and the second moiety changes due to variation in the curvature of the linker itself. Optionally, the length of a linker between a first moiety, which connects to the structured nucleic acid particle, and a second moiety which connects to an analyte, can be at least about 5 nm, 10 nm, 20 nm, 30 nm, 40 nm, 50 nm, 75 nm, 100 nm, 200 nm, 300 nm, 400 nm, 500 nm, 1 micron, 2 microns, 3 microns, 4 microns, 5 microns, or more. Alternatively or additionally, the length can be at most 5 microns, 4 microns, 3 microns, 2 microns, 1 micron, 500 nm, 400 nm, 300 nm, 200 nm, 100 nm, 75 nm, 50 nm, 40 nm, 30 nm, 20 nm, 10 nm, 5 nm or less.

A linker that attaches an analyte to a structured nucleic acid particle can be composed of nucleic acid. The nucleic acid can be single stranded, double stranded, or can have a combination of single stranded and double stranded regions. A linker can include a first moiety which connects to the structured nucleic acid particle and a second moiety which connects to the analyte. Optionally, the first moiety includes a backbone region of a nucleic acid strand that forms part of the linker and also forms part of another region in the structured nucleic acid particle. For example, the nucleotide strand can optionally pass through the linker and through a region of the structured nucleic acid particle other than the linker. Optionally, the nucleotide strand can be threaded through a linker region of a structured nucleic acid particle and another region of the structured nucleic acid particle such that the first moiety includes a plurality of non-contiguous regions in the sequence of the strand. For example, the first moiety can include at least 2, 3, 4, 5, 6 or more non-contiguous regions in the sequence of the strand. Optionally, the strand is a scaffold strand or a staple strand, wherein the structured nucleic acid particle includes nucleic acid origami.

A linker can include a continuous chain of covalent bonds from a first moiety which connects to the structured nucleic acid particle to a second moiety which connects to the analyte. For example, release of an analyte from a structured nucleic acid particle in some configurations can require breakage of at least one covalent bond in the linker. Alternatively, a linker can include one or more non-covalent bonds connecting the first moiety to the second moiety. In this configuration, the linker can be cleaved by disrupting a non-covalent bond to release an analyte from a structured nucleic acid particle. For example, the analyte can be released without disrupting any covalent bonds in the linker. In many cases, the linker can include a combination of covalent and non-covalent bonds. For example, a linker can be cleaved by breaking a combination of covalent and non-covalent bonds to release an analyte from a structured nucleic acid particle.

Particularly useful non-covalent bonds that can be used in a linker attaching a structured nucleic acid particle to an analyte include receptor-ligand interactions. Examples include, but are not limited to, antibodies which bind to antigens; (strept)avidin, or analogs thereof, which bind to biotin or analogs thereof; lectins which bind to carbohydrates; nucleic acids or analogs thereof (e.g., DNA, RNA, peptide nucleic acids), which bind to each other via complementary sequence interactions; or affibodies, affilins, affimers, affitins, alphabodies, anticalins, avimers, DARPins, monobodies, nanoCLAMPs, polypeptides, nucleic acid aptamers, or protein aptamers which bind to various epitopes. Further examples of non-covalent bonds include hydrogen bonds, ionic bonds, van der Waals forces, pi-effects, dipole-dipole interactions, hydrophobic interactions, or electrostatic interactions.

In particular configurations, a method of reversibly binding an affinity reagent to an analyte includes steps of (a) binding an analyte to an affinity reagent, wherein the analyte is attached to a structured nucleic acid particle by a nucleic acid linker strand during the binding, wherein the structured nucleic acid particle is in a first conformation, and wherein, in the first conformation, the linker further includes a second strand hybridized to the first strand; and (b) converting the structured nucleic acid particle to a second conformation, wherein the converting of the structured nucleic acid particle to the second conformation includes removing the second strand from the linker and self-hybridizing the first strand to form a hairpin conformation, wherein the analyte is more distant from a surface of the structured nucleic acid particle in the first conformation compared to the second conformation, wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation by hybridizing a third strand to the linker strand; and (d) binding a second affinity reagent to the analyte.

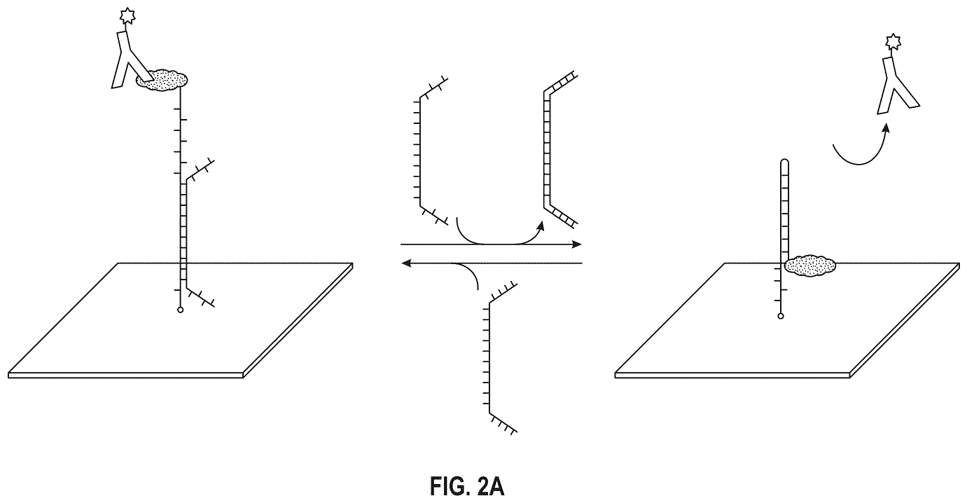

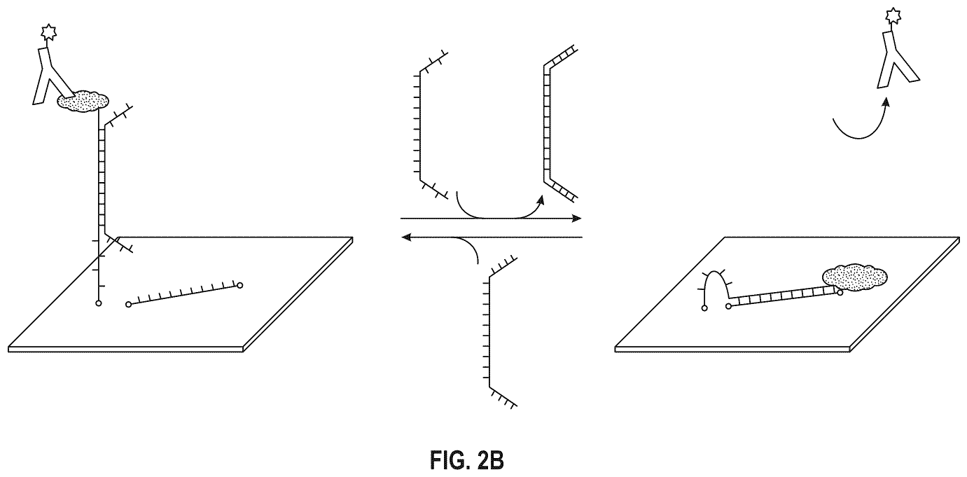

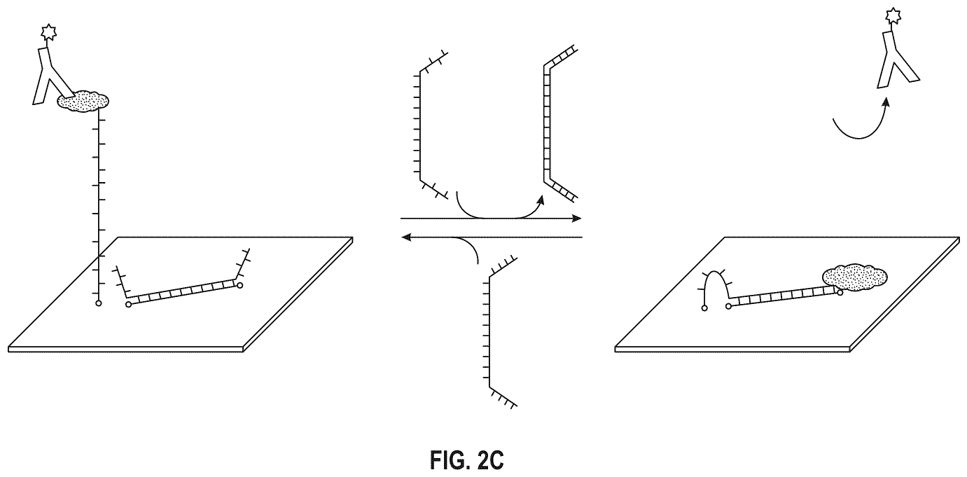

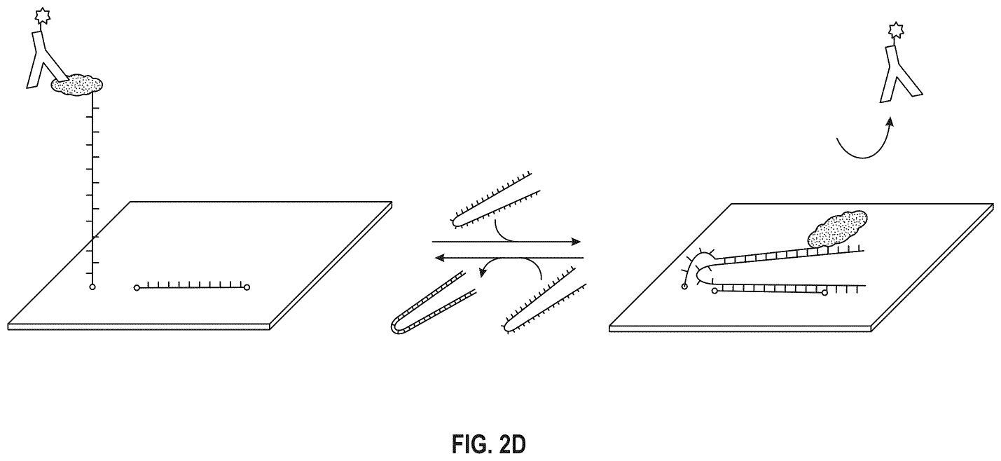

FIG. 2A shows a diagram of a reversible reaction in which a structured nucleic acid particle (shaded square tile) is attached to a protein (stippled globule) by a linker that can be toggled between an extended conformation (left side of the reaction) and a hairpin conformation (right side of the reaction). In this example, the linker is a nucleic acid strand and the extended conformation includes a first oligonucleotide (indicated by a black line with two bends) hybridized to the nucleic acid strand. In the extended conformation the protein can bind to an affinity reagent (Y-shape) having a detectable label (seven-point star). The first oligonucleotide is shown forming a ten base pair hybrid with the linker but the hybrid region can be shorter or longer, as desired. The first oligonucleotide is exemplified with two toehold regions that are not complementary to, nor hybridized to, the linker. At least one toehold region can be present to facilitate hybridization of the first oligonucleotide to a second oligonucleotide (grey line with two bends) via toehold mediated strand displacement or branch migration. As a result, the first oligonucleotide is released from the linker strand and at least part of the region that was previously hybridized to the first oligonucleotide self-hybridizes into a hairpin structure. In the hairpin conformation, the protein is proximal to the surface of the structured nucleic acid particle which inhibits the affinity reagent from binding to the protein. As such, the affinity reagent is released. The reaction is reversible such that the linker strand can be converted to extended form by hybridization to the first oligonucleotide (or another oligonucleotide that is at least partially homologous to the first oligonucleotide). Optionally, the hairpin linker can have at least one single stranded region that provides a toehold region to facilitate strand displacement or branch migration, as exemplified in the figure. The protein, being more distal from the surface of the structured nucleic acid particle, can then bind to an affinity reagent.

In particular configurations, a method of reversibly binding an affinity reagent to an analyte includes steps of (a) binding an analyte to an affinity reagent, wherein the analyte is attached to a structured nucleic acid particle by a nucleic acid linker strand during the binding, wherein the structured nucleic acid is in a first conformation, and wherein, in the first conformation, the linker further includes a second strand hybridized to the first strand; and (b) converting the structured nucleic acid particle to a second conformation, wherein the converting of the structured nucleic acid particle to the second conformation includes removing the second strand from the linker strand and hybridizing the linker strand to a nucleotide sequence of the structured nucleic acid particle, wherein the analyte is more distant from a surface of the structured nucleic acid particle in the first conformation compared to the second conformation, wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation by hybridizing a third strand to the linker strand; and (d) binding a second affinity reagent to the analyte.

FIG. 2B shows a diagram of a reversible reaction in which a structured nucleic acid particle is attached to a protein by a linker that can be toggled between a proud conformation (left side of the reaction) and a flush conformation (right side of the reaction). Components of the reaction are rendered as in FIG. 2A. The linker is a nucleic acid strand which is in an extended conformation when hybridization to a first oligonucleotide, thereby allowing the protein to bind to a labeled affinity reagent. The first oligonucleotide can include at least one toehold region to facilitate hybridization of the first oligonucleotide to a second oligonucleotide via toehold mediated strand displacement or branch migration. As a result, the first oligonucleotide is released from the linker strand and at least part of the region that was previously hybridized to the first oligonucleotide can hybridize to a single-stranded region at or near the surface of the structured nucleic acid particle. In this flush conformation, the protein is proximal to the surface of the structured nucleic acid particle which inhibits the affinity reagent from binding to the protein. As such, the affinity reagent is released. The reaction is reversible such that the linker strand can be converted to extended form by hybridization to the first oligonucleotide (or another oligonucleotide that is at least partially homologous to the first oligonucleotide). Optionally, the linker can have at least one single stranded region when hybridized in the flush conformation, thereby providing a toehold region to facilitate strand displacement or branch migration, as exemplified in the figure. The protein, being more distal from the surface of the structured nucleic acid particle, can then bind to an affinity reagent.

In particular configurations, a method of reversibly binding an affinity reagent to an analyte includes steps of (a) binding an analyte to an affinity reagent, wherein the analyte is attached to a structured nucleic acid particle by a nucleic acid linker strand during the binding, wherein the structured nucleic acid is in a first conformation, and wherein, in the first conformation, the structured nucleic acid particle includes a second strand hybridized to a third strand; and (b) converting the structured nucleic acid particle to a second conformation, wherein the converting of the structured nucleic acid particle to the second conformation includes removing the third strand from the second strand, thereby hybridizing the linker to the second strand, wherein the analyte is more distant from a surface of the structured nucleic acid particle in the first conformation compared to the second conformation, and wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation by hybridizing a fifth strand to the second strand; and (d) binding a second affinity reagent to the analyte.

FIG. 2C shows a diagram of a reversible reaction in which a structured nucleic acid particle is attached to a protein by a linker that can be toggled between a proud conformation (left side of the reaction) and a flush conformation (right side of the reaction). Components of the reaction rendered as set forth in FIG. 2B. However, in the proud conformation, the linker nucleic acid is single-stranded and a first oligonucleotide is hybridized to a nucleic acid strand that is located at or near the surface of the structured nucleic acid particle. Removal of the first oligonucleotide results in the linker strand hybridizing to the nucleic acid strand at or near the surface of the structured nucleic acid particle. In this example, the first oligonucleotide has a sequence region that is identical to a region of the linker strand that hybridizes to the structured nucleic acid particle. As exemplified above, the first oligonucleotide can include at least one toehold region to facilitate hybridization of the first oligonucleotide to a second oligonucleotide via toehold mediated strand displacement or branch migration. As a result, the first oligonucleotide is released from the structured nucleic acid particle and the structured nucleic acid particle can hybridize to the linker strand to form the flush conformation wherein the protein is proximal to the surface of the structured nucleic acid particle and inhibited from binding to the affinity reagent. As such, the affinity reagent is released. The reaction is reversible such that the nucleic acid linker can be converted to extended form by hybridization of the structured nucleic acid particle to the first oligonucleotide (or another oligonucleotide that is at least partially homologous to the first peptide). Optionally, the linker can have at least one single stranded region when hybridized in the flush conformation, thereby providing a toehold region to facilitate strand displacement or branch migration, as exemplified in the figure. The protein being more distal from the surface of the structured nucleic acid particle can then bind to an affinity reagent.

In particular configurations, a method of reversibly binding an affinity reagent to an analyte includes steps of (a) binding an analyte to an affinity reagent, wherein the analyte is attached to a structured nucleic acid particle by a nucleic acid linker strand during the binding, wherein the structured nucleic acid is in a first conformation, wherein, in the first conformation, (i) the first strand includes a single stranded nucleotide sequence, and (ii) the structured nucleic acid particle includes a second strand; and (b) converting the structured nucleic acid particle to a second conformation, wherein the converting of the structured nucleic acid particle to the second conformation includes hybridizing a splint strand to the first strand and the second strand, wherein analyte is more distant from a surface of the structured nucleic acid particle in the first conformation and less distant from the surface in the second conformation, and wherein the structured nucleic acid particle blocks binding of the analyte to the affinity reagent, thereby dissociating the affinity reagent from the analyte. Optionally, the method can further include steps of (c) after step (b), converting the structured nucleic acid particle to the first conformation by hybridizing a reset strand to the splint strand; and (d) binding a second affinity reagent to the analyte.

FIG. 2D shows a diagram of a reversible reaction in which a structured nucleic acid particle is attached to a protein by a linker that can be toggled between a proud conformation (left side of the reaction) and a flush conformation (right side of the reaction). Components of the reaction are rendered as in FIG. 2A. The linker is a nucleic acid strand which, in the proud conformation, allows the protein to bind to a labeled affinity reagent. The linker can be converted to the flush conformation by hybridization of the linker to a splint that also hybridizes to a strand located at or near the surface of the structured nucleic acid particle. The splint is indicated by a u-shaped black line in the figure. In the flush conformation, the protein is proximal to the surface of the structured nucleic acid particle which inhibits the affinity reagent from binding to the protein. As such, the affinity reagent is released. The reaction is reversible such that the nucleic acid linker can be converted to extended form by hybridization of the splint to a reset oligonucleotide (indicated by a u-shaped grey line) that is complementary to the splint oligonucleotide. Optionally, the splint oligonucleotide can have at least one single stranded region when hybridized in the flush conformation, thereby providing a toehold region to facilitate strand displacement or branch migration, as exemplified in the figure. The protein being more distal from the surface of the structured nucleic acid particle can then bind to an affinity reagent.

As exemplified by FIGS. 2A through 2D, an adjustable structured nucleic acid particle or method of reversibly binding an affinity reagent to an analyte can employ a nucleic acid linker having a first conformation in which an attached analyte is accessible to an affinity reagent and having a second conformation in which the attached analyte is inhibited from binding to the affinity reagent. The linker can be single stranded in the first conformation and double stranded in a second conformation, for example, as shown in FIGS. 2C and 2D. Alternatively, the linker can be double stranded in the first conformation and in the second conformation, for example, as shown in FIGS. 2A and 2B. Optionally, a linker strand can be hybridized to another oligonucleotide in the first conformation and self-hybridized to form a hairpin in the second conformation, for example, as shown in FIG. 2A. Alternatively, a linker strand can be self-hybridized to form a hairpin in the first conformation and hybridized to another oligonucleotide in the second conformation. Optionally, strands that are to be removed from a linker strand can include a single-stranded region that functions as a toehold for strand displacement or branch migration, for example, as shown for FIGS. 2A through 2D. A toehold region can be present in the linker strand, for example as shown in FIGS. 2A, 2B and 2C.

The present disclosure provides a method of reversibly binding an affinity reagent to an analyte, including steps of (a) binding an analyte to an affinity reagent, wherein the analyte is attached to a surface of a structured nucleic acid particle during the binding, wherein the structured nucleic acid is in a first conformation in which the surface is exposed to the exterior of the structured nucleic acid particle; and (b) converting the structured nucleic acid particle to a second conformation, wherein the surface is sequestered within the structured nucleic acid particle in the second conformation, thereby blocking binding of the analyte to the affinity reagent and dissociating the affinity reagent from the analyte. Optionally, the method can further include steps of (c) after step (b), converting the structured nucleic acid particle to the first conformation; and (d) binding a second affinity reagent to the analyte.

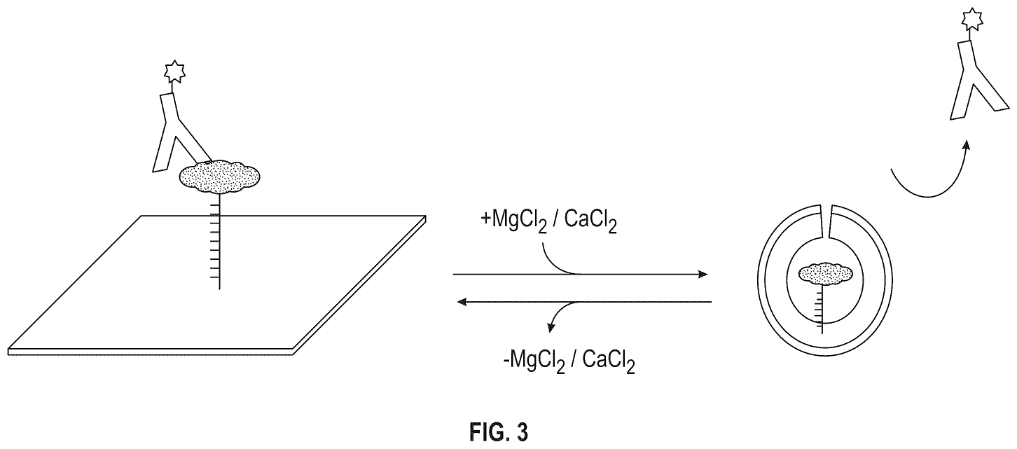

FIG. 3 shows a diagram of a reversible reaction in which a protein is attached to a surface of a structured nucleic acid particle that can be toggled between an open conformation (planar tile on the left side of the reaction) and a closed conformation (cylinder on the right side of the reaction). The surface of the structured nucleic acid particle to which the protein is attached is substantially planar in the open state and allows the protein to bind to an affinity reagent having a label. The planar structure occurs in the presence of high concentrations of MgCl₂ and/or CaCl₂. The open conformation can be converted to the closed conformation by removal or sequestration of MgCl₂ and/or CaCl₂. For example, the divalent cations can be sequestered by a chelating agent. In the absence of MgCl₂ and CaCl₂, or in the presence of low concentrations of MgCl₂ and/or CaCl₂, the structured nucleic acid particle folds to a more compact structure, exemplified by the cylinder in FIG. 3, whereby the affinity reagent is blocked from binding the protein. The cylinder in the closed conformation, as with the tile in the open conformation, is exemplary. The surface to which the protein is attached can be internalized by any of a variety of conformations taken by the structured nucleic acid particle. The structured nucleic acid particle can be engineered to toggle between any of a variety of shapes that respectively expose an attached analyte (e.g., the protein) and sequester the attached analyte. For example, a planar structure need not fully curl into a cylinder, instead forming a curved structure that interacts with a solid support surface, such that the combination of the solid support surface and curved particle surface sequesters an analyte that is attached to the particle. The closed conformation can be converted to the open conformation by exposing the structured nucleic acid particle to a sufficient concentration of MgCl₂ and/or CaCl₂.

Optionally, a structured nucleic acid particle that is toggled between an open and closed conformation can include nucleic acid origami. Conditions for converting nucleic acid origami from an open to closed state and vice versa are set forth, for example, in Hubner et al. Nanoscale 14:7898-7905 (2022), which is incorporated herein by reference. In particular configurations of methods set forth herein, the converting of a structured nucleic acid particle from an open conformation to a closed conformation includes contacting the structured nucleic acid particle with Mg²⁺, Ca²⁺, K⁺, H⁺, Na⁺, Li⁺, spermine (3+) or polycationic compounds. Cations can be present at a concentration over 1 mM, 10 mM, 50 mM, 100 mM, 500 mM or 1M to support the open conformation of a structured nucleic acid particle. Alternatively, cations can be absent or cations can be present at a concentration less than about 1M, 500 mM, 100 mM, 50 mM, 10 mM or 1 mM to support the closed conformation of a structured nucleic acid particle.

As exemplified above, a variety of methods can be used to convert a structured nucleic acid particle from a conformation that facilitates binding between a particle-attached analyte and an affinity reagent to a state that inhibits binding between the particle-attached analyte and the affinity reagent. Conversion of the particle from the state of being bound to the affinity reagent to an unbound state can occur in a fluid without necessarily changing the concentration of the affinity reagent in the fluid. For example, the affinity reagent can be present at a concentration that is greater than or equal to the equilibrium binding constant (Kₐ) for binding of the analyte to the affinity reagent. Alternatively, conversion of the particle from the state of being bound to the affinity reagent to the unbound state can further include reducing the concentration of the affinity reagent in the fluid. For example, the concentration of the affinity reagent in the fluid can be reduced to less than the equilibrium binding constant (Kₐ). The concentration of the affinity reagent can be lower than Kₐ by at least 10%, 50%, 100%, 2 fold, 5 fold, 10 fold, 100 fold or more. The concentration of the affinity reagent can be changed prior to, during or after changing the conformational state of the nucleic acid particle. In some cases, an affinity reagent that is dissociated from a particle-attached analyte can be removed from the presence of the particle-attached analyte. For example, the affinity reagent can be removed from a vessel or array that retains the particle-attached analyte.

A method of the present disclosure can include a step of contacting a particle-attached analyte with an affinity reagent. For example, the affinity reagent can be delivered to a vessel or array that retains the particle-attached analyte. Conversion of the particle from a non-bound state (i.e. not bound to an affinity reagent) to a state of being bound to an affinity reagent can occur in a fluid without necessarily changing the concentration of the affinity reagent in the fluid. For example, the affinity reagent can be present at a concentration that is greater than or equal to the Kₐ for binding of the analyte to the affinity reagent. Alternatively, conversion of the particle from the unbound state to the bound state can further include increasing the concentration of the affinity reagent in the fluid. For example, the concentration of the affinity reagent in the fluid can be increased to greater than the Kₐ. The concentration of the affinity reagent can be greater than Kₐ by at least 10%, 50%, 100%, 2 fold, 5 fold, 10 fold, 100 fold or more. The concentration of the affinity reagent can be changed prior to, during or after changing the conformational state of the nucleic acid particle.

In particular configurations of methods set forth herein, toggling of conformations for a structured nucleic acid particle to which an analyte is attached can allow multiple cycles of examining binding of the analyte to different affinity reagents. For example, a cycle of associating and dissociating a particle-attached analyte with a respective affinity reagent can be performed at least 1, 2, 3, 4, 5, 10, 25, 50, 100 or more times. Alternatively or additionally, a cycle of associating and dissociating a particle-attached analyte with a respective affinity reagent can be performed at most 100, 50, 25, 10, 5, 4, 3, 2 or 1 times.

It will be understood that the adjustable structured nucleic acid particles and methods for converting structured nucleic acid particles from one conformation to another are exemplified herein in the context of differentially exposing particle-attached analytes to affinity reagents, but can be extended to exposing analytes to other substances or materials that are known or suspected of interacting with the analytes. Other substances and materials include, for example, a reactive reagent that is capable of chemically modifying the analyte; an enzyme or other catalyst that facilitates reaction of the analyte with another molecule or cleavage of the analyte from the structured nucleic acid particle; a solid support, or surface thereof, that is capable of attaching to the analyte; another structured nucleic acid particle that is capable of attaching to the analyte, or the like.

Any of a variety of affinity reagents can be used to detect an analyte in a method set forth herein. Affinity reagents and methods for their use will be exemplified herein in the context of detecting proteins. It will be understood that the examples can be modified by those skilled in the art for detection of other analytes as well. Any molecule or other substance that is capable of specifically or reproducibly binding to a protein can be used as an affinity reagent. An affinity reagent can be larger than, smaller than or the same size as the protein to which it binds. An affinity reagent may form a reversible or irreversible bond with a protein. An affinity reagent may bind with a protein in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of a protein to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, lectins or functional fragments thereof.

An affinity reagent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g., fluorescence or phosphorescence) emission, luminescence lifetime, luminescence polarization, or the like; Rayleigh and/or Mie scattering; magnetic properties; electrical properties; charge; mass; radioactivity or the like. A label may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

A protein or other analyte that is attached to a structured nucleic acid particle can be detected in a fluid or on a solid support. For example, a structured nucleic acid particle, analyte, particle-attached analyte or other substance set forth herein can be present in fluid phase during one or more steps of a method set forth herein. The fluid can be in a vessel such as a test tube, flow cell, channel, flask, well (e.g., well in a multi-well plate), vat, reservoir or the like. Fluids can be mixed to facilitate contact between substances in the respective fluids. Alternatively, a method of the present disclosure can be configured for solid-phase processing of structured nucleic acid particles, analytes, or particle-attached analytes. For example, a structured nucleic acid particle, analyte, particle-attached analyte or other substance set forth herein can be immobilized on a solid-support during one or more steps of a method set forth herein. The solid support can be selected from those set forth herein or known in the art. A substance that is attached to a solid support can be contacted with another substance that is in a fluid during one or more steps of a method set forth herein.

A composition or method of the present disclosure can be configured for single analyte resolution, as exemplified here for proteins. Proteins that are attached to structured nucleic acid particles can be detected at single-protein resolution. Single protein resolution can be based on spatial or temporal separation of the protein from other proteins. For example, individual particle-attached proteins can be located at separate sites in an array, wherein the distance between nearest neighbor sites is resolvable by the detector used to examine the array. Alternatively to single-protein resolution, a detection method can be carried out at ensemble-resolution or bulk-resolution. Bulk-resolution configurations acquire a composite signal from a plurality of different proteins. A composite signal can be acquired from a population of multiple particle-attached proteins, for example, in a well or cuvette, or on a solid support surface.

A set of proteins can be contacted with a plurality of different affinity reagents. For example, a plurality of affinity reagents (whether configured separately or as a pool) may include at least 2, 5, 10, 25, 50, 100, 250, 300 or more different types of affinity reagents, each type of affinity reagent differing from the other types with respect to the epitope(s) recognized. Alternatively or additionally, a plurality of affinity reagents may include at most 300, 250, 100, 50, 25, 10, 5, or 2 different types of affinity reagents. Different types of affinity reagents in a pool can be uniquely labeled such that they can be distinguished from each other. In some configurations, at least two, and up to all, of the different types of affinity reagents in a pool may be indistinguishably labeled. Alternatively or additionally to the use of unique labels, different types of affinity reagents can be delivered and detected serially when evaluating polypeptides or sets of fragments from polypeptides, thereby allowing the affinity reagents to be temporally resolved.

Detection of multiple proteins can be performed in a multiplex format. In multiplexed formats, different proteins can be attached to different unique identifiers (e.g., sites in an array), and the proteins can be manipulated and detected in parallel. For example, a fluid containing one or more different affinity reagents can be delivered to an array of sites, each site having an immobilized protein, such that the sites of the array are in simultaneous contact with the affinity reagent(s). The proteins can be attached to the respective sites via structured nucleic acid particles or, alternatively, the proteins can be directly attached to the respective sites. Moreover, a plurality of sites can be observed in parallel allowing for rapid detection of binding events. Multiple proteins can be derived from a proteome or subfraction of a proteome.

A protein can be attached to a unique identifier in any of a variety of ways. A protein can be attached to a structured nucleic acid particle and the particle can be attached to a site on a solid support or to another unique identifier. Exemplary attachments include, but are not limited to, covalent or non-covalent attachments set forth herein in the context of attaching proteins to structured nucleic acid particles. Exemplary reagents and methods for attaching structured nucleic acid particles to solid supports are set forth in WO 2019/195633 A1; US Pat. App. Pub. Nos. 2021/0101930 A1 or 2022/0162684 A1; or U.S. Pat. Nos. 11,203,612 or 11,505,796, each of which is incorporated herein by reference.

Binding can be detected using any of a variety of techniques that are appropriate to the assay components used. For example, binding can be detected by acquiring a signal from a label attached to an affinity reagent when bound to a protein, acquiring a signal from a label attached to a protein when bound to an affinity reagent, or signal(s) from labels attached to an affinity reagent and protein to which the affinity reagent is bound (e.g., signals produced via Forster resonance energy transfer between a label on the affinity reagent and a label on a protein). In some configurations a complex between an affinity reagent and a protein need not be directly detected, for example, in formats where a nucleic acid tag or other moiety is created or modified as a result of binding. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or US Pat. App. Pub. No. 2022/0162684 A1, each of which is incorporated herein by reference.

A protein analyte can be detected by obtaining multiple binding measurements using different affinity reagents respectively. In particular configurations, the individual measurements may not, by themselves, be sufficiently accurate or specific to unambiguously identify the protein, but an aggregation of the multiple non-identical measurements can allow an identification to be made with a high degree of accuracy, specificity and confidence. For example, the multiple separate measurements can include subjecting the protein to reagents that are promiscuous with regard to recognizing multiple different proteins suspected of being present in a given sample from which the protein is derived. The use of promiscuous reagents can be particularly powerful in a multiplex format in which multiple different proteins are characterized in parallel. Accordingly, a first measurement carried out using a first promiscuous reagent may perceive a first subgroup of the different proteins without differentiating the identity of one protein in the subgroup from another protein in the subgroup. A second measurement carried out using a second promiscuous reagent may perceive a second subgroup of proteins, again, without differentiating the identity of one protein in the second subgroup from another protein in the second subgroup. However, a comparison of the first and second measurements can distinguish: (i) a protein that is uniquely present in the first subgroup but not the second subgroup; (ii) a protein that is uniquely present in the second subgroup but not the first subgroup; (iii) a protein that is uniquely present in both the first and second subgroups; or (iv) a protein that is uniquely absent in the first and second subgroups. The number of promiscuous reagents used, the number of separate measurements acquired, and degree of reagent promiscuity (e.g., the diversity of proteins recognized by the reagent) can be adjusted to suit the protein diversity expected for a particular sample from which the proteins are derived.

In particular configurations, an extant protein can be detected using one or more affinity reagents having known or measurable binding affinity for candidate proteins from the sample from which the extant protein is derived. For example, an affinity reagent can bind an extant protein to form a complex and a signal produced by the complex can be detected. An extant protein that is detected by binding to a known affinity reagent can be identified based on the known or predicted binding characteristics of the affinity reagent. For example, an affinity reagent that is known to selectively recognize a candidate protein suspected of being in a sample, without substantially binding to other candidate proteins in the sample, can be used to identify an extant protein in the sample as being the candidate protein merely by observing binding of the affinity reagent to the extant protein. This one-to-one correlation of affinity reagent to candidate protein can be used for identification of one or more different extant proteins. However, as the protein complexity (i.e. the number and variety of different proteins) in a sample increases, or as the number of different candidate proteins to be identified increases, the time and resources to produce a commensurate variety of affinity reagents having one-to-one specificity for fragment sets derived from the proteins approaches limits of practicality.

Methods set forth herein, can be advantageously employed to overcome these constraints. In particular configurations, the methods can be used to identify a number of different candidate proteins that exceeds the number of different affinity reagents used. For example, the number of different candidate proteins identified can be at least 5 fold, 10 fold, 25 fold, 50 fold, 100 fold or more than the number of affinity reagents used. This can be achieved, for example, by (1) using promiscuous affinity reagents that recognize multiple different candidate proteins suspected of being present in a given sample, and (2) subjecting extant proteins from the sample to a set of promiscuous affinity reagents that, taken as a whole, are expected to bind each candidate protein in a different combination, such that each protein is expected to produce a unique profile of binding and non-binding events. Promiscuity of an affinity reagent is a characteristic that can be understood relative to a given population of proteins. Promiscuity for a given affinity reagent can arise due to the affinity reagent recognizing an epitope that is known to be present in a plurality of different candidate proteins, wherein the candidate proteins are suspected of being present in the given population of proteins. For example, epitopes having relatively short amino acid lengths such as dimers, trimers, or tetramers are expected to occur in a substantial number of different proteins in the human proteome. Alternatively or additionally, a promiscuous affinity reagent can recognize different epitopes (i.e. epitopes having a variety of different structures), the different epitopes being present in a plurality of different candidate proteins. For example, a promiscuous affinity reagent that is designed or selected for its affinity toward a first trimer epitope may bind to a second epitope that has a different sequence of three amino acids when compared to the first epitope.

Although performing a single binding reaction between a promiscuous affinity reagent and proteins derived from a complex sample of proteins may yield ambiguous results regarding the identity of the different proteins to which it binds, the ambiguity can be resolved when the results are combined with other identifying information about those proteins. The identifying information can include characteristics of the protein such as length (i.e. number of amino acids), hydrophobicity, charge to mass ratio, isoelectric point, chromatographic fractionation behavior, enzymatic activity, presence or absence of post-translational modifications or the like. The identifying information can include results of binding with other promiscuous affinity reagents. For example, a plurality of different promiscuous affinity reagents can be contacted with proteins derived from a complex sample of proteins, wherein the plurality is configured to produce a different binding profile for each candidate protein suspected of being present in the population. In this example, each of the affinity reagents is distinguishable from the other affinity reagents, for example, due to unique labeling (e.g., different affinity reagents have different luminophore labels), unique spatial location (e.g., different affinity reagents are located at different sites in an array), and/or unique time of use (e.g., different affinity reagents are delivered in series to a population of polypeptides). Accordingly, the plurality of promiscuous affinity reagents produces a binding profile for an extant protein that can be decoded to identify a unique combination of epitopes present in the extant protein, and this can in turn be used to identify the extant protein as the candidate protein having the same or similar unique combination of epitopes. The binding profile can include observed binding events as well as observed non-binding events and this information can be compared to the presence and absence of epitopes, respectively, in a given candidate protein to make a positive identification.

In some configurations, distinct and reproducible binding profiles may be observed for some or even a substantial majority of extant proteins that are to be identified in a sample. However, in many cases one or more binding events produces inconclusive or even aberrant results. For example, observation of binding outcome for a single-molecule binding event can be particularly prone to ambiguities due to stochasticity in the behavior of single molecules when observed using certain detection hardware. The present disclosure provides methods that provide accurate protein identification despite ambiguities and imperfections that can arise in many contexts. In some configurations, methods for identifying, quantitating or otherwise characterizing one or more proteins in a sample utilize a binding model that evaluates the likelihood or probability that one or more candidate proteins will have produced an empirically observed binding profile. The binding model can include information regarding expected binding outcomes (e.g., binding or non-binding) for binding of one or more affinity reagent with one or more candidate polypeptides. The information can include apriori characteristics of one or more candidate proteins under particular conditions. A binding model can include information regarding the propensity or likelihood of a given candidate protein generating a false positive or false negative binding result in the presence of a particular affinity reagent. Methods set forth herein can be used to evaluate the degree of compatibility of one or more empirical binding profiles (e.g., obtained from one or more extant protein) with results computed for various candidate proteins using a binding model. For example, to identify an extant protein in a sample of many proteins, an empirical binding profile for the protein can be compared to results computed by the binding model for many or all candidate proteins suspected of being in the sample. In some configurations of the methods set forth herein, identity for the extant protein is determined based on a likelihood of the extant protein being a particular candidate protein given the empirical binding pattern, or based on the probability of the particular candidate protein generating the empirical binding pattern. Optionally a score can be determined from the measurements that are acquired for the extant protein with respect to many or all candidate proteins suspected of being in the sample. A digital or binary score that indicates one of two discrete states can be determined. In particular configurations, the score can be non-digital or non-binary. For example, the score can be a value selected from a continuum of values such that an identity is made based on the score being above or below a threshold value. Moreover, a score can be a single value or a collection of values. Particularly useful methods for identifying proteins using promiscuous reagents, serial binding measurements and/or decoding with binding models are set forth, for example, in U.S. Pat. No. 10,473,654 US Pat. App. Pub. Nos. 2020/0318101 A1 or 2023/0114905 A1 or Egertson et al., BioRxiv (2021), DOI. 10.1101/2021.10.11.463967, each of which is incorporated herein by reference.

### Example I

### Handle Design for Minimal Cross-Reactivity

Various moieties can be attached to a DNA origami via hybridization of oligonucleotides that bear the moieties to respective handle strands in the origami structure. The origami structure can be engineered to place particular handle strands at desired locations. However, cross-reactivity can result in hybridization of an oligonucleotide to the wrong handle, thereby misplacing the moiety that is attached to the oligonucleotide. This example describes a method for designing and qualifying DNA origami to handle sets that avoid unwanted cross-reactivity. The method involves performing temperature melts of single and double oligonucleotide mixes to quantify oligonucleotide-oligonucleotide interaction. For quantitative analysis of the melt data, an analysis tool was developed for the comparison of single and double oligonucleotide temperature melts.

Handle design was treated as an optimization problem. Three handles were designed each to specifically hybridize to a complementary oligonucleotide. Specificity of binding between handle sequences and intended oligonucleotides was evaluated using melt experiments on a qPCR instrument. Oligonucleotides at 0.9 μM or 9 μM were melted from 5° C. to 95° C. in the presence of Sybr-Green Lumiprobe and five data points were acquired per degree. Binding between two oligonucleotides was quantified by taking the derivative of melting curves obtained from the melt experiments as follows. Noise was flattened for each melt curve using window averaging, then the first-order derivative was calculated followed by normalization of the data (mean=0, standard deviation—1). The single oligonucleotides melt derivatives were summed and cross-correlation between double oligonucleotide melts and the sum of single oligonucleotide melts was determined. As such, data analysis involved smoothing and normalizing melt data, and comparison of double and single melt derivatives using cross-correlation, wherein a cross-correlation of one indicated no interaction and a cross-correlation less than one indicated interaction between the two oligonucleotides. The design, melt and analysis identified three useful pairs of sequences including the APT 40.2 handle strand which is specific for the TCOv40.2 strand with a melting temperature (Tₘ) of 77.4° C.; the DYE40.22.9 handle strand which is specific for the DYE647v40.22.9 strand with a Tₘ of 76° C.; and the JF20.22.9 handle strand which is specific for the JFDBCOv20.22.9 strand with a Tₘ of 64.4° C. Two other pairs were designed including Tether1 which is specific for Docker1; and Tether2 which is specific for Docker2. Sequences for the strands are listed in Table 1.

TABLE 1
Nucleotide Sequences for Handles

| | | SEQ |
|---|---|---|
| | | ID |
| Name | Nucleotide sequence | NO |
| APT 40.2 | CACTCACCTCCATCTCCACTCCTACCCATCCAACTCCCAC | 1 |
| TCOv40.2 | GTGGGAGTTGGATGGGTAGGAGTGGAGATGGAGGTGAGTG | 2 |
| DYE40.22.9 | CGTTTCTTTGCTTGTGTCTTGTCTTCGTCGTGCTTGTTCC | 3 |
| DYE647v40.22.9 | GGAACAAGCACGACGAAGACAAGACACAAGCAAAGAAACG | 4 |
| JF20.22.9 | ACAGTTATTAGCCCGCATTT | 5 |
| JFDBCOv20.22.9 | AAATGCGGGCTAATAACTGT | 6 |
| Tether 1 | CTAGATGTAT | 7 |
| Docker 1 | ATACATCTAG | 8 |
| Tether 2 | TAAGACAGAT | 9 |
| Docker 2 | TACTGTCTTA | 10 |

Optionally, a handle oligonucleotide can include a region having one of the sequences listed in Table 1 and can further include an additional region having the additional sequence GTGGATGGGTAGGAGTGGAGATGGAGGTGAGTG (SEQ TD NO: 11). The additional sequence can be on the 5′ or 3′ end of a sequence listed in Table 1. As a further option, a linker sequence TTT can link a sequence of Table 1 to the additional sequence.

The DNA origami Tile was prepared as follows. A master mix was formed including 120 μl of 500 nM staple nucleic acids (listed in Table 2), 60 μl of 1 μM attachment handle nucleic acids, 40 μl of 100 nM p7249 m13 ssDNA scaffold (Tilibit Nanosystems, Germany), 40 μl of 10×FOB (50 mM NaCl, 10 mM EDTA, 50 mM Tris-HCl pH 8.0), 25 μl of 200 mM MgCl₂ and 115 μl water. The master mix was folded in strip tubes in a PCR instrument using the following cycle: 2 min at 95° C. followed by cooling from 90° C. to 20° C. at a rate of 1° C. per minute.

TABLE 2
Oligonucleotide Sequences for DNA Origami Tile

| | | SEQ |
|---|---|---|
| | | ID |
| Sequence Name | Sequence | NO |
| 1-Reg-T1R01C6 | TCATTTGCTAATAGTAGTAGCATT | 12 |
| 2-Reg-T1R03C5 | CAACTAAAGTACGGTGGGATGGCT | 13 |
| 3-Reg-T1R09C2 | CATTATTAGCAAAAGAAGTTTTGC | 14 |
| 4-Reg-T2R01C6 | ACCCTCATTCAGGGATAGCAAGCC | 15 |
| 5-Reg-T2R03C5 | TTAGGATTAGCGGGGTGGAACCTA | 16 |
| 6-Reg-T2R09C2 | AGGCCGGAACCAGAGCCACCACCG | 17 |
| 7-Reg-T3R01C6 | AGAATATCAGACGACGACAATAAA | 18 |
| 8-Reg-T3R03C5 | TCATATGCGTTATACAAAGGCGTT | 19 |
| 9-Reg-T3R09C2 | CGGGAGAATTTAATGGAAACAGTA | 20 |
| 10-Reg-T4R01C6 | GCGCGTACTTTCCTCGTTAGAATC | 21 |
| 11-Reg-T4R03C5 | AAAGCCGGCGAACGTGTGCCGTAA | 22 |
| 12-Reg-T4R09C2 | AATTCCACGTTTGCGTATTGGGCG | 23 |
| 13-Reg-T1R06C3 | TTAAGAGGGTCCAATACTGCGGATAGCGAG | 24 |
| 14-Reg-T1R08C3 | AGGCTTTTCAGGTAGAAAGATTCAATTACC | 25 |
| 15-Reg-T1R10C3 | TTATGCGATTGACAAGAACCGGAGGTCAAT | 26 |
| 16-Reg-T1R12C3 | CATAAGGGACACTAAAACACTCACATTAAA | 27 |
| 17-Reg-T1R14C3 | CGGGTAAAATTCGGTCGCTGAGGAATGACA | 28 |
| 18-Reg-T2R06C3 | GTCTCTGACACCCTCAGAGCCACATCAAAA | 29 |
| 19-Reg-T2R08C3 | TCACCGGAAACGTCACCAATGAATTATTCA | 30 |
| 20-Reg-T2R10C3 | TTAAAGGTACATATAAAAGAAACAAACGCA | 31 |
| 21-Reg-T2R12C3 | ATAATAACTCAGAGAGATAACCCGAAGCGC | 32 |
| 22-Reg-T2R14C3 | ATTAGACGGAGCGTCTTTCCAGAGCTACAA | 33 |
| 23-Reg-T3R06C3 | TATATAACGTAAATCGTCGCTATATTTGAA | 34 |
| 24-Reg-T3R08C3 | TTACCTTTACAATAACGGATTCGCAAAATT | 35 |
| 25-Reg-T3R10C3 | ATTTGCACCATTTTGCGGAACAAATTTGAG | 36 |
| 26-Reg-T3R12C3 | GATTTAGATTGCTGAACCTCAAAGTATTAA | 37 |
| 27-Reg-T3R14C3 | CACCGCCTGAAAGCGTAAGAATACATTCTG | 38 |
| 28-Reg-T4R06C3 | TGAGTGTTCAGCTGATTGCCCTTGCGCGGG | 39 |
| 29-Reg-T4R08C3 | GAGAGGCGACAACATACGAGCCGCTGCAGG | 40 |
| 30-Reg-T4R10C3 | TCGACTCTGAAGGGCGATCGGTGCGGCCTC | 41 |
| 31-Reg-T4R12C3 | AGGAAGATCATTAAATGTGAGCGTTTTTAA | 42 |
| 32-Reg-T4R14C3 | CCAATAGGAAACTAGCATGTCAAGGAGCAA | 43 |
| 33-Reg-T1R04C5 | TAGAGCTTCAGACCGGAAGCAAACCTATTATA | 44 |
| 34-Reg-T1R06C5 | GTCAGAAGATTGAATCCCCCTCAACCTCGTTT | 45 |
| 35-Reg-T1R07C4 | AAATATTCCAAAGCGGATTGCATCGAGCTTCA | 46 |
| 36-Reg-T1R08C5 | ACCAGACGGAATACCACATTCAACGAGATGGT | 47 |
| 37-Reg-T1R09C4 | AGATTTAGACGATAAAAACCAAAAATCGTCAT | 48 |
| 38-Reg-T1R10C1 | AGTCAGGACATAGGCTGGCTGACCTTTGAAAG | 49 |
| 39-Reg-T1R10C5 | TTAATTTCCAACGTAACAAAGCTGTCCATGTT | 50 |
| 40-Reg-T1R11C2 | GAGTAATCTTTTAAGAACTGGCTCCGGAACAA | 51 |
| 41-Reg-T1R11C4 | ACCCAAATAACTTTAATCATTGTGATCAGTTG | 52 |
| 42-Reg-T1R12C5 | ACTTAGCCATTATACCAAGCGCGAGAGGACTA | 53 |
| 43-Reg-T1R13C2 | AAAAGAATAACCGAACTGACCAACTTCATCAA | 54 |
| 44-Reg-T1R13C4 | CCCCAGCGGGAACGAGGCGCAGACTATTCATT | 55 |
| 45-Reg-T1R14C5 | AAGACTTTGGCCGCTTTTGCGGGATTAAACAG | 56 |
| 46-Reg-T1R15C4 | GAGTTAAATTCATGAGGAAGTTTCTCTTTGAC | 57 |
| 47-Reg-T1R16C5 | CTTGATACTGAAAATCTCCAAAAAAGCGGAGT | 58 |
| 48-Reg-T1R17C4 | TTTCACGTCGATAGTTGCGCCGACCTTGCAGG | 59 |
| 49-Reg-T2R04C5 | TTATTCTGACTGGTAATAAGTTTTAACAAATA | 60 |
| 50-Reg-T2R06C5 | AATCCTCAACCAGAACCACCACCAGCCCCCTT | 61 |
| 51-Reg-T2R07C4 | GAGCCGCCTTAAAGCCAGAATGGAGATGATAC | 62 |
| 52-Reg-T2R08C5 | ATTAGCGTCCGTAATCAGTAGCGAATTGAGGG | 63 |
| 53-Reg-T2R10C1 | GCCATTTGCAAACGTAGAAAATACCTGGCATG | 64 |
| 54-Reg-T2R10C5 | AGGGAAGGATAAGTTTATTTTGTCAGCCGAAC | 65 |
| 55-Reg-T2R11C2 | AGGTGGCAGAATTATCACCGTCACCATTAGCA | 66 |
| 56-Reg-T2R12C5 | AAAGTTACGCCCAATAATAAGAGCAGCCTTTA | 67 |
| 57-Reg-T2R13C2 | CGCTAATAGGAATACCCAAAAGAAATACATAA | 68 |
| 58-Reg-T2R14C5 | CAGAGAGAACAAAATAAACAGCCATTAAATCA | 69 |
| 59-Reg-T2R16C5 | AGATTAGTATATAGAAGGCTTATCCAAGCCGT | 70 |
| 60-Reg-T2R17C4 | CAAATCAGTGCTATTTTGCACCCAGCCTAATT | 71 |
| 61-Reg-T3R04C5 | AAATAAGAACTTTTTCAAATATATCTGAGAGA | 72 |
| 62-Reg-T3R06C5 | CTACCTTTAGAATCCTTGAAAACAAGAAAACA | 73 |
| 63-Reg-T3R07C4 | TTTCCCTTTTAACCTCCGGCTTAGCAAAGAAC | 74 |
| 64-Reg-T3R08C5 | AAATTAATACCAAGTTACAAAATCCTGAATAA | 75 |
| 65-Reg-T3R09C4 | CTTTGAATTACATTTAACAATTTCTAATTAAT | 76 |
| 66-Reg-T3R10C1 | GTAGATTTGTTATTAATTTTAAAAAACAATTC | 77 |
| 67-Reg-T3R10C5 | TGGAAGGGAGCGGAATTATCATCAACTAATAG | 78 |
| 68-Reg-T3R11C2 | AACATTATGTAAAACAGAAATAAATTTTACAT | 79 |
| 69-Reg-T3R11C4 | CCAGAAGGTTAGAACCTACCATATCCTGATTG | 80 |
| 70-Reg-T3R12C5 | ATTAGAGCAATATCTGGTCAGTTGCAGCAGAA | 81 |
| 71-Reg-T3R13C2 | GCATCACCAGTATTAGACTTTACAGTTTGAGT | 82 |
| 72-Reg-T3R13C4 | CCTCAATCCGTCAATAGATAATACAGAAACCA | 83 |
| 73-Reg-T3R14C5 | GATAAAACTTTTTGAATGGCTATTTTCACCAG | 84 |
| 74-Reg-T3R15C4 | AGACAATAAGAGGTGAGGCGGTCATATCAAAC | 85 |
| 75-Reg-T3R16C5 | TCACACGATGCAACAGGAAAAACGGAAGAACT | 86 |
| 76-Reg-T3R17C4 | CCAGCCATCCAGTAATAAAAGGGACGTGGCAC | 87 |
| 77-Reg-T4R04C5 | AGCACTAAAAAGGGCGAAAAACCGAAATCCCT | 88 |
| 78-Reg-T4R06C5 | TATAAATCGAGAGTTGCAGCAAGCGTCGTGCC | 89 |
| 79-Reg-T4R07C4 | GGCCCTGAAAAAGAATAGCCCGAGCGTGGACT | 90 |
| 80-Reg-T4R08C5 | AGCTGCATAGCCTGGGGTGCCTAAGTAAAACG | 91 |
| 81-Reg-T4R09C4 | AAGTGTAATAATGAATCGGCCAACCACCGCCT | 92 |
| 82-Reg-T4R10C1 | GAATTCGTGCCATTCGCCATTCAGTTCCGGCA | 93 |
| 83-Reg-T4R10C5 | ACGGCCAGTACGCCAGCTGGCGAACATCTGCC | 94 |
| 84-Reg-T4R11C2 | ACTGTTGGAGAGGATCCCCGGGTACCGCTCAC | 95 |
| 85-Reg-T4R11C4 | TTCGCTATTGCCAAGCTTGCATGCGAAGCATA | 96 |
| 86-Reg-T4R12C5 | AGTTTGAGATTCTCCGTGGGAACAATTCGCAT | 97 |
| 87-Reg-T4R13C2 | TTCATCAACGCACTCCAGCCAGCTGCTGCGCA | 98 |
| 88-Reg-T4R13C4 | CCCGTCGGGGGACGACGACAGTATCGGGCCTC | 99 |
| 89-Reg-T4R14C5 | TAAATTTTTGATAATCAGAAAAGCACAAAGGC | 100 |
| 90-Reg-T4R15C4 | ACCCCGGTTGTTAAATCAGCTCATAGTAACAA | 101 |
| 91-Reg-T4R16C5 | TATCAGGTAAATCACCATCAATATCAATGCCT | 102 |
| 92-Reg-T4R17C4 | AGACAGTCCATTGCCTGAGAGTCTTCATATGT | 103 |
| 93-Reg-T2R10C4 | GACGGAAAACCATCGATAGCAGCATTGCCATC | 104 |
| | TTTTCATACACCCTCA | |
| 94-Reg-T2R15C4 | TGCCAGTTATAACATAAAAACAGGACAAGAAT | 105 |
| | TGAGTTAACAGAAGGA | |
| 95-Edg-T1R00C7-T3.5p- | TTTGGTGGCATCAATTCTAGGGCGCGAGCTGA | 106 |
| T3.3p-DYE40.22.9.3p | AAATTTCGTTTCTTTGCTTGTGTCTTGTCTTCGT | |
| | CGTGCTTGTTCC | |
| 96-Edg-T1R02C7-T3.5p- | TTTTCCCAATTCTGCGAACCCATATAACAGTTG | 107 |
| T3.3p-DYE40.22.9.3p | ATTTTCGTTTCTTTGCTTGTGTCTTGTCTTCGTC | |
| | GTGCTTGTTCC | |
| 97-Edg-T1R04C7-T3.5p- | TTTATTGCTCCTTTTGATATTAGAGAGTACCTT | 108 |
| T3.3p-DYE40.22.9.3p | TATTTCGTTTCTTTGCTTGTGTCTTGTCTTCGTC | |
| | GTGCTTGTTCC | |
| 98-Edg-T1R06C7-T3.5p- | TTTCCATAAATCAAAAATCCAGAAAACGAGAA | 109 |
| T3.3p-DYE40.22.9.3p | TGATTTCGTTTCTTTGCTTGTGTCTTGTCTTCGT | |
| | CGTGCTTGTTCC | |
| 99-Edg-T1R08C7-T3.5p- | TTTCGAGGCATAGTAAGAGACGCCAAAAGGAA | 110 |
| T3.3p-DYE40.22.9.3p | TTATTTCGTTTCTTTGCTTGTGTCTTGTCTTCGT | |
| | CGTGCTTGTTCC | |
| 100-Edg-T1R10C7- | TTTGAAACACCAGAACGAGAGGCTTGCCCTGA | 111 |
| T3.5p-T3.3p- | CGATTTCGTTTCTTTGCTTGTGTCTTGTCTTCGT | |
| DYE40.22.9.3p | CGTGCTTGTTCC | |
| 101-Edg-T1R12C7- | TTTCTGATAAATTGTGTCGAGATTTGTATCATC | 112 |
| T3.5p-T3.3p- | GCTTTCGTTTCTTTGCTTGTGTCTTGTCTTCGTC | |
| DYE40.22.9.3p | GTGCTTGTTCC | |
| 102-Edg-T1R14C7- | TTTGAACGAGGGTAGCAACGCGAAAGACAGCA | 113 |
| T3.5p-T3.3p- | TCGTTTCGTTTCTTTGCTTGTGTCTTGTCTTCGT | |
| DYE40.22.9.3p | CGTGCTTGTTCC | |
| 103-Edg-T1R16C7- | TTTGGTTTATCAGCTTGCTAGCCTTTAATTGTA | 114 |
| T3.5p-T3.3p- | TCTTTCGTTTCTTTGCTTGTGTCTTGTCTTCGTC | |
| DYE40.22.9.3p | GTGCTTGTTCC | |
| 104-Edg-T1R18C7- | TTTGGGATTTTGCTAAACAAATGAATTTTCTGT | 115 |
| T3.5p-T3.3p- | ATTTTCGTTTCTTTGCTTGTGTCTTGTCTTCGTC | |
| DYE40.22.9.3p | GTGCTTGTTCC | |
| 105-Edg-T1R20C7- | TTTACAAACTACAACGCCTGAGTTTCGTCACCA | 116 |
| T3.5p-T3.3p- | GTTTTCGTTTCTTTGCTTGTGTCTTGTCTTCGTC | |
| DYE40.22.9.3p | GTGCTTGTTCC | |
| 106-Edg-T2R00C7- | TTTAGCCACCACCCTCATTGAACCGCCACCCTC | 117 |
| T3.5p-T3.3p- | AGTTTCGTTTCTTTGCTTGTGTCTTGTCTTCGTC | |
| DYE40.22.9.3p | GTGCTTGTTCC | |
| 107-Edg-T2R02C7- | TTTGAGAGGGTTGATATAAGCGGATAAGTGCC | 118 |
| T3.5p-T3.3p- | GTCTTTCGTTTCTTTGCTTGTGTCTTGTCTTCGT | |
| DYE40.22.9.3p | CGTGCTTGTTCC | |
| 108-Edg-T2R04C7- | TTTGTATAAACAGTTAATGTTGAGTAACAGTGC | 119 |
| T3.5p-T3.3p- | CCTTTCGTTTCTTTGCTTGTGTCTTGTCTTCGTC | |
| DYE40.22.9.3p | GTGCTTGTTCC | |
| 109-Edg-T2R06C7- | TTTGCAGGTCAGACGATTGTTGACAGGAGGTT | 120 |
| T3.5p-T3.3p- | GAGTTTCGTTTCTTTGCTTGTGTCTTGTCTTCGT | |
| DYE40.22.9.3p | CGTGCTTGTTCC | |
| 110-Edg-T2R08C7- | TTTTAGCGCGTTTTCATCGCTTTAGCGTCAGAC | 121 |
| T3.5p-T3.3p- | TGTTTCGTTTCTTTGCTTGTGTCTTGTCTTCGTC | |
| DYE40.22.9.3p | GTGCTTGTTCC | |
| 111-Edg-T2R10C7- | TTTGCGCCAAAGACAAAAGTTCATATGGTTTA | 122 |
| T3.5p-T3.3p- | CCATTTCGTTTCTTTGCTTGTGTCTTGTCTTCGT | |
| DYE40.22.9.3p | CGTGCTTGTTCC | |
| 112-Edg-T2R12C7- | TTTCCGAAGCCCTTTTTAAAGCAATAGCTATCT | 123 |
| T3.5p-T3.3p- | TATTTCGTTTCTTTGCTTGTGTCTTGTCTTCGTC | |
| DYE40.22.9.3p | GTGCTTGTTCC | |
| 113-Edg-T2R14C7- | TTTTTTTTTGTTTAACGTCTCCAAATAAGAAAC | 124 |
| T3.5p-T3.3p- | GATTTCGTTTCTTTGCTTGTGTCTTGTCTTCGTC | |
| DYE40.22.9.3p | GTGCTTGTTCC | |
| 114-Edg-T2R16C7- | TTTAACCTCCCGACTTGCGGCGAGGCGTTTTAG | 125 |
| T3.5p-T3.3p- | CGTTTCGTTTCTTTGCTTGTGTCTTGTCTTCGTC | |
| DYE40.22.9.3p | GTGCTTGTTCC | |
| 115-Edg-T2R18C7- | TTTTAAACCAAGTACCGCATTCCAAGAACGGG | 126 |
| T3.5p-T3.3p- | TATTTTCGTTTCTTTGCTTGTGTCTTGTCTTCGT | |
| DYE40.22.9.3p | CGTGCTTGTTCC | |
| 116-Edg-T2R20C7- | TTTAGATAAGTCCTGAACACCTGTTTATCAACA | 127 |
| T3.5p-T3.3p- | ATTTTCGTTTCTTTGCTTGTGTCTTGTCTTCGTC | |
| DYE40.22.9.3p | GTGCTTGTTCC | |
| 117-Edg-T3R00C7- | TTTGTAAAGTAATTCTGTCAAAGTACCGACAA | 128 |
| T3.5p-T3.3p- | AAGTTTCGTTTCTTTGCTTGTGTCTTGTCTTCGT | |
| DYE40.22.9.3p | CGTGCTTGTTCC | |
| 118-Edg-T3R02C7- | TTTAGTAGGGCTTAATTGAAAAGCCAACGCTC | 129 |
| T3.5p-T3.3p- | AACTTTCGTTTCTTTGCTTGTGTCTTGTCTTCGT | |
| DYE40.22.9.3p | CGTGCTTGTTCC | |
| 119-Edg-T3R04C7- | TTTAATGGTTTGAAATACCCTTCTGACCTAAAT | 130 |
| T3.5p-T3.3p- | TTTTTCGTTTCTTTGCTTGTGTCTTGTCTTCGTC | |
| DYE40.22.9.3p | GTGCTTGTTCC | |
| 120-Edg-T3R06C7- | TTTAGTCAATAGTGAATTTTTAAGACGCTGAGA | 131 |
| T3.5p-T3.3p- | AGTTTCGTTTCTTTGCTTGTGTCTTGTCTTCGTC | |
| DYE40.22.9.3p | GTGCTTGTTCC | |
| 121-Edg-T3R08C7- | TTTTGAGCAAAAGAAGATGATTCATTTCAATTA | 132 |
| T3.5p-T3.3p- | CCTTTCGTTTCTTTGCTTGTGTCTTGTCTTCGTC | |
| DYE40.22.9.3p | GTGCTTGTTCC | |
| 122-Edg-T3R10C7- | TTTCAATATAATCCTGATTGATGATGGCAATTC | 133 |
| T3.5p-T3.3p- | ATTTTCGTTTCTTTGCTTGTGTCTTGTCTTCGTC | |
| DYE40.22.9.3p | GTGCTTGTTCC | |
| 123-Edg-T3R12C7- | TTTGTTATCTAAAATATCTAAAGGAATTGAGG | 134 |
| T3.5p-T3.3p- | AAGTTTCGTTTCTTTGCTTGTGTCTTGTCTTCGT | |
| DYE40.22.9.3p | CGTGCTTGTTCC | |
| 124-Edg-T3R14C7- | TTTACATCGCCATTAAAAAAACTGATAGCCCT | 135 |
| T3.5p-T3.3p- | AAATTTCGTTTCTTTGCTTGTGTCTTGTCTTCGT | |
| DYE40.22.9.3p | CGTGCTTGTTCC | |
| 125-Edg-T3R16C7- | TTTTCGTCTGAAATGGATTACATTTTGACGCTC | 136 |
| T3.5p-T3.3p- | AATTTCGTTTCTTTGCTTGTGTCTTGTCTTCGTC | |
| DYE40.22.9.3p | GTGCTTGTTCC | |
| 126-Edg-T3R18C7- | TTTTTGATTAGTAATAACATTGTAGCAATACTT | 137 |
| T3.5p-T3.3p- | CTTTTCGTTTCTTTGCTTGTGTCTTGTCTTCGTC | |
| DYE40.22.9.3p | GTGCTTGTTCC | |
| 127-Edg-T3R20C7- | TTTAGGAACGGTACGCCAGTAAAGGGATTTTA | 138 |
| T3.5p-T3.3p- | GACTTTCGTTTCTTTGCTTGTGTCTTGTCTTCGT | |
| DYE40.22.9.3p | CGTGCTTGTTCC | |
| 128-Edg-T4R00C7- | TTTGAGCACGTATAACGTGCTATGGTTGCTTTG | 139 |
| T3.5p-T3.3p- | ACTTTCGTTTCTTTGCTTGTGTCTTGTCTTCGTC | |
| DYE40.22.9.3p | GTGCTTGTTCC | |
| 129-Edg-T4R02C7- | TTTCGGGCGCTAGGGCGCTAAGAAAGCGAAAG | 140 |
| T3.5p-T3.3p- | GAGTTTCGTTTCTTTGCTTGTGTCTTGTCTTCGT | |
| DYE40.22.9.3p | CGTGCTTGTTCC | |
| 130-Edg-T4R04C7- | TTTATCACCCAAATCAAGTGCCCACTACGTGA | 141 |
| T3.5p-T3.3p- | ACCTTTCGTTTCTTTGCTTGTGTCTTGTCTTCGT | |
| DYE40.22.9.3p | CGTGCTTGTTCC | |
| 131-Edg-T4R06C7- | TTTATCCTGTTTGATGGTGGCCCCAGCAGGCGA | 142 |
| T3.5p-T3.3p- | AATTTCGTTTCTTTGCTTGTGTCTTGTCTTCGTC | |
| DYE40.22.9.3p | GTGCTTGTTCC | |
| 132-Edg-T4R08C7- | TTTGCTCACTGCCCGCTTTACATTAATTGCGTT | 143 |
| T3.5p-T3.3p- | GCTTTCGTTTCTTTGCTTGTGTCTTGTCTTCGTC | |
| DYE40.22.9.3p | GTGCTTGTTCC | |
| 133-Edg-T4R10C7- | TTTGTAACGCCAGGGTTTTAAGGCGATTAAGTT | 144 |
| T3.5p-T3.3p- | GGTTTCGTTTCTTTGCTTGTGTCTTGTCTTCGTC | |
| DYE40.22.9.3p | GTGCTTGTTCC | |
| 134-Edg-T4R12C7- | TTTCGTTGGTGTAGATGGGGTAATGGGATAGG | 145 |
| T3.5p-T3.3p- | TCATTTCGTTTCTTTGCTTGTGTCTTGTCTTCGT | |
| DYE40.22.9.3p | CGTGCTTGTTCC | |
| 135-Edg-T4R14C7- | TTTTTTAAATTGTAAACGTATTGTATAAGCAAA | 146 |
| T3.5p-T3.3p- | TATTTCGTTTCTTTGCTTGTGTCTTGTCTTCGTC | |
| DYE40.22.9.3p | GTGCTTGTTCC | |
| 136-Edg-T4R16C7- | TTTGCCGGAGAGGGTAGCTTAGCTGATAAATT | 147 |
| T3.5p-T3.3p- | AATTTTCGTTTCTTTGCTTGTGTCTTGTCTTCGT | |
| DYE40.22.9.3p | CGTGCTTGTTCC | |
| 137-Edg-T4R18C7- | TTTAAATTTTTAGAACCCTTTCAACGCAAGGAT | 148 |
| T3.5p-T3.3p- | AATTTCGTTTCTTTGCTTGTGTCTTGTCTTCGTC | |
| DYE40.22.9.3p | GTGCTTGTTCC | |
| 138-Edg-T4R20C7- | TTTTAAGCAATAAAGCCTCAAAGAATTAGCAA | 149 |
| T3.5p-T3.3p- | AATTTTCGTTTCTTTGCTTGTGTCTTGTCTTCGT | |
| DYE40.22.9.3p | CGTGCTTGTTCC | |
| 139-Bri-T1R14C2 | TGCCACTACTTTTTTTGCCACCCTC | 150 |
| 140-Bri-T2R14C2 | AACTGAACATTTTTTTTGAATAACC | 151 |
| 141-Bri-T3R14C2 | GCCACGCTGTTTTTTTACCAGTGAG | 152 |
| 142-Bri-T4R14C2 | CAAAAATAATTTTTTTTGTTTAGAC | 153 |
| 143-Bri-T1R02C5 | GATACATTTCGCTTTTTTGACCCTGTAAT | 154 |
| 144-Bri-T2R02C5 | ACCGTACTCAGGTTTTTGATCTAAAGTTT | 155 |
| 145-Bri-T3R02C5 | AACATGTAATTTTTTTTGAAACCAATCAA | 156 |
| 146-Bri-T4R02C5 | GCGTAACCACCATTTTTGAGTAAAAGAGT | 157 |
| 147-Bri-T1R08C1 | CAGAGGGGGTTTTGCCTTCCTGTAGCCAGCT | 158 |
| 148-Bri-T2R08C1 | GAACCGCCTCTTTACCTAAAACGAAAGAGGC | 159 |
| 149-Bri-T3R08C1 | CATAAATCAATTTAGTCAGAGGGTAATTGAG | 160 |
| 150-Bri-T4R08C1 | CCAGGGTGGTTTTGCAAATGAAAAATCTAAA | 161 |
| 151-Bri-T1R16C3 | ACAACCATTTTTTCATACATGGCTTTTAAGCGCA | 162 |
| 152-Bri-T2R16C3 | TTTTATCTTTTTTATCCAATCGCAAGAGTTGGGT | 163 |
| 153-Bri-T3R16C3 | GCCAACATTTTTTCCACTATTAAAGAAATAGGGT | 164 |
| 154-Bri-T4R16C3 | ACAAGAGTTTTTTTCGCGTTTTAATTCAAAAAGA | 165 |
| 155-Bri-T1R07C2 | TGGATAGCAAGCCCGATTTTTAATCGTAAACG | 166 |
| | CCAT | |
| 156-Bri-T2R07C2 | AGAACCGCATTTACCGTTTTACCGATATATACG | 167 |
| | TAA | |
| 157-Bri-T3R07C2 | TTGCTTCTTATATGTATTTTACGCTAACGGAGA | 168 |
| | ATT | |
| 158-Bri-T4R07C2 | ACGGGCAAGTTCCAGTTTTTTCTGACCTGCAAC | 169 |
| | AGT | |
| 159-Bri-T1R18C5 | GAGAATAGAAAGGAACAACTATTTTCTCAAGA | 170 |
| | GAAGGA | |
| 160-Bri-T2R18C5 | TTTTATTTTCATCGTAGGAATTTTTAGCCTGTTT | 171 |
| | AGTA | |
| 161-Bri-T3R18C5 | CAAACTATCGGCCTTGCTGGTTTTTGAGCTTGA | 172 |
| | CGGGG | |
| 162-Bri-T4R18C5 | GAGTAATGTGTAGGTAAAGATTTTTTGTTTTAA | 173 |
| | ATATG | |
| 163-Bri-T1R12C1 | AGGACAGATGATTTTTTCACCAGTAGCACCATT | 174 |
| | ACCGACTTGA | |
| 164-Bri-T2R12C1 | ATTAAGACTCCTTTTTAATATACAGTAACAGTA | 175 |
| | CCGAAATTGC | |
| 165-Bri-T3R12C1 | GACAACTCGTATTTTTTCCTGTGTGAAATTGTT | 176 |
| | ATCCGAGCTC | |
| 166-Bri-T4R12C1 | CCGCTTCTGGTTTTTTCGTTAATAAAACGAACT | 177 |
| | AAATTATACC | |
| 167-Bri-T1R19C5 | TGTCGTCTCAGCCCTCATATTTTTTTCGCCACC | 178 |
| | CTCAGGTGTATC | |
| 168-Bri-T2R19C5 | TAATCGGCCATCCTAATTTTTTTTTTTTTTCGAG | 179 |
| | CCAACAACGCC | |
| 169-Bri-T3R19C5 | CTGTCCATTTTTATAATCATTTTTTTCTTAATGC | 180 |
| | GCCCACGCTGC | |
| 170-Bri-T4R19C5 | ACTTTTGCATCGGTTGTACTTTTTTTAACCTGTT | 181 |
| | TAGGACCATTA | |
| 171-Bri-T1R05C4 | AAGCGAACAATTGCTGAATATAATGCTGTATT | 182 |
| | TTTTTGTGAGAAAGGCCGG | |
| 172-Bri-T2R05C4 | AGGAGTGTAAACATGAAAGTATTAAGAGGCTT | 183 |
| | TTTTTGCGAATAATAATTT | |
| 173-Bri-T3R05C4 | GCGAGAAAATAAACACCGGAATCATAATTATT | 184 |
| | TTTTTCGCCCAATAGCAAG | |
| 174-Bri-T4R05C4 | CCAACGTCATCGGAACCCTAAAGGGAGCCCTT | 185 |
| | TTTTTGAACAATATTACCG | |
| 175-Bri-T2R10C0 | GGAATTAGAGCTTTTTTTTCAGACCAGGCGCGT | 186 |
| | TGGGAAGATTTTTTTTCCAGGCAAAGC | |
| 176-Bri-T4R10C0 | AATCATGGTCATTTTTTTTTTTGCCCGAACTCA | 187 |
| | GGTTTAACTTTTTTTTCAGTATGTTAG | |
| 177-Reg-TIR05C6- | GTCAGGAAGAGGTCATTTTTGCTCTGGAAGTTT | 188 |
| T3.3p-JF20.22.9.3p | ACAGTTATTAGCCCGCATTT | |
| 178-Reg-TIR09C6- | ATACATACAACACTATCATAACATGCTTTATTT | 189 |
| T3.3p-JF20.22.9.3p | ACAGTTATTAGCCCGCATTT | |
| 179-Reg-TIR13C6- | ACAACGGAAATCCGCGACCTGCCTCATTCATTT | 190 |
| T3.3p-JF20.22.9.3p | ACAGTTATTAGCCCGCATTT | |
| 180-Reg-TIR17C6- | CAAAAGGTTCGAGGTGAATTTCTCGTCACCTTT | 191 |
| T3.3p-JF20.22.9.3p | ACAGTTATTAGCCCGCATTT | |
| 181-Reg-T2R05C6- | CAGTGCCCCCCCTGCCTATTTCTTTGCTCATTT | 192 |
| T3.3p-JF20.22.9.3p | ACAGTTATTAGCCCGCATTT | |
| 182-Reg-T2R09C6- | AGTTTGCGCATTTTCGGTCATAGAGCCGCCTTT | 193 |
| T3.3p-JF20.22.9.3p | ACAGTTATTAGCCCGCATTT | |
| 183-Reg-T2R13C6- | ATGAAATGAAAAGTAAGCAGATACAATCAATT | 194 |
| T3.3p-JF20.22.9.3p | TACAGTTATTAGCCCGCATTT | |
| 184-Reg-T2R17C6- | TAAGAACGGAGGTTTTGAAGCCTATTATTTTTT | 195 |
| T3.3p-JF20.22.9.3p | ACAGTTATTAGCCCGCATTT | |
| 185-Reg-T3R05C6- | ATTTCATGACCGTGTGATAAATAATTCTTATTT | 196 |
| T3.3p-JF20.22.9.3p | ACAGTTATTAGCCCGCATTT | |
| 186-Reg-T3R09C6- | GCGAATTATGAAACAAACATCATAGCGATATT | 197 |
| T3.3p-JF20.22.9.3p | TACAGTTATTAGCCCGCATTT | |
| 187-Reg-T3R13C6- | ACAGTTGTTAGGAGCACTAACATATTCCTGTTT | 198 |
| T3.3p-JF20.22.9.3p | ACAGTTATTAGCCCGCATTT | |
| 188-Reg-T3R17C6- | AATACCTATTTACATTGGCAGAAGTCTTTATTT | 199 |
| T3.3p-JF20.22.9.3p | ACAGTTATTAGCCCGCATTT | |
| 189-Reg-T4R05C6- | GGCGATGTTTTTGGGGTCGAGGGCGAGAAATT | 200 |
| T3.3p-JF20.22.9.3p | TACAGTTATTAGCCCGCATTT | |
| 190-Reg-T4R09C6- | CTAACTCCCAGTCGGGAAACCTGGTCCACGTTT | 201 |
| T3.3p-JF20.22.9.3p | ACAGTTATTAGCCCGCATTT | |
| 191-Reg-T4R13C6- | ATTGACCCGCATCGTAACCGTGAGGGGGATTT | 202 |
| T3.3p-JF20.22.9.3p | TACAGTTATTAGCCCGCATTT | |
| 192-Reg-T4R17C6- | ACCGTTCATTTTTGAGAGATCTCCCAAAAATTT | 203 |
| T3.3p-JF20.22.9.3p | ACAGTTATTAGCCCGCATTT | |
| 193-Reg-T1R21C5- | CCATGTACCGTAACACTGTAGCATTCCACAGA | 204 |
| T3.3p-JF20.22.9.3p | TTCCAGACTTTACAGTTATTAGCCCGCATTT | |
| 194-Reg-T2R21C5- | AGCTAATGCAGAACGCGAGAAAAATAATATCC | 205 |
| T3.3p-JF20.22.9.3p | TGTCTTTCTTTACAGTTATTAGCCCGCATTT | |
| 195-Reg-T3R21C5- | CTAAACAGGAGGCCGATAATCCTGAGAAGTGT | 206 |
| T3.3p-JF20.22.9.3p | CACGCAAATTTACAGTTATTAGCCCGCATTT | |
| 196-Reg-T4R21C5- | AATCATACAGGCAAGGCAGAGCATAAAGCTAA | 207 |
| T3.3p-JF20.22.9.3p | GGGAGAAGTTTACAGTTATTAGCCCGCATTT | |
| 197-Reg-T1R03C6 | TTTCATTGAGTAGATTTAGTTTCTATATTT | 208 |
| 198-Reg-T1R07C6 | AACAGTTAGGTCTTTACCCTGATCCAACAG | 209 |
| 199-Reg-T1R11C6 | GTGAATATAGTAAATTGGGCTTTAATGCAG | 210 |
| 200-Reg-T1R15C6 | CTCAGCAGGCTACAGAGGCTTTAACAAAGT | 211 |
| 201-Reg-T1R19C6 | GTTAGTAACTTTCAACAGTTTCAAAGGCTC | 212 |
| 202-Reg-T2R03C6 | GTACCAGGTATAGCCCGGAATAGAACCGCC | 213 |
| 203-Reg-T2R07C6 | GCCAGCAGCCTTGATATTCACAAACGGGGT | 214 |
| 204-Reg-T2R11C6 | TAGAAAAGGCGACATTCAACCGCAGAATCA | 215 |
| 205-Reg-T2R15C6 | ATCCCAAAAAAATGAAAATAGCAAGAAACA | 216 |
| 206-Reg-T2R19C6 | CTTATCACTCATCGAGAACAAGCGGTATTC | 217 |
| 207-Reg-T3R03C6 | CCAGTATGAATCGCCATATTTAGTAATAAG | 218 |
| 208-Reg-T3R07C6 | GCTTAGAATCAAAATCATAGGTTTTAGTTA | 219 |
| 209-Reg-T3R11C6 | ATTATCAGTTTGGATTATACTTGCGCAGAG | 220 |
| 210-Reg-T3R15C6 | ATGCGCGTACCGAACGAACCACGCAAATCA | 221 |
| 211-Reg-T3R19C6 | TTAACCGTCACTTGCCTGAGTACTCATGGA | 222 |
| 212-Reg-T4R03C6 | GGAAGGGGGCAAGTGTAGCGGTGCTACAGG | 223 |
| 213-Reg-T4R07C6 | CTGGTTTGTTCCGAAATCGGCATCTATCAG | 224 |
| 214-Reg-T4R11C6 | GTGCTGCCCCAGTCACGACGTTTGAGTGAG | 225 |
| 215-Reg-T4R15C6 | CAGGAAGTAATATTTTGTTAAAAACGGCGG | 226 |
| 216-Reg-T4R19C6 | CCTTTATCATATATTTTAAATGGATATTCA | 227 |
| 217-Reg-T2R12C4- | AACCGAGGGCAAAGACACCACGGATAAATATT | 228 |
| T3.3p-APT40.2.3p | TTTCACTCACCTCCATCTCCACTCCTACCCATC | |
| | CAACTCCCAC | |

DNA Origami is engineered to include one APT 40.2 strand, forty-four DYE40.22.9 strands and twenty JF20.22.9 strands. The single APT40.2 strand is hybridized to a TCOv40.2 oligonucleotide that is derivatized with a transcyclooctene (TCO) click reagent, thereby forming a double stranded handle having the TCO moiety. A protein is reacted with N-hydroxy succinimide methyltetrazine (mtz) which reacts with amines of lysine residues to produce an mtz-protein derivative, and the mtz-protein derivative is attached to the double stranded handle via a click reaction between mtz and TCO moieties. Because the DNA origami contains no more than one TCO moiety, only a single protein is attached to the DNA origami. The DYE40.22.9 handle strands are hybridized to DYE647v40.22.9 strands, each of which is attached to a luminophore label. A plurality of JF20.22.9 strands are hybridized to respective JFDBCOv20.22.9 which are attached to the surface of a solid support, thereby immobilizing the DNA origami on the solid support.

The handles are placed at the positions diagramed in FIG. 1B. The DYE40.22.9 handle strands (dotted lines) are located at the perimeter of a tile shaped DNA origami (eight are shown but the number can vary), JF20.22.9 handles (solid lines) are positioned on a first face of the tile (nine are shown but the number can vary), and a single APT40.2 handle (dashed line) is positioned on the face of the tile opposite the face having the JF20.22.9 handles. The handles are hybridized with their respective oligonucleotide complements to produce an origami DNA attached to (i) a single protein analyte (globule) via a APT40.2-TCOv40.2 hybrid (dashed double line), (ii) a plurality of labels (seven-point stars) via DYE40.22.9-DYE647v40.22.9 hybrids (dotted double lines) and (iii) to a solid support via a plurality of JF20.22.9-JFDBCOv20.22.9 hybrids (solid double lines). An exemplary configuration is shown in FIG. 1C.

### Example II

### Increasing DNA Origami Stability with Photo-Cross-Links

The stability and integrity of DNA origami structures is influenced by the fluidic environment. Origami tile structures unfold as temperatures increase, Mg⁺² concentrations decrease or denaturant (e.g., Urea) concentrations increase. For example, atomic force microscope images show unfolding of shorter staples on corners of DNA origami tiles under denaturing conditions. This example describes covalent cross-linking of DNA origami tiles to increase stability. Covalent cross-linking can be extended to other nucleic acid origami structures by appropriate choice of type and location of moieties that are capable of being cross-linked.

Two ultraviolet radiation (UV) cross-linking strategies were tested and used to enhance DNA origami tile structural stability. The first strategy used 310 nm UV to cross-link DNA origami with specially designed staples. In this strategy, staple sequences were designed to include thymine bases at specific locations, such as at or near the staple strand termini or at or near strand crossover sites. The designed DNA origami tile (dTile) was formed by folding the scaffold strand and staples, including the redesigned staples, as follows. A master mix was formed including 120 μl of 500 nM staple nucleic acids, 60 μl of 1 μM attachment handle nucleic acids, 40 μl of 100 nM p7249 m13 ssDNA scaffold (Tilibit Nanosystems, Germany), 40 μl of 10×FOB (50 mM NaCl, 10 mM EDTA, 50 mM Tris-HCl pH 8.0), 25 μl of 200 mM MgCl₂ and 115 μl water. The master mix was folded in strip tubes in a PCR instrument using the following cycle: 2 min at 95° C. followed by cooling from 90° C. to 20° C. at a rate of 1° C. per minute. The dTile was then irradiated with 310 nm UV light under conditions set forth below to cross-link the thymine bases. A listing of oligonucleotide sequences for the dTile is provided in Table 3.

TABLE 3
Oligonucleotide Sequences for dTile

| Sequence | | SEQ ID |
|---|---|---|
| Name | Sequence | NO |
| 1-Reg-T1R01C6 | TTCATTTGTTCTAATAGTAGTAGCATT | 229 |
| 2-Reg-T2R01C6 | TACCCTCATTTTCAGGGATAGCAAGCC | 230 |
| 3-Reg-T3R01C6 | TAGAATATTTCAGACGACGACAATAAA | 231 |
| 4-Reg-T4R01C6 | TGCGCGTATTCTTTCCTCGTTAGAATC | 232 |
| 5-Reg-T1R03C5 | TCAACTAAAGTACGGTGTTGGATGGCTT | 233 |
| 6-Reg-T1R09C2 | TCATTATTATTGCAAAAGAAGTTTTGCT | 234 |
| 7-Reg-T2R03C5 | TTTAGGATTAGCGGGGTTTGGAACCTAT | 235 |
| 8-Reg-T2R09C2 | TAGGCCGGATTACCAGAGCCACCACCGT | 236 |
| 9-Reg-T3R03C5 | TTCATATGCGTTATACATTAAGGCGTTT | 237 |
| 10-Reg-T3R09C2 | TCGGGAGAATTTTTAATGGAAACAGTAT | 238 |
| 11-Reg-T4R03C5 | TAAAGCCGGCGAACGTGTTTGCCGTAAT | 239 |
| 12-Reg-T4R09C2 | TAATTCCACTTGTTTGCGTATTGGGCGT | 240 |
| 13-Reg-T1R06C3 | TTTAAGAGGTTGTCCAATACTGCGGATTTAGCGAG | 241 |
| | T | |
| 14-Reg-T1R08C3 | TAGGCTTTTTTCAGGTAGAAAGATTCTTAATTACCT | 242 |
| 15-Reg-T1R10C3 | TTTATGCGATTTTGACAAGAACCGGATTGGTCAAT | 243 |
| | T | |
| 16-Reg-T1R12C3 | TCATAAGGGTTACACTAAAACACTCATTCATTAAA | 244 |
| | T | |
| 17-Reg-T1R14C3 | TCGGGTAAATTATTCGGTCGCTGAGGTTAATGACA | 245 |
| | T | |
| 18-Reg-T2R06C3 | TGTCTCTGATTCACCCTCAGAGCCACTTATCAAAA | 246 |
| | T | |
| 19-Reg-T2R08C3 | TTCACCGGATTAACGTCACCAATGAATTTTATTCA | 247 |
| | T | |
| 20-Reg-T2R10C3 | TTTAAAGGTTTACATATAAAAGAAACTTAAACGCA | 248 |
| | T | |
| 21-Reg-T2R12C3 | TATAATAACTTTCAGAGAGATAACCCTTGAAGCGC | 249 |
| | T | |
| 22-Reg-T2R14C3 | TATTAGACGTTGAGCGTCTTTCCAGATTGCTACAA | 250 |
| | T | |
| 23-Reg-T3R06C3 | TTATATAACTTGTAAATCGTCGCTATTTATTTGAAT | 251 |
| 24-Reg-T3R08C3 | TTTACCTTTTTACAATAACGGATTCGTTCAAAATTT | 252 |
| 25-Reg-T3R10C3 | TATTTGCACTTCATTTTGCGGAACAATTATTTGAGT | 253 |
| 26-Reg-T3R12C3 | TGATTTAGATTTTGCTGAACCTCAAATTGTATTAAT | 254 |
| 27-Reg-T3R14C3 | TCACCGCCTTTGAAAGCGTAAGAATATTCATTCTG | 255 |
| | T | |
| 28-Reg-T4R06C3 | TTGAGTGTTTTCAGCTGATTGCCCTTTTGCGCGGGT | 256 |
| 29-Reg-T4R08C3 | TGAGAGGCGTTACAACATACGAGCCGTTCTGCAG | 257 |
| | GT | |
| 30-Reg-T4R10C3 | TTCGACTCTTTGAAGGGCGATCGGTGTTCGGCCTC | 258 |
| | T | |
| 31-Reg-T4R12C3 | TAGGAAGATTTCATTAAATGTGAGCGTTTTTTTAA | 259 |
| | T | |
| 32-Reg-T4R14C3 | TCCAATAGGTTAAACTAGCATGTCAATTGGAGCAA | 260 |
| | T | |
| 33-Reg-T1R04C5 | TTAGAGCTTTTCAGACCGGAAGCAAACTTCTATTA | 261 |
| | TAT | |
| 34-Reg-T1R06C5 | TGTCAGAAGTTATTGAATCCCCCTCAATTCCTCGTT | 262 |
| | TT | |
| 35-Reg-T1R07C4 | TAAATATTCTTCAAAGCGGATTGCATCTTGAGCTT | 263 |
| | CAT | |
| 36-Reg-T1R08C5 | TACCAGACGTTGAATACCACATTCAACTTGAGATG | 264 |
| | GTT | |
| 37-Reg-T1R09C4 | TAGATTTAGTTACGATAAAAACCAAAATTATCGTC | 265 |
| | ATT | |
| 38-Reg-T1R10C1 | TAGTCAGGATTCATAGGCTGGCTGACCTTTTTGAA | 266 |
| | AGT | |
| 39-Reg-T1R10C5 | TTTAATTTCTTCAACGTAACAAAGCTGTTTCCATGT | 267 |
| | TT | |
| 40-Reg-T1R11C2 | TGAGTAATCTTTTTTAAGAACTGGCTCTTCGGAAC | 268 |
| | AAT | |
| 41-Reg-T1R11C4 | TACCCAAATTTAACTTTAATCATTGTGTTATCAGTT | 269 |
| | GT | |
| 42-Reg-T1R12C5 | TACTTAGCCTTATTATACCAAGCGCGATTGAGGAC | 270 |
| | TAT | |
| 43-Reg-T1R13C2 | TAAAAGAATTTAACCGAACTGACCAACTTTTCATC | 271 |
| | AAT | |
| 44-Reg-T1R14C5 | TAAGACTTTTTGGCCGCTTTTGCGGGATTTTAAAC | 272 |
| | AGT | |
| 45-Reg-T1R15C4 | TGAGTTAAATTTTCATGAGGAAGTTTCTTTCTTTGA | 273 |
| | CT | |
| 46-Reg-T1R16C5 | TCTTGATACTTTGAAAATCTCCAAAAATTAGCGGA | 274 |
| | GTT | |
| 47-Reg-T1R17C4 | TTTTCACGTTTCGATAGTTGCGCCGACTTCTTGCAG | 275 |
| | GT | |
| 48-Reg-T2R04C5 | TTTATTCTGTTACTGGTAATAAGTTTTTTAACAAAT | 276 |
| | AT | |
| 49-Reg-T2R06C5 | TAATCCTCATTACCAGAACCACCACCATTGCCCCC | 277 |
| | TTT | |
| 50-Reg-T2R07C4 | TGAGCCGCCTTTTAAAGCCAGAATGGATTGATGAT | 278 |
| | ACT | |
| 51-Reg-T2R08C5 | TATTAGCGTTTCCGTAATCAGTAGCGATTATTGAG | 279 |
| | GGT | |
| 52-Reg-T2R10C1 | TGCCATTTGTTCAAACGTAGAAAATACTTCTGGCA | 280 |
| | TGT | |
| 53-Reg-T2R10C5 | TAGGGAAGGTTATAAGTTTATTTTGTCTTAGCCGA | 281 |
| | ACT | |
| 54-Reg-T2R11C2 | TAGGTGGCATTGAATTATCACCGTCACTTCATTAG | 282 |
| | CAT | |
| 55-Reg-T2R12C5 | TAAAGTTACTTGCCCAATAATAAGAGCTTAGCCTT | 283 |
| | TAT | |
| 56-Reg-T2R13C2 | TCGCTAATATTGGAATACCCAAAAGAATTATACAT | 284 |
| | AAT | |
| 57-Reg-T2R14C5 | TCAGAGAGATTACAAAATAAACAGCCATTTTAAAT | 285 |
| | CAT | |
| 58-Reg-T2R16C5 | TAGATTAGTTTATATAGAAGGCTTATCTTCAAGCC | 286 |
| | GTT | |
| 59-Reg-T2R17C4 | TCAAATCAGTTTGCTATTTTGCACCCATTGCCTAAT | 287 |
| | TT | |
| 60-Reg-T3R04C5 | TAAATAAGATTACTTTTTCAAATATATTTCTGAGA | 288 |
| | GAT | |
| 61-Reg-T3R06C5 | TCTACCTTTTTAGAATCCTTGAAAACATTAGAAAA | 289 |
| | CAT | |
| 62-Reg-T3R07C4 | TTTTCCCTTTTTTAACCTCCGGCTTAGTTCAAAGAA | 290 |
| | CT | |
| 63-Reg-T3R08C5 | TAAATTAATTTACCAAGTTACAAAATCTTCTGAAT | 291 |
| | AAT | |
| 64-Reg-T3R09C4 | TCTTTGAATTTTACATTTAACAATTTCTTTAATTAA | 292 |
| | TT | |
| 65-Reg-T3R10C1 | TGTAGATTTTTGTTATTAATTTTAAAATTAACAATT | 293 |
| | CT | |
| 66-Reg-T3R10C5 | TTGGAAGGGTTAGCGGAATTATCATCATTACTAAT | 294 |
| | AGT | |
| 67-Reg-T3R11C2 | TAACATTATTTGTAAAACAGAAATAAATTTTTTAC | 295 |
| | ATT | |
| 68-Reg-T3R11C4 | TCCAGAAGGTTTTAGAACCTACCATATTTCCTGAT | 296 |
| | TGT | |
| 69-Reg-T3R12C5 | TATTAGAGCTTAATATCTGGTCAGTTGTTCAGCAG | 297 |
| | AAT | |
| 70-Reg-T3R13C2 | TGCATCACCTTAGTATTAGACTTTACATTGTTTGAG | 298 |
| | TT | |
| 71-Reg-T3R13C4 | TCCTCAATCTTCGTCAATAGATAATACTTAGAAAC | 299 |
| | CAT | |
| 72-Reg-T3R14C5 | TGATAAAACTTTTTTTGAATGGCTATTTTTTCACCA | 300 |
| | GT | |
| 73-Reg-T3R15C4 | TAGACAATATTAGAGGTGAGGCGGTCATTTATCAA | 301 |
| | ACT | |
| 74-Reg-T3R16C5 | TTCACACGATTTGCAACAGGAAAAACGTTGAAGA | 302 |
| | ACTT | |
| 75-Reg-T3R17C4 | TCCAGCCATTTCCAGTAATAAAAGGGATTCGTGGC | 303 |
| | ACT | |
| 76-Reg-T4R04C5 | TAGCACTAATTAAAGGGCGAAAAACCGTTAAATC | 304 |
| | CCTT | |
| 77-Reg-T4R06C5 | TTATAAATCTTGAGAGTTGCAGCAAGCTTGTCGTG | 305 |
| | CCT | |
| 78-Reg-T4R07C4 | TGGCCCTGATTAAAAGAATAGCCCGAGTTCGTGGA | 306 |
| | CTT | |
| 79-Reg-T4R08C5 | TAGCTGCATTTAGCCTGGGGTGCCTAATTGTAAAA | 307 |
| | CGT | |
| 80-Reg-T4R09C4 | TAAGTGTAATTTAATGAATCGGCCAACTTCACCGC | 308 |
| | CTT | |
| 81-Reg-T4R10C1 | TGAATTCGTTTGCCATTCGCCATTCAGTTTTCCGGC | 309 |
| | AT | |
| 82-Reg-T4R10C5 | TACGGCCAGTTTACGCCAGCTGGCGAATTCATCTG | 310 |
| | CCT | |
| 83-Reg-T4R11C2 | TACTGTTGGTTAGAGGATCCCCGGGTATTCCGCTC | 311 |
| | ACT | |
| 84-Reg-T4R11C4 | TTTCGCTATTTTGCCAAGCTTGCATGCTTGAAGCAT | 312 |
| | AT | |
| 85-Reg-T4R12C5 | TAGTTTGAGTTATTCTCCGTGGGAACATTATTCGC | 313 |
| | ATT | |
| 86-Reg-T4R13C2 | TTTCATCAATTCGCACTCCAGCCAGCTTTGCTGCG | 314 |
| | CAT | |
| 87-Reg-T4R13C4 | TCCCGTCGGTTGGGACGACGACAGTATTTCGGGCC | 315 |
| | TCT | |
| 88-Reg-T4R14C5 | TTAAATTTTTTTGATAATCAGAAAAGCTTACAAAG | 316 |
| | GCT | |
| 89-Reg-T4R15C4 | TACCCCGGTTTTGTTAAATCAGCTCATTTAGTAAC | 317 |
| | AAT | |
| 90-Reg-T4R16C5 | TTATCAGGTTTAAATCACCATCAATATTTCAATGC | 318 |
| | CTT | |
| 91-Reg-T4R17C4 | TAGACAGTCTTCATTGCCTGAGAGTCTTTTCATATG | 319 |
| | TT | |
| 92-Reg-T2R10C4 | TGACGGAAATTACCATCGATAGCAGCATTTTGCCA | 320 |
| | TCTTTTCATATTCACCCTCAT | |
| 93-Reg-T2R15C4 | TTGCCAGTTTTATAACATAAAAACAGGTTACAAGA | 321 |
| | ATTGAGTTAATTCAGAAGGAT | |
| 94-Edg- | TTTGGTGGCATCAATTCTATTGGGCGCGAGCTGAA | 322 |
| T1R00C7-T3.5p- | AATTTAACTACTCCCACTCTCACCCTCACCCTACTC | |
| T3.3p- | CAACTCAAC | |
| DYE40.2.3p | | |
| 95-Edg- | TTTTCCCAATTCTGCGAACTTCCATATAACAGTTG | 323 |
| T1R02C7-T3.5p- | ATTTTAACTACTCCCACTCTCACCCTCACCCTACTC | |
| T3.3p- | CAACTCAAC | |
| DYE40.2.3p | | |
| 96-Edg- | TTTATTGCTCCTTTTGATATTTTAGAGAGTACCTTT | 324 |
| T1R04C7-T3.5p- | ATTTAACTACTCCCACTCTCACCCTCACCCTACTCC | |
| T3.3p- | AACTCAAC | |
| DYE40.2.3p | | |
| 97-Edg- | TTTCCATAAATCAAAAATCTTCAGAAAACGAGAAT | 325 |
| T1R06C7-T3.5p- | GATTTAACTACTCCCACTCTCACCCTCACCCTACTC | |
| T3.3p- | CAACTCAAC | |
| DYE40.2.3p | | |
| 98-Edg- | TTTCGAGGCATAGTAAGAGTTACGCCAAAAGGAA | 326 |
| T1R08C7-T3.5p- | TTATTTAACTACTCCCACTCTCACCCTCACCCTACT | |
| T3.3p- | CCAACTCAAC | |
| DYE40.2.3p | | |
| 99-Edg- | TTTGAAACACCAGAACGAGTTAGGCTTGCCCTGAC | 327 |
| T1R10C7-T3.5p- | GATTTAACTACTCCCACTCTCACCCTCACCCTACTC | |
| T3.3p- | CAACTCAAC | |
| DYE40.2.3p | | |
| 100-Edg- | TTTCTGATAAATTGTGTCGTTAGATTTGTATCATCG | 328 |
| T1R12C7-T3.5p- | CTTTAACTACTCCCACTCTCACCCTCACCCTACTCC | |
| T3.3p- | AACTCAAC | |
| DYE40.2.3p | | |
| 101-Edg- | TTTGAACGAGGGTAGCAACTTGCGAAAGACAGCA | 329 |
| T1R14C7-T3.5p- | TCGTTTAACTACTCCCACTCTCACCCTCACCCTACT | |
| T3.3p- | CCAACTCAAC | |
| DYE40.2.3p | | |
| 102-Edg- | TTTGGTTTATCAGCTTGCTTTAGCCTTTAATTGTAT | 330 |
| T1R16C7-T3.5p- | CTTTAACTACTCCCACTCTCACCCTCACCCTACTCC | |
| T3.3p- | AACTCAAC | |
| DYE40.2.3p | | |
| 103-Edg- | TTTGGGATTTTGCTAAACATTAATGAATTTTCTGTA | 331 |
| T1R18C7-T3.5p- | TTTTAACTACTCCCACTCTCACCCTCACCCTACTCC | |
| T3.3p- | AACTCAAC | |
| DYE40.2.3p | | |
| 104-Edg- | TTTACAAACTACAACGCCTTTGAGTTTCGTCACCA | 332 |
| T1R20C7-T3.5p- | GTTTTAACTACTCCCACTCTCACCCTCACCCTACTC | |
| T3.3p- | CAACTCAAC | |
| DYE40.2.3p | | |
| 105-Edg- | TTTAGCCACCACCCTCATTTTGAACCGCCACCCTC | 333 |
| T2R00C7-T3.5p- | AGTTTAACTACTCCCACTCTCACCCTCACCCTACTC | |
| T3.3p- | CAACTCAAC | |
| DYE40.2.3p | | |
| 106-Edg- | TTTGAGAGGGTTGATATAATTGCGGATAAGTGCCG | 334 |
| T2R02C7-T3.5p- | TCTTTAACTACTCCCACTCTCACCCTCACCCTACTC | |
| T3.3p- | CAACTCAAC | |
| DYE40.2.3p | | |
| 107-Edg- | TTTGTATAAACAGTTAATGTTTTGAGTAACAGTGC | 335 |
| T2R04C7-T3.5p- | CCTTTAACTACTCCCACTCTCACCCTCACCCTACTC | |
| T3.3p- | CAACTCAAC | |
| DYE40.2.3p | | |
| 108-Edg- | TTTGCAGGTCAGACGATTGTTTTGACAGGAGGTTG | 336 |
| T2R06C7-T3.5p- | AGTTTAACTACTCCCACTCTCACCCTCACCCTACTC | |
| T3.3p- | CAACTCAAC | |
| DYE40.2.3p | | |
| 109-Edg- | TTTTAGCGCGTTTTCATCGTTCTTTAGCGTCAGACT | 337 |
| T2R08C7-T3.5p- | GTTTAACTACTCCCACTCTCACCCTCACCCTACTCC | |
| T3.3p- | AACTCAAC | |
| DYE40.2.3p | | |
| 110-Edg- | TTTGCGCCAAAGACAAAAGTTTTCATATGGTTTAC | 338 |
| T2R10C7-T3.5p- | CATTTAACTACTCCCACTCTCACCCTCACCCTACTC | |
| T3.3p- | CAACTCAAC | |
| DYE40.2.3p | | |
| 111-Edg- | TTTCCGAAGCCCTTTTTAATTAGCAATAGCTATCTT | 339 |
| T2R12C7-T3.5p- | ATTTAACTACTCCCACTCTCACCCTCACCCTACTCC | |
| T3.3p- | AACTCAAC | |
| DYE40.2.3p | | |
| 112-Edg- | TTTTTTTTTGTTTAACGTCTTTCCAAATAAGAAACG | 340 |
| T2R14C7-T3.5p- | ATTTAACTACTCCCACTCTCACCCTCACCCTACTCC | |
| T3.3p- | AACTCAAC | |
| DYE40.2.3p | | |
| 113-Edg- | TTTAACCTCCCGACTTGCGTTGCGAGGCGTTTTAG | 341 |
| T2R16C7-T3.5p- | CGTTTAACTACTCCCACTCTCACCCTCACCCTACTC | |
| T3.3p- | CAACTCAAC | |
| DYE40.2.3p | | |
| 114-Edg- | TTTTAAACCAAGTACCGCATTTTCCAAGAACGGGT | 342 |
| T2R18C7-T3.5p- | ATTTTAACTACTCCCACTCTCACCCTCACCCTACTC | |
| T3.3p- | CAACTCAAC | |
| DYE40.2.3p | | |
| 115-Edg- | TTTAGATAAGTCCTGAACATTCCTGTTTATCAACA | 343 |
| T2R20C7-T3.5p- | ATTTTAACTACTCCCACTCTCACCCTCACCCTACTC | |
| T3.3p- | CAACTCAAC | |
| DYE40.2.3p | | |
| 116-Edg- | TTTGTAAAGTAATTCTGTCTTAAAGTACCGACAAA | 344 |
| T3R00C7-T3.5p- | AGTTTAACTACTCCCACTCTCACCCTCACCCTACTC | |
| T3.3p- | CAACTCAAC | |
| DYE40.2.3p | | |
| 117-Edg- | TTTAGTAGGGCTTAATTGATTAAAGCCAACGCTCA | 345 |
| T3R02C7-T3.5p- | ACTTTAACTACTCCCACTCTCACCCTCACCCTACTC | |
| T3.3p- | CAACTCAAC | |
| DYE40.2.3p | | |
| 118-Edg- | TTTAATGGTTTGAAATACCTTCTTCTGACCTAAATT | 346 |
| T3R04C7-T3.5p- | TTTTAACTACTCCCACTCTCACCCTCACCCTACTCC | |
| T3.3p- | AACTCAAC | |
| DYE40.2.3p | | |
| 119-Edg- | TTTAGTCAATAGTGAATTTTTTTAAGACGCTGAGA | 347 |
| T3R06C7-T3.5p- | AGTTTAACTACTCCCACTCTCACCCTCACCCTACTC | |
| T3.3p- | CAACTCAAC | |
| DYE40.2.3p | | |
| 120-Edg- | TTTTGAGCAAAAGAAGATGTTATTCATTTCAATTA | 348 |
| T3R08C7-T3.5p- | CCTTTAACTACTCCCACTCTCACCCTCACCCTACTC | |
| T3.3p- | CAACTCAAC | |
| DYE40.2.3p | | |
| 121-Edg- | TTTCAATATAATCCTGATTTTGATGATGGCAATTCA | 349 |
| T3R10C7-T3.5p- | TTTTAACTACTCCCACTCTCACCCTCACCCTACTCC | |
| T3.3p- | AACTCAAC | |
| DYE40.2.3p | | |
| 122-Edg- | TTTGTTATCTAAAATATCTTTAAAGGAATTGAGGA | 350 |
| T3R12C7-T3.5p- | AGTTTAACTACTCCCACTCTCACCCTCACCCTACTC | |
| T3.3p- | CAACTCAAC | |
| DYE40.2.3p | | |
| 123-Edg- | TTTACATCGCCATTAAAAATTAACTGATAGCCCTA | 351 |
| T3R14C7-T3.5p- | AATTTAACTACTCCCACTCTCACCCTCACCCTACTC | |
| T3.3p- | CAACTCAAC | |
| DYE40.2.3p | | |
| 124-Edg- | TTTTCGTCTGAAATGGATTTTACATTTTGACGCTCA | 352 |
| T3R16C7-T3.5p- | ATTTAACTACTCCCACTCTCACCCTCACCCTACTCC | |
| T3.3p- | AACTCAAC | |
| DYE40.2.3p | | |
| 125-Edg- | TTTTTGATTAGTAATAACATTTTGTAGCAATACTTC | 353 |
| T3R18C7-T3.5p- | TTTTAACTACTCCCACTCTCACCCTCACCCTACTCC | |
| T3.3p- | AACTCAAC | |
| DYE40.2.3p | | |
| 126-Edg- | TTTAGGAACGGTACGCCAGTTTAAAGGGATTTTAG | 354 |
| T3R20C7-T3.5p- | ACTTTAACTACTCCCACTCTCACCCTCACCCTACTC | |
| T3.3p- | CAACTCAAC | |
| DYE40.2.3p | | |
| 127-Edg- | TTTGAGCACGTATAACGTGTTCTATGGTTGCTTTGA | 355 |
| T4R00C7-T3.5p- | CTTTAACTACTCCCACTCTCACCCTCACCCTACTCC | |
| T3.3p- | AACTCAAC | |
| DYE40.2.3p | | |
| 128-Edg- | TTTCGGGCGCTAGGGCGCTTTAAGAAAGCGAAAG | 356 |
| T4R02C7-T3.5p- | GAGTTTAACTACTCCCACTCTCACCCTCACCCTACT | |
| T3.3p- | CCAACTCAAC | |
| DYE40.2.3p | | |
| 129-Edg- | TTTATCACCCAAATCAAGTTTGCCCACTACGTGAA | 357 |
| T4R04C7-T3.5p- | CCTTTAACTACTCCCACTCTCACCCTCACCCTACTC | |
| T3.3p- | CAACTCAAC | |
| DYE40.2.3p | | |
| 130-Edg- | TTTATCCTGTTTGATGGTGTTGCCCCAGCAGGCGA | 358 |
| T4R06C7-T3.5p- | AATTTAACTACTCCCACTCTCACCCTCACCCTACTC | |
| T3.3p- | CAACTCAAC | |
| DYE40.2.3p | | |
| 131-Edg- | TTTGCTCACTGCCCGCTTTTTACATTAATTGCGTTG | 359 |
| T4R08C7-T3.5p- | CTTTAACTACTCCCACTCTCACCCTCACCCTACTCC | |
| T3.3p- | AACTCAAC | |
| DYE40.2.3p | | |
| 132-Edg- | TTTGTAACGCCAGGGTTTTTTAAGGCGATTAAGTT | 360 |
| T4R10C7-T3.5p- | GGTTTAACTACTCCCACTCTCACCCTCACCCTACTC | |
| T3.3p- | CAACTCAAC | |
| DYE40.2.3p | | |
| 133-Edg- | TTTCGTTGGTGTAGATGGGTTGTAATGGGATAGGT | 361 |
| T4R12C7-T3.5p- | CATTTAACTACTCCCACTCTCACCCTCACCCTACTC | |
| T3.3p- | CAACTCAAC | |
| DYE40.2.3p | | |
| 134-Edg- | TTTTTTAAATTGTAAACGTTTATTGTATAAGCAAAT | 362 |
| T4R14C7-T3.5p- | ATTTAACTACTCCCACTCTCACCCTCACCCTACTCC | |
| T3.3p- | AACTCAAC | |
| DYE40.2.3p | | |
| 135-Edg- | TTTGCCGGAGAGGGTAGCTTTTAGCTGATAAATTA | 363 |
| T4R16C7-T3.5p- | ATTTTAACTACTCCCACTCTCACCCTCACCCTACTC | |
| T3.3p- | CAACTCAAC | |
| DYE40.2.3p | | |
| 136-Edg- | TTTAAATTTTTAGAACCCTTTTTCAACGCAAGGAT | 364 |
| T4R18C7-T3.5p- | AATTTAACTACTCCCACTCTCACCCTCACCCTACTC | |
| T3.3p- | CAACTCAAC | |
| DYE40.2.3p | | |
| 137-Edg- | TTTTAAGCAATAAAGCCTCTTAAAGAATTAGCAAA | 365 |
| T4R20C7-T3.5p- | ATTTTAACTACTCCCACTCTCACCCTCACCCTACTC | |
| T3.3p- | CAACTCAAC | |
| DYE40.2.3p | | |
| 138-Bri-T1R14C2 | TTGCCACTACTTTTTTTGCCACCCTCT | 366 |
| 139-Bri-T2R14C2 | TAACTGAACATTTTTTTTGAATAACCT | 367 |
| 140-Bri-T3R14C2 | TGCCACGCTGTTTTTTTACCAGTGAGT | 368 |
| 141-Bri-T4R14C2 | TCAAAAATAATTTTTTTTGTTTAGACT | 369 |
| 142-Bri-T1R02C5 | TGATACATTTCGCTTTTTTGACCCTGTAATT | 370 |
| 143-Bri-T2R02C5 | TACCGTACTCAGGTTTTTGATCTAAAGTTTT | 371 |
| 144-Bri-T3R02C5 | TAACATGTAATTTTTTTTGAAACCAATCAAT | 372 |
| 145-Bri-T4R02C5 | TGCGTAACCACCATTTTTGAGTAAAAGAGTT | 373 |
| 146-Bri-T1R08C1 | TCAGAGGGGGTTTTGCCTTCCTGTAGCCAGCTT | 374 |
| 147-Bri-T2R08C1 | TGAACCGCCTCTTTACCTAAAACGAAAGAGGCT | 375 |
| 148-Bri-T3R08C1 | TCATAAATCAATTTAGTCAGAGGGTAATTGAGT | 376 |
| 149-Bri-T4R08C1 | TCCAGGGTGGTTTTGCAAATGAAAAATCTAAAT | 377 |
| 150-Bri-T1R16C3 | TACAACCATTTTTTCATACATGGCTTTTTTAAGCGC | 378 |
| | AT | |
| 151-Bri-T2R16C3 | TTTTTATCTTTTTTATCCAATCGCAAGATTGTTGGG | 379 |
| | TT | |
| 152-Bri-T3R16C3 | TGCCAACATTTTTTCCACTATTAAAGAATTATAGG | 380 |
| | GTT | |
| 153-Bri-T4R16C3 | TACAAGAGTTTTTTTCGCGTTTTAATTCTTAAAAAG | 381 |
| | AT | |
| 154-Bri-T1R18C5 | TGAGAATAGAAAGGAACAACTATTTTCTCAAGAG | 382 |
| | AAGGAT | |
| 155-Bri-T2R18C5 | TTTTTATTTTCATCGTAGGAATTTTTAGCCTGTTTA | 383 |
| | GTAT | |
| 156-Bri-T3R18C5 | TCAAACTATCGGCCTTGCTGGTTTTTGAGCTTGAC | 384 |
| | GGGGT | |
| 157-Bri-T4R18C5 | TGAGTAATGTGTAGGTAAAGATTTTTTGTTTTAAA | 385 |
| | TATGT | |
| 158-Bri-T1R07C2 | TTGGATAGCTTAAGCCCGATTTTTAATCGTATTAA | 386 |
| | CGCCATT | |
| 159-Bri-T2R07C2 | TAGAACCGCTTATTTACCGTTTTACCGATATTTATA | 387 |
| | CGTAAT | |
| 160-Bri-T3R07C2 | TTTGCTTCTTTTATATGTATTTTACGCTAACTTGGA | 388 |
| | GAATTT | |
| 161-Bri-T4R07C2 | TACGGGCAATTGTTCCAGTTTTTTCTGACCTTTGCA | 389 |
| | ACAGTT | |
| 162-Bri-T1R12C1 | TAGGACAGATGATTTTTTCACCAGTAGCACCATTA | 390 |
| | CTTCGACTTGAT | |
| 163-Bri-T2R12C1 | TATTAAGACTCCTTTTTAATATACAGTAACAGTAC | 391 |
| | CTTGAAATTGCT | |
| 164-Bri-T3R12C1 | TGACAACTCGTATTTTTTCCTGTGTGAAATTGTTAT | 392 |
| | TTCCGAGCTCT | |
| 165-Bri-T4R12C1 | TCCGCTTCTGGTTTTTTCGTTAATAAAACGAACTAA | 393 |
| | TTATTATACCT | |
| 166-Bri-T1R19C5 | TTGTCGTCTTTCAGCCCTCATATTTTTTTCGCCACC | 394 |
| | CTCATTGGTGTATCT | |
| 167-Bri-T2R19C5 | TTAATCGGCTTCATCCTAATTTTTTTTTTTTTTCGAG | 395 |
| | CCATTACAACGCCT | |
| 168-Bri-T3R19C5 | TCTGTCCATTTTTTTATAATCATTTTTTTCTTAATGC | 396 |
| | GCCTTCACGCTGCT | |
| 169-Bri-T4R19C5 | TACTTTTGCTTATCGGTTGTACTTTTTTTAACCTGTT | 397 |
| | TAGTTGACCATTAT | |
| 170-Bri-T1R05C4 | TAAGCGAACTTAATTGCTGAATATAATGCTGTATT | 398 |
| | TTTTTGTGAGAAAGGCCGGT | |
| 171-Bri-T2R05C4 | TAGGAGTGTTTAAACATGAAAGTATTAAGAGGCTT | 399 |
| | TTTTTGCGAATAATAATTTT | |
| 172-Bri-T3R05C4 | TGCGAGAAATTATAAACACCGGAATCATAATTATT | 400 |
| | TTTTTCGCCCAATAGCAAGT | |
| 173-Bri-T4R05C4 | TCCAACGTCTTATCGGAACCCTAAAGGGAGCCCTT | 401 |
| | TTTTTGAACAATATTACCGT | |
| 174-Bri-T2R10C0 | TGGAATTAGAGCTTTTTTTTCAGACCAGGCGTTCG | 402 |
| | TTGGGAAGATTTTTTTTCCAGGCAAAGCT | |
| 175-Bri-T4R10C0 | TAATCATGGTCATTTTTTTTTTTGCCCGAACTTTCA | 403 |
| | GGTTTAACTTTTTTTTCAGTATGTTAGT | |
| 176-Reg- | TTTTCATTTTGAGTAGATTTAGTTTTTCTATATTTT | 404 |
| T1R03C6 | | |
| 177-Reg- | TAACAGTTTTAGGTCTTTACCCTGATTTCCAACAGT | 405 |
| T1R07C6 | | |
| 178-Reg- | TGTGAATATTTAGTAAATTGGGCTTTTTAATGCAG | 406 |
| T1R11C6 | T | |
| 179-Reg- | TCTCAGCATTGGCTACAGAGGCTTTTTAACAAAGT | 407 |
| T1R15C6 | T | |
| 180-Reg- | TGTTAGTATTACTTTCAACAGTTTCTTAAAGGCTCT | 408 |
| T1R19C6 | | |
| 181-Reg- | TGTACCAGTTGTATAGCCCGGAATATTGAACCGCC | 409 |
| T2R03C6 | T | |
| 182-Reg- | TGCCAGCATTGCCTTGATATTCACATTAACGGGGT | 410 |
| T2R07C6 | T | |
| 183-Reg- | TTAGAAAATTGGCGACATTCAACCGTTCAGAATCA | 411 |
| T2R11C6 | T | |
| 184-Reg- | TATCCCAATTAAAAATGAAAATAGCTTAAGAAAC | 412 |
| T2R15C6 | AT | |
| 185-Reg- | TCTTATCATTCTCATCGAGAACAAGTTCGGTATTCT | 413 |
| T2R19C6 | | |
| 186-Reg- | TCCAGTATTTGAATCGCCATATTTATTGTAATAAGT | 414 |
| T3R03C6 | | |
| 187-Reg- | TGCTTAGATTATCAAAATCATAGGTTTTTTAGTTAT | 415 |
| T3R07C6 | | |
| 188-Reg- | TATTATCATTGTTTGGATTATACTTTTGCGCAGAGT | 416 |
| T3R11C6 | | |
| 189-Reg- | TATGCGCGTTTACCGAACGAACCACTTGCAAATCA | 417 |
| T3R15C6 | T | |
| 190-Reg- | TTTAACCGTTTCACTTGCCTGAGTATTCTCATGGAT | 418 |
| T3R19C6 | | |
| 191-Reg- | TGGAAGGGTTGGCAAGTGTAGCGGTTTGCTACAGG | 419 |
| T4R03C6 | T | |
| 192-Reg- | TCTGGTTTTTGTTCCGAAATCGGCATTTCTATCAGT | 420 |
| T4R07C6 | | |
| 193-Reg- | TGTGCTGCTTCCCAGTCACGACGTTTTTGAGTGAG | 42 |
| T4R11C6 | T | |
| 194-Reg- | TCAGGAAGTTTAATATTTTGTTAAATTAACGGCGG | 422 |
| T4R15C6 | T | |
| 195-Reg- | TCCTTTATTTCATATATTTTAAATGTTGATATTCAT | 423 |
| T4R19C6 | | |
| 196-Reg- | TGTCAGGATTAGAGGTCATTTTTGCTTTCTGGAAG | 424 |
| T1R05C6 | T | |
| 197-Reg- | TATACATATTCAACACTATCATAACTTATGCTTTAT | 425 |
| T1R09C6 | | |
| 198-Reg- | TACAACGGTTAAATCCGCGACCTGCTTCTCATTCA | 426 |
| T1R13C6 | T | |
| 199-Reg- | TCAAAAGGTTTTCGAGGTGAATTTCTTTCGTCACCT | 427 |
| T1R17C6 | | |
| 200-Reg- | TATTTCATTTGACCGTGTGATAAATTTAATTCTTAT | 428 |
| T3R05C6 | | |
| 201-Reg- | TGCGAATTTTATGAAACAAACATCATTTAGCGATA | 429 |
| T3R09C6 | T | |
| 202-Reg- | TACAGTTGTTTTAGGAGCACTAACATTTATTCCTGT | 430 |
| T3R13C6 | | |
| 203-Reg- | TAATACCTTTATTTACATTGGCAGATTAGTCTTTAT | 431 |
| T3R17C6 | | |
| 204-Reg- | CCATGTACCGTAACACTTTGTAGCATTCCACAGAT | 432 |
| T1R21C5 | TTTCCAGACT | |
| 205-Reg- | CTAAACAGGAGGCCGATTTAATCCTGAGAAGTGTT | 433 |
| T3R21C5 | TCACGCAAAT | |
| 206-Reg- | TCAGTGCCTTCCCCCTGCCTATTTCTTTTTGCTCAT | 434 |
| T2R05C6 | | |
| 207-Reg- | TAGTTTGCTTGCATTTTCGGTCATATTGAGCCGCCT | 435 |
| T2R09C6 | | |
| 208-Reg- | TATGAAATTTGAAAAGTAAGCAGATTTACAATCAA | 436 |
| T2R13C6 | T | |
| 209-Reg- | TTAAGAACTTGGAGGTTTTGAAGCCTTTATTATTTT | 437 |
| T2R17C6 | | |
| 210-Reg- | TGGCGATGTTTTTTTGGGGTCGAGGTTGCGAGAAA | 438 |
| T4R05C6 | T | |
| 211-Reg- | TCTAACTCTTCCAGTCGGGAAACCTTTGGTCCACG | 439 |
| T4R09C6 | T | |
| 212-Reg- | TATTGACCTTCGCATCGTAACCGTGTTAGGGGGAT | 440 |
| T4R13C6 | T | |
| 213-Reg- | TACCGTTCTTATTTTTGAGAGATCTTTCCCAAAAAT | 441 |
| T4R17C6 | | |
| 214-Reg- | AGCTAATGCAGAACGCGTTAGAAAAATAATATCC | 442 |
| T2R21C5 | TTTGTCTTTCT | |
| 215-Reg- | AATCATACAGGCAAGGCTTAGAGCATAAAGCTAA | 443 |
| T4R21C5 | TTGGGAGAAGT | |
| 216-Reg- | TCCCCAGCGTTGGAACGAGGCGCAGACTTTATTCA | 444 |
| T1R13C4-T2.3p- | TTTTTCACTCACCTCCATCTCCACTCCTACCCATCC | |
| APT40.2.3p | AACTCCCAC | |
| 217-Reg- | TAACCGAGGTTGCAAAGACACCACGGATTTAAAT | 445 |
| T2R12C4-T2.3p- | ATTTTTCACTCACCTCCATCTCCACTCCTACCCATC | |
| APT40.2.3p | CAACTCCCAC | |

The second cross-linking strategy included folding a DNA origami tile (i.e. “Tile2” having standard staples without thymines at positions specifically engineered for cross-linking) under the conditions set forth above for the dTile. The folded Tile was then irradiated with 365 nm UV in the presence of a psoralen cross-linker (8-Methoxypsoralen, aka 8-MOP) under conditions set forth below to covalently link pyrimidine bases (e.g., thymine and cytosine). A listing of oligonucleotide sequences for Tile2 is provided in Table 4.

TABLE 4

| Oligonucleotide | | |
|---|---|---|
| Sequences for | | |
| Tile2Sequence | | SEQ ID |
| Name | Sequence | NO |
| 1-Reg-T1R01C6 | TCATTTGCTAATAGTAGTAGCATT | 12 |
| 2-Reg-T1R03C5 | CAACTAAAGTACGGTGGGATGGCT | 13 |
| 3-Reg-T1R09C2 | CATTATTAGCAAAAGAAGTTTTGC | 14 |
| 4-Reg-T2R01C6 | ACCCTCATTCAGGGATAGCAAGCC | 15 |
| 5-Reg-T2R03C5 | TTAGGATTAGCGGGGTGGAACCTA | 16 |
| 6-Reg-T2R09C2 | AGGCCGGAACCAGAGCCACCACCG | 17 |
| 7-Reg-T3R01C6 | AGAATATCAGACGACGACAATAAA | 18 |
| 8-Reg-T3R03C5 | TCATATGCGTTATACAAAGGCGTT | 19 |
| 9-Reg-T3R09C2 | CGGGAGAATTTAATGGAAACAGTA | 20 |
| 10-Reg-T4R01C6 | GCGCGTACTTTCCTCGTTAGAATC | 21 |
| 11-Reg-T4R03C5 | AAAGCCGGCGAACGTGTGCCGTAA | 22 |
| 12-Reg-T4R09C2 | AATTCCACGTTTGCGTATTGGGCG | 23 |
| 13-Reg-T1R03C6 | TTTCATTGAGTAGATTTAGTTTCTATATTT | 208 |
| 14-Reg-T1R05C6 | GTCAGGAAGAGGTCATTTTTGCTCTGGAAG | 446 |
| 15-Reg-T1R06C3 | TTAAGAGGGTCCAATACTGCGGATAGCGAG | 24 |
| 16-Reg-T1R07C6 | AACAGTTAGGTCTTTACCCTGATCCAACAG | 209 |
| 17-Reg-T1R08C3 | AGGCTTTTCAGGTAGAAAGATTCAATTACC | 25 |
| 18-Reg-T1R09C6 | ATACATACAACACTATCATAACATGCTTTA | 447 |
| 19-Reg-T1R10C3 | TTATGCGATTGACAAGAACCGGAGGTCAAT | 26 |
| 20-Reg-T1R11C6 | GTGAATATAGTAAATTGGGCTTTAATGCAG | 210 |
| 21-Reg-T1R12C3 | CATAAGGGACACTAAAACACTCACATTAAA | 27 |
| 22-Reg-T1R13C6 | ACAACGGAAATCCGCGACCTGCCTCATTCA | 448 |
| 23-Reg-T1R14C3 | CGGGTAAAATTCGGTCGCTGAGGAATGACA | 28 |
| 24-Reg-T1R15C6 | CTCAGCAGGCTACAGAGGCTTTAACAAAGT | 211 |
| 25-Reg-T1R17C6 | CAAAAGGTTCGAGGTGAATTTCTCGTCACC | 449 |
| 26-Reg-T1R19C6 | GTTAGTAACTTTCAACAGTTTCAAAGGCTC | 212 |
| 27-Reg-T2R03C6 | GTACCAGGTATAGCCCGGAATAGAACCGCC | 213 |
| 28-Reg-T2R05C6 | CAGTGCCCCCCCTGCCTATTTCTTTGCTCA | 450 |
| 29-Reg-T2R06C3 | GTCTCTGACACCCTCAGAGCCACATCAAAA | 29 |
| 30-Reg-T2R07C6 | GCCAGCAGCCTTGATATTCACAAACGGGGT | 214 |
| 31-Reg-T2R08C3 | TCACCGGAAACGTCACCAATGAATTATTCA | 30 |
| 32-Reg-T2R09C6 | AGTTTGCGCATTTTCGGTCATAGAGCCGCC | 45 |
| 33-Reg-T2R10C3 | TTAAAGGTACATATAAAAGAAACAAACGCA | 31 |
| 34-Reg-T2R11C6 | TAGAAAAGGCGACATTCAACCGCAGAATCA | 215 |
| 35-Reg-T2R12C3 | ATAATAACTCAGAGAGATAACCCGAAGCGC | 32 |
| 36-Reg-T2R13C6 | ATGAAATGAAAAGTAAGCAGATACAATCAA | 452 |
| 37-Reg-T2R14C3 | ATTAGACGGAGCGTCTTTCCAGAGCTACAA | 33 |
| 38-Reg-T2R15C6 | ATCCCAAAAAAATGAAAATAGCAAGAAACA | 216 |
| 39-Reg-T2R17C6 | TAAGAACGGAGGTTTTGAAGCCTATTATTT | 453 |
| 40-Reg-T2R19C6 | CTTATCACTCATCGAGAACAAGCGGTATTC | 217 |
| 41-Reg-T3R03C6 | CCAGTATGAATCGCCATATTTAGTAATAAG | 218 |
| 42-Reg-T3R05C6 | ATTTCATGACCGTGTGATAAATAATTCTTA | 454 |
| 43-Reg-T3R06C3 | TATATAACGTAAATCGTCGCTATATTTGAA | 34 |
| 44-Reg-T3R07C6 | GCTTAGAATCAAAATCATAGGTTTTAGTTA | 219 |
| 45-Reg-T3R08C3 | TTACCTTTACAATAACGGATTCGCAAAATT | 35 |
| 46-Reg-T3R09C6 | GCGAATTATGAAACAAACATCATAGCGATA | 455 |
| 47-Reg-T3R10C3 | ATTTGCACCATTTTGCGGAACAAATTTGAG | 36 |
| 48-Reg-T3R11C6 | ATTATCAGTTTGGATTATACTTGCGCAGAG | 220 |
| 49-Reg-T3R12C3 | GATTTAGATTGCTGAACCTCAAAGTATTAA | 37 |
| 50-Reg-T3R13C6 | ACAGTTGTTAGGAGCACTAACATATTCCTG | 456 |
| 51-Reg-T3R14C3 | CACCGCCTGAAAGCGTAAGAATACATTCTG | 38 |
| 52-Reg-T3R15C6 | ATGCGCGTACCGAACGAACCACGCAAATCA | 221 |
| 53-Reg-T3R17C6 | AATACCTATTTACATTGGCAGAAGTCTTTA | 457 |
| 54-Reg-T3R19C6 | TTAACCGTCACTTGCCTGAGTACTCATGGA | 222 |
| 55-Reg-T4R03C6 | GGAAGGGGGCAAGTGTAGCGGTGCTACAGG | 223 |
| 56-Reg-T4R05C6 | GGCGATGTTTTTGGGGTCGAGGGCGAGAAA | 458 |
| 57-Reg-T4R06C3 | TGAGTGTTCAGCTGATTGCCCTTGCGCGGG | 39 |
| 58-Reg-T4R07C6 | CTGGTTTGTTCCGAAATCGGCATCTATCAG | 224 |
| 59-Reg-T4R08C3 | GAGAGGCGACAACATACGAGCCGCTGCAGG | 40 |
| 60-Reg-T4R09C6 | CTAACTCCCAGTCGGGAAACCTGGTCCACG | 459 |
| 61-Reg-T4R10C3 | TCGACTCTGAAGGGCGATCGGTGCGGCCTC | 41 |
| 62-Reg-T4R11C6 | GTGCTGCCCCAGTCACGACGTTTGAGTGAG | 225 |
| 63-Reg-T4R12C3 | AGGAAGATCATTAAATGTGAGCGTTTTTAA | 42 |
| 64-Reg-T4R13C6 | ATTGACCCGCATCGTAACCGTGAGGGGGAT | 460 |
| 65-Reg-T4R14C3 | CCAATAGGAAACTAGCATGTCAAGGAGCAA | 43 |
| 66-Reg-T4R15C6 | CAGGAAGTAATATTTTGTTAAAAACGGCGG | 226 |
| 67-Reg-T4R17C6 | ACCGTTCATTTTTGAGAGATCTCCCAAAAA | 461 |
| 68-Reg-T4R19C6 | CCTTTATCATATATTTTAAATGGATATTCA | 227 |
| 69-Reg-T1R04C5 | TAGAGCTTCAGACCGGAAGCAAACCTATTATA | 44 |
| 70-Reg-T1R06C5 | GTCAGAAGATTGAATCCCCCTCAACCTCGTTT | 45 |
| 71-Reg-T1R07C4 | AAATATTCCAAAGCGGATTGCATCGAGCTTCA | 46 |
| 72-Reg-T1R08C5 | ACCAGACGGAATACCACATTCAACGAGATGGT | 47 |
| 73-Reg-T1R09C4 | AGATTTAGACGATAAAAACCAAAAATCGTCAT | 48 |
| 74-Reg-T1R10C1 | AGTCAGGACATAGGCTGGCTGACCTTTGAAAG | 49 |
| 75-Reg-T1R10C5 | TTAATTTCCAACGTAACAAAGCTGTCCATGTT | 50 |
| 76-Reg-T1R11C2 | GAGTAATCTTTTAAGAACTGGCTCCGGAACAA | 51 |
| 77-Reg-T1R11C4 | ACCCAAATAACTTTAATCATTGTGATCAGTTG | 52 |
| 78-Reg-T1R12C5 | ACTTAGCCATTATACCAAGCGCGAGAGGACTA | 53 |
| 79-Reg-T1R13C2 | AAAAGAATAACCGAACTGACCAACTTCATCAA | 54 |
| 80-Reg-T1R14C5 | AAGACTTTGGCCGCTTTTGCGGGATTAAACAG | 56 |
| 81-Reg-T1R15C4 | GAGTTAAATTCATGAGGAAGTTTCTCTTTGAC | 57 |
| 82-Reg-T1R16C5 | CTTGATACTGAAAATCTCCAAAAAAGCGGAGT | 58 |
| 83-Reg-T1R17C4 | TTTCACGTCGATAGTTGCGCCGACCTTGCAGG | 59 |
| 84-Reg-T2R04C5 | TTATTCTGACTGGTAATAAGTTTTAACAAATA | 60 |
| 85-Reg-T2R06C5 | AATCCTCAACCAGAACCACCACCAGCCCCCTT | 61 |
| 86-Reg-T2R07C4 | GAGCCGCCTTAAAGCCAGAATGGAGATGATAC | 62 |
| 87-Reg-T2R08C5 | ATTAGCGTCCGTAATCAGTAGCGAATTGAGGG | 63 |
| 88-Reg-T2R10C1 | GCCATTTGCAAACGTAGAAAATACCTGGCATG | 64 |
| 89-Reg-T2R10C5 | AGGGAAGGATAAGTTTATTTTGTCAGCCGAAC | 65 |
| 90-Reg-T2R11C2 | AGGTGGCAGAATTATCACCGTCACCATTAGCA | 66 |
| 91-Reg-T2R12C5 | AAAGTTACGCCCAATAATAAGAGCAGCCTTTA | 67 |
| 92-Reg-T2R13C2 | CGCTAATAGGAATACCCAAAAGAAATACATAA | 68 |
| 93-Reg-T2R14C5 | CAGAGAGAACAAAATAAACAGCCATTAAATCA | 69 |
| 94-Reg-T2R16C5 | AGATTAGTATATAGAAGGCTTATCCAAGCCGT | 70 |
| 95-Reg-T2R17C4 | CAAATCAGTGCTATTTTGCACCCAGCCTAATT | 71 |
| 96-Reg-T3R04C5 | AAATAAGAACTTTTTCAAATATATCTGAGAGA | 72 |
| 97-Reg-T3R06C5 | CTACCTTTAGAATCCTTGAAAACAAGAAAACA | 73 |
| 98-Reg-T3R07C4 | TTTCCCTTTTAACCTCCGGCTTAGCAAAGAAC | 74 |
| 99-Reg-T3R08C5 | AAATTAATACCAAGTTACAAAATCCTGAATAA | 75 |
| 100-Reg- | CTTTGAATTACATTTAACAATTTCTAATTAAT | 76 |
| T3R09C4 | | |
| 101-Reg- | GTAGATTTGTTATTAATTTTAAAAAACAATTC | 77 |
| T3R10C1 | | |
| 102-Reg- | TGGAAGGGAGCGGAATTATCATCAACTAATAG | 78 |
| T3R10C5 | | |
| 103-Reg- | AACATTATGTAAAACAGAAATAAATTTTACAT | 79 |
| T3R11C2 | | |
| 104-Reg- | CCAGAAGGTTAGAACCTACCATATCCTGATTG | 80 |
| T3R11C4 | | |
| 105-Reg- | ATTAGAGCAATATCTGGTCAGTTGCAGCAGAA | 81 |
| T3R12C5 | | |
| 106-Reg- | GCATCACCAGTATTAGACTTTACAGTTTGAGT | 82 |
| T3R13C2 | | |
| 107-Reg- | CCTCAATCCGTCAATAGATAATACAGAAACCA | 83 |
| T3R13C4 | | |
| 108-Reg- | GATAAAACTTTTTGAATGGCTATTTTCACCAG | 84 |
| T3R14C5 | | |
| 109-Reg- | AGACAATAAGAGGTGAGGCGGTCATATCAAAC | 85 |
| T3R15C4 | | |
| 110-Reg- | TCACACGATGCAACAGGAAAAACGGAAGAACT | 86 |
| T3R16C5 | | |
| 111-Reg- | CCAGCCATCCAGTAATAAAAGGGACGTGGCAC | 87 |
| T3R17C4 | | |
| 112-Reg- | AGCACTAAAAAGGGCGAAAAACCGAAATCCCT | 88 |
| T4R04C5 | | |
| 113-Reg- | TATAAATCGAGAGTTGCAGCAAGCGTCGTGCC | 89 |
| T4R06C5 | | |
| 114-Reg- | GGCCCTGAAAAAGAATAGCCCGAGCGTGGACT | 90 |
| T4R07C4 | | |
| 115-Reg- | AGCTGCATAGCCTGGGGTGCCTAAGTAAAACG | 91 |
| T4R08C5 | | |
| 116-Reg- | AAGTGTAATAATGAATCGGCCAACCACCGCCT | 92 |
| T4R09C4 | | |
| 117-Reg- | GAATTCGTGCCATTCGCCATTCAGTTCCGGCA | 93 |
| T4R10C1 | | |
| 118-Reg- | ACGGCCAGTACGCCAGCTGGCGAACATCTGCC | 94 |
| T4R10C5 | | |
| 119-Reg- | ACTGTTGGAGAGGATCCCCGGGTACCGCTCAC | 95 |
| T4R11C2 | | |
| 120-Reg- | TTCGCTATTGCCAAGCTTGCATGCGAAGCATA | 96 |
| T4R11C4 | | |
| 121-Reg- | AGTTTGAGATTCTCCGTGGGAACAATTCGCAT | 97 |
| T4R12C5 | | |
| 122-Reg- | TTCATCAACGCACTCCAGCCAGCTGCTGCGCA | 98 |
| T4R13C2 | | |
| 123-Reg- | CCCGTCGGGGGACGACGACAGTATCGGGCCTC | 99 |
| T4R13C4 | | |
| 124-Reg- | TAAATTTTTGATAATCAGAAAAGCACAAAGGC | 100 |
| T4R14C5 | | |
| 125-Reg- | ACCCCGGTTGTTAAATCAGCTCATAGTAACAA | 101 |
| T4R15C4 | | |
| 126-Reg- | TATCAGGTAAATCACCATCAATATCAATGCCT | 102 |
| T4R16C5 | | |
| 127-Reg- | AGACAGTCCATTGCCTGAGAGTCTTCATATGT | 103 |
| T4R17C4 | | |
| 128-Reg- | CCATGTACCGTAACACTGTAGCATTCCACAGATTCC | 462 |
| T1R21C5 | AGAC | |
| 129-Reg- | AGCTAATGCAGAACGCGAGAAAAATAATATCCTGTC | 463 |
| T2R21C5 | TTTC | |
| 130-Reg- | CTAAACAGGAGGCCGATAATCCTGAGAAGTGTCACG | 464 |
| T3R21C5 | CAAA | |
| 131-Reg- | AATCATACAGGCAAGGCAGAGCATAAAGCTAAGGG | 465 |
| T4R21C5 | AGAAG | |
| 132-Reg- | GACGGAAAACCATCGATAGCAGCATTGCCATCTTTT | 104 |
| T2R10C4 | CATACACCCTCA | |
| 133-Reg- | TGCCAGTTATAACATAAAAACAGGACAAGAATTGA | 105 |
| T2R15C4 | GTTAACAGAAGGA | |
| 134-Edg-2nt-Rec- | CGGGCGCTAGGGCGCTAAGAAAGCGAAAGG | 466 |
| T4R02C7 | | |
| 135-Edg-2nt-Rec- | ATCACCCAAATCAAGTGCCCACTACGTGAA | 467 |
| T4R04C7 | | |
| 136-Edg-2nt-Rec- | ATCCTGTTTGATGGTGGCCCCAGCAGGCGA | 468 |
| T4R06C7 | | |
| 137-Edg-2nt-Rec- | GCTCACTGCCCGCTTTACATTAATTGCGTT | 469 |
| T4R08C7 | | |
| 138-Edg-2nt-Rec- | CGTTGGTGTAGATGGGGTAATGGGATAGGT | 470 |
| T4R12C7 | | |
| 139-Edg-2nt-Rec- | TTTAAATTGTAAACGTATTGTATAAGCAAA | 471 |
| T4R14C7 | | |
| 140-Edg-2nt-Rec- | GCCGGAGAGGGTAGCTTAGCTGATAAATTA | 472 |
| T4R16C7 | | |
| 141-Edg-2nt-Rec- | AAATTTTTAGAACCCTTTCAACGCAAGGAT | 473 |
| T4R18C7 | | |
| 142-Edg- | GTGTCGTAGACACGGTGGCATCAATTCTAGGGCGCG | 474 |
| T1R00C7-DHP | AGCTGAAAAGTGTCGTAGACAC | |
| 143-Edg- | GTGTCGTAGACACTCCCAATTCTGCGAACCCATATA | 475 |
| T1R02C7-DHP | ACAGTTGATGTGTCGTAGACAC | |
| 144-Edg- | GTGTCGTAGACACATTGCTCCTTTTGATATTAGAGA | 476 |
| T1R04C7-DHP | GTACCTTTAGTGTCGTAGACAC | |
| 145-Edg- | GTGTCGTAGACACCCATAAATCAAAAATCCAGAAAA | 477 |
| T1R06C7-DHP | CGAGAATGAGTGTCGTAGACAC | |
| 146-Edg- | GTGTCGTAGACACCGAGGCATAGTAAGAGACGCCA | 478 |
| T1R08C7-DHP | AAAGGAATTAGTGTCGTAGACAC | |
| 147-Edg- | GTGTCGTAGACACGAAACACCAGAACGAGAGGCTT | 479 |
| T1R10C7-DHP | GCCCTGACGAGTGTCGTAGACAC | |
| 148-Edg- | GTGTCGTAGACACCTGATAAATTGTGTCGAGATTTG | 480 |
| T1R12C7-DHP | TATCATCGCGTGTCGTAGACAC | |
| 149-Edg- | GTGTCGTAGACACGAACGAGGGTAGCAACGCGAAA | 481 |
| T1R14C7-DHP | GACAGCATCGGTGTCGTAGACAC | |
| 150-Edg- | GTGTCGTAGACACGGTTTATCAGCTTGCTAGCCTTTA | 482 |
| T1R16C7-DHP | ATTGTATCGTGTCGTAGACAC | |
| 151-Edg- | GTGTCGTAGACACGGGATTTTGCTAAACAAATGAAT | 483 |
| T1R18C7-DHP | TTTCTGTATGTGTCGTAGACAC | |
| 152-Edg- | GTGTCGTAGACACACAAACTACAACGCCTGAGTTTC | 484 |
| T1R20C7-DHP | GTCACCAGTGTGTCGTAGACAC | |
| 153-Edg- | GTGTCGTAGACACAGCCACCACCCTCATTGAACCGC | 485 |
| T2R00C7-DHP | CACCCTCAGGTGTCGTAGACAC | |
| 154-Edg- | GTGTCGTAGACACGAGAGGGTTGATATAAGCGGATA | 486 |
| T2R02C7-DHP | AGTGCCGTCGTGTCGTAGACAC | |
| 155-Edg- | GTGTCGTAGACACGTATAAACAGTTAATGTTGAGTA | 487 |
| T2R04C7-DHP | ACAGTGCCCGTGTCGTAGACAC | |
| 156-Edg- | GTGTCGTAGACACGCAGGTCAGACGATTGTTGACAG | 488 |
| T2R06C7-DHP | GAGGTTGAGGTGTCGTAGACAC | |
| 157-Edg- | GTGTCGTAGACACTAGCGCGTTTTCATCGCTTTAGCG | 489 |
| T2R08C7-DHP | TCAGACTGGTGTCGTAGACAC | |
| 158-Edg- | GTGTCGTAGACACGCGCCAAAGACAAAAGTTCATAT | 490 |
| T2R10C7-DHP | GGTTTACCAGTGTCGTAGACAC | |
| 159-Edg- | GTGTCGTAGACACCCGAAGCCCTTTTTAAAGCAATA | 491 |
| T2R12C7-DHP | GCTATCTTAGTGTCGTAGACAC | |
| 160-Edg- | GTGTCGTAGACACTTTTTTGTTTAACGTCTCCAAATA | 492 |
| T2R14C7-DHP | AGAAACGAGTGTCGTAGACAC | |
| 161-Edg- | GTGTCGTAGACACAACCTCCCGACTTGCGGCGAGGC | 493 |
| T2R16C7-DHP | GTTTTAGCGGTGTCGTAGACAC | |
| 162-Edg- | GTGTCGTAGACACTAAACCAAGTACCGCATTCCAAG | 494 |
| T2R18C7-DHP | AACGGGTATGTGTCGTAGACAC | |
| 163-Edg- | GTGTCGTAGACACAGATAAGTCCTGAACACCTGTTT | 495 |
| T2R20C7-DHP | ATCAACAATGTGTCGTAGACAC | |
| 164-Edg- | GTGTCGTAGACACGTAAAGTAATTCTGTCAAAGTAC | 496 |
| T3R00C7-DHP | CGACAAAAGGTGTCGTAGACAC | |
| 165-Edg- | GTGTCGTAGACACAGTAGGGCTTAATTGAAAAGCCA | 497 |
| T3R02C7-DHP | ACGCTCAACGTGTCGTAGACAC | |
| 166-Edg- | GTGTCGTAGACACAATGGTTTGAAATACCCTTCTGA | 498 |
| T3R04C7-DHP | CCTAAATTTGTGTCGTAGACAC | |
| 167-Edg- | GTGTCGTAGACACAGTCAATAGTGAATTTTTAAGAC | 499 |
| T3R06C7-DHP | GCTGAGAAGGTGTCGTAGACAC | |
| 168-Edg- | GTGTCGTAGACACTGAGCAAAAGAAGATGATTCATT | 500 |
| T3R08C7-DHP | TCAATTACCGTGTCGTAGACAC | |
| 169-Edg- | GTGTCGTAGACACCAATATAATCCTGATTGATGATG | 501 |
| T3R10C7-DHP | GCAATTCATGTGTCGTAGACAC | |
| 170-Edg- | GTGTCGTAGACACGTTATCTAAAATATCTAAAGGAA | 502 |
| T3R12C7-DHP | TTGAGGAAGGTGTCGTAGACAC | |
| 171-Edg- | GTGTCGTAGACACACATCGCCATTAAAAAAACTGAT | 503 |
| T3R14C7-DHP | AGCCCTAAAGTGTCGTAGACAC | |
| 172-Edg- | GTGTCGTAGACACTCGTCTGAAATGGATTACATTTT | 504 |
| T3R16C7-DHP | GACGCTCAAGTGTCGTAGACAC | |
| 173-Edg- | GTGTCGTAGACACTTGATTAGTAATAACATTGTAGC | 505 |
| T3R18C7-DHP | AATACTTCTGTGTCGTAGACAC | |
| 174-Edg- | GTGTCGTAGACACAGGAACGGTACGCCAGTAAAGG | 506 |
| T3R20C7-DHP | GATTTTAGACGTGTCGTAGACAC | |
| 175-Bri-T1R14C2 | TGCCACTACTTTTTTTGCCACCCTC | 150 |
| 176-Bri-T2R14C2 | AACTGAACATTTTTTTTGAATAACC | 151 |
| 177-Bri-T3R14C2 | GCCACGCTGTTTTTTTACCAGTGAG | 152 |
| 178-Bri-T4R14C2 | CAAAAATAATTTTTTTTGTTTAGAC | 153 |
| 179-Bri-T1R02C5 | GATACATTTCGCTTTTTTGACCCTGTAAT | 154 |
| 180-Bri-T2R02C5 | ACCGTACTCAGGTTTTTGATCTAAAGTTT | 155 |
| 181-Bri-T3R02C5 | AACATGTAATTTTTTTTGAAACCAATCAA | 156 |
| 182-Bri-T4R02C5 | GCGTAACCACCATTTTTGAGTAAAAGAGT | 157 |
| 183-Bri-T1R08C1 | CAGAGGGGGTTTTGCCTTCCTGTAGCCAGCT | 158 |
| 184-Bri-T2R08C1 | GAACCGCCTCTTTACCTAAAACGAAAGAGGC | 159 |
| 185-Bri-T3R08C1 | CATAAATCAATTTAGTCAGAGGGTAATTGAG | 160 |
| 186-Bri-T4R08C1 | CCAGGGTGGTTTTGCAAATGAAAAATCTAAA | 161 |
| 187-Bri-T1R16C3 | ACAACCATTTTTTCATACATGGCTTTTAAGCGCA | 162 |
| 188-Bri-T2R16C3 | TTTTATCTTTTTTATCCAATCGCAAGAGTTGGGT | 163 |
| 189-Bri-T3R16C3 | GCCAACATTTTTTCCACTATTAAAGAAATAGGGT | 164 |
| 190-Bri-T4R16C3 | ACAAGAGTTTTTTTCGCGTTTTAATTCAAAAAGA | 165 |
| 191-Bri-T1R07C2 | TGGATAGCAAGCCCGATTTTTAATCGTAAACGCCAT | 166 |
| 192-Bri-T2R07C2 | AGAACCGCATTTACCGTTTTACCGATATATACGTAA | 167 |
| 193-Bri-T3R07C2 | TTGCTTCTTATATGTATTTTACGCTAACGGAGAATT | 168 |
| 194-Bri-T4R07C2 | ACGGGCAAGTTCCAGTTTTTTCTGACCTGCAACAGT | 169 |
| 195-Bri-T1R18C5 | GAGAATAGAAAGGAACAACTATTTTCTCAAGAGAA | 170 |
| | GGA | |
| 196-Bri-T2R18C5 | TTTTATTTTCATCGTAGGAATTTTTAGCCTGTTTAGT | 171 |
| | A | |
| 197-Bri-T3R18C5 | CAAACTATCGGCCTTGCTGGTTTTTGAGCTTGACGG | 172 |
| | GG | |
| 198-Bri-T4R18C5 | GAGTAATGTGTAGGTAAAGATTTTTTGTTTTAAATAT | 173 |
| | G | |
| 199-Bri-T1R12C1 | AGGACAGATGATTTTTTCACCAGTAGCACCATTACC | 174 |
| | GACTTGA | |
| 200-Bri-T2R12C1 | ATTAAGACTCCTTTTTAATATACAGTAACAGTACCG | 175 |
| | AAATTGC | |
| 201-Bri-T3R12C1 | GACAACTCGTATTTTTTCCTGTGTGAAATTGTTATCC | 176 |
| | GAGCTC | |
| 202-Bri-T4R12C1 | CCGCTTCTGGTTTTTTCGTTAATAAAACGAACTAAAT | 177 |
| | TATACC | |
| 203-Bri-T1R19C5 | TGTCGTCTCAGCCCTCATATTTTTTTCGCCACCCTCA | 178 |
| | GGTGTATC | |
| 204-Bri-T2R19C5 | TAATCGGCCATCCTAATTTTTTTTTTTTTTCGAGCCA | 179 |
| | ACAACGCC | |
| 205-Bri-T3R19C5 | CTGTCCATTTTTATAATCATTTTTTTCTTAATGCGCCC | 180 |
| | ACGCTGC | |
| 206-Bri-T4R19C5 | ACTTTTGCATCGGTTGTACTTTTTTTAACCTGTTTAG | 181 |
| | GACCATTA | |
| 207-Bri-T1R05C4 | AAGCGAACAATTGCTGAATATAATGCTGTATTTTTTT | 182 |
| | GTGAGAAAGGCCGG | |
| 208-Bri-T2R05C4 | AGGAGTGTAAACATGAAAGTATTAAGAGGCTTTTTT | 183 |
| | TGCGAATAATAATTT | |
| 209-Bri-T3R05C4 | GCGAGAAAATAAACACCGGAATCATAATTATTTTTT | 184 |
| | TCGCCCAATAGCAAG | |
| 210-Bri-T4R05C4 | CCAACGTCATCGGAACCCTAAAGGGAGCCCTTTTTT | 185 |
| | TGAACAATATTACCG | |
| 211-Bri-T2R10C0 | GGAATTAGAGCTTTTTTTTCAGACCAGGCGCGTTGG | 186 |
| | GAAGATTTTTTTTCCAGGCAAAGC | |
| 212-Bri-T4R10C0 | AATCATGGTCATTTTTTTTTTTGCCCGAACTCAGGTT | 187 |
| | TAACTTTTTTTTCAGTATGTTAG | |
| 213-Reg- | CCCCAGCGGGAACGAGGCGCAGACTATTCATT | 55 |
| T1R13C4 | | |
| 214-Reg- | AACCGAGGGCAAAGACACCACGGATAAATATT | 507 |
| T2R12C4 | | |

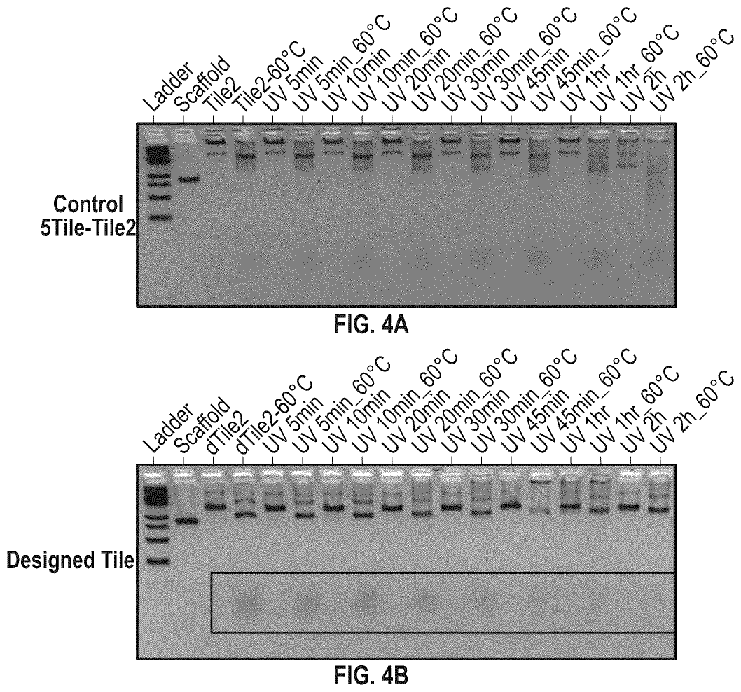

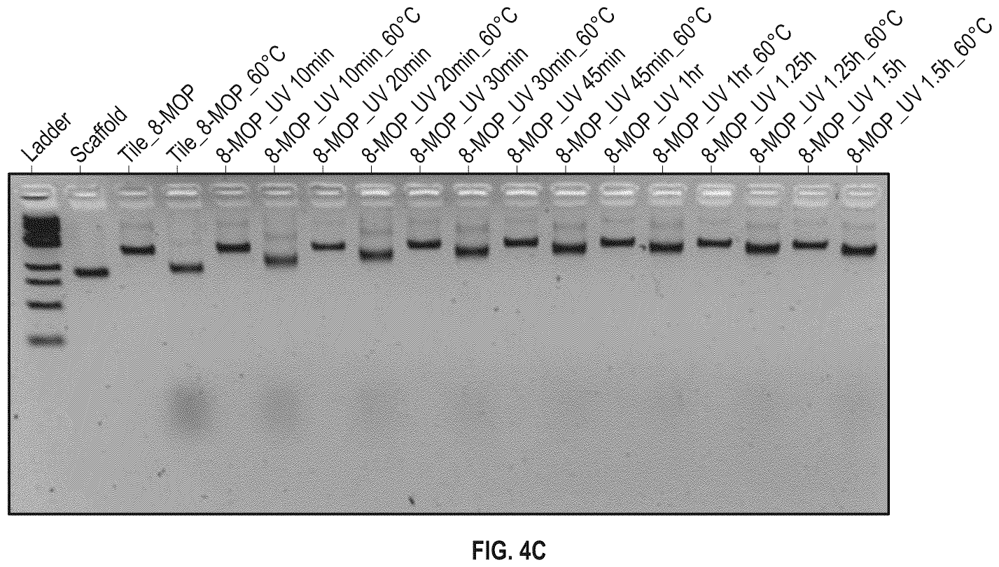

The dTile was irradiated with 310 nm UV light for various lengths of time (5, 10, 20, 30, 45, 60 or 120 minutes), an aliquot was removed from the sample for each irradiation period (non-melt control) and a second aliquot from each irradiated sample was then incubated at 60° C. for 30 min. As controls, samples of Tile2 were similarly treated. The dTile and Tile2 aliquots were loaded on respective gels (2%0 agarose gel with SYBR Safe stain (ThermoFisher, Waltham, MA)) and electrophoresed at 70 V for 180 min. An image of the gel loaded with Tile2 control samples is shown in FIG. 4A with the contents of each lane indicated including a 1 Kb Plus DNA ladder (lane 1), scaffold strand for Tile2 (lane 2), Tile2 no-melt control (lane 3), Tile2 incubated at 60° C., and several pairs of lanes for Tile2 irradiated with UV for the time indicated with and without subsequent incubation at 60° C. FIG. 4B shows a gel loaded with the dTile samples, with lanes loaded as indicated. The results of FIG. 4A indicate that increasing UV irradiation time resulted in increased smearing for Tile2 whether or not melted at 60° C. This may be due to increased UV damage to the DNA as irradiation increased. The results of FIG. 4B indicate that increasing radiation time resulted in the mobility of the 60° C. melted tiles approaching the mobility of the non-melted tiles. Also, the amount of disassembled staple strands decreased with increased UV irradiation time (see bands in the boxed region of the gel), indicating increased structural stability for the tiles as UV irradiation levels increased.

Tile2 was irradiated with 365 nm UV light in the presence of 500 μM 8-MOP for various lengths of time (10, 20, 30, 45, 60, 75 or 90 minutes), an aliquot was removed from the sample for each irradiation period (non-melt control) and a second aliquot from each irradiated sample was then incubated at 60° C. for 30 min. The aliquots were run on a gel, stained and imaged as set forth above in connection with FIGS. 4A and 4B. The gel is shown in FIG. 4B and the results indicate that increasing radiation time resulted in the mobility of the 60° C. melted tiles approaching the mobility of the non-melted tiles and resulted in decreased loss of staple strands.

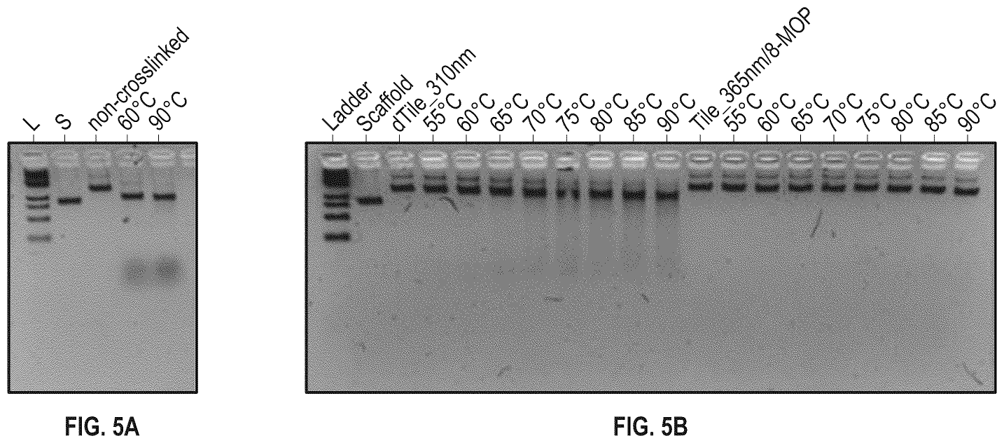

A melting experiment was performed to evaluate the stability of cross-linked DNA origami tiles at high temperatures. A dTile sample and Tile2 sample were folded as set forth above. Cross-linking was then performed by irradiating the dTile with 310 nm UV for 2 hours, and irradiating Tile2 with 365 nm UV in the presence of 500 μM 8-MOP for 50 minutes. Following cross-linking aliquots of both tiles were incubated for 30 min. at 55° C., 60° C., 65° C., 70° C., 75° C., 80° C., 85° C. or 90° C. The aliquots were then loaded on a gel, electrophoresed and stained as set forth above. An image of the gel loaded with cross-linked Tiles is shown in FIG. 5B. Aliquots of a non-cross-linked Tile2 sample were also incubated for 30 min. at 60° C. or 90° C., respectively, loaded on a gel, electrophoresed and stained under the same conditions. An image of the gel loaded with non-cross-linked Tile2 is shown in FIG. 5A. Comparison of the results for the two cross-linked tiles indicates that both strategies increased stability of the DNA origami tile at all temperatures tested in comparison to the non-cross-linked by testing cross-linked tile. The second strategy, using 365 nm UV radiation in the presence of 8-MOP, produced the more temperature resistant tiles among the two strategies tested.

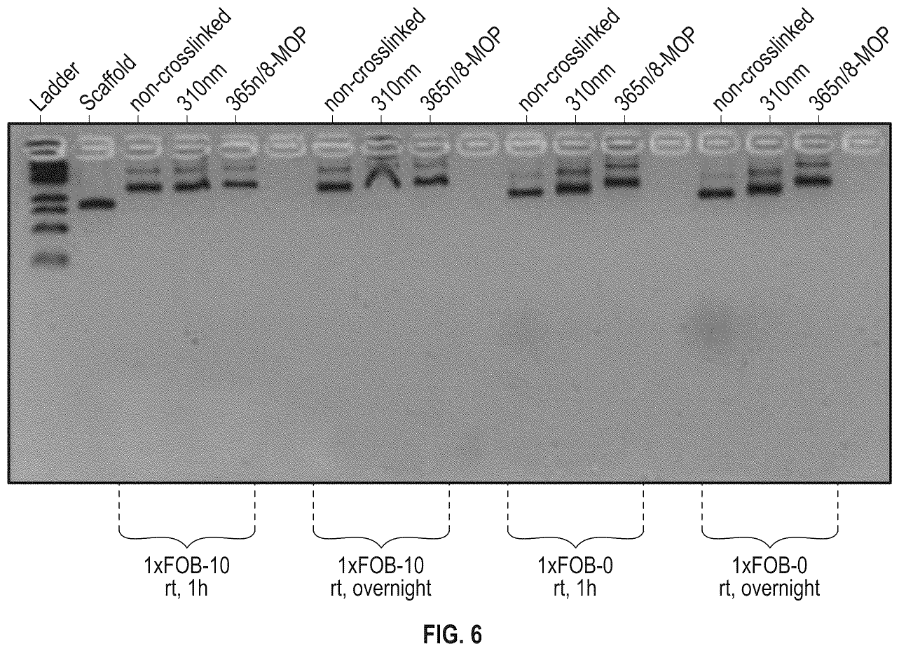

A magnesium depletion experiment was performed to evaluate the stability of cross-linked DNA origami tiles to low concentrations of divalent cation. A dTile sample and Tile2 sample were folded and cross-linked as set forth above for the temperature stability experiment. A Tile2 sample that was not cross-linked was also used as a control. Aliquots of the cross-linked dTile, cross-linked Tile2 and non-cross-linked Tile2 were separately treated under the following four conditions: (1) 1 hour, room temperature incubation in 1×FOB including 10 mM Mg⁺², (2) overnight, room temperature incubation in 1× folding buffer including 10 mM Mg⁺², (3) 1 hour, room temperature incubation in 1× folding buffer without Mg⁺², or (4) overnight, room temperature incubation in 1× folding buffer without Mg⁺². The aliquots were loaded on a gel, electrophoresed and stained as set forth above. An image of the gel is shown in FIG. 6 with lanes loaded as indicated, wherein non-cross-linked Tile2 is indicated by “non-cross-linked”, cross-linked dTile is indicated by “310 nm”, cross-linked Tile2 is indicated by “365n/8-MOP”, aliquots treated in condition (1) are indicated by “1×FOB-10 rt, 1 h”, aliquots treated in condition (2) are indicated by “1×FOB-10 rt, overnight”, aliquots treated in condition (3) are indicated by “1×FOB-0 rt, 1 h”, and aliquots treated in condition (4) are indicated by “1×FOB-0 rt, overnight”. The results of FIG. 6 indicate that all samples incubated in the presence of 10 mM magnesium remained stable. While in the absence of magnesium, non-cross-linked Tiles disassembled, losing staple strands whereas cross-linked tiles remained largely intact.

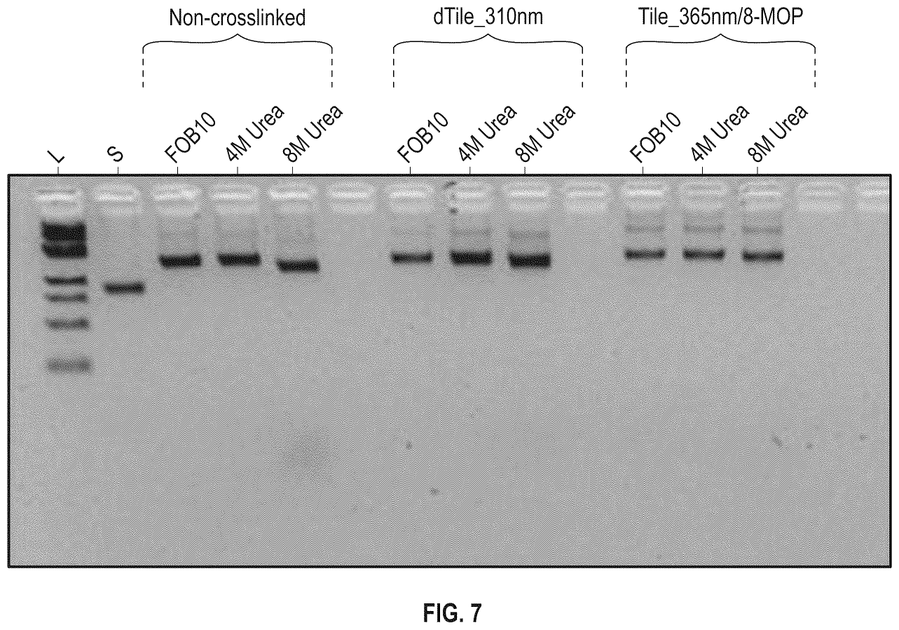

Stability of cross-linked and non-cross-linked DNA origami tiles was evaluated in denaturing concentrations of Urea. A dTile sample and Tile2 sample were folded and cross-linked as set forth above for the temperature stability experiment. A Tile2 sample that was not cross-linked was also used as a control. Aliquots of the cross-linked dTile, cross-linked Tile2 and non-cross-linked Tile2 were separately treated under the following three conditions: (1) overnight, room temperature incubation in 1× folding buffer including 10 mM Mg⁺², (2) overnight, room temperature incubation in 1× folding buffer including 10 mM Mg⁺² and 4M urea, or (3) overnight, room temperature incubation in 1× folding buffer including 10 mM Mg⁺² and 8M urea. The aliquots were loaded on a gel, electrophoresed and stained as set forth above. An image of the gel is shown in FIG. 7 with lanes loaded as indicated, wherein non-cross-linked Tile2 is indicated by “non-cross-linked”, cross-linked dTile is indicated by “dTile_310nm”, cross-linked Tile2 is indicated by “Tile_365nm/8-MOP”, condition (1) is indicated by “FOB10”, condition (2) is indicated by “4M Urea”, and condition (3) is indicated by “8M Urea”. Under 4M Urea, the non-cross-linked tile was largely intact, whereas under the 8M Urea the non-cross-linked tile structure was partially disassembled. In contrast, the cross-linked DNA origami tiles generated by both strategies were stable in 8M urea.

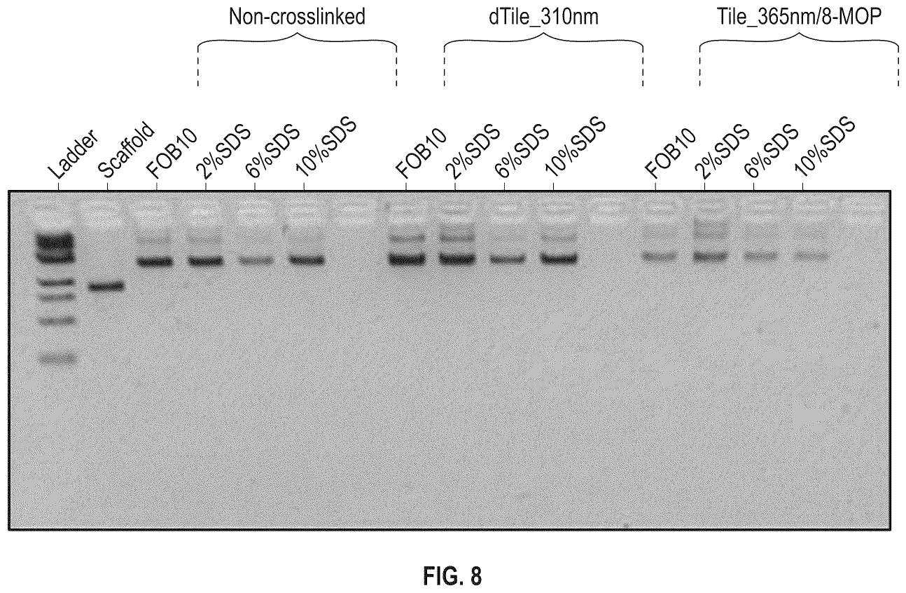

Stability of cross-linked and non-cross-linked DNA origami tiles was evaluated in denaturing concentrations of sodium dodecyl sulfate (SDS). A dTile sample and Tile2 sample were folded and cross-linked as set forth above for the temperature stability experiment. A Tile2 sample that was not cross-linked was also used as a control. Aliquots of the cross-linked dTile, cross-linked Tile2 and non-cross-linked Tile2 were separately treated under the following three conditions: (1) overnight, room temperature incubation in 1× folding buffer including 10 mM Mg⁺², (2) overnight, room temperature incubation in 1× folding buffer including 10 mM Mg⁺² and 2% SDS, (3) overnight, room temperature incubation in 1× folding buffer including 10 mM Mg⁺² and 6% SDS, or (4) overnight, room temperature incubation in 1× folding buffer including 10 mM Mg⁺² and 10% SDS. After buffer exchange to remove SDS, the aliquots were loaded on a gel, electrophoresed and stained as set forth above. An image of the gel is shown in FIG. 8 with lanes loaded as indicated, wherein non-cross-linked Tile2 is indicated by “non-cross-linked”, cross-linked dTile is indicated by “dTile_310nm”, cross-linked Tile2 is indicated by “Tile_365nm/8-MOP”, condition (1) is indicated by “FOB10”, and condition (2) through (4) are indicated “2% SDS”, “6% SDS”, and “10% SDS”, respectively. All tiles, whether or not cross-linked, were stable under all SDS concentrations tested.

The above results confirmed that cross-linking the DNA origami tile increased stability against denaturing conditions such as high temperatures, low magnesium or 8M urea compared to non-cross-linked DNA origami tiles. The second strategy was better than the first strategy for stabilizing a one-layer origami tile structure. Additionally, the second strategy did not adversely impact reactivity of handles on DNA origami tiles when hybridizing to oligonucleotides derivatized with various moieties of interest.

### Example III

### Cryopreservation of DNA Origami Particles

This example demonstrates high quality and performance of probes consisting of a DNA origami particle attached to a plurality of luminescent labels and a plurality of antibodies after storage in a frozen state and thawing. Surprisingly, the probes when frozen without additives and cryopreservatives typically required for preserving other biological substances, can be thawed and used at a level of performance comparable to probes that had not been frozen.

Probes including Tile2 (see Example II) having 44 handles each attached to a 488 nm Alexa™ fluorophore and having handles each attached to an anti-T181 antibody were dissolved in Lobe Binding Buffer (6 mg/ml Dextran Sulphate, 0.001% v/v Tween-20 and 0.001% v/v Lipidure)) and subjected to one of two conditions: (1) storage at 4° C. for 10 days followed by warming on a laboratory bench top to room temperature, or (2) storage at −20° C. followed by thawing on a laboratory bench top to room temperature. The probes were then incubated in a flow cell having immobilized T181 peptides. The flow cell was washed to remove unbound probes and the flow cell was imaged on a custom fluorescence microscope. Binding results are shown in FIG. 9. The results indicated that freezing and thawing the probes had only a small effect on binding rates. The data demonstrated that DNA origami probes can be frozen and thawed without substantial adverse impact on quality and performance of the probes.

### Example IV

### Posts for Attachment of Analytes to Nucleic Acid Origami Structures

This example describes nucleic acid origami structures for presenting attached analytes to solution phase reagents. The nucleic acid origami structures are configured to have a surface and a post protruding from the surface. The area of the surface can be configured to accommodate attachment of various moieties such as labels, linkers or others set forth herein. The area of the surface can also be configured to provide steric exclusion when attached to a surface (e.g., on a particle or at a site on the surface of an array). As such the nucleic acid origami, once attached to the surface, will preclude other nucleic acid origami from also attaching. In this example, the surface is planar. However, the surface need not be planar and can instead, for example, be curved, concave, convex or corrugated. The posts in this example are particularly well suited for presenting macromolecular analytes such as proteins and nucleic acids since the posts set the macromolecules apart from the origami surface, thereby increasing accessibility of the macromolecule to solution-phase reagents.

FIG. 11 shows a diagram of a nucleic acid origami in profile view. The origami includes a planar region (10) and a post (11). The post is attached to an analyte (star 1). The aspect ratio of the post determines the solid angle of accessibility, shown by the transparent circle labeled θ. The larger the aspect ratio of the post for a given analyte attachment distance, the higher the solid angle. This, in turn improves accessibility of the attached analyte to affinity reagents or other reagents in solution phase. Increasing the absolute distance between the planar surface and analyte improves boundary layer mixing and reduces inhibitory interactions between the analyte and the planar surface of the origami structure. This is evident by comparison to the second analyte (star 2) which has both a smaller solid angle of accessibility and increased interaction with the origami surface when compared to the star 1 analyte.

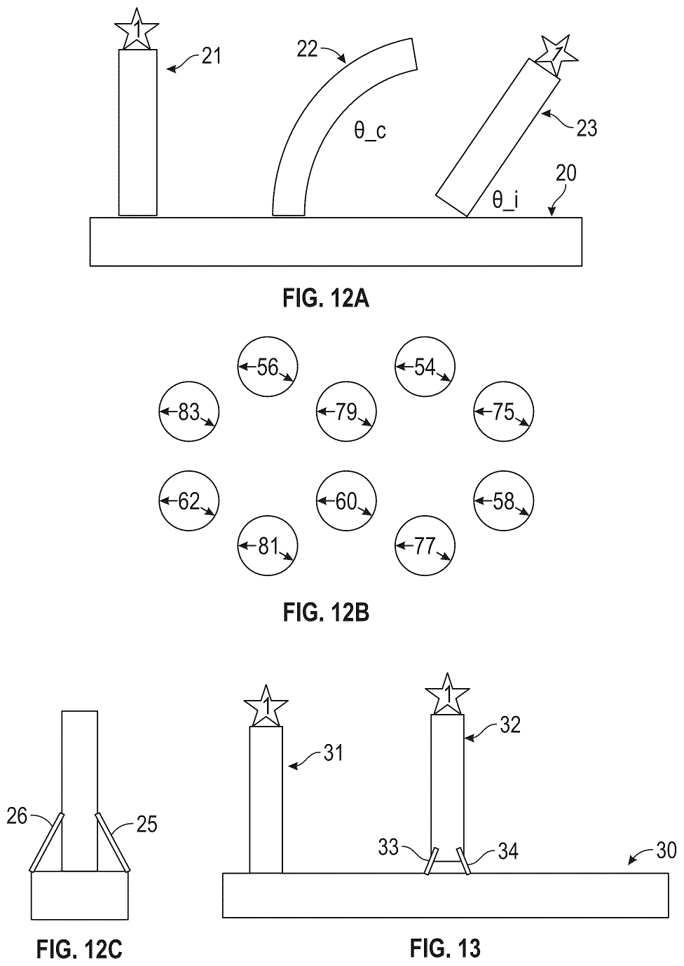

FIG. 12A shows a diagram demonstrating modes of flexibility or rigidity for a post relative to an origami surface to which it is attached. Generally, high rigidity (or low flexibility) of a post benefits fluid-phase accessibility of an analyte attached thereto. Rigidity and flexibility can vary due to the extent of conformational variability along the length of the post or at the interface where the post attaches to the origami surface. In the example shown, a post is attached at its proximal end to an origami surface (20) and at its distal end to an analyte (star 1). The post, when in the proud configuration 21, positions the analyte at a distance from the origami surface that is the largest allowable by the post. In this configuration, the distance is equivalent to the length of the post. Any flexibility along the length of the post, exemplified by configuration 22, and measured by θ_c, reduces the effective distance between the analyte and origami surface compared to the proud configuration 21. Post rigidity can be increased by increasing the number of helices in the post. Increasing θ_c increases rigidity of the post. For example, increased post rigidity can be achieved by increasing the number of helices in the post. FIG. 12B is a perspective view of a cross-section of post 21, wherein the cross-section is parallel to the surface plane 20. The post includes a ten-helix bundle running along the length of the post. This provides increased rigidity compared to posts having fewer helices. An exemplary origami having a post made from a ten-helix bundle of a nucleic acid scaffold is the CBPost origami set forth in Example V.

Returning to FIG. 12A, interface flexibility is demonstrated by the post in configuration 23 and measured by θ_i. Any interface flexibility will reduce the effective distance between the analyte and origami surface compared to the proud configuration 21. FIG. 12C shows the use of nucleic acid trusses to support the post design by enforcing constraint on θ_i. Providing at least 3 constraints is useful for increasing rigidity at the interface plane.

Post designs can be configured to accommodate constraints on DNA conformation which, in turn, constrain the overall structure of an origami fold. One such constraint is the direction that a post will extrude from a face produced by a given origami lattice, or the conditions under which an origami will fold. FIG. 13 shows a first configuration 31 and a second configuration 32 for a post extending from an origami surface 30. In configuration 31, the post is present near the edge of the origami surface. The structure of the post and its connection to the rest of the structure imposes certain physical constraints during origami folding. Positioning the post closer or further from the edge of the structure may alter folding dynamics. Folding dynamics can be modified, for example to achieve a desired structure, using one or more scaffold jumpers. The post in configuration 32 is ‘floated’ relative to the origami surface 30 by use of single stranded scaffold jumpers 33 and 34. The scaffold jumpers reduce the interaction between the post and platform during folding. The number and/or length of scaffold jumpers can be modified to increase independent of folding between the post and other domains of an origami structure.

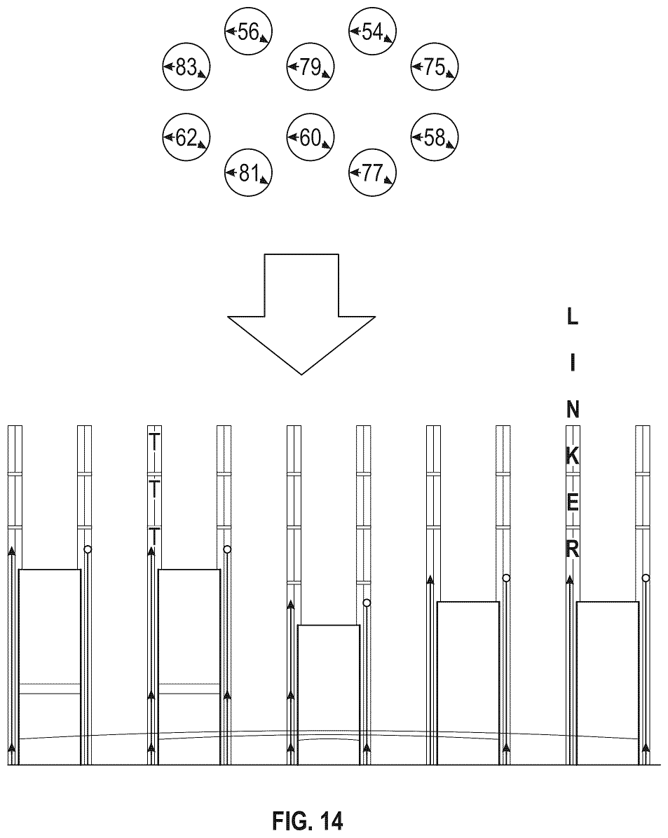

The end of a post provides a location for attachment of a protein or other analyte. FIG. 14, in the upper view, shows the cross-section of the ten-helix bundle of the CBPost origami. The lower view provides a profile view of the helices in the post. The helices (dark lines) run along the length of the post and are held in place by staples (grey lines). The structure is superimposed on a reference grid. The staples are routed such that their ends are fully exposed at the end of the post. This can offer several advantages. First, the interactivity of the post with other components can be reduced by designing these oligo ends suitably. In the design shown, single stranded (polyT) extensions have been added to inactivate the blunt end of the structure and prevent base stacking. The end of the post can be coated with a polymer such as PEG or other inactivating group in other configurations. Second, the end of the post also provides a linker functionalization that can be used to attach other moieties of interest. Here, the end of the post is a well-exposed surface which is useful for its potential activity rather than inactivity.

### Example V

### DNA Origami Structures Having Hexagonal Lattices

A DNA origami (CBFlat) was designed to have a hexagonal lattice structure. The origami has a single lattice layer. The CBFlat origami is formed using the p7249 m13 ssDNA scaffold (Tilibit Nanosystems, Germany) and the oligonucleotides listed in Table 5.

TABLE 5
Oligonucleotide Sequences for CBFlat Origami

| Sequence | | SEQ |
|---|---|---|
| Name | Sequence | ID NO |
| 010209 | CATATTTAACAACGTTAATTTCATCTTC | 508 |
| 022104 | AATGGAAAGCGCAGTAACAGTGCCCGTA | 509 |
| 022230 | ATCAGTGAGGCCACCTACAGGGCGCGTA | 510 |
| 039231 | CATCAATAAGCCTCGTAGGTAAAATTAA | 511 |
| 029041 | TTTCGGGATCGTCACCCTCAGCAGTACAACG | 512 |
| 010153 | AGAGAATATAAAGTACCGACAGGGTATTAAACCAA | 513 |
| 018111 | CAACCGAGTGGCAAATAGCCGTATCAGATTTTTTG | 514 |
| 020195 | GAAAGCGGCGAACTAGTTGAAGATTAGATATAATC | 515 |
| 024104 | AGGCGGATAAGTGCACCGCCACCCTCAGTTAGTAA | 516 |
| 024230 | GAAAGCCGGCGAACCCAACGTCAAAGGGTAAATCA | 517 |
| 026167 | GCATTCCACAGACATGCTTTCGACCAGGGTGGTTT | 518 |
| 039126 | AATTCTGCCAACAGAGCCCGATTAGACTATAAAAA | 519 |
| 021181 | TTGCCTGAATTAACCGTTGTAGACGCTGCGCGTAAC | 520 |
| | AAG | |
| 010174 | GGCATTTTCGAGCCAGTAATAACTCATCGAGAACGC | 521 |
| | GAGAAA | |
| 012174 | TCGCAAGACAAAGAACAAGCATAATTTGCCTTCATT | 522 |
| | TGAATT | |
| 014174 | ACATTTAACAATAGTTACAAAAAGAAACAATAATGG | 523 |
| | AAGGGT | |
| 016174 | TATACTTCTGAATGAAATAGCTGTCACAATATCTTTA | 524 |
| | GGAGC | |
| 017063 | TTACGCATAATAACACTGAACAGAGAATGAAGCCTG | 525 |
| | AACGCG | |
| 018146 | TGGTTTACCAGCGCATCGATAGCAGCACTTAGCGTC | 526 |
| | AGACTG | |
| 018174 | GTTATCTAAAATCAATAGAAAAGTAGCGACTTTGAA | 527 |
| | TGGCTA | |
| 019217 | TTAAAAATACCGAACATTCTGGCCAACATGGTAATA | 528 |
| | TCCAGA | |
| 019231 | CGAACCATATCAAATATTAGATATCATCAGATTTTTT | 529 |
| | CATTT | |
| 019259 | GTGAGGCGGTCAGTCATTGGCAGATTCAGAAAAACG | 530 |
| | CTCATG | |
| 019273 | ATTAACAAGCAAATTTGCCCGTATCATTCCTTTTATG | 531 |
| | CTTTG | |
| 019091 | ACCATTACCATTAGATAGCCCCCTTATTTCAGAGCC | 532 |
| | ACCACC | |
| 020174 | ACAGACAATATTAGAATCAAGGGAGGTTGAGATTA | 533 |
| | GTAATAA | |
| 021133 | GAGCCGCCGCCAGCGGCCTTGATATTCAGGAACCTA | 534 |
| | TTATTC | |
| 022174 | CAATACTTCTTTGGCAGGTCATGAAAGTATAGTGTA | 535 |
| | GCGGTC | |
| 022272 | AGACAGGAACGGTACGTGCTTTCCTCGTATCGGAAC | 536 |
| | CCTAAA | |
| 028174 | GCGTATTGGGCGGGTGAATTTTAAAATACGTCATGG | 537 |
| | TCATAG | |
| 030104 | ACTCATCTTTGACCCGACCTGCTCCATGCAAGAGTA | 538 |
| | ATCTTG | |
| 030174 | TCGAATTCGTAATAATGCCACTGACCAACTGCGCCA | 539 |
| | TTCGCC | |
| 030230 | CAGTGCCAAGCTTGCTCTTCGCTATTACTCGGCCTCA | 540 |
| | GGAAG | |
| 031252 | GTGCTGCCCCAGTCGGGTGCCGCGCTCAACGCTGGG | 541 |
| | TGGTTC | |
| 032153 | GACAGATACCGAACTACGAAGAAACGGGCTTAAAC | 542 |
| | ATCAGCT | |
| 032174 | AAACCAGGCAAATTGAAAGAGAAGAACTGGAGCCA | 543 |
| | GCTTTCA | |
| 034174 | TGGCCTTCCTGTCTCATTATAGCAAAAGAACAAACA | 544 |
| | AGAGAA | |
| 036174 | GAGAGTCTGGAGGTTTTGCCATAATTCGAGTTATTTC | 545 |
| | AACGC | |
| 038174 | CGGGAGAAGCCTCTTCAAAGCACCATTAGATACATT | 546 |
| | TCGCAA | |
| 039084 | TACGGTGGATAAGATAGTCAGTTCATTGAGTAAGAA | 547 |
| | CAGGTA | |
| 018188 | AGGAATTGAGGAAGTTAGTCTTTAATGCTAAGAATA | 548 |
| | CGTGGCCATCAC | |
| 030180 | CCGAGCATTCAGGCTGCGCAGCTTCTGGTGCCGGTC | 549 |
| | AACATTAAATGT | |
| 013119 | ATCTTACCAAATAAGAAACGAGAGATAACCCACAA | 550 |
| | AGAAAAGTAAGCAG | |
| 013203 | TCAATATATGATGAAACAAACTATTTGCACGTAAAT | 551 |
| | GGCAATTCATCAA | |
| 013245 | CTATTAAGCAGAGGCGAATTACAGGTTTAACGTCAA | 552 |
| | GAAGGAGCGGAAT | |
| 013077 | AGATTAGAGCAGCCTTTACAGACCCTGAACAAAGTC | 553 |
| | CGAGGAAACGCAA | |
| 019119 | TCACCAATTTTCATCGGCATTCCGCCACCAGAACCA | 554 |
| | TAAATCCTCATTA | |
| 019203 | CTAAAACAACCCTTCTGACCTAACTCAAACTATCGA | 555 |
| | AGAGTCTGTCCAT | |
| 019245 | AAGATAACACGACCAGTAATATACCGCCAGCCATTA | 556 |
| | TCCTGAGAAGTGT | |
| 019077 | AAATCACGCCATCTTTTCATACCACCCTCAGAACCA | 557 |
| | TTTACCGTTCCAG | |
| 024216 | GTGGCGATGCGCCGCGAGTAAGCCTTGCGAGATAGA | 558 |
| | TCGCCACTGGTCA | |
| 025105 | AGCCACCCAGTACCTAAACAGAAGCCAGCTCAGAGT | 559 |
| | TCGGTCCAAGGCC | |
| 025119 | TTTTCAGGTTTTGTCGTCTTTAAGGCTCCAAAAGGCC | 560 |
| | GACAATGACAAC | |
| 025203 | AAAGAACGATAGGGTTGAGTGGCAACAGCTGATTGT | 561 |
| | AATGAATCGGCCA | |
| 025245 | TCAGGGCCGAAATCGGCAAAAGCAGCAAGCGGTCC | 562 |
| | CTGCCCGCTTTCCA | |
| 025077 | CCGCCACTGGGATTTTGCTAAATTGCGAATAATAAT | 563 |
| | CGGTCGCTGAGGC | |
| 026139 | CGTAACGAATTGTATCGGTTTAGCTTGATACCGATA | 564 |
| | GGAAGTTTCCATT | |
| 026174 | GCCTGTATTTCGTCAGGGCGCTGGCATAAGAGGCTA | 565 |
| | CCGTAACACTGAG | |
| 026223 | AAAGAATCCGCCTGGCCCTGAAACCTGTCGTGCCAA | 566 |
| | ACATACGAGCCGG | |
| 026097 | ATGAATTACGTTGAAAATCTCGCCCACGCATAACCG | 567 |
| | CAACGGCTACAGA | |
| 031119 | CGAGGCGCATAGGCTGGCTGATAATTTCAACTTTAA | 568 |
| | CGTTAATAAAACG | |
| 031203 | CGATCGGTCCAGCCAGCTTTCTAACAACCCGTCGGA | 569 |
| | ACCAATAGGAACG | |
| 031210 | TGCGGGCCATGCCTTCCACACGCTGCATCCCTTCAA | 570 |
| | GCCCGAGTGGACT | |
| 031245 | GGGGGATTTTGAGGGGACGACACCGTAATGGGATAT | 571 |
| | TAAAATTCGCATT | |
| 031077 | TGTGTCGCCGGATATTCATTAGAGAAACACCAGAAG | 572 |
| | AAAGATTCATCAG | |
| 032111 | CCTTCATTTACTTACTAAAACGGCTTTGAACCATCCA | 573 |
| | AAAAACCAGACG | |
| 034146 | GGACGTTGGGAAGACCAAAATAGCGAGATAATAGT | 574 |
| | AAAATGTAAGACTT | |
| 034188 | AAATAATTCGCGTCTCGATGAACGGTAATCAGGTCA | 575 |
| | TTGCCTAAGGATA | |
| 039105 | TCCATATGTACCTTGCATCAAACTGCGGACCCTCGA | 576 |
| | ACTAACGATGGTT | |
| 039147 | TAGTTTGGAACCAGCGCGTTTGAGGGGGGGCTTTTC | 577 |
| | CAGTCACGATTTT | |
| 039189 | GTTTAGCTATGACCTTAGAACAAGGCTATCGTAAAC | 578 |
| | CATCAAGAGCGAG | |
| 032195 | CGGCACCACTGTTGGAGGATCAAATTGTACGCGCGG | 579 |
| | AGACGGTTGTTCCAGTT | |
| 010188 | GTAATTTAGGCAGAACTTTTTCAAATATTGATGCAA | 580 |
| | ATCCAAACCTTTTTTAATGG | |
| 013105 | CAATTTTTAGCAAGGGCTGTCAGACGACGACAATAA | 581 |
| | ACAACAACCAATCAATAATC | |
| 013231 | TCTGTAACCTTTTTAAATACCTCAACAGTAGGGCTTA | 582 |
| | ATTGAAATTTAATGGTTTG | |
| 013273 | TGAAAACAGTCAATACACCGGTCATATGCGTTATAC | 583 |
| | AAATTCTTAAATAAGAATAA | |
| 014146 | GCCATATTATTTATGTTAAGCCCAATAAATCTTACCG | 584 |
| | AAGCCCACCACGGAATAAG | |
| 014188 | AAACAAAATTAATTTAGAACCTACCATACTGATTGT | 585 |
| | TTGGATACTAACAACTAATA | |
| 018132 | CAAAGACGCAAAGACTTTTTAGAATTGACCCAATCC | 586 |
| | AACGCTAACGAGCGTCTTTC | |
| 018153 | ATTCATATTTATTTAATAGCTTAAGAGCATAAACAC | 587 |
| | AGAGCCAGCCGTTGTACCGC | |
| 025252 | GATGGCCGGGAGCCCGTATAACGCCAGAGCAACAG | 588 |
| | CCAGTCAAACAGAGATCACCT | |
| 025259 | CACTACGTGAACCATCACCCAGCACTAATAGAATCG | 589 |
| | GATTTTGAAATACTTATTTA | |
| 025063 | CGCCACCTATAGCCTAAGTTTTAAGCGTAGAGCCGA | 590 |
| | TCAAAAAATTAGATCATTAA | |
| 025084 | CCTCAGACGTCGAGCCTTGAGTCTCTGAGCCACCCA | 591 |
| | GCGTTTCAGTAGCGTAAATA | |
| 026132 | ATCTAAAGGATAGCTAGGATTCTATTTCCAAACAAA | 592 |
| | CCACCATAGCGCGTGAAACC | |
| 026153 | GCCCTCACCCATGTGAGACTCTGAAACAGACGATTA | 593 |
| | TTGACATTTGCCTCGTAATC | |
| 027231 | GAGAGTTTCCCTTACGAAAAATGACGGGCTATGGTT | 594 |
| | TTTATAACAATATAAAGGGA | |
| 030146 | GCACCAACCTAAAATCAATCATAAGGGAGAACGGT | 595 |
| | GTACAGATGAATTACCTTATG | |
| 033084 | CGAGTAGACAAGAAAAATCCGCCCAGCGGAGGGTA | 596 |
| | GATATATTTTTTTCTTCTGTA | |
| 034132 | AAAATCTATCATTGCCAGGCGCAGACGGCGAAAGAT | 597 |
| | TTCATGAGTTGCGAGCCTTT | |
| 035231 | TCAGAAAAAATTTTCGGATTGGACAGTAGCCAGCTC | 598 |
| | GACGGCAAGCATAGTCGGGA | |
| 039210 | GGGCGCGAATCGGTAATGCAAGCTATTTCATATGTA | 599 |
| | TTTTTTATTCTCCATCGCAC | |
| 039252 | TAGCATTAATTAGCTGAGAAATATTCAAGAAGATTT | 600 |
| | ATTTTGGGTCACGCTGCCAG | |
| 014219 | GCATAAAGAAATTGCGTATATTCCTGATTATTGAGG | 601 |
| | ATTTAGAAGCCCTCAATCAATAT | |
| 014261 | ACAATACAGTAACAGTATTGCGGAACAAAGATCGTA | 602 |
| | TTAAATCCTGAAAAATCTAAAGC | |
| 014093 | AAATAATTGAGCGCTAAAACAAAGTTACCAGATACA | 603 |
| | TACATAAAGTTGAGGGAGGGAAG | |
| 017210 | ATACATTCAGATGAACAGAAAAAAGAAGATGTGAG | 604 |
| | TGAATAACCTTGCTCAATTACCTGA | |
| 017252 | CGACAACAACCACCGATGAATAAATCGCTTAATTTT | 605 |
| | CCCTTAGAATCCTAATACCAAGTT | |
| 017084 | GTAGAAAAAGGAAACAGAGGGTGAAAATTTGCTAT | 606 |
| | TTTGCACCCAGCTATTTAACGTCAA | |
| 024185 | GAGCGGGCGCTACCAGTACAGGAACAAGAGTCAAA | 607 |
| | GCGACACCACACACGCAAGTAGAAG | |
| 026184 | TAACTACAACTTCTTTTCACCAGTGGGAGAGGCGGT | 608 |
| | TTCTGTTTCCTGTGTGCCCGGGTA | |
| 033217 | GTGGGAACAAACGGTGTTAAATCAGCTCACCCCGGT | 609 |
| | TGATAATGCCGGAGAGGGTATGCC | |
| 033091 | TAAATTGGGCTTGAGGAACAACATTATTGCAACACT | 610 |
| | ATCATAAATCGTCATAAATAAAGC | |
| 034066 | AGATCCCTGACCCCAAATATCGCCTGAAACAAAGCG | 611 |
| | AAATTGCAGGTAAAGGAACAACTT | |
| 039041 | TTTTAATGCTGTAGCTCAACATGTTTTAAATGGCTTA | 612 |
| | GGTCTTGCTTTAACCAAAAGTTG | |
| 024146- | CTCAAGAGAAGGATAAGCCCAATAGGAATAGTTAG | 613 |
| APT40.2.3p | CACTCACCTCCATCTCCACTCCTACCCATCCAACTCC | |
| | CAC | |
| 031054- | ATTTGTATCCAACGTAACAAAGCTGCTCTCGTTTCTT | 614 |
| DYE40.22.9. | TGCTTGTGTCTTGTCTTCGTCGTGCTTGTTCC | |
| 3p | | |
| 011063- | TCCCATCGCCTGTTTATCAACAATAGATAAGTCTTTC | 615 |
| DYE40.22.9. | GTTTCTTTGCTTGTGTCTTGTCTTCGTCGTGCTTGTTC | |
| 3p | C | |
| 010298- | TTTAAAAGCCTGTTTAGTAAATCATAATTACTAGATT | 616 |
| DYE40.22.9. | TCGTTTCTTTGCTTGTGTCTTGTCTTCGTCGTGCTTGT | |
| 3p | TCC | |
| 012298- | TTTTTAAGACGCTGAGAAGATAGCGATAGCTTAGAT | 617 |
| DYE40.22.9. | TTCGTTTCTTTGCTTGTGTCTTGTCTTCGTCGTGCTTG | |
| 3p | TTCC | |
| 014298- | TTTAACGGATTCGCCTGATCATCGGGAGAAACAATT | 618 |
| DYE40.22.9. | TTCGTTTCTTTGCTTGTGTCTTGTCTTCGTCGTGCTTG | |
| 3p | TTCC | |
| 016298- | TTTAAAGTTTGAGTAACATAACGTTATTAATTTTATT | 619 |
| DYE40.22.9. | TCGTTTCTTTGCTTGTGTCTTGTCTTCGTCGTGCTTGT | |
| 3p | TCC | |
| 018298- | TTTCACGCTGAGAGCCAGCCCGCCTGCAACAGTGCT | 620 |
| DYE40.22.9. | TTCGTTTCTTTGCTTGTGTCTTGTCTTCGTCGTGCTTG | |
| 3p | TTCC | |
| 020298- | TTTAATCGTCTGAAATGGACTACATTTTGACGCTCTT | 621 |
| DYE40.22.9. | TCGTTTCTTTGCTTGTGTCTTGTCTTCGTCGTGCTTGT | |
| 3p | TCC | |
| 022298- | TTTAGGAGGCCGATTAAAGAGAGCGGGAGCTAAAC | 622 |
| DYE40.22.9. | TTTCGTTTCTTTGCTTGTGTCTTGTCTTCGTCGTGCTT | |
| 3p | GTTCC | |
| 024298- | TTTGTCGAGGTGCCGTAAAAATCAAGTTTTTTGGGTT | 623 |
| DYE40.22.9. | TCGTTTCTTTGCTTGTGTCTTGTCTTCGTCGTGCTTGT | |
| 3p | TCC | |
| 026277- | TTTAAAATCCTGTTTGATGTTTGCCCCAGCAGGCGTT | 624 |
| DYE40.22.9. | TCGTTTCTTTGCTTGTGTCTTGTCTTCGTCGTGCTTGT | |
| 3p | TCC | |
| 028277- | TTTTCACATTAATTGCGTTTAATGAGTGAGCTAACTT | 625 |
| DYE40.22.9 | TCGTTTCTTTGCTTGTGTCTTGTCTTCGTCGTGCTTGT | |
| 3p. | TCC | |
| 030277- | TTTGTAACGCCAGGGTTTTAAGGCGATTAAGTTGGT | 144 |
| DYE40.22.9. | TTCGTTTCTTTGCTTGTGTCTTGTCTTCGTCGTGCTTG | |
| 3p | TTCC | |
| 032277- | TTTCATCGTAACCGTGCATTTGGTGTAGATGGGCGTT | 626 |
| DYE40.22.9. | TCGTTTCTTTGCTTGTGTCTTGTCTTCGTCGTGCTTGT | |
| 3p | TCC | |
| 034277- | TTTAAATTGTAAACGTTAAGTATAAGCAAATATTTTT | 627 |
| DYE40.22.9. | TCGTTTCTTTGCTTGTGTCTTGTCTTCGTCGTGCTTGT | |
| 3p | TCC | |
| 036277- | TTTATCACCATCAATATGAGGCCGGAGACAGTCAAT | 628 |
| DYE40.22.9. | TTCGTTTCTTTGCTTGTGTCTTGTCTTCGTCGTGCTTG | |
| 3p | TTCC | |
| 038277- | TTTTACAGGCAAGGCAAAGAACATCCAATAAATCAT | 629 |
| DYE40.22.9. | TTCGTTTCTTTGCTTGTGTCTTGTCTTCGTCGTGCTTG | |
| 3p | TTCC | |
| 011043- | TCTGAACAAGAAAAATAATAAGGCGTTTTAGCGAAC | 630 |
| DYE40.22.9. | CTCTCGTTTCTTTGCTTGTGTCTTGTCTTCGTCGTGCT | |
| 3p | TGTTCC | |
| 013043- | TCCGACTTGCGGGAGGTTTTAACATAAAAACAGGGA | 631 |
| DYE40.22.9. | AGCTCGTTTCTTTGCTTGTGTCTTGTCTTCGTCGTGCT | |
| 3p | TGTTCC | |
| 015043- | TGCATTAGACGGGAGAATTAGGAATACCCAAAAGA | 632 |
| DYE40.22.9. | ACTGTCGTTTCTTTGCTTGTGTCTTGTCTTCGTCGTGC | |
| 3p | TTGTTCC | |
| 017043- | TGCATGATTAAGACTCCTTAAGGTGAATTATCACCG | 633 |
| DYE40.22.9. | TCATCGTTTCTTTGCTTGTGTCTTGTCTTCGTCGTGCT | |
| 3p | TGTTCC | |
| 019043- | TCCGACTTGAGCCATTTGGGTCACCGGAACCAGAGC | 634 |
| DYE40.22.9. | CACTCGTTTCTTTGCTTGTGTCTTGTCTTCGTCGTGCT | |
| 3p | TGTTCC | |
| 021043- | TCACCGGAACCGCCTCCCTCCATACATGGCTTTTGAT | 635 |
| DYE40.22.9. | GATCGTTTCTTTGCTTGTGTCTTGTCTTCGTCGTGCTT | |
| 3p | GTTCC | |
| 023043- | TTACAGGAGTGTACTGGTAACGGAATAGGTGTATCA | 636 |
| DYE40.22.9. | CCGTCGTTTCTTTGCTTGTGTCTTGTCTTCGTCGTGCT | |
| 3p | TGTTCC | |
| 025043- | TTACTCAGGAGGTTTAGTACTCAACAGTTTCAGCGG | 637 |
| DYE40.22.9. | AGTTCGTTTCTTTGCTTGTGTCTTGTCTTCGTCGTGCT | |
| 3p | TGTTCC | |
| 027043- | TGAGAATAGAAAGGAACAACGAGTTAAAGGCCGCT | 638 |
| DYE40.22.9. | TTTGTCGTTTCTTTGCTTGTGTCTTGTCTTCGTCGTGC | |
| 3p | TTGTTCC | |
| 033043- | TATTCAGTGAATAAGGCTTGTTAGGAATACCACATT | 639 |
| DYE40.22.9. | CAATCGTTTCTTTGCTTGTGTCTTGTCTTCGTCGTGCT | |
| 3p | TGTTCC | |
| 035043- | TCTAATGCAGATACATAACGACAGTTCAGAAAACGA | 640 |
| DYE40.22.9. | GAATCGTTTCTTTGCTTGTGTCTTGTCTTCGTCGTGCT | |
| 3p | TGTTCC | |
| 037043- | TTGACCATAAATCAAAAATCAGAGCTTAATTGCTGA | 641 |
| DYE40.22.9. | ATATCGTTTCTTTGCTTGTGTCTTGTCTTCGTCGTGCT | |
| 3p | TGTTCC | |
| 015189- | TCAAAATATCAAGAAAACAGTACATAAATTTACAGT | 642 |
| T3.3p- | TATTAGCCCGCATTT | |
| JF20.22.9.3p | | |
| 017112- | CATATAAAAGAAACAAAAGGGCGACATTGGAAACG | 643 |
| T3.3p- | TTTACAGTTATTAGCCCGCATTT | |
| JF20.22.9.3p | | |
| 017196- | GCCGTCAATAGATAGTTGGCAAATCAACGATAGCCT | 644 |
| T3.3p- | TTACAGTTATTAGCCCGCATTT | |
| JF20.22.9.3p | | |
| 017238- | CTTTACAAACAATTTGCTGAACCTCAAACCAGCAGT | 645 |
| T3.3p- | TTACAGTTATTAGCCCGCATTT | |
| JF20.22.9.3p | | |
| 017070- | GTATGTTAGCAAACTTGACGGAAATTATGCCAGCAT | 646 |
| T3.3p- | TTACAGTTATTAGCCCGCATTT | |
| JF20.22.9.3p | | |
| 023112- | TTAATGCCCCCTGCAGCGGGGTTTTGCTACCCTCATT | 647 |
| T3.3p- | TACAGTTATTAGCCCGCATTT | |
| JF20.22.9.3 | | |
| 023196- | CCCGCCGCGCTTAAGAAAGGAAGGGAAGCACTATTT | 648 |
| T3.3p- | TTACAGTTATTAGCCCGCATTT | |
| JF20p.22.9.3p | | |
| 023238- | TGCTTTGACGAGCACCCGATTTAGAGCTCCGTCTATT | 649 |
| T3.3p- | TACAGTTATTAGCCCGCATTT | |
| JF20.22.9.3p | | |
| 023070- | TAACGGGGTCAGTGAGGGTTGATATAAGCTCAGAAT | 650 |
| T3.3p- | TTACAGTTATTAGCCCGCATTT | |
| JF20.22.9.3p | | |
| 029112- | AGGACTAAAGACTTGGCAAAAGAATACAGCCGGAA | 651 |
| T3.3p- | TTTACAGTTATTAGCCCGCATTT | |
| JF20.22.9.3p | | |
| 029196- | TATCCGCTCACAATGCAGGTCGACTCTAGGAAGGGT | 652 |
| T3.3p- | TTACAGTTATTAGCCCGCATTT | |
| JF20.22.9.3p | | |
| 029238- | AAGTGTAAAGCCTGACGACGTTGTAAAAGGCGAAA | 653 |
| T3.3p- | TTTACAGTTATTAGCCCGCATTT | |
| JF20.22.9.3p | | |
| 029070- | GACAGCATCGGAACATTATACCAAGCGCGATAAATT | 654 |
| T3.3p- | TTACAGTTATTAGCCCGCATTT | |
| JF20.22.9.3p | | |
| 035112- | TTTACCAGACGACGGGATAGCGTCCAATAAAGATTT | 655 |
| T3.3p- | TTACAGTTATTAGCCCGCATTT | |
| JF20.22.9.3p | | |
| 035196- | ACTAGCATGTCAATTTGAGAGATCTACACCTCATAT | 656 |
| T3.3p- | TTACAGTTATTAGCCCGCATTT | |
| JF20.22.9.3p | | |
| 035238- | AGCCCCAAAAACAGCCGTTCTAGCTGATAAGATTCT | 657 |
| T3.3p- | TTACAGTTATTAGCCCGCATTT | |
| JF20.22.9.3p | | |
| 035070- | GAATTACGAGGCATAATCCCCCTCAAATTACCCTGT | 658 |
| T3.3p- | TTACAGTTATTAGCCCGCATTT | |
| JF20.22.9.3p | | |
| 010125- | AAGTAATTCTGTCCTTTCCTTATCATTCATTACCGCG | 659 |
| T3.3p- | CCCAAATCCTGATTTACAGTTATTAGCCCGCATTT | |
| JF20.22.9.3p | | |
| 010251- | TATAAAGCCAACGCGACCGTGTGATAAAGGTCTGAG | 660 |
| T3.3p- | AGACTAATCGTCGTTTACAGTTATTAGCCCGCATTT | |
| JF20.22.9.3p | | |
| 010083- | CTAATGCAGAACGCCTAATTTACGAGCAATCCGGTA | 661 |
| T3.3p- | TTCTAATAAATCATTTACAGTTATTAGCCCGCATTT | |
| JF20.22.9.3p | | |
| 012104- | CAAATCAGATATAGAAGGCTTTGTAGAATGTTCAGT | 662 |
| T2.3p- | TCGTTTCTTTGCTTGTGTCTTGTCTTCGTCGTGCTTGT | |
| DYE40.22.9. | TCC | |
| 3p | | |
| 012146- | TTTATTTTCATCGTAGGAATCCAAGAACAAAGGTAT | 663 |
| T2.3p- | TCGTTTCTTTGCTTGTGTCTTGTCTTCGTCGTGCTTGT | |
| DYE40.22.9. | TCC | |
| 3p | | |
| 012209- | TATATAACTATATGTAAATGCATTTTAGCCAACATTT | 664 |
| T2.3p- | CGTTTCTTTGCTTGTGTCTTGTCTTCGTCGTGCTTGTT | |
| DYE40.22.9. | CC | |
| 3p | | |
| 012230- | AACCTCCGGCTTAGGTTGGGTTGACCTAGAATCGCT | 665 |
| T2.3p- | TCGTTTCTTTGCTTGTGTCTTGTCTTCGTCGTGCTTGT | |
| DYE40.22.9. | TCC | |
| 3p | | |
| 012272- | AGTGAATTTATCAAAATCATATAAGGCGTTACCAGT | 666 |
| T2.3p- | TCGTTTCTTTGCTTGTGTCTTGTCTTCGTCGTGCTTGT | |
| DYE40.22.9. | TCC | |
| 3p | | |
| 037119- | AAGAGGAGTCAGGATTAGAGAAACAGTTGATTCCCT | 667 |
| T2.3p- | TCGTTTCTTTGCTTGTGTCTTGTCTTCGTCGTGCTTGT | |
| DYE40.22.9. | TCC | |
| 3p | | |
| 037140- | CAAATATACCGGAAGCAAACTCGAACGAGTAGATTT | 668 |
| T2.3p- | TCGTTTCTTTGCTTGTGTCTTGTCTTCGTCGTGCTTGT | |
| DYE40.22.9. | TCC | |
| 3p | | |
| 037182- | AAAATTTCTGTAATACTTTTGATGGTCAATAACCTTT | 669 |
| T2.3p- | CGTTTCTTTGCTTGTGTCTTGTCTTCGTCGTGCTTGTT | |
| DYE40.22.9. | CC | |
| 3p | | |
| 037203- | TATTTTATGTACCAAAAACATTATATTTTCATTTGTT | 670 |
| T2.3p- | CGTTTCTTTGCTTGTGTCTTGTCTTCGTCGTGCTTGTT | |
| DYE40.22.9. | CC | |
| 3p | | |
| 037245- | AAAAGGGAAAATTAAGCAATATCTACTAATAGTAGT | 671 |
| T2.3p- | TCGTTTCTTTGCTTGTGTCTTGTCTTCGTCGTGCTTGT | |
| DYE40.22.9. | TCC | |
| 3p | | |
| 037077- | ACTATTAGGTCATTTTTGCGGATATGCAACTAAAGTT | 672 |
| T2.3p- | CGTTTCTTTGCTTGTGTCTTGTCTTCGTCGTGCTTGTT | |
| DYE40.22.9. | CC | |
| 3p | | |
| 037221- | TGAGTAATGTAGAGCATAAAGCTAAGCTGAAAAGG | 673 |
| T2.3p- | TGGTTCGTTTCTTTGCTTGTGTCTTGTCTTCGTCGTGC | |
| DYE40.22.9. | TTGTTCC | |
| 3p | | |
| 037095- | AAAGCGGATTTAATTGCTCCTTTTTCTGGAAGTTTCA | 674 |
| T2.3p- | TTTCGTTTCTTTGCTTGTGTCTTGTCTTCGTCGTGCTT | |
| DYE40.22.9. | GTTCC | |
| 3p | | |

A second DNA origami (CBPost) was designed to have a hexagonal lattice structure. The CBPost is formed using the p8064 ssDNA scaffold (Tilibit Nanosystems, Germany) and the oligonucleotides listed in Table 6. The p8064 scaffold forms a flat structure having a planar surface and is also integrated into a post that protrudes from the surface. The post includes 10 helices formed from the scaffold and is diagrammed in FIGS. 12B and 14.

TABLE 6
Oligonucleotide Sequences for CBPost Origami

| Sequence | | SEQ |
|---|---|---|
| Name | Sequence | ID NO |
| 026155 | CAGCTTTCATCAACATGTTTTAAATATGCA | 675 |
| 024155 | TATTATAGTCCAAAAGTAGTAAACCCTGACCCGCGA | 676 |
| | CATCGCCT | |
| 030195 | ACTAACATAGACTTAATCCTGAAATTATATATTCCAT | 677 |
| | ATAACAGTTG | |
| 021181 | TTAATGGTCTTTTTCAAATATAGCAACACTATCATAT | 678 |
| | AATAGTAAAAT | |
| 000139 | AGAGTCTAAAAATCAGGTCTTTACCCTGAC | 679 |
| 002139 | ACAGTTCTTCGCGTCTGGCCTTCCTGTAGC | 680 |
| 006155 | ATTCCCAATTCTGCGAACGAGTAGATTTCTAAGGAA | 681 |
| | TCGTCATA | |
| 008155 | GTTTAGACTGGATAGCGTCCAATACTGCTGGTAGCT | 682 |
| | ATTTTTGA | |
| 010209 | TCCCTCAGAGCCGCGCCGGAAACGTCAC | 683 |
| 022104 | TTACCTTATGCGATAGATTCATCAGTTG | 684 |
| 032188 | TACCGAACGAACCAGAAATGGATTATTT | 685 |
| 039189 | CAGTTTGGATTTAGAAAGGAGGCCGATT | 686 |
| 039231 | ACGTCAAGGTGCCGCTGCGCGCGTGCTT | 687 |
| 007091 | AAGGATAAAAATTTTTGTACCAAAAACA | 688 |
| 000155 | ATCAGGTCATTGCCTGGAGATCTACAAAGGCT | 689 |
| 002155 | CCCTCAAATGCTTTAAAATATTCATTGAATCC | 690 |
| 004155 | TGGTCAATAACCTGTTAGATACATTTCGCAAA | 691 |
| 000111 | GAACGGTAATCGTATCTAGCTGTAAAGATTCAAAA | 692 |
| 010153 | CGCCACCAGAACCACCACCAGGGGGTCAGTGCCTT | 693 |
| 018111 | GGACTAATCGGTCGAGGCTCCTGGGATTCCGTAAC | 694 |
| 020195 | ATAAGAGAACAACAATCATTAGAACGCGACGTCAA | 695 |
| 024104 | CGAAAGACTTCAAAAAGAGGTCATTTTTGTGGGAA | 696 |
| 024146 | CAGAAGCAAAGCGGTATAATGCTGTAGCTCAACAT | 697 |
| 035189 | AATCAGTGAAAAACACATTGGTTAAAAAAACACCG | 698 |
| 039126 | CCGAAATGCCAACGATTGCGTGTGAGCCTGCGGCG | 699 |
| 005119 | CAATAAAGCAGTTTGACCATTTAGCTATATTTTCA | 700 |
| 005091 | GGCAAGGCAATTCTACTAATAGTAGTAGCATTATTT | 701 |
| 001072 | TTTACCCCGGTTGATAATCCATGTCAATCATATGTTT | 702 |
| | T | |
| 003072 | TTTATTAAATTTTTGTTAATTTTGTTAAAATTCGCTTT | 703 |
| 009065 | TTTCAAATCACCATCAATAAAAGGCCGGAGACAGTT | 704 |
| | TT | |
| 010174 | CAGAGCCACCACCCTCAGAGCAGTGCCCGTAAAATC | 705 |
| | ACCAGT | |
| 012174 | ATTAGAGCCAGCATAAACAGTCCACCCTCAGATTAA | 706 |
| | GACTCC | |
| 014174 | AAGAACTGGCATGAGCCACCAAAGGAACAAAGAAT | 707 |
| | AACATAA | |
| 016174 | GCCTTTACAGAGCTAAAGGAACACCCTCAGTATAGA | 708 |
| | AGGCTT | |
| 017063 | CCGACAAGCTTTCGGTTTTGTGTAGCATAGGGTTGA | 709 |
| | TTAGCG | |
| 018146 | CGGAACGAGGGTAGCGGAGATTTGTATCCTGCTCCA | 710 |
| | TGTTAC | |
| 018174 | AAGCAAATCAGACAGCGAAAGGATAAATTGGTAAT | 711 |
| | TCTGTCC | |
| 019217 | GCCTGTTTATCAACCATGTAATTTAGGCGCGTTAAAT | 712 |
| | AAGAA | |
| 019231 | AATAGATCTCATCGTTTGAAGTATTATTGGGTAATC | 713 |
| | AGATAG | |
| 019259 | ATATCCCATCCTAACAGTAGGGCTTAATAAGCCTGT | 714 |
| | TTAGTA | |
| 019273 | TTTACGATTCCTTACTACAATTCTTTCCTGAGTTAAG | 715 |
| | CAATA | |
| 019091 | TTTGACCCCCAGCGTCATAAGGGAACCGGATATTCA | 716 |
| | TTACCC | |
| 020174 | ACAAAAGGTAAATGTCGAAATGAGAAACACTCATCT | 717 |
| | TCTGAC | |
| 021133 | GTGAATAAGGCTTGTTGGGCTTGAGATGGCAGATAC | 718 |
| | ATAACG | |
| 022174 | TTTTAGTTAATTCAGAACGAGGAATTACGAGGCATA | 719 |
| | GTAAGA | |
| 022272 | TTTAACCTCCGGCTAAGACGCTGAGAAGGAATAACC | 720 |
| | TTGCTT | |
| 029154 | TTACCAGTCTAATACATTTGAACTTCTGAATAACCAT | 721 |
| | TCAGG | |
| 029175 | GGATTTAGAAGTATACTAATAGATTAGAAGGCGGTC | 722 |
| | AGTATT | |
| 030104 | GAGACGCAGAAACACAGGCGGCCTTTAGACTTGTAG | 723 |
| | AACGTC | |
| 030174 | GCCGTCAATAGACCGGAATTTGTCCGTTTTAAAACA | 724 |
| | GAGGTG | |
| 030230 | AATTGAGGAAGGTTGAGCCAGCAGCAAATTAATGC | 725 |
| | GCGAACT | |
| 031252 | TTGCTGAGTCAGTTAAGTTTGCACCAGAGAATATAA | 726 |
| | TAACGG | |
| 032153 | TCGCTGGAAACGCGGTGAGAGGCCATGTCTGCGCAG | 727 |
| | GCAAAG | |
| 032174 | CCAGCAGAAGATTTCGTCTCGTATGAGCCGGCTCAA | 728 |
| | TCGTCT | |
| 034174 | CTACATTTTGACGGTCACTGTGGGGGTTTCGCCAGA | 729 |
| | ATCCTG | |
| 036174 | CAGGAACGGTACTGCCAGCACAGTCGGGAATGGCG | 730 |
| | AGAAAGG | |
| 038174 | AGCCGGCGAACGACCTGTCGTAAAAGAATAGCCCG | 731 |
| | AGATAGG | |
| 039084 | CTGGTTTGTGGTTTTGTAAAGAATTCGTTGTCACTTG | 732 |
| | TTCAG | |
| 003091 | ATCAGCTCATTTTTGTGGCATCAAAGAATTAGCAAA | 733 |
| | AATCGG | |
| 029041 | TTTCCAGTCACGACGTTGTAAATGCCGCCAGCAGTT | 734 |
| | TTGTGTA | |
| 005069 | TTTACATCCAATAAATCATACATTATGACTCAACGC | 735 |
| | GGGTGAG | |
| 018188 | CCGCGCCCAATAGCAGACGACGACAATAAATATAA | 736 |
| | AGTACCGCTAAAT | |
| 000132 | GGAGCAAACAAGAGGGAGAGGCAATGCCTGAGTAA | 737 |
| | CCTCATATATTTTA | |
| 013119 | TACCGCCTAGGAACCCATGTATTGCTAAACAACTTA | 738 |
| | ATCTCCAAAAAAA | |
| 013203 | AAAATACAAACCGAGGAAACGGGGAGAATTAACTG | 739 |
| | CGATTTTTTGTTTA | |
| 013245 | AGACACCTTAAGAAAAGTAAGTGAGCGCTAATATCA | 740 |
| | AAATAAACAGCCA | |
| 013077 | TATAGCCAAACTACAACGCCTCGTCTTTCCAGACGC | 741 |
| | GGTTTATCAGCTT | |
| 019119 | GAAACAAGAACGAGGCGCAGACGTAACAAAGCTGC | 742 |
| | TTCAACTTTAATCA | |
| 019203 | CTAATGCTTTTCGAGCCAGTAACCGACCGTGTGATA | 743 |
| | AGACAAAGAACGC | |
| 019245 | GAACAAGCGCCATATTTAACACGGAATCATAATTAG | 744 |
| | GTTATATAACTAT | |
| 019077 | CTAAAACCAACTTTGAAAGAGGAGTAATCTTGACAA | 745 |
| | CTGGCTCATTATA | |
| 024216 | TTTGCAAATAAAAACAATCGCAAATAAGAGAGGCA | 746 |
| | AGAACGCGTTTTTA | |
| 025105 | GCGGATGGGAAGCCAGATTTATTGTGAAAAATCAAC | 747 |
| | GGTCAAATTATAC | |
| 025119 | GCTTAATTAACAACCCGTCGGCAGCTTTCCGGCACT | 748 |
| | GCGGGCCTCTTCG | |
| 025203 | CTGAGCAGAGGCGAATTATTCTTGCACGTAAAACAC | 749 |
| | AATTCATCAATAT | |
| 025245 | ATTAATTATTCGCCTGATTGCGTTTAACGTCAGATAG | 750 |
| | GAGCGGAATTAT | |
| 025077 | TTGCTCCACCGTAATGGGATAGGGACGACGACAGTG | 751 |
| | TGCTGCAAGGCGA | |
| 026139 | TAAATGTGGTGCCGGAAACCAACTGTTGGGAAGGG | 752 |
| | GCGAAACGTACAGC | |
| 026097 | CAAACGGTCAGGAAGATCGCAGCCAGCTGGCGAAA | 753 |
| | TGGAGCCGCCACGG | |
| 027154 | CGCCATTCGTGGAAGGGTTAGAACCTACCATATCAA | 754 |
| | TTGTTTGGATTAT | |
| 031119 | GTTAAACCCTCATAACGGAACTTGCAGGCGCTTTCT | 755 |
| | TCTTTGCTCGTCA | |
| 031203 | CAGTGCCCTAAAACATCGCCACAGATTCACCAGTCA | 756 |
| | GCCATTGCAACAG | |
| 031210 | ACGCTGAATCTAAACAACTCGATGATGGGAAATAAT | 757 |
| | CGCGCAAAAGAAG | |
| 031245 | CATCACCTTTTTGAATGGCTAGGCCAACAGAGATAA | 758 |
| | CTATCGGCCTTGC | |
| 031077 | TAAAAAATGCTGGTCTGGTCACAACCAGCTTACGGC | 759 |
| | AAATCGTTAACGG | |
| 032111 | GTGCCGGTGATGAAGGAAAAAGAACGGACTATTAC | 760 |
| | CTCCAGCATTCTCC | |
| 034146 | CGGCTGGTAATGGGGGCCGTTTTCACGGTGTTCTTC | 761 |
| | GCGTCCTGCGCTC | |
| 034188 | GCTCATGGAAATACAGAAGTGTTTTTATAAAGGGAT | 762 |
| | TTTAGAAAGGGAA | |
| 039105 | AAATCCTGTTTGCGTGAGTGAGATCCCCTCTGTGGT | 763 |
| | AAACATGGGGTCA | |
| 039147 | ATAAATCGCCAGCTGCTTTCCGCGTGCCTCATACCTG | 764 |
| | CCCTGGTTGCGG | |
| 003126 | AAAATAAAGAAAACGAGAAAAGCAAATATAGGAAC | 765 |
| | GCCATCATTTGGGG | |
| 009084 | TGATATTCAACCGTAAACTAGAGAAAAGCCCCAAAT | 766 |
| | GTAAACGTTAATA | |
| 010188 | AGAACCGCCACCCTAGCACCATTACCATTGAGCCAT | 767 |
| | TTGGGATTATTACGCAGTAT | |
| 013105 | CTCAGGAGAAAGTACAGGAGTGAGGTTGAGGCAGG | 768 |
| | TCAGACGGGCTTTTGATGATA | |
| 013231 | TAAAAGAATATTGACACCGTACGGAACCAGAGCCA | 769 |
| | CCACCGGACCATCGATAGCAG | |
| 013273 | ACAATCAGACAAAACAGACTGCCCCCTTATTAGCGT | 770 |
| | TTGCCATTTGCCTTTAGCGT | |
| 014146 | TTTCAGGGATAGCATTTCAGCGGAGTGATAATAATT | 771 |
| | TTTTCAAGGCCGCTTTTGCG | |
| 014188 | AACGGAATACCCAAAAACAGGGAAGCGCAAATGAA | 772 |
| | AATAGCAATCCGGTATTCTAA | |
| 018132 | CAACGGCGAGTTAACGTTGAATCAACAGAGCCCAA | 773 |
| | ACCCTCAGAACCGCCACCCTC | |
| 018153 | ACAGCATGGATCGTTTGCGAAGAATAGACCCTCATA | 774 |
| | GAACCGTAATGCCGAGTAAC | |
| 022230 | TGCTGATGCAAATCCCAAAATAGAGAATCCTTGAAA | 775 |
| | TGTAAATAAACACACGCCAA | |
| 025168 | ACTAAAGTACGGTGTCTGGAAGTTTCTTGAGGGGGA | 776 |
| | CCCTCGGAGAAAATTGAAAT | |
| 025252 | ACATTTACTGTAAATTAGATTTAGGTTGCTAGAAAT | 777 |
| | GAGAATAAAAATAGGTATTA | |
| 025259 | ACAATTTCATTTGAATTACCTTGTGAGTAGTCAATCT | 778 |
| | ACCTTTCATATGCGCTCAA | |
| 025063 | GAGAGTATCAAAGCTAACGGACCAGTCATCATCAAG | 779 |
| | ACAGATGGCAAAAAAAATAC | |
| 025084 | TTTTGATTATCGCGGGTAGAATTTAAGAAGAACCGA | 780 |
| | ACTGACACTCATCGGAAGTT | |
| 026132 | GAGCGAGTGCTGAAATTGCATAACTAATGTTTAATT | 781 |
| | CATTCATTAGCCGAGTACAA | |
| 026230 | ACCAAGTTACAAAAAGAAATTGCGTAGATTCCTGAT | 782 |
| | TATCAGTATTAAATCCTTTG | |
| 027231 | TTTTCAGTTTGAATATCAAGATAATTTTCCCTTCGAG | 783 |
| | AGGCTATGATGAAACAAAC | |
| 030146 | ATAGACTTTCTCCGATTGCCGTTCCGGCCAGCCTCCG | 784 |
| | GCCAGATCCGCCGGGCGCG | |
| 033084 | CTGGAGGAGCGTGGAGCCGCAGCGGATCTCAGAGG | 785 |
| | GGGGGATATCGGCCCGGATTG | |
| 034132 | TAAAGGTGCACTCAAGCACATGATGCTGTGGTGAAA | 786 |
| | CAATCGCGATCGGCGCTTCT | |
| 035231 | AATTAACTGGTAATACATTCTTTAGTCTTGAAAAAT | 787 |
| | GAAAGGCCCGAACCATCATA | |
| 039210 | TATTAAAGGAACCCCTGGCAAAGAGCGGAAAGAGT | 788 |
| | TACCGCCACACGACGATAGCC | |
| 039252 | TCTATCAAATCAAGGCCGCGCTGCTTTGTTCTTTGAA | 789 |
| | CTCAAGAACCCTGACAATA | |
| 006125 | CAGAGCATAAAGCTAATTAAGCGCGAGCTGAAAAG | 790 |
| | TAACCAATTTAAATAACAGGA | |
| 007105 | TTAGAACTGTGTAGGATAAATTAATGCCAATCGATA | 791 |
| | GATTGTATTGACCATAAATC | |
| 014219 | TACTGAACAAAGTCAGATATCCCAATCCAAAGACTT | 792 |
| | GCGGGAGGTAGAACAAGCAAGCC | |
| 014261 | GAATAACCCACAAGAATAGAGCCTAATTTGCTATTT | 793 |
| | TGCACCCAGTCATTCCAAGAACG | |
| 014093 | TCAATGAATTTTCTGTAAAAAGGAGCCTTTACATAA | 794 |
| | CCGATATATAGACTTTTTCATGA | |
| 017210 | ACCTCCCTAAGAAAAACACCCCAGAAGGATACATA | 795 |
| | AAGGTGGCAACATACCGAACAAAGT | |
| 017252 | TAGTTGCCAGTTACAGAGAGAGCCCTTTACGGAATA | 796 |
| | AGTTTATTTTGTCGCTATCTTACC | |
| 017084 | GCCCACGATTGTATTTAGTAACCAGTACCGGAATAG | 797 |
| | GTGTATCACCGTAACTGAGTTTCG | |
| 033217 | CAGTAATAAAAGGGATCCAGAACAATATCTGTCCAT | 798 |
| | CACGCATCCTCGTTAGAATCGTGT | |
| 033091 | TGTCCAGCATCAGCCCCTTACACTGGTGGCGCGCCT | 799 |
| | GTGCACGGGTACCGAGCTCGCCTG | |
| 034066 | CAGATCCCACGGCAGCAAATAAAAAGCTCATTACGA | 800 |
| | CGGTTAAGTTGTTTGAGGGTCACG | |
| 039041 | TTTCACCGCCTGGCCCTGAGAGAGTTGCAGAGACGG | 801 |
| | ATACGAGCTGTTTCGCATCAGCAT | |
| 007065- | TTTGGGAGAAGCCTTTATTCCTGTAATACTTTTGCTT | 802 |
| APT40.2.3p | TCACTCACCTCCATCTCCACTCCTACCCATCCAACTC | |
| | CCAC | |
| 031056- | CATCGACCCGCAAGAATGCCAACGGCTTTCGTTTCT | 803 |
| DYE40.22.9. | TTGCTTGTGTCTTGTCTTCGTCGTGCTTGTTCC | |
| 3p | | |
| 011063- | ATTTACCCAAATAAATCCTCATTAAAGCCAGAATTT | 804 |
| DYE40.22.9. | CGTTTCTTTGCTTGTGTCTTGTCTTCGTCGTGCTTGTT | |
| 3p | CC | |
| 010298- | TTTGCATTTTCGGTCATAGTAGCGCGTTTTCATCGTT | 805 |
| DYE40.22.9. | TCGTTTCTTTGCTTGTGTCTTGTCTTCGTCGTGCTTGT | |
| 3p | TCC | |
| 012298- | TTTGTTTACCAGCGCCAAAATAGAAAATTCATATGT | 806 |
| DYE40.22.9. | TTCGTTTCTTTGCTTGTGTCTTGTCTTCGTCGTGCTTG | |
| 3p | TTCC | |
| 014298- | TTTCAAGAAACAATGAAATAGCCCAATAATAAGAGT | 807 |
| DYE40.22.9. | TTCGTTTCTTTGCTTGTGTCTTGTCTTCGTCGTGCTTG | |
| 3p | TTCC | |
| 016298- | TTTCCAACGCTAACGAGCGTTTATCCTGAATCTTATT | 808 |
| DYE40.22.9. | TCGTTTCTTTGCTTGTGTCTTGTCTTCGTCGTGCTTGT | |
| 3p | TCC | |
| 018298- | TTTCAATAATCGGCTGTCTGCATGTAGAAACCAATTT | 809 |
| DYE40.22.9. | TCGTTTCTTTGCTTGTGTCTTGTCTTCGTCGTGCTTGT | |
| 3p | TCC | |
| 020298- | TTTACCAGTATAAAGCCAACGTTATACAAATTCTTTT | 810 |
| DYE40.22.9. | TCGTTTCTTTGCTTGTGTCTTGTCTTCGTCGTGCTTGT | |
| 3p | TCC | |
| 022298- | TTTTCATAGGTCTGAGAGAAGTGAATTTATCAAAAT | 811 |
| DYE40.22.9. | TTCGTTTCTTTGCTTGTGTCTTGTCTTCGTCGTGCTTG | |
| 3p | TTCC | |
| 024298- | TTTTACATAAATCAATATATTTTTAATGGAAACAGTT | 812 |
| DYE40.22.9. | TCGTTTCTTTGCTTGTGTCTTGTCTTCGTCGTGCTTGT | |
| 3p | TCC | |
| 026277- | TTTTACATCGGGAGAAACACAGTAACAGTACCTTTT | 813 |
| DYE40.22.9. | TTCGTTTCTTTGCTTGTGTCTTGTCTTCGTCGTGCTTG | |
| 3p | TTCC | |
| 028277- | TTTTGCGGAACAAAGAAACAGTAACATTATCATTTT | 814 |
| DYE40.22.9. | TTCGTTTCTTTGCTTGTGTCTTGTCTTCGTCGTGCTTG | |
| 3p | TTCC | |
| 030277- | TTTCCTCAATCAATATCTGACCTCAAATATCAAACTT | 815 |
| DYE40.22.9. | TCGTTTCTTTGCTTGTGTCTTGTCTTCGTCGTGCTTGT | |
| 3p | TCC | |
| 032277- | TTTAAGAATACGTGGCACATCTGACCTGAAAGCGTT | 816 |
| DYE40.22.9. | TTCGTTTCTTTGCTTGTGTCTTGTCTTCGTCGTGCTTG | |
| 3p | TTCC | |
| 034277- | TTTCTTGCCTGAGTAGAAGATTAGTAATAACATCATT | 817 |
| DYE40.22.9. | TCGTTTCTTTGCTTGTGTCTTGTCTTCGTCGTGCTTGT | |
| 3p | TCC | |
| 036277- | TTTGGGCGCGTACTATGGTTTAATGCGCCGCTACATT | 818 |
| DYE40.22.9. | TCGTTTCTTTGCTTGTGTCTTGTCTTCGTCGTGCTTGT | |
| 3p | TCC | |
| 038277- | TTTCGTGAACCATCACCCAGGGCGATGGCCCACTAT | 819 |
| DYE40.22.9. | TTCGTTTCTTTGCTTGTGTCTTGTCTTCGTCGTGCTTG | |
| 3p | TTCC | |
| 011043- | TTGGAAAGCGCAGTCTCTGAGGGTTTTGCTCAGTAC | 820 |
| DYE40.22.9. | CAGTCGTTTCTTTGCTTGTGTCTTGTCTTCGTCGTGCT | |
| 3p | TGTTCC | |
| 013043- | TGCGGATAAGTGCCGTCGAGTCCACAGACAGCCCTC | 821 |
| DYE40.22.9. | ATATCGTTTCTTTGCTTGTGTCTTGTCTTCGTCGTGCT | |
| 3p | TGTTCC | |
| 015043- | TGTTAGCGTAACGATCTAAAAGGTGAATTTCTTAAA | 822 |
| DYE40.22.9. | CAGTCGTTTCTTTGCTTGTGTCTTGTCTTCGTCGTGCT | |
| 3p | TGTTCC | |
| 017043- | TCTTGATACCGATAGTTGCGGTAATGCCACTACGAA | 823 |
| DYE40.22.9. | GGCTCGTTTCTTTGCTTGTGTCTTGTCTTCGTCGTGCT | |
| 3p | TGTTCC | |
| 019043- | TACCAACCTAAAACGAAAGAGAACGGTGTACAGAC | 824 |
| DYE40.22.9. | CAGGTCGTTTCTTTGCTTGTGTCTTGTCTTCGTCGTG | |
| 3p | CTTGTTCC | |
| 021043- | TCGCATAGGCTGGCTGACCTGGACGTTGGGAAGAAA | 825 |
| DYE40.22.9. | AATTCGTTTCTTTGCTTGTGTCTTGTCTTCGTCGTGCT | |
| 3p | TGTTCC | |
| 023043- | TCTACGTTAATAAAACGAACGAACCAGACCGGAAG | 826 |
| DYE40.22.9. | CAAATCGTTTCTTTGCTTGTGTCTTGTCTTCGTCGTG | |
| 3p | CTTGTTCC | |
| 025043- | TCTCCAACAGGTCAGGATTATTGGTGTAGATGGGCG | 827 |
| DYE40.22.9. | CATTCGTTTCTTTGCTTGTGTCTTGTCTTCGTCGTGCT | |
| 3p | TGTTCC | |
| 027043- | TCGTAACCGTGCATCTGCCAGGGTAACGCCAGGGTT | 828 |
| DYE40.22.9. | TTCTCGTTTCTTTGCTTGTGTCTTGTCTTCGTCGTGCT | |
| 3p | TGTTCC | |
| 033043- | TAGCACCGTCGGTGGTGCCATGCCGGGTTACCTGCA | 829 |
| DYE40.22.9. | GCCTCGTTTCTTTGCTTGTGTCTTGTCTTCGTCGTGCT | |
| 3p | TGTTCC | |
| 035043- | TAGCGGTGCCGGTGCCCCCTCTGTGTGAAATTGTTAT | 830 |
| DYE40.22.9. | CCTCGTTTCTTTGCTTGTGTCTTGTCTTCGTCGTGCTT | |
| 3p | GTTCC | |
| 037043- | TGCTCACAATTCCACACAACGCAACAGCTGATTGCC | 831 |
| DYE40.22.9. | CTTTCGTTTCTTTGCTTGTGTCTTGTCTTCGTCGTGCT | |
| 3p | TGTTCC | |
| 015189- | ATTAGACCAATAATGTTAGCAAACGTAGTTTACAGT | 832 |
| T3.3p- | TATTAGCCCGCATTT | |
| JF20.22.9.3p | | |
| 017112- | CTGAGGCTTGCAGGTACAGAGGCTTTGACAAGCGCT | 833 |
| T3.3p- | TTACAGTTATTAGCCCGCATTT | |
| JF20.22.9.3p | | |
| 017196- | AGGCGTTTTAGCGATTTTCATCGTAGGATGTTCAGTT | 834 |
| T3.3p- | TACAGTTATTAGCCCGCATTT | |
| JF20.22.9.3p | | |
| 017238- | CCTTAAATCAAGATAACCAAGTACCGCAAAGTCCTT | 835 |
| T3.3p- | TTACAGTTATTAGCCCGCATTT | |
| JF20.22.9.3p | | |
| 017070- | TGACAACAACCATCTCCATTAAACGGGTGAATACAT | 836 |
| T3.3p- | TTACAGTTATTAGCCCGCATTT | |
| JF20.22.9.3p | | |
| 023112- | GGAATACCACATTCCAAAAAGATTAAGAGCTTAGAT | 837 |
| T3.3p- | TTACAGTTATTAGCCCGCATTT | |
| JF20.22.9.3p | | |
| 023196- | TTTACCAGACGACGAAGAAGTTTTGCCACAATTACT | 838 |
| T3.3p- | TTACAGTTATTAGCCCGCATTT | |
| JF20.22.9.3p | | |
| 023238- | AACATAGCGATAGCTCGTCGCTATTAATAAACAAAT | 839 |
| T3.3p- | TTACAGTTATTAGCCCGCATTT | |
| JF20.22.9.3p | | |
| 023070- | ACAACATTATTACATTTTAATTCGAGCTCCTTTAATT | 840 |
| T3.3p- | TACAGTTATTAGCCCGCATTT | |
| JF20.22.9.3p | | |
| 029112- | TAACCTCACCGGAAGGGATAGCTCTCACGGGTAAAT | 841 |
| T3.3p- | TTACAGTTATTAGCCCGCATTT | |
| JF20.22.9.3p | | |
| 029196- | TACAAACAATTCGAATATCTTTAGGAGCCCTGCAAT | 842 |
| T3.3p- | TTACAGTTATTAGCCCGCATTT | |
| JF20.22.9.3p | | |
| 029238- | GTTATTAATTTTAAGGCAAATCAACAGTTCTAAAGT | 843 |
| T3.3p- | TTACAGTTATTAGCCCGCATTT | |
| JF20.22.9.3p | | |
| 029070- | CCAGTGCCAAGCTTAAACTTAAATTTCTAATCCCGTT | 844 |
| T3.3p- | TACAGTTATTAGCCCGCATTT | |
| JF20.22.9.3p | | |
| 035112- | TGCTGCGGCCAGAATCCTCACAGTTGAGGCTAACTT | 845 |
| T3.3p- | TTACAGTTATTAGCCCGCATTT | |
| JF20.22.9.3p | | |
| 035196- | GAGGCCACCGAGTAGAGCTAAACAGGAGCGGGCGC | 846 |
| T3.3p- | TTTACAGTTATTAGCCCGCATTT | |
| JF20.22.9.3p | | |
| 035238- | CGTTGTAGCAATACACGAGCACGTATAATAACCACT | 847 |
| T3.3p- | TTACAGTTATTAGCCCGCATTT | |
| JF20.22.9.3p | | |
| 035070- | ACGATCCAGCGCAGAATCATGGTCATAGCCGGAAGT | 848 |
| T3.3p- | TTACAGTTATTAGCCCGCATTT | |
| JF20.22.9.3p | | |
| 010125- | GCCAGCATTGACAGGTACTGGTAATAAGTTATTCTG | 849 |
| T3.3p- | AAACATGGTTTAGTTTACAGTTATTAGCCCGCATTT | |
| JF20.22.9.3p | | |
| 010251- | ATAATCAAAATCACATCAGTAGCGACAGGGGAGGG | 850 |
| T3.3p- | AAGGTAAAACGCAATTTACAGTTATTAGCCCGCATT | |
| JF20.22.9.3p | T | |
| 010083- | TTGATATTCACAAAGTTCCAGTAAGCGTAGAGAAGG | 851 |
| T3.3p- | ATTAGGATATAAGTTTACAGTTATTAGCCCGCATTT | |
| JF20.22.9.3p | | |
| 012104- | TTAAGAGGCTGAGACTCCTCACATACATATTGGCCT | 852 |
| T2.3p- | TCGTTTCTTTGCTTGTGTCTTGTCTTCGTCGTGCTTGT | |
| DYE40.22.9. | TCC | |
| 3p | | |
| 012146- | CCCTGCCTATTTCGGAACCTATTTTAACAGCCGCCTT | 853 |
| T2.3p- | CGTTTCTTTGCTTGTGTCTTGTCTTCGTCGTGCTTGTT | |
| DYE40.22.9. | CC | |
| 3p | | |
| 012209- | GAATTATCACCGTCACCGACTTAGCAAGCACCCTCT | 854 |
| T2.3p- | TCGTTTCTTTGCTTGTGTCTTGTCTTCGTCGTGCTTGT | |
| DYE40.22.9. | TCC | |
| 3p | | |
| 012230- | CGGAAATTATTCATTAAAGGTCAATGAAAACCGCCT | 855 |
| T2.3p- | TCGTTTCTTTGCTTGTGTCTTGTCTTCGTCGTGCTTGT | |
| DYE40.22.9. | TCC | |
| 3p | | |
| 012272- | GGGCGACATTCAACCGATTGAAATCAAGTCTTTTCT | 856 |
| T2.3p- | TCGTTTCTTTGCTTGTGTCTTGTCTTCGTCGTGCTTGT | |
| DYE40.22.9. | TCC | |
| 3p | | |
| 037119- | CACATTACGCGGGGAGAGGCGGTTTGATGGTGGTTT | 857 |
| T2.3p- | TCGTTTCTTTGCTTGTGTCTTGTCTTCGTCGTGCTTGT | |
| DYE40.22.9. | TCC | |
| 3p | | |
| 037140- | ACTGCCCGCATTAATGAATCGCGGCAAAATCCCTTT | 858 |
| T2.3p- | TCGTTTCTTTGCTTGTGTCTTGTCTTCGTCGTGCTTGT | |
| DYE40.22.9. | TCC | |
| 3p | | |
| 037182- | GAAAGCGAGCTTGACGGGGAAGTTGAGTGTTGTTCT | 859 |
| T2.3p- | TCGTTTCTTTGCTTGTGTCTTGTCTTCGTCGTGCTTGT | |
| DYE40.22.9. | TCC | |
| 3p | | |
| 037203- | TAGGGCGTAAAGGGAGCCCCCGAACAAGAGTCCAC | 860 |
| T2.3p- | TTCGTTTCTTTGCTTGTGTCTTGTCTTCGTCGTGCTTG | |
| DYE40.22.9. | TTCC | |
| 3p | | |
| 037245- | CACACCCTTTTTTGGGGTCGAAGGGCGAAAAACCGT | 861 |
| T2.3p- | TCGTTTCTTTGCTTGTGTCTTGTCTTCGTCGTGCTTGT | |
| DYE40.22.9. | TCC | |
| 3p | | |
| 037077- | CATAAAGTTCTTTTCACCAGTGCAAGCGGTCCACGT | 862 |
| T2.3p- | TCGTTTCTTTGCTTGTGTCTTGTCTTCGTCGTGCTTGT | |
| DYE40.22.9. | TCC | |
| 3p | | |
| 037221- | AGCGGTCACGTAAAGCACTAAATCGAACGTGGACTC | 863 |
| T2.3p- | CATTCGTTTCTTTGCTTGTGTCTTGTCTTCGTCGTGCT | |
| DYE40.22.9. | TGTTCC | |
| 3p | | |
| 037095- | GGGTGCCTAATATTGGGCGCCAGGGCCCCAGCAGGC | 864 |
| T2.3p- | GATTCGTTTCTTTGCTTGTGTCTTGTCTTCGTCGTGCT | |
| DYE40.22.9. | TGTTCC | |
| 3p | | |

The CBFlat and CBPost origami structures are folded as set forth in Examples I and II for square lattice origami.

### Example VI

### DNA Origami Having Isotropic Three-Dimensional Structures

Nucleic acid origami structures can be engineered into any of a variety of three-dimensional shapes. Particular origami shapes can be engineered to benefit particular applications. For example, origami shapes can be engineered to have a desired surface area or shape. In this regard, it is typically easier for an origami having a planar surface to assemble with or otherwise interact with a planar surface of a solid support or other particle compared to an origami having a curved surface since two planar surfaces will have a higher contact area than curved surfaces. Flexibility in terms of shapes that can be designed including relatively complex shapes such as polygons, star polygons or the like. Additionally or alternatively to shape, a nucleic acid origami can be engineered to have any of a variety of properties such as rigidity, flexibility, anisotropy, symmetry. Such properties can be imparted for example by including more or fewer layers with an origami structure or by orienting layers to interact in a variety of orientations (e.g., parallel helices, antiparallel helices, or helices having angular offsets such as orthogonal helices).

Tile shapes such as rectangles, squares or disks can be useful, for example, for occupying sites on arrays having complementary footprints. For example, tiles can be configured to have a size, shape and surface that fits into or onto an array feature. The foregoing shapes can be anisotropic, for example, having one or more sides or surfaces that are larger or smaller than other surfaces in the same structure. A large surface can be useful for orienting an attached analyte on an array surface for interaction with an affinity reagent or other assay reagent. However, in some cases it may be desirable for a nucleic acid origami to have an isotropic structure. For example, a nucleic acid origami to which an affinity moiety, or other functional moiety, is attached for use in an assay can benefit from an isotropic shape due to improved diffusion in fluid media.

This example demonstrates the design of a new DNA origami structure having a cubic shape, 96 helices in total, and dimensions of 24 nm×24 nm×24 nm. The structure is further designed to be used as a retaining component for attachment of several different functional moieties including antibodies, labels and tethers. Having six facets, this DNA origami structure provides multiple options for display of antibodies, labels and tethers on any of a variety of the facets and in any of a variety of combinations. Therefore, different constructs are possible, where functional components are displayed on the same facet or on different facets, respectively.

This example also describes a protocol for synthesis, including the use of different temperature ramps and positively charged ions for origami assembly. While nucleic acid origami structures are typically assembled in the presence of Mg²⁺ at concentrations of 12.5 mM or higher, the present example demonstrates folding of nucleic acid origami at low concentrations of Mg²⁺ and Na⁺. This can be beneficial for downstream uses of the origami where high salt concentrations are undesirable such as reactions for attaching moieties to the origami or use of the origami in detection assays.

### Design of an Affinity Reagent Having a DNA Origami Cube Structure

Cube-shaped DNA origami structures allow presentation of antibody moieties and tether moieties on multiple facets for improved binding to analytes having epitopes recognized by the antibodies, and having dockers recognized the tethers. Moreover, antibodies, tethers and labels can be attached to spatially defined locations on the surface of the cube.

Design criteria for the cube-shaped DNA origami included (1) a substantially three-dimensionally isotropic shape having six sides, (2) use of the M13mp18 circular plasmid (7249 bp) as a scaffold, which is commercially available, (3) attachment of multiple fluorescent dyes (Alexa 647) for brightness, (4) attachment of multiple antibodies at sites that are separated by at least ten nanometers to reduce steric hindrance between neighboring antibodies which have a width of about 14 nm, (5) attachment of multiple oligonucleotide tethers to provide an avidity effect when an antibody moiety is bound to an analyte that is associated with oligonucleotide dockers that complement the tethers, and (6) at least one to two crossovers between different helices to provide conformational stability to the origami structure, and including staples having lengths between eighteen and 50 nucleotides.

The origami was designed using CaDNAno v2.4 software and the hexagonal lattice option. The structure included 96 helices distributed in 8 different layers, in order to obtain a structure 24 nm in length along all axes as diagrammed in FIG. 15A. FIG. 15B shows a schematic of the helix packing structure and corresponding dimensions across the different sides (also referred to as “planes”). An exemplary helix is shown in FIG. 15C.

Three different configurations for display of antibodies, tethers and fluorophores on the cube-shaped origami structure are provided below. The configurations are described with reference to the diagrammatic representation of the DNA origami shape in FIG. 15A, wherein Plane B is opposite Plane A, Plane E is opposite Plane C, and Plane F is opposite Plane D.

### Configuration 1: Antibodies and Tethers on all Six Planes, and Labels on Two Opposing Planes

In Configuration 1, antibodies and tethers were attached to all planes while AF647 (Alexa 647) fluorophores were attached to Planes A and B. Table 7 lists the number of antibodies, tethers and dyes on each plane of the origami structure. The handle strands were functionalized at the 3′ end for attachment to AF647 and at the 5′ end for antibodies, whereas tethers are located at the 3′ ends of handles.

TABLE 7
Moieties attached to the origami structure in Configuration 1

| | AF647 | | |
|---|---|---|---|
| Plane | fluorophores | Antibodies | Tethers |
| A | 16 | 5 | 2 |
| B | 17 | 4 | 4 |
| C | 0 | 5 | 4 |
| D | 0 | 2 | 5 |
| E | 0 | 4 | 4 |
| F | 0 | 3 | 3 |
| Total | 33 | 23 | 22 |

As indicated in Table 7, the total number of AF647 fluorophores in the structure was 33, the total number of antibodies was 23, and the total number of tethers was 22.

The AF647 fluorophores were attached to staples located at helical ends on Planes A and B. The helices are numbered in FIG. 16. The AF647 fluorophores of Plane A were attached to staples located at the ends of helices 14, 16, 18, 20, 22, 40, 44, 46, 64, 68, 70, 86, 88, 90, 92 and 94. The AF647 fluorophores of Plane B were attached to staples located at the ends of helices 5, 7, 9, 11, 25, 27, 29, 31, 33, 47, 51, 55, 73, 75, 77, 79 and 81.

The antibodies of Plane A were attached to staples located at the ends of helices 23, 37, 47, 61 and 71. The antibodies of Plane B were attached to staples located at the ends of helices 2, 18, 54 and 72. The antibodies of Plane C were attached to staples hybridized to helices 11 (2 antibodies attached), 35, 59 and 83. The antibodies of Plane D were attached to staples hybridized to helices 5 and 7. The antibodies of Plane E were attached to staples hybridized to helices 23, 47, 71 and 95. The antibodies of Plane F were attached to staples hybridized to helices 91, 85 and 87.

The tethers of Plane A were attached to staples located at the ends of helices 42 and 66. The tethers of Plane B were attached to staples located at the ends of helices 13, 21, 49 and 57. The tethers of Plane C were attached to staples hybridized to helices 12, 36, 59 and 83. The tethers of Plane D were attached to staples hybridized to helices 1, 3, 5, 7 and 9. The tethers of Plane E were attached to staples hybridized to helices 24, 48 and 72. The tethers of Plane F were attached to staples hybridized to helices 86, 89 and 92.

### Configuration 2: Antibodies and Tethers on a First Plane and Labels on an Opposing Plane

In Configuration 2, antibodies and tethers were displayed on a single plane (Plane A) and labels (AF647 fluorophores) were displayed on a single plane (Plane B) which is opposite the plane displaying the antibodies. Table 8 lists the numbers of antibodies, tethers and dyes displayed on each plane.

TABLE 8
Moieties attached to the origami structure in Configuration 2

| | AF647 | | |
|---|---|---|---|
| Plane | fluorophores | Antibodies | Tethers |
| A | 0 | 8 | 10 |
| B | 30 | 0 | 0 |
| C | 0 | 0 | 0 |
| D | 0 | 0 | 0 |
| E | 0 | 0 | 0 |
| F | 0 | 0 | 0 |
| Total | 30 | 8 | 10 |

Referring to the helix numbering of FIG. 16, antibodies were attached to Plane A at staples located at the ends of helices 13, 17, 23, 35, 47, 63, 71 and 89; tethers are attached to Plane A at staples located at the ends of helices 12, 16, 20, 38, 42, 46, 60, 68, 86 and 94; and AF647 fluorophores were attached to Plane B at staples located at the ends of helices 1, 5, 7, 9, 11, 13, 15, 17, 19, 21, 25, 27, 29, 31, 33, 35, 47, 49, 51, 53, 55, 57, 59, 71, 73, 75, 77, 79, 81 and 83. The total number of AF647 fluorophores in the structure was 30, the total number of antibodies was 8, and the total number of tethers was 10. Planes C, D, E and F did not display any labels, antibodies or tethers.

### Configuration 3: Antibodies and Tethers on Two Planes and Labels on all Planes

In Configuration 3, antibodies and tethers were displayed on opposing planes (Planes A and B), while AF647 fluorophores were displayed on all planes. Table 9 lists the numbers of antibodies, tethers and dyes displayed on each plane.

TABLE 9A
Moieties attached to the origami structure in Configuration 3

| | AF647 | | |
|---|---|---|---|
| Plane | fluorophores | Antibodies | Tethers |
| A | 6 | 6 | 6 |
| B | 11 | 6 | 6 |
| C | 4 | 0 | 0 |
| D | 5 | 0 | 0 |
| E | 3 | 0 | 0 |
| F | 3 | 0 | 0 |
| Total | 32 | 12 | 12 |

As indicated in Table 9A, the total number of AF647 fluorophores in the structure was 32, the total number of antibodies was 12, and the total number of tethers was 12.

The AF647 fluorophores were displayed on planes via staples hybridized to helices which are numbered in FIG. 16. The AF647 fluorophores of Plane A were attached to staples located at the ends of helices 38, 40, 42, 44, 46 and 48. The AF647 fluorophores of Plane B were attached to staples located at the ends of helices 13, 15, 17, 19, 21, 47, 49, 51, 53, 55 and 57. The AF647 fluorophores of Plane C were attached to staples hybridized to helices 12, 36, 59 and 83. The AF647 fluorophores of Plane D were attached to staples hybridized to helices 1, 3, 5, 7 and 9. The AF647 fluorophores of Plane E were attached to staples hybridized to helices 24, 48 and 72. The AF647 fluorophores of Plane F were attached to staples hybridized to helices 86, 89 and 92.

The antibodies of Plane A were attached to staples located at the ends of helices 13, 23, 59, 71, 83 and 93. The antibodies of Plane B were attached to staples located at the ends of helices 2, 10, 26, 36, 72 and 82.

The tethers of Plane A were attached to staples located at the ends of the helices 16, 20, 64, 68, 88, 92. The tethers of Plane B were attached to staples located at the ends of helices 13, 21, 49 and 57.

### Synthesis of Null Version of the DNA Origami Structure (No Antibodies, No Tethers) in the Presence of Mg²⁺ Ions or Na⁺ Ions

A null version of the Cube DNA origami structure (i.e. lacking antibodies and tethers) was folded in the presence of various concentrations of Mg²⁺ or Na⁺ ions. The ‘Cube Lobe’ structure includes 230 staples in total, each one of them at an initial concentration of 150 uM. Nucleotide sequences for the Cube Lobe are listed in Table 9B.

TABLE 9B
Oligonucleotide Sequences for Cube Lobe Origami

| Sequence | | SEQ ID |
|---|---|---|
| Name | Sequence | NO |
| 1-016120 | TAATCAGTACATTTTCGG | 865 |
| 2-020120 | GGAAATTATGCCAGCAAA | 866 |
| 3-046075 | AAAAATAGAAGCCTTGAGAG | 867 |
| 4-013056 | TTTTTCAGGAACGAGCCGCTT | 868 |
| 5-019056 | GCGCATAACTGACCCTTTCAA | 869 |
| 6-039084 | CAGTTACGAATCTTCTTCAAG | 870 |
| 7-036091 | AGAGAGAAACGATTTGAAACGG | 871 |
| 8-039070 | AGAGCCTTAGCCAGGAACAAAC | 872 |
| 9-043070 | TATTCTACAATCATGCAAATAT | 873 |
| 10-093063 | TTGCAACCTCATGGAGTAATAA | 874 |
| 11-001044 | CCACCCTCATTTTCAACTTTGAATAA | 875 |
| 12-025044 | ACGGAACAACATTATATTTACCCTGT | 876 |
| 13-027044 | AATTACGAGGCAAAGCTAATTAAGCA | 877 |
| 14-029044 | AAGTTTTGCCAGATCATACCATTTGG | 878 |
| 15-031044 | CAAATGCTTTAAAAATGGTAGTTTGA | 879 |
| 16-003044 | GCATTCCACAGACCAAATCAGACCAG | 880 |
| 17-049044 | CAATATGATATTCGAGCCGTCCGCTC | 881 |
| 18-051044 | CTGAGAGTCTGGGGTCATACCAAGCT | 882 |
| 19-053044 | GCCCCAAAAACACAGTCACAAGTTGG | 883 |
| 20-055044 | GCTCATTTTTTATGGCGAACGGGCCT | 884 |
| 21-057044 | GTAACAACCCGTGCTGCGCAGGCAAA | 885 |
| 22-005044 | TTTGCTAAACAAAACTTTGGACCTGC | 886 |
| 23-073044 | TCGTGCCAGCTGTACATTTATTACCG | 887 |
| 24-075044 | GACGGGCAACAGTATCGGCTTTGATT | 888 |
| 25-077044 | CTGTTTGATGGTTCACGCAGTGTTTT | 889 |
| 26-079044 | AAGAGTCCACTAAGACAGGAGAATCA | 890 |
| 27-007044 | ATAATTTTTTCATCATCGCTAAAACA | 891 |
| 28-081044 | AAATCAAGTTTTTGACGAGCGCCGCG | 892 |
| 29-009044 | TTCTTAAACAGCAACCTAATAAAGAC | 893 |
| 30-011085 | AGAGCCACACCATCAACAGATCGAGCC | 894 |
| 31-013092 | AAATCACCGGAACCCGCCACTCAGAGC | 895 |
| 32-038109 | TTTATCCCAATCGCGCTAACTGAACAA | 896 |
| 33-040109 | CCAGCTACAATTAAGAAACTTGAGTTA | 897 |
| 34-042109 | TGCGGGAGGTTTGCAGATACCGAAGCC | 898 |
| 35-044109 | TCATTACCGCGCATACCCAACCGAGGA | 899 |
| 36-046109 | CCGCACTCATCGGTAGAAACTTATTAC | 900 |
| 37-059064 | GGACGATTGGTGTTCCGTGGCTTTCAT | 901 |
| 38-062109 | GATGCAAATCCAAATTCTTTGTTTAGT | 902 |
| 39-068075 | GAGCTCTCGACTCGTCCACGCAAAATC | 903 |
| 40-070109 | TATTCATTTCAAACAAGAATTTATCAA | 904 |
| 41-086109 | TTGAGGAAGGTTTATCATTTTAATTTT | 905 |
| 42-088109 | CTTGCTGAACCTTGATTATAGGAGCGG | 906 |
| 43-090109 | TCAGTATTAACACTGAATAAATCCTGA | 907 |
| 44-092109 | TAGCCCTAAAACAAATAAATCAAAATT | 908 |
| 45-094109 | CTGAAAGCGTAAGTACCTTAACGTCAG | 909 |
| 46-015105 | TCATCGGGCGACAGATCGATAAGCACCA | 910 |
| 47-040069 | GCGTCACGCTGAAATTAACATCTTCCGG | 911 |
| 48-042075 | GTAGCAAGGTGGCTGTTAAACATCAAAA | 912 |
| 49-053084 | TGTAAAGACAAACTTCGTAGAATGGGTA | 913 |
| 50-055078 | ATAATTCTTCCTGAATTTGCTGTCTGGA | 914 |
| 51-062090 | GACAAAGGGGTTATAATCATAAGCTTAG | 915 |
| 52-064069 | CCTTTTACATCCTTTAGAAGGCTGCAGG | 916 |
| 53-068109 | CCTTTTTTAATGCAACATGATTCTGTCC | 917 |
| 54-079084 | GGCGATCGGAAAGAAATCAATACTTGCA | 918 |
| 55-007084 | AAAAGCATAACAGTACAGTTAGGTTGTC | 919 |
| 56-088090 | CAAACCCAGCAAATCAACAGTCAGAAGA | 920 |
| 57-089078 | GCCAGCTCAATCAATGGCCGCAGGAATC | 921 |
| 58-093078 | GAATGGTACCGAACGTGAGTTTGCCAAC | 922 |
| 59-036070 | GGTCATTTTTTTAAAATTCCATATTTTCC | 923 |
| 60-064109 | TCAATAGTGAATTATTTAAAGTAGGGCTT | 924 |
| 61-066109 | CCTTGCTTCTGTAGAATATGAGGCATTTT | 925 |
| 62-073077 | GCGGGGAGCGTATTAATTAATCGGGTACC | 926 |
| 63-075077 | GCCCTGAGCAAGCGTAGAGGAAATTAATT | 927 |
| 64-077077 | CCTTATAATAGCCCTAGCGATGGTCTGAG | 928 |
| 65-012120 | ACCGCCTCCCTCAGAGCAGAGCCCCTTATT | 929 |
| 66-060071 | ATCGCACTCTCAAATATTTTCGCATTTAAC | 930 |
| 67-011044 | AGGGAGTTAAAGGGGTAGCAACGGCTACAGA | 931 |
| 68-035044 | CCAACAGGTCAGTGGCTTAGAGCTTAATTGC | 932 |
| 69-036109 | GAAAATAGCAGCGCGCATTAGACGGGAGAAT | 933 |
| 70-059044 | GTGCATCTGCCACTTTCCGGCACCGCTTCTG | 934 |
| 71-060109 | TTGAAATACCGAAACACCGGAATCATAATTA | 935 |
| 72-064075 | AGACTACTCCGGCCAGGGCGCCGTAAAGCAC | 936 |
| 73-083044 | GCGAACGTGGCGTAGCGGTCACGCTGCGCGT | 937 |
| 74-038075 | AGTTTCTATGCAAGGTCACGCGACAGTGGAAG | 938 |
| 75-082096 | AAAAGTATTCGACAACTCGTATTAAATCCTTT | 939 |
| 76-019093 | GAGCCATTTGGGTTACCATCTGCCTAGTCAGTGC | 940 |
| 77-021093 | GATTGAGGGAGGAGGTGAAAGCGGGGACTCCTCA | 941 |
| 78-053078 | TTAAATAATATTTATCAATTACCAACGCTAACGA | 942 |
| 79-058096 | ATCATAAAGAATACCGTGTGATAAATATCATCTT | 943 |
| 80-066075 | TTCCCTGAAAACAGAGATAGCTCCAACGTCAAAG | 944 |
| 81-010096 | GGTCAGACCACCCCCTCAGAACCGCCAGGTAATCA | 945 |
| 82-015092 | CTGTAGCGCGTTTAGCGTTTTGATATTTTGAGGCA | 946 |
| 83-017092 | CACCAATGAAACCAATCAAGTCATACATCTGAATT | 947 |
| 84-051071 | CGATGAACGGTAAAGCATGTAGAACGCTAATAGTA | 948 |
| 85-062076 | GAAAACTTATTTTAAGCCCCCGAAAGCGGCGCTAG | 949 |
| 86-068069 | GAATTCGCATCAAGAAAACAAGGGCGCCCCGCCTG | 950 |
| 87-006096 | CTTGAGGTAAGCGTTTGCCTTTAGCGCAGAAACGT | 951 |
| 88-008096 | TACCGTTTGGCCTGCCATCTTTTCATAAAGTCAGA | 952 |
| 89-002096 | GAGAGGTAGGATTTTATCACCGTCATAGATTCAACC | 953 |
| 90-049077 | TAATGCCGCTATTTTTTATTTATCAGATATAGAAGG | 954 |
| 91-049084 | GGAGAAACAATCAATAATCGGTTATCATGCAAGGAT | 955 |
| 92-004096 | AGAGAAATGCCCCTAGCAAGGCCGGACAGCCGACTT | 956 |
| 93-055063 | GGAACGCATTCGCATGTATAAATGTACCAAGAGAAT | 957 |
| 94-057078 | GGCGGATGGGATACTAAAGTTATTTTGGCTCCACAT | 958 |
| 95-054094 | CGAGATCGCCATTATCAAATAACTATATGTAAATGCT | 959 |
| 96-056094 | AATTTTATACAATCGCAACTGACCTAAATTTAATGGT | 960 |
| 97-028069 | AAAATAGAGCAACATAATGCAGGTAGAAAGATTCATCA | 961 |
| 98-033044 | TTGCATCAAAAATTGATTCTGTAGCTCGCATCGTAACC | 962 |
| 99-052095 | AGACGTAATAAGAAATCGTATTAAGACGCTGAGAAGAG | 963 |
| 100-081081 | TAAATCGGGACAAAAACCGTCTATTTAGGTTAACGCGA | 964 |
| 101-018068 | AACCGAGGCTGGCTGACCTTAAAAACCAAATGTTTAGAC | 965 |
| 102-020068 | GGATATCCTGACGAGAAACAATTCAACCTATCATAACCC | 966 |
| 103-026096 | GCAGTATAACGGACCAATAGTTTAGCGAACCTCCCGACT | 967 |
| 104-028096 | AACGCAAAAGTAATGAAGCCATTAGTTGCTATTTTGCAC | 968 |
| 105-030096 | CTTTTTTAAGAGCTTATCCTAAAATAAACAGCCATATTA | 969 |
| 106-032096 | AGCCCATAATTGACAAATAATTTGTTTAACGTCAAAAAT | 970 |
| 107-050096 | CAATAGCAATAAAGAAACAGAATATATGTGAGTGAATA | 971 |
| | A | |
| 108-074096 | ATGAATAAAACAGATCGCCATAAAACAGAGGTGAGGCG | 972 |
| | G | |
| 109-076096 | ATTTGCTATACTTCCGCCTGGAAAAATCTAAAGCATCAC | 973 |
| 110-078096 | TTGTTTATATTCCCAAATATAATCAACAGTTGAAAGGAA | 974 |
| 111-080096 | AATTATGTAACATATCTAAAAGAGCCGTCAATAGATAAT | 975 |
| 112-014069 | ACTACGATTGACCCCCAGCGATACCCTGCCGAAAGACTT | 976 |
| | C | |
| 113-015056 | CTCATCTAGGCACCTTGATACTTCGGTCGCTGAGGCTTGC | 977 |
| 114-016069 | AACGGAGTACTTAGCCGGAACCGGAATCGAGAATGACC | 978 |
| | AT | |
| 115-017056 | TCCATGTATTTGTACGTTGAAGCTTGCTTTCGAGGTGAAT | 979 |
| 116-021056 | GGCTTGCTCATTACCAGCCCTTGAATTTTCTGTATGGGAT | 980 |
| 117-002069 | CACCAGTTAGCAAGCCTCAGAACCGCCACCCTCAGAGCC | 981 |
| | A | |
| 118-039056 | CCATTAGATAACAGGATTAAGACCAGACCGGAAGCAAA | 982 |
| | CT | |
| 119-041056 | GGCGCGAATTTCGCACAGTTCTAGTCAGAAGCAAAGCGG | 983 |
| | A | |
| 120-043056 | ATAAAGCCCAATAAAGGGGGTATATTCATTGAATCCCCC | 984 |
| | T | |
| 121-045056 | AATACTTGAGCATATAGTAAGCGAGAGGCTTTTGCAAAA | 985 |
| | G | |
| 122-065056 | GTAACGCCGCCAGCACCAATACAACATTAAATGTGAGCG | 986 |
| | A | |
| 123-067056 | TGCATGCGTTTTCCGGAAGATTTAAATTTTTGTTAAATCA | 987 |
| 124-069056 | ACAATTCTAATCATAGCAAACCCGGTTGATAATCAGAAA | 988 |
| | A | |
| 125-072097 | TGATTGCAGTAACAGAATACGTCTTTAATGCGCGAACTG | 989 |
| | A | |
| 126-083086 | AAACATTAACCCTAAAGGGGTTAATTAGCCTCAATCGGG | 990 |
| | C | |
| 127-087056 | GAGCGGGGTTGCTTTTGGGGTAGCTTGACGGGGAAAGCC | 991 |
| | G | |
| 128-089056 | TATAATCGGATTTTTTAAAGAACTACGTGAACCATCACC | 992 |
| | C | |
| 129-008069 | TTTATCAAATCTCCAGGAACAACTAAAGGAATTGCGAAT | 993 |
| | A | |
| 130-091056 | AGTAATACTGTCCAGGTTCCGTGTTGTTCCAGTTTGGAAC | 994 |
| 131-093056 | CCAGCCACTCAAACCTGATTGGCCCCAGCAGGCGAAAAT | 995 |
| | C | |
| 132-035086 | AAAAAAGGCGTTTTAATTCTTAAACGCCAGCAGCCCTCC | 996 |
| | TC | |
| 133-012070 | ACAGCATCTGAGGAAGTTTCCAGAGCTTCTTTAATTATA | 997 |
| | AGA | |
| 134-042090 | TTAAAACGAGGCGTCAAGCAACACCGTTGCAAGACTCCA | 998 |
| | AGAA | |
| 135-019105 | AATTAGATCATTAAGAAGGTACCAGCGCATCAATAGAAA | 999 |
| | ATTCA | |
| 136-031082 | AAATCAAAAAACGTCCAATACTGGAGGCGCGCGAAACA | 1000 |
| | AAGTAC | |
| 137-033082 | AAATATCGGATAAATCAGGTCTTTTATACCAAATACGTA | 1001 |
| | ATGCC | |
| 138-072085 | ATACCGTTGAATTGCAGAAGATGATGAAACAAACACAA | 1002 |
| | GCTAAC | |
| 139-027082 | TCGTTTAAATGTAGGAATACCACCCAGAACATCTTGACA | 1003 |
| | AGAACC | |
| 140-029082 | TGGATAGGAATCCAGACGACGATCATCAAGGGTCAATCA | 1004 |
| | TAAGGG | |
| 141-025080 | GTTGAGATTTTAAGACACCAACGTTTCAGGCGAGTAAAT | 1005 |
| | TGGGCTT | |
| 142-084109 | ACATTTGAGGATTTAGAAGTATTAGAATAGATTATATCT | 1006 |
| | TGTTGGCA | |
| 143-092090 | TTAAAAACTATTAGTGGCACAGCCAACAGAGATAGAACC | 1007 |
| | CTTCTGAC | |
| 144-024116 | AGGTGGCAACATATAAAAGTGTCACACAAAGACTATAG | 1008 |
| | CCGTGCCGTC | |
| 145-044068 | CTTACATTGCGGGTTTTAGAAAAGGGTGATTTCCCTGTCG | 1009 |
| | AGATAAAT | |
| 146-072063 | CTTTCCAGATGAGTGCAACATACAACCGTAAGGCTATCA | 1010 |
| | GGTCATTGC | |
| 147-048064 | CGGAGACAGAGATTCAACCCTCATATTACAGATACATAA | 1011 |
| | CGCCAAAAGG | |
| 148-066090 | CGCTATTTCAAATCTACATCCTACATTTAGCAAAAAGTTA | 1012 |
| | CAAAATCGC | |
| 149-024065 | TCTACGTTAAAGAACTGTTTAATTTCAGGGAACAAACTA | 1013 |
| | CAACGCCTGTA | |
| 150-063056 | CTTCGCTCATTCAGCGGATTCAGATGGGCAACATGTTGC | 1014 |
| | GGAGATTAGAG | |
| 151-000116 | ATCACCGTACTCAGGAGGTTTAGTACATATAAGAAAAGG | 1015 |
| | GCGACAGTGAATAAGT | |
| 152-095037 | GTCTGAAATGGATTATTTACATTGGCAAATACCCATTAA | 1016 |
| | TTTTTTCTTTTCACCAGTGA | |
| 153-018120 | ATCACCAGTGCAGCACCG | 1017 |
| 154-011063 | TTGCGGGCGATATACGATAGT | 1018 |
| 155-005063 | CAGTTTCTAGTAAACATAGTT | 1019 |
| 156-071070 | TCACATTCGCTCACCCAACGC | 1020 |
| 157-085056 | CTTAATGGCAAGTGAGAAAGGAA | 1021 |
| 158-002116 | CCAGGCGGATAACGGAATAGGTGT | 1022 |
| 159-037037 | TGAATATAATGCCCAATTCTGCGA | 1023 |
| 160-054116 | GTAATTTAGGCAAAAGTACCGACA | 1024 |
| 161-061037 | GTGCCGGAAACCAACTGTTGGGAA | 1025 |
| 162-011105 | CGCCACCCAGGAGGCACAAACCGCAGTC | 1026 |
| 163-087078 | TGGTCATAGGAGCACGCGCGACAGGTAA | 1027 |
| 164-091078 | CACCAGGCCACGCTGGAGTACCACCAGA | 1028 |
| 165-023037 | ATTACCTTATGCGATTTTATAAAACGAACTA | 1029 |
| 166-047037 | CTGAGTAATGTGTAGGTAATCAAATCACCAT | 1030 |
| 167-071037 | GTAAAGCCTGGGGTGCCTATCGGGAAACCTG | 1031 |
| 168-072116 | AAACAATAACGGATTCGCCGCAGAGGCGAAT | 1032 |
| 169-095071 | AAGGGACATTCTGGACAATATTAAACGAGGAATTT | 1033 |
| 170-007105 | TGGCTTTTAACGGGTTTCGGAGGCTGAGTTTTGCTCAGTA | 1034 |
| 171-083065 | GGGAAGATTTAGCGAGGTGATGGCCCACGTGGAGGTTG | 1035 |
| | AG | |
| 172-059084 | GTTAAATTGCGTAATTGACCGGACTGGCCGCGTGACCAC | 1036 |
| | GTT | |
| 173-023092 | TTATTTAAACGCAAGCAAACAGAACAATTTATTTTCATC | 1037 |
| | GTAGGAA | |
| 174-047092 | CGGGTATAGAAACGTCCTGATTACCTGAACAATTTCATT | 1038 |
| | TGAATTA | |
| 175-035064 | AGTACCAAAGCGAAGGAAGCACTATTAAGAAAACGTCA | 1039 |
| | TAAAATAGTA | |
| 176-013037 | GGCTTTGAGGACAACGAAAGAGGC | 1040 |
| 177-015037 | AAAAGAATACACCTGATAAATTGT | 1041 |
| 178-017037 | GTCGAAATCCGCAAAGAGGACAGA | 1042 |
| 179-019037 | TGAACGGTGTACAACGTAACAAAG | 1043 |
| 180-021037 | CTGCTCATTCAGTAATCATTGTGA | 1044 |
| 181-026116 | TGATTAAGACTCATACATACATAA | 1045 |
| 182-028116 | TACCAGAAGGAAAAAGAACTGGCA | 1046 |
| 183-030116 | ATAGCTATCTTAGCCGAACAAAGT | 1047 |
| 184-032116 | AACCCACAAGAAAATGAAATAGCA | 1048 |
| 185-034116 | TAACTGAACACCTATCAGAGAGAT | 1049 |
| 186-039037 | ACGAGTAGATTTCAATAACCTGTT | 1050 |
| 187-043037 | GAATTAGCAAAAATCGGTTGTACC | 1051 |
| 188-045037 | AAAAACATTATGTAAATGCAATGC | 1052 |
| 189-052116 | AAAGGTAAAGTATTCAGCTAATGC | 1053 |
| 190-056116 | CCAACGCTCAACCAACGCCAACAT | 1054 |
| 191-063037 | GGGCGATCGGTGAGGGGGATGTGC | 1055 |
| 192-067037 | CGACGGCCAGTGGCTGTTTCCTGT | 1056 |
| 193-069037 | GTGAAATTGTTAGAAGCATAAAGT | 1057 |
| 194-074116 | ATTTTCAGGTTTTTACATCGGGAG | 1058 |
| 195-076116 | GAACCTACCATAGAAATTGCGTAG | 1059 |
| 196-078116 | ATTCATCAATATATGGAAGGGTTA | 1060 |
| 197-080116 | GAAACCACCAGACAGATGATGGCA | 1061 |
| 198-082116 | GCCCGAACGTTATTGCGGAACAAA | 1062 |
| 199-085037 | AACCACCACACCCACGTATAACGT | 1063 |
| 200-087037 | GCTTTCCTCGTTAACGGTACGCCA | 1064 |
| 201-089037 | GAATCCTGAGAAAATTAACCGTTG | 1065 |
| 202-091037 | TAGCAATACTTCCTTGCTGGTAAT | 1066 |
| 203-093037 | ATCCAGAACAATTGACGCTCAATC | 1067 |
| 204-048116 | ATCCTAATTTACGAGCATGTTAAACCAAGTA | 1068 |
| 205-010127 | CCGCCGCCAGCATTGAAGAACCACCACCAGAG | 1069 |
| 206-004127 | CATGAAAGTATTAAGAACCTATTATTCTGAAA | 1070 |
| 207-006127 | TACTGGTAATAAGTTTTGATGATACAGGAGTG | 1071 |
| 208-008127 | AAGCCAGAATGGAAAGAAATAAATCCTCATTA | 1072 |
| 209-014120 | TCATAGCCCACCACCGGA | 1073 |
| 210-022120 | TATGGTTTAAATATTGAC | 1074 |
| 211-034096 | AGTCAGCAGGGAACTTTAC | 1075 |
| 212-050069 | ATCTACATCTAGCTAAGGC | 1076 |
| 213-084069 | GGCGCTGCGCCGCTTACTATG | 1077 |
| 214-087063 | AGCTAAAATTAAAGAGTGAGG | 1078 |
| 215-090069 | AAAGAGTACATCACAGAAGAA | 1079 |
| 216-061056 | GCGCCATCAGCCAGGTTTGAGG | 1080 |
| 217-085078 | CAACTACTAGCGGAAAGGTTAC | 1081 |
| 218-041037 | TAGCTATATTTTAGGCAAGGCAAA | 1082 |
| 219-050116 | AGAACGCGCCTGAAATAATATCCC | 1083 |
| 220-058116 | CTAGAAAAAGCCACCAGTATAAAG | 1084 |
| 221-065037 | TGCAAGGCGATTGACGTTGTAAAA | 1085 |
| 222-075084 | GAGAGGTATGGTTTGAGGCACTTTGA | 1086 |
| 223-001075 | TAGGAACCCACACTGAGTTTCGTAGCGT | 1087 |
| 224-003075 | AACGATCTAGTCTTTCCAGACGTAGCGG | 1088 |
| 225-005075 | AGTGTATAAGCCCGAGAATAGAAAAAAA | 1089 |
| 226-007075 | AAAGGCTCCTTTAATTGTATCGGTGCGC | 1090 |
| 227-022069 | GAGATGGGCTCATTATACCAGGGGAAGAAAAA | 1091 |
| 228-009075 | CGACAATGACGCCCACGCATAACATCGTCACGAAAG | 1092 |
| 229-077063 | AAATCGGCTGGTTTCCCTTCAAGGGTGGGAATCGGTGCC | 1093 |
| | CG | |
| 230-003084 | AAGTTATGTCCGTAATGTATGCGCCACCCTCAGAACCGC | 1094 |
| | CACCCCAA | |

To fold the Cube Lobe structure, a ‘staples mix’ solution was prepared by mixing 20 μL of each staple (at 150 uM initial concentration) to a final concentration of 652 nM. For the screening with Mg²⁺, 200 uL of folding solution was prepared by mixing 20 uL of 10× Folding Origami Buffer (50 mM Tris, pH 8.0, 50 mM NaCl, 10 mM EDTA), 61 uL of the staples mix solution (final concentration of 200 nM), 9 uL ssDNA 7249 scaffold solution to a final concentration of 20 nM, and MgCl₂ to a final concentration set forth below. Nuclease-free H₂O was added to reach 200 uL final total volume.

The following MgCl₂ concentrations were tested: 8 mM, 10 mM, 12 mM, 14 mM, 16 mM, 18 mM, 20 mM, and 22 mM. The 200 uL solution was split into aliquots and transferred to PCR tubes to be run in a Thermocycler. Two different folding conditions were tested. For the first folding condition (referred to as the ‘short folding condition’), the samples were incubated in the Thermocycler at 95° C. for 5 minutes. Then, a temperature ramp was applied from 90° C. to 20° C., incubating the sample for 60 seconds at every 1° C. decrement. For the second folding condition tested (referred to as the ‘long folding condition’), the samples were incubated at 65° C. for 10 minutes. Then, a temperature ramp was applied from 60° C. to 40° C., incubating the sample for 60 minutes at every 1° C. decrement. From 40° C., the temperature was further decreased to 20° C., and the samples were incubated at this temperature for 1 hour.

After the folding, 5 uL of each aliquot were mixed with 5 uL of 1×OB (5 mM Tris-HCL, pH 8, 200 mM NaCl, 12.5 mM MgCl₂, 1 mM EDTA) and 2 uL of Gel loading dye and run on a 2% agarose gel at 70V for 3 hours in an ice bath. Ten microliters of a 1 kb DNA ladder diluted in 1×OB (5 uL of ladder+5 uL of 1×OB) was added to the gel with 10 uL of a 10 nM solution of p7249 ssDNA scaffold, used as control.

The same folding protocol and gel electrophoresis assay were used in the screening of different NaCl concentrations. For these tests, the MgCl₂ solution was replaced with NaCl (initial concentration 200 mM) testing the following final concentrations: 8 mM, 10 mM, 12 mM, 14 mM, 16 mM, 18 mM, 20 mM, 22 mM.

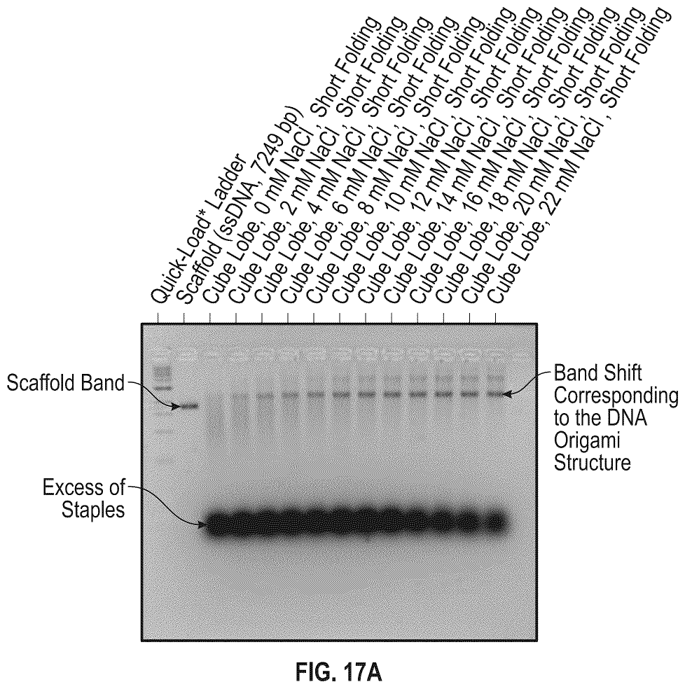

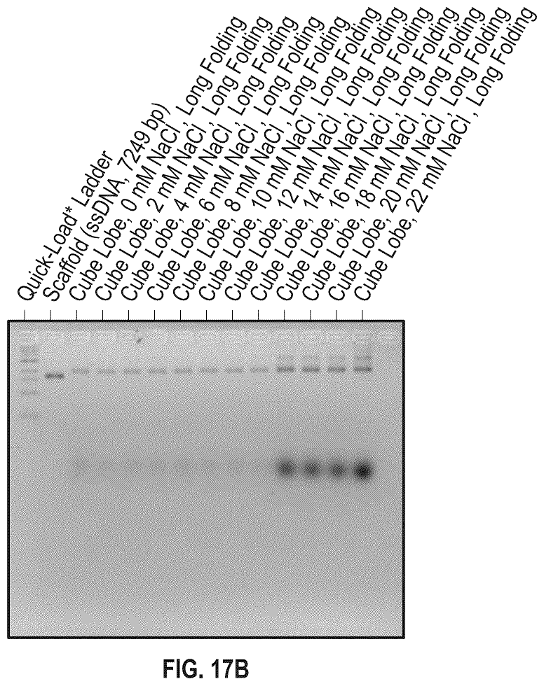

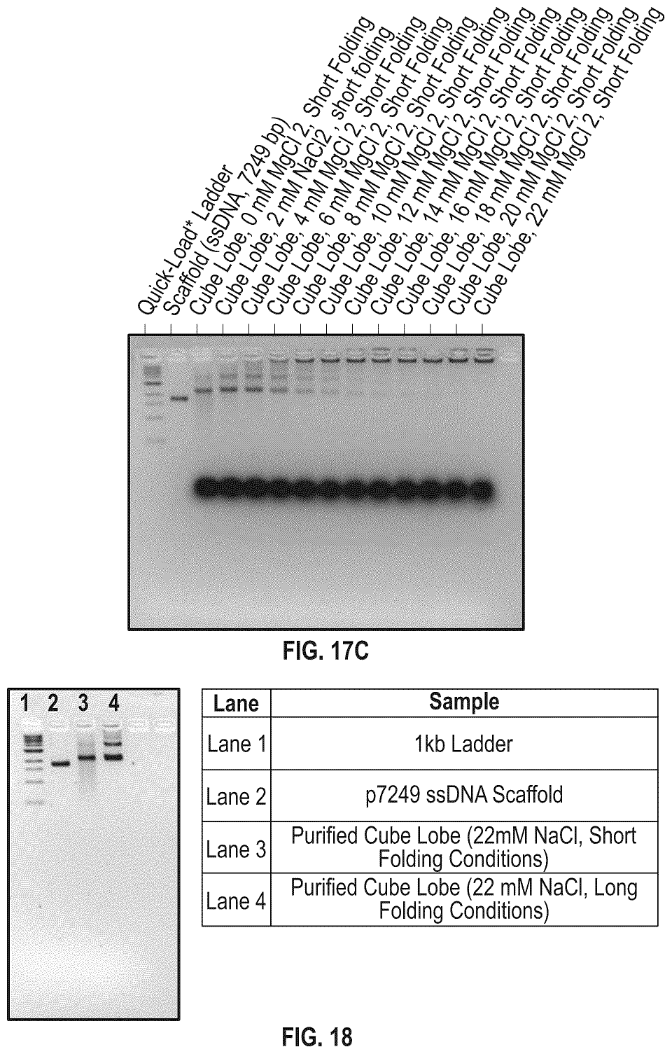

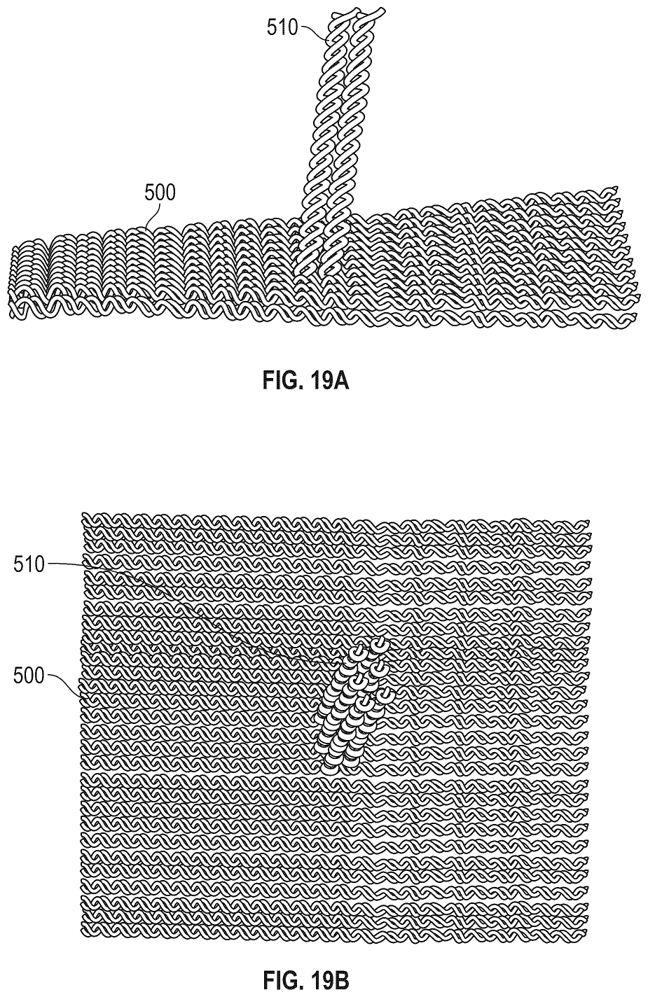

FIG. 17A shows results of gel electrophoresis of the Cube Lobe samples folded using different concentrations of NaCl and short folding conditions. The excess of staples (10× excess compared to the scaffold) appears as a large band that runs at low molecular weight values. The folded DNA origami structure appears as a single band characterized by an upper shift compared to the scaffold band and featuring the appearance of more faded bands at higher molecular weights values (FIG. 17A lane 14, Cube Lobe 22 mM NaCl short folding conditions). The smearing of the band visible for the DNA origami samples folded using lower NaCl concentrations (lanes 3-13, Cube Lobe samples folded using NaCl concentrations ranging from 0 mM to 20 mM) can be attributed to misfolded or unfolded samples. The results from this gel electrophoresis suggest that, if short folding conditions are used, concentrations of NaCl>22 mM are more likely to lead to correct folding of the structures. No smearing of the bands is visible when longer folding conditions are used (FIG. 17B) and lower concentrations of NaCl (ranging from 16 mM to 22 mM NaCl) suffice for a correct folding of the structures (FIG. 17B, lanes 11-14). The absence of the higher-molecular weight bands in the remaining DNA origami samples (FIG. 17B, lanes 3-10) may be related to incomplete folding of the structures, with partial formation of dsDNA that causes the upper shift of the band when compared to the ssDNA scaffold (FIG. 17B, lanes 2) but without the presence of other bands this may suggest the presence of larger structures. The results from these two gels suggests that the folding of the Cube Lobe DNA origami structure occurs when either short or long folding conditions are used and with concentrations of NaCl>16 mM. The same experiments were repeated substituting NaCl with MgCl₂ for the folding of the DNA origami structures. The corresponding gel electrophoresis analysis is shown in FIG. 17C. While the use of long folding conditions caused aggregation of the structures for the range of MgCl₂ concentrations screened (image not shown), the use of short folding conditions and MgCl₂ concentrations comprised between 2 mM and 6 mM led to a successful folding of the structures (FIG. 17C).

The successful purification of the Cube Lobe structure from the extra staples present in solution was demonstrated for the Cube Lobe folded using short folding conditions and long folding conditions in the presence of 22 mM of NaCl. For the purification, Amicon™ 100K, 2 mL spin filter columns were used. Briefly, the spin filter columns were first loaded with 2 mL of 1×TE solution pH 8 containing 22 mM of NaCl. The columns were centrifuged (4 min, 4000 rcf). Then, 2 mL of the DNA origami solution was loaded to each column. The columns were centrifuged (12 min, 4000 rcf) and the flow through was discarded. Fresh 1×TE pH 8, 22 mM NaCl solution was added to the filter columns to a total volume of ˜2 mL. The samples were centrifuged again (12 min, 4000 rcf) and the flow-through discarded. The same process was repeated ˜20 times. The samples were concentrated (no additional solution added) centrifuging for 2 min at 4000 rcf. The sample solution was collected by centrifuging for 2 min at 4000 rcf. After purification, 12 uL of the samples at 10 nM final concentration were loaded to a 0.8% gel containing SYBR SAFE. 10 uL of a 1 kb DNA ladder diluted in 1×OB (5 uL of ladder+5 uL of 1×OB) was added to the gel with 10 uL of a 10 nM solution of p7249 ssDNA scaffold used as control. The gel was run in 1×OB in an ice bath at 70V for 3 hours. As shown in FIG. 18, gel electrophoresis showed a successful purification of the structures from the extra staples using filter columns. While the use of filter columns produced a smearing of the band corresponding to the sample folded using 22 mM NaCl and short folding conditions, the samples folded using 22 mM NaCl and long folding conditions did not seem to be adversely affected by the purification method, resulting in high yield and high concentration of the DNA origami monomer fraction.

### Example VII

### Multiplex Assembly of a Plurality of Nucleic Acid Origami Structures

This example describes construction of ten nucleic acid origami particles, each with one variant staple, wherein the variants differ with regard to placement of a docker handle at one of ten different locations on a structured nucleic acid particle composed of nucleic acid origami. For ease of illustration, an origami with eleven staples is described.

Wells of a multi-well plate are loaded with beads attached to locked pulldown staples (LPS), named S1* through S10*. The LPSs include locked bases or other non-natural bases that form stronger Watson-Crick base pairs than nucleotides having naturally occurring bases. The plate contains relatively deep wells each having a relatively large surface area for high thermal exchange (e.g., “popsicle” shape wells). Wells are labeled A1-A10 as listed in Table 10.

TABLE 10

| A1 | A2 | A3 | A4 | A5 | A6 | A7 | A8 | A9 | A10 |
|---|---|---|---|---|---|---|---|---|---|
| S1* | S2* | S3* | S4* | S5* | S6* | S7* | S8* | S9* | S10* |

All ten standard staples S1-S10 are added to each well of the plate, and a scaffold nucleic acid is also added to each well of the plate. The same scaffold is added to each plate. The plate loading scheme is shown in Table 11.

TABLE 11

| A1 | A2 | A3 | A4 | A5 | A6 | A7 | A8 | A9 | A10 |
|---|---|---|---|---|---|---|---|---|---|
| S1*, | S2*, | S3*, | S4*, | S5*, | S6*, | S7*, | S8*, | S9*, | S10*, |
| S1-S10, | S1-S10, | S1-S10, | S1-S10, | S1-S10, | S1-S10, | S1-S10, | S1-S10, | S1-S10, | S1-S10, |
| Scaffold | Scaffold | Scaffold | Scaffold | Scaffold | Scaffold | Scaffold | Scaffold | Scaffold | Scaffold |

The wells of the plate are then covered and subjected to a thermal ramp. The reaction in each well yields nucleic acid origami structures immobilized on beads via hybridization of the LPSs to the scaffold nucleic acids. The wells also include excess staples. The excess staples are separated from the immobilized by centrifugal sedimentation of the beads followed by aspiration of the fluid-phase staples. The beads are then washed be delivery of wash fluid, centrifugation of the beads and aspiration of the wash fluid. The isolated, immobilized origami structures are ready for subsequent modification or use.

### Example VIII

### Assembly and Ion Exchange Purification of Nucleic Acid Origami Structures

DNA origami pegboards were prepared as follows. A master mix was formed including 5 mM NaCl, 1 mM EDTA, 5 mM Tris-HCl pH 8.0, 20 mM MgCl₂, 20 nM p8064 Scaffold DNA (Tilibit Nanosystems, Munich Germany) and 200 nM staple DNA. Contents were mixed in a 50 mL Nalgene bottle by gently inverting the bottle. To each of 12 falcon tubes was added 40 mL of the mixed solution. DNA was denatured by incubation for 12 minutes at 75° C. in a sous vide water bath followed by incubation overnight at 25° C. The sequences for the staple DNA species are listed in Table 12.

TABLE 12

| Sequence | | SEQ ID |
|---|---|---|
| Name | Sequence | NO |
| 1-028062 | TAACCCTATACACTAAAACAC | 1095 |
| 2-032062 | TTAAACAAATCTCCAAAAAAA | 1096 |
| 3-038083 | GCGGCCATGCCCCCTGCCTAT | 1097 |
| 4-044062 | GTAGCATTTGAGCCATTTGGG | 1098 |
| 5-020097 | ATTTGTAGCGCATAAAGATAAGAGCCAGTTTCACTCA | 1099 |
| | CCTCCATCTCCACTCCTACCCATCCAACTCCCACTTTC | |
| | TAGATGTAT | |
| 6-020118 | ATTGCGTATATTCCTACCGAATCTAAAGTTTCACTCAC | 1100 |
| | CTCCATCTCCACTCCTACCCATCCAACTCCCACTTTCT | |
| | AGATGTAT | |
| 7-020160 | TACCATACTGATTGTTAATGCATCAATATTTCACTCAC | 1101 |
| | CTCCATCTCCACTCCTACCCATCCAACTCCCACTTTCT | |
| | AGATGTAT | |
| 8-047140 | AGGCAAAGCAAGGCAACAGCCATATTAT | 1102 |
| 9-029073 | ATTTACATTGGGTGAGGCGGTGTACAGACCAG | 1103 |
| 10-033073 | CGAACGTGGCGTTTTAGACCTCAGCAGCGAAA | 1104 |
| 11-002055 | TTGAGGGCACCGACTAACATCTCAATTCTACTA | 1105 |
| 12-000090 | ACGCAATGTCAAATCACCATCAGCCCCAGTTAAAATT | 1106 |
| | TCACTCACCTCCATCTCCACTCCTACCCATCCAACTCC | |
| | CACTTTCTAGATGTAT | |
| 13-000132 | AAGCAGAAAATTAATGCCGGAACTAGCATAACCAA | 1107 |
| 14-001098 | AATATGATATTCAACCGTTCTACCCCGGTTGTTAATTT | 1108 |
| | CACTCACCTCCATCTCCACTCCTACCCATCCAACTCCC | |
| | ACTTTCTAGATGTAT | |
| 15-001140 | GAGGGTAGCTATTTTTGAGAGTCGATGAAAAATAATT | 1109 |
| | TCACTCACCTCCATCTCCACTCCTACCCATCCAACTCC | |
| | CACTTTCTAGATGTAT | |
| 16-016118 | ATCGTCGAAAGAAGAGAGCGGAAAGAGTCTGTCCA | 1110 |
| 17-022097 | AATCTTGTGAATTATTTTAAGAACTGGCTCATTAT | 1111 |
| 18-022139 | AAGAAACACAAACAACTAACAACTAATAGATTAGA | 1112 |
| 19-022160 | ACATTATATTAAATATCTAAAATATCTTACCCTCA | 1113 |
| 20-031133 | AATTAACCGTTGTAATCCAGAAGTAACAGTACCTT | 1114 |
| 21-033112 | CGGGCGCTAGGGCGTAGAATCATGATGAAACAAAC | 1115 |
| 22-035133 | AGTCCACTATTAAAAATCAAGAACATAGCGATAGC | 1116 |
| 23-036132 | GAGCCGGAGCCTCCCAGACGAAGGTTTCACGCAAC | 1117 |
| 24-037112 | TTAATGAATCGGCCGCGGTCCTAAATGCTGATGCA | 1118 |
| 25-039049 | TAAGAGGTCATTTTAGACCGGAGGTGTATCACCGT | 1119 |
| 26-039133 | TCACAGTTGAGGATTCCACACCTAGAAAAAGCCTG | 1120 |
| 27-041112 | CTGGTAATGGGTAATCCAGCGAGGCAGAGGCATTT | 1121 |
| 28-041154 | TTACACTGGTGTGTTTACCTGACCGACAAAAGGTA | 1122 |
| 29-043049 | CCATTAGATACATTGAAGTTTTTGAGGCAGGTCAG | 1123 |
| 30-043133 | CTCCGGCCAGAGCAGGTGGTGAAACCAATCAATAA | 1124 |
| 31-045084 | GATAACCGACGGCCCTCAGGAGTAACCGATATTTT | 1125 |
| 32-045112 | ACGTACAGCGCCATTACATCGTATAGAAGGCTTAT | 1126 |
| 33-045154 | TAGACTTTCTCCGTTTAAATTAGCGAACCTCCCGA | 1127 |
| 34-045168 | GGTGAAGACGCCAGGCGCAACGTAACAACTGGCCTTT | 1128 |
| | TCACTCACCTCCATCTCCACTCCTACCCATCCAACTCC | |
| | CACTTTCTAGATGTAT | |
| 35-037069 | CACTGCCCGCTTTCCGATGGTGAGCGTAACGATCTA | 1129 |
| 36-010055bio | /5Biosg/TTTGGGGTTTCCGGAATAAGCAAACGAGCTTC | 1130 |
| | AAAGCGAACGCT | |
| 37-000076 | GAATACCCAAAGACGCCAGTTTGAGGAAATATTTAAA | 1131 |
| | TTGTATTTCACTCACCTCCATCTCCACTCCTACCCATC | |
| | CAACTCCCACTTTCTAGATGTAT | |
| 38-000097 | CGAGGAATTATTTTGCGCATCAGATCGCACTCCAGCG | 1132 |
| | ACGTT | |
| 39-001056 | TTCAAAAGGGTGAGAAAGGCCGTATAAGCAAATAAA | 1133 |
| | AATTTTCACTCACCTCCATCTCCACTCCTACCCATCCA | |
| | ACTCCCACTTTCTAGATGTAT | |
| 40-002097 | TGGTTTACAGTAGCGTAAAACTCACCGGAAACAATCG | 1134 |
| | TAAAA | |
| 41-004076bio | /5Biosg/TTTGATAGCACGTTTGCAGTGATGAAGGGGCA | 1135 |
| | AATGGTCAATAAC | |
| 42-004097 | AACGTCACAAAATCAAAGCCGTCCGGCAAACGCGGC | 1136 |
| | AGCATC | |
| 43-006097 | ACCGCCTAAACAAAAGCGGGGGGGGTCACTGTTGCGC | 1137 |
| | CTGTG | |
| 44-008076bio | /5Biosg/TTTACCGTTCCAGTTAAGAATGCGGCGGGCGG | 1138 |
| | ATGGCTTAGAGCT | |
| 45-008097 | GAAAGCGTTCGGAACACTCTGTCTGCCAGCACGCGGG | 1139 |
| | GTGCC | |
| 46-010097 | ATTAAGACACCCTCTAATGAGAAACCTGTCGTGCCCA | 1140 |
| | GCAGG | |
| 47-011042 | GTGCCTTTTTGATGGCATTGACCACCCTGCATTTTGAA | 1141 |
| | TCAA | |
| 48-012097 | ACCCTCAAAGTTTTCGAAAATTAGCCCGAGATAGGGG | 1142 |
| | AACCC | |
| 49-014097 | TGAATTTATTGTATTAAAGGGAAGGGAAGAAAGCGA | 1143 |
| | CAGGAG | |
| 50-016097 | TGAATTTGACAGCAGCCGATTAATCAGTGAGGCCAGC | 1144 |
| | TCATG | |
| 51-017042 | TTTTTCAGAGTGAGACGCCTGACCCATGGTATAGCTG | 1145 |
| | CTCAG | |
| 52-018097 | CAGAGGCTATACCAGAAATACACCAGTCACACGACCC | 1146 |
| | AGCAG | |
| 53-022076 | TTCATTATAATTTCACCAGTCAGGACGTAGCACCGCC | 1147 |
| | TGCAATTTCACTCACCTCCATCTCCACTCCTACCCATC | |
| | CAACTCCCACTTTCTAGATGTAT | |
| 54-025098 | CCTTAACATTTGAGGATTTAGGCCGTCAATAGATAAT | 1148 |
| | TGCGA | |
| 55-031081 | GTGTTGACGCTCAATCGTCTGACAGGGCCAGAATCCT | 1149 |
| | GAGAA | |
| 56-031084 | TTTTTATAAAGGGAAGAAAGGAGCCCCCAAAAGAAC | 1150 |
| | CTGTTT | |
| 57-032083 | GATTTAGAGCTTGACGGGGCTAAGCAAAATCCCTTAT | 1151 |
| | AAATC | |
| 58-036083 | ACTCACATTAATTGCGTTGCCTGCCGTTTTCACGGTCA | 1152 |
| | TACC | |
| 59-037105 | AGCTGCAAAGCCTGTGCCTGTACTGCGCCCTGCGGAG | 1153 |
| | GTGTC | |
| 60-040083 | AGGCGCTTTCGCACTCAATTGTCTAAAGTTAAACGAT | 1154 |
| | GCTGA | |
| 61-044083 | AGTGCCAAGCTTTCAGAGGTATAGGACGACGACAGTA | 1155 |
| | TCGGC | |
| 62-045061 | ATAGTGGAGCCGCCACGGGAACGGGCCTTTCATCTTT | 1156 |
| | TCATAAT | |
| 63-014055 | TTCAGCGCGTTGAAGTTCAGAGAATCCCCCTCAAATG | 1157 |
| | AAAGCCGG | |
| 64-018055 | CATTAAACAAAAGACGTTTACGTAAGAGCAACACTAT | 1158 |
| | AATGGATT | |
| 65-000118 | ACAAAGTCCCTGAAAGGTCACTCCGGCACCGCTTCAC | 1159 |
| | GCCAGGGTTTTC | |
| 66-000160 | TCTTACCAGATAACGATTCTCTCGCCATTCAGGCTCTG | 1160 |
| | GCGAAAGGGGG | |
| 67-002139 | GAAGCGCCAAAATAGATTAAGAGTCCCGGAATTTGGC | 1161 |
| | CAGCAGTTGGGC | |
| 68-004118 | GAGCGTCAATCAGAACATAAATTTCGTCTCGTCGCCA | 1162 |
| | GCTTACGGCTGG | |
| 69-004160 | GCACCCAGCGTTTTTCTGCTCATAACGGAACGTGCAA | 1163 |
| | TGCCAACGGCAG | |
| 70-006139 | GTAGGAACATGTAGCCATCCCTTTGCTCGTCATAAGG | 1164 |
| | TGCCCCCTGCAT | |
| 71-008118 | AAGAAAAGTAATTTCAGTGTCTCTTCGCGTCCGTGAA | 1165 |
| | GCATAAAGTGTA | |
| 72-008160 | TGCAGAAATAAAGTCAGCCAGTACCGAGCTCGAATA | 1166 |
| | AATTGTTATCCGC | |
| 73-010139 | TTGAGAAATAATTAAACATACGGGGAGAGGCGGTTG | 1167 |
| | CCCTGAGAGAGTT | |
| 74-012118 | TAAGGCGCTATATGACGCTGGGTTGTTCCAGTTTGGG | 1168 |
| | TGCCGTAAAGCA | |
| 75-012160 | TGACCTAACGCGAGCCCTTCAGACTCCAACGTCAACA | 1169 |
| | CTACGTGAACCA | |
| 76-014139 | TTTTAACCCTTGAATTTTTTGGTGTAGCGGTCACGCGT | 1170 |
| | ATAACGTGCTT | |
| 77-016160 | ACATAAAACATTTATGCTTTGTTCTTTGATTAGTAACT | 1171 |
| | ATCGGCCTTGC | |
| 78-020076 | ATTGTGTGATGAACGGTCAGTATTAAATTTAGGAATA | 1172 |
| | CCACAAGATTCA | |
| 79-022048 | TGCTCATCCGAACTTGTTACTAAAGAGGCGGGTAACA | 1173 |
| | GGGAGAACCATC | |
| 80-022055 | ACAAAGCTAAATTGAAAAATCTACGTTAGGTAGAATT | 1174 |
| | CAACTAGGCATA | |
| 81-028111 | GAAAAACCCGAGTAGAGCTAAAAAGGAGCTAAATCG | 1175 |
| | TTGAGTTTTGCCC | |
| 82-028125 | AGCCATTGCAACAGAAAAGGGACATTCTTTAAAAATG | 1176 |
| | ATTATCAGATGA | |
| 83-043105 | TCCGTTTAAAATCCCGGCGAACCAGTCACCAGCTTGT | 1177 |
| | TGGTGTAGATGG | |
| 84-043126 | TGGCAGCGGTTGTGGTTTACCTTGGGTATGGTGCCGA | 1178 |
| | CCGTACATTTTT | |
| 85-005042 | TCACCGTAGGGAAGATAAAGGGACTCCTTGTGTAGGT | 1179 |
| | AAAGATAGAACCATTTCAA | |
| 86-006055 | AGAGCCACAGGAGGCATTCCAACTAAAGTACGGTGTC | 1180 |
| | CCGCCGGGCGCGGTTGCGG | |
| 87-012076 | ATTTTCACATAGTTGTTCCGAAATCGAGCGGATTGCA | 1181 |
| | TCAAATTATAGTCAGAAGC | |
| 88-016076 | TACCGATTCGTCACCAGGAACGGTACTAATAGTAAAA | 1182 |
| | TGTTTGTTTTGCCAGAGGG | |
| 89-018139 | GAGGCGAAATATACACAATATAGAGATAGAACCCTG | 1183 |
| | ATAGCCCTAAAACACCTCAA | |
| 90-026153 | GCGAACTTCTGACCTGGTAATGCAATACACGAGCACT | 1184 |
| | GCGCGTCACCCAGAACGTG | |
| 91-028132 | TACCGCCTCACGCATCCTCGTCTGGCAAGGGTCGAGA | 1185 |
| | ACAAGGCAGCAAAACGCGC | |
| 92-034048 | AAGATTATTTAATTCTCCAACCTTTTGATAATTGCATA | 1186 |
| | TGCATATAACAGTTGATT | |
| 93-034153 | CCGCCTGTGCGTATTCACAATCCCCGGGCGGTGCCAC | 1187 |
| | ATCCCCACCGTCCATCCTC | |
| 94-037084 | AGTCGGGTGAGCTAGGGGGTTTGGTGCTTATGAGCTC | 1188 |
| | ATTGCTTGCCGTCACAGGC | |
| 95-042153 | ATTTGCCTGAGAGAATGTGCTGCGCCATCGTGGGAGC | 1189 |
| | CATCAACGGTAATCGTAAA | |
| 96-003020 | TTTAAACGTAGAAAAGACCCTGTATTT | 1190 |
| 97-005020 | TTTATTCATTAAAGGGGCAAGGCATTT | 1191 |
| 98-007020 | TTTCTGTAGCGCGTTTTTCATTTGTTT | 1192 |
| 99-009020 | TTTACCACCAGAGCCCCCAATTCTTTT | 1193 |
| 100-011020 | TTTATAAGTTTTAACAATGCTGTATTT | 1194 |
| 101-013020 | TTTGTCGAGAGGGTTGATTAGAGATTT | 1195 |
| 102-015020 | TTTGTCACCAGTACAGCCCGAAAGTTT | 1196 |
| 103-017020 | TTTAGGAATTGCGAAATAAATCAATTT | 1197 |
| 104-019020 | TTTATTCGGTCGCTGCCAATACTGTTT | 1198 |
| 105-021020 | TTTAAGGCACCAACCAACCAAAATTTT | 1199 |
| 106-023020 | TTTACGGTCAATCATATACATAACTTT | 1200 |
| 107-025020 | TTTCTGACGAGAAACGAACTAACGTTT | 1201 |
| 108-040196 | TTTCTGGTCTGGTCAACGGGTATTTTT | 1202 |
| 109-042196 | TTTAGAGACGCAGAAGAGGTTTTGTTT | 1203 |
| 110-044196 | TTTTGCGGGCCTCTTTTTGTTTAATTT | 1204 |
| 111-046196 | TTTCAACATTAAATGCAATAATAATTT | 1205 |
| 112-000174 | AATAGCAAAGGCTATCAGGTCATTGCTTT | 1206 |
| 113-009035 | GCCGCCAATACAGGAGTGTACTGGTATTT | 1207 |
| 114-012182 | TTTTTTAGTTAATTTCGTTATACAAATTTT | 1208 |
| 115-016182 | TTTCTTTTTTAATGGTGAGAAGAGTCATTT | 1209 |
| 116-020182 | TTTTAATGGAAGGGTACAATAACGGATTTT | 1210 |
| 117-043023 | TTTGCGAACGAGTAGATTTAGTTTGACTGTTTA | 1211 |
| 118-008193 | TTTACGACGACAATAAACAAAGTAATTCTGTCCAGTT | 1212 |
| | T | |
| 119-026186 | TTTTATTTTTGAATGGCTATACGTGGCACAGACAATTT | 1213 |
| 120-028186 | TTTGAGTAGAAGAACTCAAATAACATCACTTGCCTTT | 1214 |
| | T | |
| 121-030186 | TTTCGCTACAGGGCGCGTAGCCGCGCTTAATGCGCTT | 1215 |
| | T | |
| 122-032186 | TTTTATCAGGGCGATGGCCAGGGCGAAAAACCGTCTT | 1216 |
| | T | |
| 123-034186 | TTTGTGAGACGGGCAACAGGTTTTTCTTTTCACCATTT | 1217 |
| 124-036186 | TTTAGCTGTTTCCTGTGTGTCGTAATCATGGTCATTTT | 1218 |
| 125-038186 | TTTGGCATCAGATGCCGGGTCAGCAAATCGTTAACTT | 1219 |
| | T | |
| 126-001025 | TTTTTATGCCTGAGTAATATTACGCAGTATGTTAGCTT | 1220 |
| | T | |
| 127-048191 | TTTTTCTGAGAGTCTGGTCCTGTAGCCAGCTTTCATTT | 1221 |
| | T | |
| 128-024188 | TTTTTGTTGAAAGGAATTGAGAGTTGGCAAATCAACA | 1222 |
| | TTT | |
| 129-033023 | TTTCGGAATCGTCATAAATATTCATTAAACGAGCTGA | 1223 |
| | CTA | |
| 130-027021 | TTTTTGAACAACATTATTACAATAAAACACCAGAACG | 1224 |
| | AGTAG | |
| 131-029154 | TGAAAGCGTAAGAATTAGTCTTTTGGATTATACTTCTG | 1225 |
| | AATTT | |
| 132-033154 | TAACCACCACACCCCTATGGTACAATTTCATTTGAATT | 1226 |
| | ACTTT | |
| 133-037154 | TGGGCGCCAGGGTGCTGATTGAAAACTTTTTCAAATA | 1227 |
| | TATTTT | |
| 134-006193 | TTTAAACCAAGTACCGCACTCCAAGAGCAGCAACCGC | 1228 |
| | AAGCGGACTTATCAAAC | |
| 135-000193 | TTTGAGCAAGAAACAATGATTAAGCCTGAGCGATGTT | 1229 |
| | GGGAAGGGCGATCGGTTT | |
| 136-002193 | TTTCGTCAAAAATGAAAATACGATTTCGCTATTGGAT | 1230 |
| | AGCTCTCACGGAAAATTT | |
| 137-004193 | TTTAAGCCTTAAATCAAGACTTGCGGACAGCGGGTAG | 1231 |
| | AACGTCAGCGTGGTGTTT | |
| 138-029023 | TTTGCCAAAAGGAATTACGAATGCAGAAGGGAATCA | 1232 |
| | GTGAATAAGGCTTGCCTTT | |
| 139-031023 | TTTAGCGAGAGGCTTTTGCCGATAAATAAAACGTAGC | 1233 |
| | CGGAACGAGGCGCAGTTT | |
| 140-035023 | TTTAAATCAGGTCTTTACCAATGACCTAATAATGCCC | 1234 |
| | ACGCATAACCGATATTTT | |
| 141-037023 | TTTACTTCAAATATCGCGTAGAGGAAAACTACAAATA | 1235 |
| | GAAAGGAACAACTAATTT | |
| 142-039023 | TTTGTACCTTTAATTGCTCAGGTCAGGATATAATACCG | 1236 |
| | TAACACTGAGTTTCTTT | |
| 143-041023 | TTTGCTCAACATGTTTTAATGAATATGGGGTCATACCA | 1237 |
| | GGCGGATAAGTGCCTTT | |
| 144-045023 | TTTGGGCGCGAGCTGAAAAGCTATATTTCATCGCAGA | 1238 |
| | GCCGCCACCAGAACCTTT | |
| 145-047023 | TTTAAGAATTAGCAAAATTTCATACATGAATTAGTTT | 1239 |
| | GCCTTTAGCGTCAGATTT | |
| 146-049023 | TTTATACTTTTGCGGGAGAACATTATTACATACGTAA | 1240 |
| | ATATTGACGGAAATTTTT | |
| 147-028048 | CAGACGAAAAAGAAAGACTGGATAGCGTAGGCTTGA | 1241 |
| | ATACGTAATGCCACTACGTTT | |
| 148-045042 | GGTGGCACAATAAAAAGCAATACCAAAAAGCCTTTCT | 1242 |
| | CATATATTTTAAATGCATTT | |
| 149-010182- | TTTTCTTACCAGTATAAAGCCATTTCGTTTCTTTGCTT | 1243 |
| T3.3p- | GTGTCTTGTCTTCGTCGTGCTTGTTCC | |
| DYE40.22.9.3p | | |
| 150-014182- | TTTATAGTGAATTTATCAAAATTTTCGTTTCTTTGCTT | 1244 |
| T3.3p- | GTGTCTTGTCTTCGTCGTGCTTGTTCC | |
| DYE40.22.9.3p | | |
| 151-018182- | TTTTCGCCTGATTGCTTTGAATTTTCGTTTCTTTGCTTG | 1245 |
| T3.3p- | TGTCTTGTCTTCGTCGTGCTTGTTCC | |
| DYE40.22.9.3p | | |
| 152-022182- | TTTAATTTTAAAAGTTTGAGTATTTCGTTTCTTTGCTTG | 1246 |
| T3.3p- | TGTCTTGTCTTCGTCGTGCTTGTTCC | |
| DYE40.22.9.3p | | |
| 153-002069- | CGACATTAGAAACGCAAAAGAACTGGCATTTCGTTTC | 1247 |
| T3.3p- | TTTGCTTGTGTCTTGTCTTCGTCGTGCTTGTTCC | |
| DYE40.22.9.3p | | |
| 154-002153- | AACATAAATCAGAGGAAGCCCTTTTTAATTTCGTTTCT | 1248 |
| T3.3p- | TTGCTTGTGTCTTGTCTTCGTCGTGCTTGTTCC | |
| DYE40.22.9.3p | | |
| 155-003098- | GTCACAATCAATCATACCAGAAGGAAACTTTCGTTTC | 1249 |
| T3.3p- | TTTGCTTGTGTCTTGTCTTCGTCGTGCTTGTTCC | |
| DYE40.22.9.3p | | |
| 156-004090- | CCAATGAAAATCACCCAGCGCCAAAGACTTTCGTTTC | 1250 |
| T3.3p- | TTTGCTTGTGTCTTGTCTTCGTCGTGCTTGTTCC | |
| DYE40.22.9.3p | | |
| 157-004132- | TTACCAACCAGTTAATTAGACGGGAGAATTTCGTTTC | 1251 |
| T3.3p- | TTTGCTTGTGTCTTGTCTTCGTCGTGCTTGTTCC | |
| DYE40.22.9.3p | | |
| 158-004174- | TTAGTTGATAAGAAAGCAGCCTTTACAGTTTCGTTTCT | 1252 |
| T3.3p- | TTGCTTGTGTCTTGTCTTCGTCGTGCTTGTTCC | |
| DYE40.22.9.3p | | |
| 159-006069- | GAACCGCTTATTAGGCACCGTAATCAGTTTTCGTTTCT | 1253 |
| T3.3p- | TTGCTTGTGTCTTGTCTTCGTCGTGCTTGTTCC | |
| DYE40.22.9.3p | | |
| 160-006153- | TTTTTATACGCGAGGCTACAATTTTATCTTTCGTTTCTT | 1254 |
| T3.3p- | TGCTTGTGTCTTGTCTTCGTCGTGCTTGTTCC | |
| DYE40.22.9.3p | | |
| 161-007098- | ACCGGAACCAGACATTAGCAAGGCCGGATTTCGTTTC | 1255 |
| T3.3p- | TTTGCTTGTGTCTTGTCTTCGTCGTGCTTGTTCC | |
| DYE40.22.9.3p | | |
| 162-008090- | CAGTCTCTATTCACCCCTCAGAGCCGCCTTTCGTTTCT | 1256 |
| T3.3p- | TTGCTTGTGTCTTGTCTTCGTCGTGCTTGTTCC | |
| DYE40.22.9.3p | | |
| 163-008132- | GATAAGTTTACGAGTCATTACCGCGCCCTTTCGTTTCT | 1257 |
| T3.3p- | TTGCTTGTGTCTTGTCTTCGTCGTGCTTGTTCC | |
| DYE40.22.9.3p | | |
| 164-008174- | ACATGTTTTATCATTCATCGAGAACAAGTTTCGTTTCT | 1258 |
| T3.3p- | TTGCTTGTGTCTTGTCTTCGTCGTGCTTGTTCC | |
| DYE40.22.9.3p | | |
| 165-010069- | GGATTAGGTATAAACAGTAAGCGTCATATTTCGTTTC | 1259 |
| T3.3p- | TTTGCTTGTGTCTTGTCTTCGTCGTGCTTGTTCC | |
| DYE40.22.9.3p | | |
| 166-010153- | ACAGTAGAGAGAATCGCGCCTGTTTATCTTTCGTTTCT | 1260 |
| T3.3p- | TTGCTTGTGTCTTGTCTTCGTCGTGCTTGTTCC | |
| DYE40.22.9.3p | | |
| 167-011098- | CCTATTATTCTGATATAAAGCCAGAATGTTTCGTTTCT | 1261 |
| T3.3p- | TTGCTTGTGTCTTGTCTTCGTCGTGCTTGTTCC | |
| DYE40.22.9.3p | | |
| 168-012090- | GAGCCACGTACCGCGGCTGAGACTCCTCTTTCGTTTCT | 1262 |
| T3.3p- | TTGCTTGTGTCTTGTCTTCGTCGTGCTTGTTCC | |
| DYE40.22.9.3p | | |
| 169-012132- | GACCGTGCGGAATCTCGCCATATTTAACTTTCGTTTCT | 1263 |
| T3.3p- | TTGCTTGTGTCTTGTCTTCGTCGTGCTTGTTCC | |
| DYE40.22.9.3p | | |
| 170-014069- | CAACTTTCAGCCCTGGGATAGCAAGCCCTTTCGTTTCT | 1264 |
| T3.3p- | TTGCTTGTGTCTTGTCTTCGTCGTGCTTGTTCC | |
| DYE40.22.9.3p | | |
| 171-014153- | CTGAGAGACAAAGAAATTTAATGGTTTGTTTCGTTTCT | 1265 |
| T3.3p- | TTGCTTGTGTCTTGTCTTCGTCGTGCTTGTTCC | |
| DYE40.22.9.3p | | |
| 172-015098- | GTCGTCTTTCCAAATTCTCAGAACCGCCTTTCGTTTCT | 1266 |
| T3.3p- | TTGCTTGTGTCTTGTCTTCGTCGTGCTTGTTCC | |
| DYE40.22.9.3p | | |
| 173-016090- | CTTAAACGCCTTTATCTGTATGGGATTTTTTCGTTTCTT | 1267 |
| T3.3p- | TGCTTGTGTCTTGTCTTCGTCGTGCTTGTTCC | |
| DYE40.22.9.3p | | |
| 174-016132- | CCTTGCTTTAGAATCTCCGGCTTAGGTTTTTCGTTTCTT | 1268 |
| T3.3p- | TGCTTGTGTCTTGTCTTCGTCGTGCTTGTTCC | |
| DYE40.22.9.3p | | |
| 175-018069- | CATGAGGTGCGGGAAGTTGCGCCGACAATTTCGTTTC | 1269 |
| T3.3p- | TTTGCTTGTGTCTTGTCTTCGTCGTGCTTGTTCC | |
| DYE40.22.9.3p | | |
| 176-018153- | TACAAAAATTAATTTCAATATATGTGAGTTTCGTTTCT | 1270 |
| T3.3p- | TTGCTTGTGTCTTGTCTTCGTCGTGCTTGTTCC | |
| DYE40.22.9.3p | | |
| 177-019098- | TCGGAACGAGGGCACTTTGCTTTCGAGGTTTCGTTTCT | 1271 |
| T3.3p- | TTGCTTGTGTCTTGTCTTCGTCGTGCTTGTTCC | |
| DYE40.22.9.3p | | |
| 178-020090- | TCATCGCCAGCGATTTTGAGGACTAAAGTTTCGTTTCT | 1272 |
| T3.3p- | TTGCTTGTGTCTTGTCTTCGTCGTGCTTGTTCC | |
| DYE40.22.9.3p | | |
| 179-020132- | ACAGAAATCAGATGATTATTCATTTCAATTTCGTTTCT | 1273 |
| T3.3p- | TTGCTTGTGTCTTGTCTTCGTCGTGCTTGTTCC | |
| DYE40.22.9.3p | | |
| 180-022069- | CCCAAATGAGGACACGAAATCCGCGACCTTTCGTTTC | 1274 |
| T3.3p- | TTTGCTTGTGTCTTGTCTTCGTCGTGCTTGTTCC | |
| DYE40.22.9.3p | | |
| 181-022153- | CATTTTGTATAATCTCAAAATTATTTGCTTTCGTTTCTT | 1275 |
| T3.3p- | TGCTTGTGTCTTGTCTTCGTCGTGCTTGTTCC | |
| DYE40.22.9.3p | | |
| 182-023098- | GGCTGGCTGACCTCAGAGTACAACGGAGTTTCGTTTC | 1276 |
| T3.3p- | TTTGCTTGTGTCTTGTCTTCGTCGTGCTTGTTCC | |
| DYE40.22.9.3p | | |
| 183-025077- | AACTTTAATCATTGACAAGAACCGGATATTTCGTTTCT | 1277 |
| T3.3p- | TTGCTTGTGTCTTGTCTTCGTCGTGCTTGTTCC | |
| DYE40.22.9.3p | | |
| 184-025119- | AAGTATTAGACTTTCACCAGAAGGAGCGTTTCGTTTC | 1278 |
| T3.3p- | TTTGCTTGTGTCTTGTCTTCGTCGTGCTTGTTCC | |
| DYE40.22.9.3p | | |
| 185-002118 | TTAACTGAAAGAAAATTCATA | 1279 |
| 186-006118 | AATAGCAAGGCCACCACCGGA | 1280 |
| 187-010118 | AACGCCAACAAACATGAAAGT | 1281 |
| 188-014118 | GGGTTATATGACGTTAGTAAA | 1282 |
| 189-018118 | TTACCTGAGTAGCAACGGCTA | 1283 |
| 190-022118 | GAATTATCATTCATCAAGAGT | 1284 |
| 191-000055 | TGATTAATGGCAACATATAAACAACCGA | 1285 |
| 192-000139 | GAAAAGTAATTGAGCGCTAATAAACAGG | 1286 |
| 193-002076 | AAAAGGGAATTAGAGCCAGCAAACCATC | 1287 |
| 194-002160 | AGAGAATTTATCCCAATCCAACTATTTT | 1288 |
| 195-004055 | AGCGACACGGTCATAGCCCCCCACCCTC | 1289 |
| 196-004139 | CTGAATCCCGGTATTCTAAGATTTCATC | 1290 |
| 197-006076 | ACCCTCAACGATTGGCCTTGATGAATTT | 1291 |
| 198-006160 | CAAGCCGTCGGCTGTCTTTCCCAGCTAA | 1292 |
| 199-008055 | CATGGCTGAGTAACAGTGCCCGATTAGC | 1293 |
| 200-008139 | AACAATATCGAGCCAGTAATAGGCTTAA | 1294 |
| 201-010076 | AAGAGAAACTCAGGAGGTTTACACCCTC | 1295 |
| 202-010160 | ACGCTCATTTAGTATCATATGCATCTTC | 1296 |
| 203-012055 | AATAGGATAGCATTCCACAGACAACAGT | 1297 |
| 204-012139 | AAATACCAATCCAATCGCAAGACTACCT | 1298 |
| 205-014076 | TGCTAAAAGGCTCCAAAAGGAAGCTTGA | 1299 |
| 206-014160 | CATAGGTTTAGATTAAGACGCAAACAGT | 1300 |
| 207-016055 | TGACAACTTAAAGGCCGCTTTAAGTTTC | 1301 |
| 208-016139 | TGAATAAATCAAGAAAACAAATCGCGCA | 1302 |
| 209-018076 | ACTTTTTTCATCTTTGACCCCCTGATAA | 1303 |
| 210-018160 | ACCAAGTTTACATCGGGAGAATAGAACC | 1304 |
| 211-020055 | TGCTCCAGACCAACTTTGAAACAACGTA | 1305 |
| 212-020139 | ACGTAAATGGCAATTCATCAACGGAACA | 1306 |
| 213-048090 | AAAACAGGAAGATTGGAGACAAATAACG | 1307 |
| 214-048132 | TGTCAATCATATGTAGCTGATTAGCCGA | 1308 |
| 215-048174 | AGCAAACAAGAGAAATCTACAATAGCTA | 1309 |
| 216-005098 | ACCATTACCATTTCCAGAGCCTAATTTGCGCTAAC | 1310 |
| 217-009098 | TAAATCCTCATTAATATCCCATCCTAATCCTGAAC | 1311 |
| 218-013098 | AGAACCGCCACCAAATAAGAATAAACACTGATAAA | 1312 |
| 219-017098 | CGGTTTATCAGCATTAATTAATTTTCCCTCTGTAA | 1313 |
| 220-021098 | AGCGCGAAACAAATTTTCAGGTTTAACGTAAAGAA | 1314 |
| 221-027056- | /5Biosg/TTTTCAGTTGTGGGAAGGGCTTGAGATGGTT | 1315 |
| T3.5p-BIO.5p | | |
| 222-027098- | /5Biosg/TTTCAGCAAATGAAAAACGAACCACAGTAAT | 1316 |
| T3.5p-BIO.5p | | |
| 223-027140- | /5Biosg/TTTATATCAATAGGAGCATTCGACAACTCGT | 1317 |
| T3.5p-BIO.5p | | |
| 224-049098- | /5Biosg/TTTTTCGCATTAAATTTTTGATAATCAGAAA | 1318 |
| T3.5p-BIO.5p | | |
| 225-049140- | /5Biosg/TTTTAGGAACACAAACGGCGGATTGGAAACC | 1319 |
| T3.5p-BIO.5p | | |
| 226-049056- | /5Biosg/TTTCGCAAGGGCTAAATCGGTTGTAAAGCCTC | 1320 |
| T3.5p-BIO.5p | AGAGCA | |
| 227-027119- | /5Biosg/TTTCATCACCTTGCTGAATCGCCAGGCCAAC | 1321 |
| T3.5p-BIO.5p | | |
| 228-049077- | /5Biosg/TTTAACGTTATGCATCTACCACGGAATAAGT | 1322 |
| T3.5p-BIO.5p | | |
| 229-049119- | /5Biosg/TTTATCAGCTATGGGATCAAAGTCAGAGGGT | 1323 |
| T3.5p-BIO.5p | | |
| 230-049161- | /5Biosg/TTTTTCGCGTCCCGTCGCCACAAGAATTGAG | 1324 |
| T3.5p-BIO.5p | | |
| 231-027077- | /5Biosg/TTTCAGTGCCACGCTGAAACAGAGCAGATTCC | 1325 |
| T3.5p-BIO.5p | TACATT | |
| 232-027161- | /5Biosg/TTTTCTGGTCGAAGGTTCCTTTGCCCGAACGTT | 1326 |
| T3.5p-BIO.5p | ATTTTT | |
| 221-027056- | TTTTCAGTTGTGGGAAGGGCTTGAGATGGTT | 1327 |
| T3.5p-NULL.5p | | |
| 222-027098- | TTTCAGCAAATGAAAAACGAACCACAGTAAT | 1328 |
| T3.5p-NULL.5p | | |
| 223-027140- | TTTATATCAATAGGAGCATTCGACAACTCGT | 1329 |
| T3.5p-NULL.5p | | |
| 224-049098- | TTTTTCGCATTAAATTTTTGATAATCAGAAA | 1330 |
| T3.5p-NULL.5p | | |
| 225-049140- | TTTTAGGAACACAAACGGCGGATTGGAAACC | 1331 |
| T3.5p-NULL.5p | | |
| 226-049056- | TTTCGCAAGGGCTAAATCGGTTGTAAAGCCTCAGAGC | 1332 |
| T3.5p-NULL.5p | A | |
| 227-027119- | TTTCATCACCTTGCTGAATCGCCAGGCCAAC | 1333 |
| T3.5p-NULL.5p | | |
| 228-049077- | TTTAACGTTATGCATCTACCACGGAATAAGT | 1334 |
| T3.5p-NULL.5p | | |
| 229-049119- | TTTATCAGCTATGGGATCAAAGTCAGAGGGT | 1335 |
| T3.5p-NULL.5p | | |
| 230-049161- | TTTTTCGCGTCCCGTCGCCACAAGAATTGAG | 1336 |
| T3.5p-NULL.5p | | |
| 231-027077- | TTTCAGTGCCACGCTGAAACAGAGCAGATTCCTACAT | 1337 |
| T3.5p-NULL.5p | T | |
| 232-027161- | TTTTCTGGTCGAAGGTTCCTTTGCCCGAACGTTATTTT | 1338 |
| T3.5p-NULL.5p | T | |

DNA origami pegboards were separated from staples and other reaction components as follows. Anion exchange columns (Qtip 200 columns, Qiagen, Hilden Germany) were washed with 4 CV (column volume=5.5 mL) of 1×OB (5 mM Tris, pH 8.0, 200 mM NaCl, 12.5 mM MgCl₂, 1 mM EDTA). About 30 mL of the fluid from the DNA origami pegboard assembly reaction was loaded onto the column via gravity flow and the eluate was collected. The column was then washed with 2 CV of 1×OB and collected with the eluate (also referred to as the flow through fraction). Four CV of 1×OB+0.85 M NaCl (5 mM Tris, pH 8.0, 850 mM NaCl, 12.5 mM MgCl₂, 1 mM EDTA) was flowed through the column and the eluate collected. Then four CV of 1×OB+1.35 M NaCl (5 mM Tris, pH 8.0, 1350 mM NaCl, 12.5 mM Mg Cl₂, 1 mM EDTA) was flowed through the column and the eluate collected. The three eluate samples were analyzed on an agarose gel which demonstrated elution of relatively pure DNA origami pegboards on both the flow through fractions and 1.35 M NaCl eluates. The 0.85 M eluates contained the majority of the staples.

### Example IX

### Parallelepipedon Design for Attachment of Analytes to Nucleic Acid Origami Structures

This example describes nucleic acid origami structures for presenting attached analytes to solution phase reagents and overcomes multi-occupancy issues of the landing pad. In some instances, multiple occupancy is due to the possibility of two or more DNA origami structures occupying the same space upon deposition. This design includes re-routing the scaffold structure (ssDNA necessary for synthesis of the double-helices that constitute the DNA origami structure) and decreasing the height of the post that can display and present macromolecular analytes such as proteins and nucleic acids. The design also includes an additional design unit that increases the surface area of the entire nucleic acid origami structure. In many cases, the additional design unit functions as a base for attachment of various moieties such as labels, linkers or others set forth herein. The table below depicts structural differences between two nucleic acid origami structures.

TABLE 13

| | Design 1 - | Design 2 - |
|---|---|---|
| Structure Feature | Post | Short Post |
| Number of dye handles | 50 | 50 |
| Number of dockers | 0-4 | 0-4 |
| Number of jellyfish legs | 22 | 22 |
| Height of the post | About 30 nm | About 20 nm |
| Dimension of the additional | None present | About 13 nm × about |
| structural piece | | 8 nm (surface) |

The nucleic acid origami structures are configured to have a surface, a post protruding from the surface, and an additional parallelepipedon unit. The posts in this example are shorter than about 30 nm and particularly well suited for presenting macromolecular analytes such as proteins and nucleic acids since the posts set the macromolecules apart from the origami surface, thereby increasing accessibility of the macromolecule to solution-phase reagents.

The area of the surface can be configured to accommodate attachment of various moieties such as labels, linkers or others set forth herein. The area of the surface can also be configured to provide steric exclusion when attached to a surface (e.g., on a particle or at a site on the surface of an array). As such the nucleic acid origami, once attached to the surface, will preclude other nucleic acid origami from also attaching. In this example, the surface is planar. However, the surface need not be planar and can instead, for example, be curved, concave, convex or corrugated.

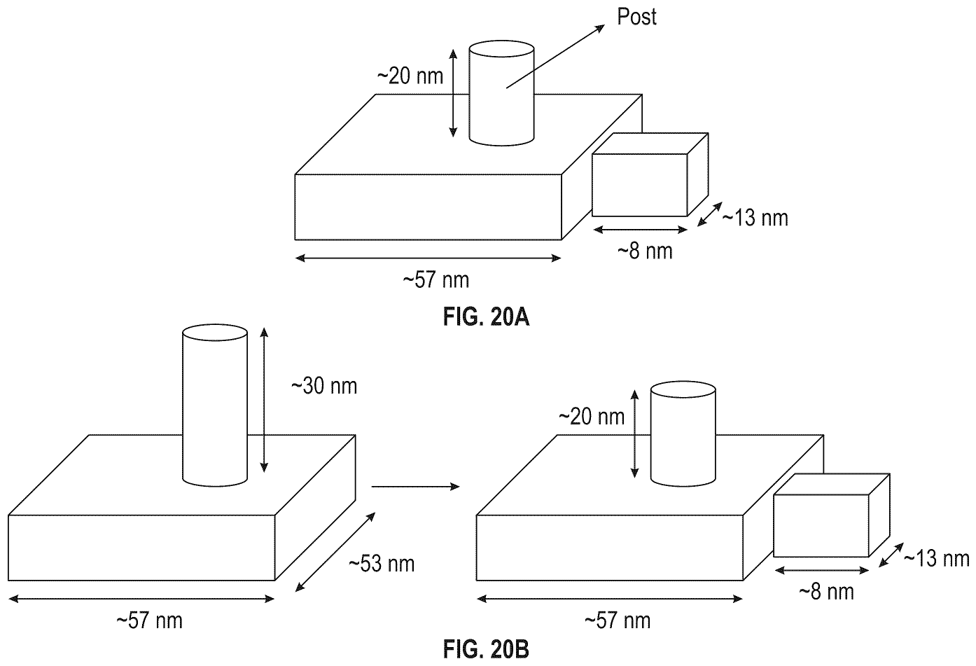

The origami was designed using CaDNAno v2.4 software and the hexagonal lattice option. The structure included 68 helices distributed in 2 different layers, in order to obtain a structure as diagrammed in FIG. 20A.

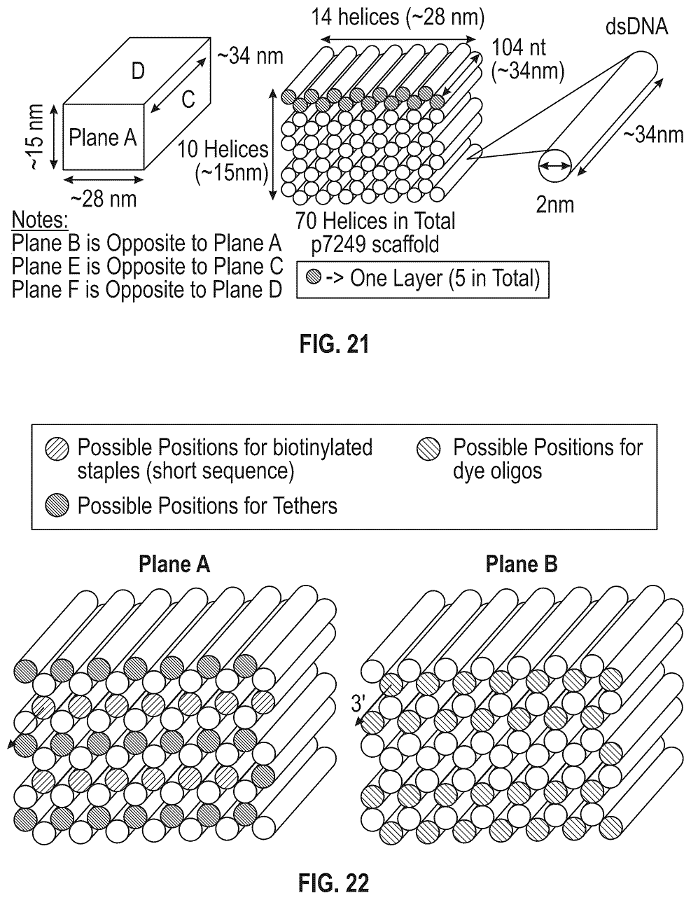

Compared to other designs, the design described herein has a shorter post for the target display and it has a larger surface area due to the presence of an additional parallelepipedon unit. FIG. 21B shows a comparison between the two structures and highlights the main differences between the two.

With the parallelepipedon design described herein, the protein analyte is attached closer to the surface of the base. Such a design opens the possibility to display nucleic acids such as dockers for Lobe interaction also on the base of the structure. By situating the protein analyte closer to the surface of the DNA origami structure, dockers present on the base of the origami may interact more easily with tethers on a Lobe (such as, but not limited to, a cube Lobe or brick Lobe), compared to when the post has a greater height such as about 30 nm.

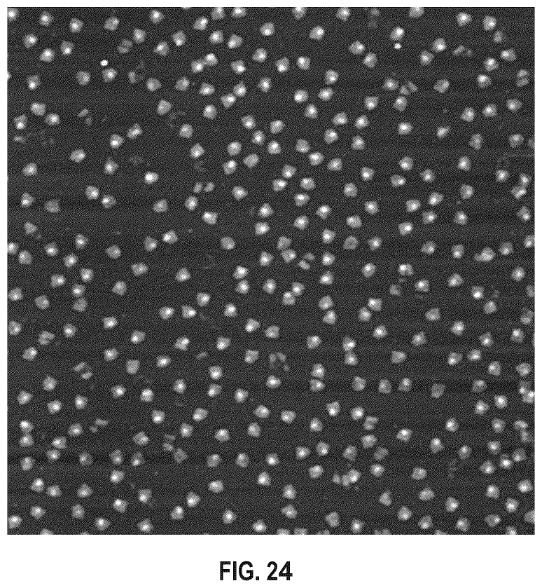

FIG. 24 shows an atomic force microscopy (AFM) image of nucleic acid origamis having parallelepipedon structures and protruding post structures.

### Example X

### Reducing Aggregation and Increasing Stability by Cross-Linking

Methods were used to prevent aggregation of DNA origami based on stability enhancement of the structure using cross-linking strategies as described above and in Example II. It has been described how aggregation of DNA origami structures can be caused by design-related features present in both well-folded and misfolded structures (e.g., presence of blunt ends, scaffold loops, etc.). Moreover, techniques used to strengthen the DNA origami structures (e.g., UV-crosslinking, addition of intercalators, etc.) are well-known in the art for preventing misfolding.

Specific misfolding mechanisms of DNA origami structures with posts have been observed during formation. For instance, an increase in the population of the misfolded samples, with the presence of free staples was seen. Loss of staples in a number of areas of the DNA origami structure was observed such as at an edge of the structure and areas close to the post. In some instances, the integrity and stability of the post and the DNA origami were lost.

Using imaging analysis, DNA origami structures in aggregation were seen as well as misfolded structures and free staples in solution. The images were taken under AFM in dry conditions of the sample after four days of incubation in 0.5λTBE buffer+11 mM MgCl₂. The images showed an increase in the population of the misfolded samples, with the presence of free staples. Analysis also showed a progression of the instability of the DNA origami upon loss of staple strands.

Loss of staple strands in some regions of the structure promoted binding between unhybridized regions of the structure. Formation of dimers or higher-order assemblies were observed. Aggregates can be caused by unspecific interactions between misfolded monomer structures. Formation of aggregates between the structures can occur between edges, corners, or more centered parts of the origami, thereby generating different possible aggregate configurations. Some observed aggregate configurations included complete overlapping of multiple DNA origami structures, interactions at the edges of multiple DNA origami structures, and interactions at corners and overlapping multiple DNA origami structures.

Aggregate formation of multimeric structures can occur between misfolded structures from staple loss in different regions of the structures include, but not limited to edges, corners, sides, and regions close to the post. In some instances, dimeric or multimeric aggregates form between misfolded structures.

Increasing structural stability of DNA origami structures by cross-linking methods prevents loss of staple strands, and also may consequently prevent aggregate formation as shown, for example, in Example II.

### Example XI

### Increasing Docker Handles on Nucleic Acid Origami Structures

This example describes a nucleic acid origami structure design with increased surface area of a post structure. The structure design also allows for increases in the number of locations for docker handles and target analyte display on the top surface of the post.

The origami was designed using CaDNAno v2.4 software and the hexagonal lattice option. The structure included 64 helices distributed in 2 different layers. 14 of these helices form a post structure. A schematic of the nucleic acid origami structure and corresponding dimensions across the different planes are presented in FIGS. 20A-20B.

This nucleic acid origami structure design has a post of about 15 nm height (shorter than a post of about 30 nm) and with a larger surface area from the addition of extra helices to the post structure. The height and surface area of post allows for a higher number of locations for docker handles and target analyte display on the post surface.

FIGS. 20A-20B show two structures and highlights the main differences between the two.

The design allows additional dockers to be located on the top surface of the post structure. Also, it allows an analyte target (e.g., a protein target) to be closer to the base of the DNA origami. In some cases, docker handles for Lobe interaction are also displayed on this part of the structure. With the target analyte closer to the surface of the DNA origami structure, dockers present on the base of the origami can interact more easily with the tethers on the Lobe, compared to a situation where the post is about 30 nm in height.

### Example XII

### Three-Dimensional and Asymmetrical DNA Origami Design for Antibodies, Dyes and Tethers Display

This example describes a three-dimensional DNA origami structure having the shape of a brick. The brick shape provides multiple sites for different functional components, such as different affinity reagents, label components, and other assay reagents (e.g., tether handles) to be displayed on different planes of the structure. In this example, the DNA origami structure has a brick shape with dimensions of about 34 nm×about 28 nm×about 15 nm. The structure includes 70 helices in total. As noted above, this structure can be used to simultaneously display multiple different types of reagent groups, such as affinity reagents, like antibodies, aptamers, nucleic acid groups, etc., labeling groups, attachment groups, and other functional moieties on any of the surfaces of the multiple planes. In the example shown, affinity reagents and tethers can be attached on one or more planes of the Lobe.

Advantages of the three-dimensional DNA origami structure described herein can include small dimensions, optimal presentation of different reagents on the surface(s) of the DNA origami structure, adequate number of affinity reagents, tethers and other assay reagents on the structure, reduced steric hindrance, and increased accessibility of the three-dimensional DNA origami structure to a target analyte.

This example describes a brick-shape DNA origami structure including DNA helices arranged in a hexagonal lattice of the ssDNA scaffold M13mp18 (7249 bp). The origami was designed using CaDNAno v2.4 software and the hexagonal lattice option. The structure included 70 helices distributed in 5 different layers, in order to obtain a structure with dimensions of 34 nm×about 28 nm×about 15 nm as diagrammed in FIG. 21.

Two different configurations for display of affinity reagents (e.g., antibodies), tethers and dye labels (e.g., fluorophores) on the brick-shaped origami structure are provided below.

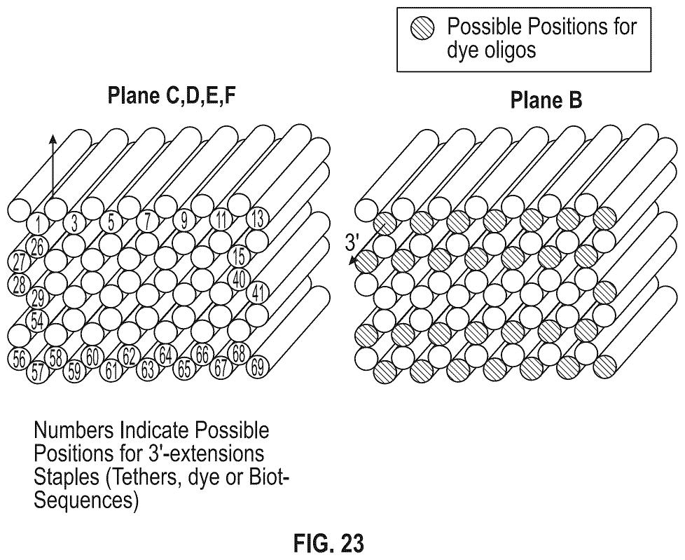

FIG. 22 depicts Configuration 1. In Configuration 1, antibodies and tethers are displayed on Plane A while fluorescent dye groups are displayed on Plane B (refer to FIG. 23 for specifications and labeling of the planes). The total number of functionalized staples based on the locations highlighted in the design would be: 13 biotinylated staples, 22 tethers and 28 fluorescent dye groups.

FIG. 23 depicts Configuration 2. Antibodies and tethers are displayed on all planes while fluorescent dyes will be distributed between plane A and B (refer to FIG. 23 for specifications and labeling of the planes). The total number of functionalized staples based on the locations highlighted in the design would be: 29 tethers/biotins and 28 fluorescent dye groups.

### Example XIII

### Synthesizing Nucleic Acid Origami Structures Using 19 kb ssDNA Scaffold Strands

This example describes nucleic acid origami structures created to have a much larger footprint, to increase the likelihood of single occupancy of landing pads by an origami particle. In particular, by increasing a particle's lateral cross sectional dimension(s) to greater than 50 nm, greater than 60 nm, greater than 70 nm, greater than 80 nm, one may be able to ensure mush higher single occupancy rates for landing pads on an array. In this example, an origami structure is synthesized from ssDNA scaffold strands that are about 19 kb. The resulting nucleic acid origami structures generated can be about 83 nm×about 83 nm×about 2.5 nm in dimension.

The length (or number of nucleotides) of the ssDNA used as scaffold for DNA origami synthesis is the major factor in determining the dimensions of the DNA origami structure. The most common scaffold for the synthesis of DNA origami structures is the ssDNA derived from M13mp18 genome, 7249 bp long. Using M13mp18 scaffold sequence for designing single-layer symmetrical DNA origami structures enables the synthesis of structures that are at maximum about 83 nm×83 nm×1.5 nm in dimensions. These dimensions significantly decrease if, a double layer structure is designed. This example describes using a single reaction step for the folding of a DNA nanostructure from a 19 kb ssDNA to synthesize larger nucleic acid origami structures. The DNA nanostructure can be a structured nucleic acid particle with a post having a footprint of about 83 nm×83 nm×2.5 nm.

The 19 kb ssDNA scaffold was synthesized using an about 19,060 bp (SEQ ID NO: 1339) plasmid having a unique sequence and converted to ssDNA by M13KO7 helper phage infection. Extraction of ssDNA from the phage was performed by phenol-chloroform extraction and further purification was performed using standard techniques recognized in the art.

Notwithstanding the appended claims, the disclosure set forth herein is also defined by the following clauses:
1. A method of reversibly binding an affinity reagent to an analyte, comprising
   (a) binding an analyte to an affinity reagent, wherein the analyte is attached to a structured nucleic acid particle during the binding, wherein the structured nucleic acid is in a first conformation; and
   (b) converting the structured nucleic acid particle to a second conformation, wherein the structured nucleic acid particle blocks binding of the analyte to the affinity reagent, thereby dissociating the affinity reagent from the analyte.
2. The method of clause 1, wherein the analyte comprises a protein.
3. The method of clause 2, wherein the protein is denatured.
4. The method of clause 1, wherein the structured nucleic acid particle is attached to a solid support.
5. The method of clause 4, wherein the solid support comprises an array of sites and wherein the structured nucleic acid particle is attached to the solid support at a site of the array.
6. The method of clause 5, wherein individual sites of the array are each attached to a different analyte via a structured nucleic acid particles.
7. The method of any one of the preceding clauses, further comprising
   (c) after step (b), converting the structured nucleic acid particle to the first conformation; and
   (d) binding the analyte to a second affinity reagent while the structured nucleic acid particle is in the first conformation.
8. The method of clause 7, wherein step (b) further comprises removing the affinity reagent from a vessel containing the structured nucleic acid particle.
9. The method of clause 7, wherein removing the affinity reagent from the vessel comprises delivering to the vessel a wash fluid and removing the wash fluid from the vessel.
10. The method of any one of clauses 7 through 9, comprising repeating steps (a) and (b) using a third affinity reagent instead of the affinity reagent and repeating steps (c) and (d) using a fourth affinity reagent instead of the second affinity reagent.
11. The method of any one of the preceding clauses, wherein the analyte is connected to the structured nucleic acid particle by a linker, wherein the analyte is more distant from a surface of the structured nucleic acid particle in the first conformation compared to the second conformation.
12. The method of clause 11, wherein the linker comprises a nucleic acid having a first strand.
13. The method of clause 12, wherein, in the first conformation, the linker further comprises a second strand hybridized to the first strand.
14. The method of clause 13, wherein the converting of the structured nucleic acid particle to the second conformation comprises removing the second strand from the linker.
15. The method of clause 14, wherein the second strand comprises a single-stranded toehold sequence, and wherein the removing the second strand from the linker comprises hybridizing the second strand to a third strand by toehold-mediated strand displacement.
16. The method of clause 14 or 15, wherein the converting of the structured nucleic acid particle to the second conformation comprises self-hybridizing the first strand to form a hairpin.
17. The method of clause 16, wherein, in the second conformation, the first strand comprises a toehold sequence and the hairpin.
18. The method of any one of clauses 13 through 17, further comprising: (c) after step (b), converting the structured nucleic acid particle to the first conformation by hybridizing a third strand to the first strand by toehold-mediated strand displacement; and
(d) binding the analyte to a second affinity reagent while the structured nucleic acid particle is in the first conformation.
19. The method of clause 14 or 15, wherein the converting of the structured nucleic acid particle to the second conformation comprises hybridizing the first strand to a nucleotide sequence of the structured nucleic acid particle.
20. The method of clause 19, wherein the nucleotide sequence is located on a surface of the structured nucleic acid particle.
21. The method of clause 20, wherein, in the second conformation, the first strand comprises a toehold sequence.
22. The method of any one of clauses 19 through 21, further comprising:
   (c) after step (b), converting the structured nucleic acid particle to the first conformation by hybridizing a third strand to the first strand by toehold-mediated strand displacement; and
   (d) binding the analyte to a second affinity reagent while the structured nucleic acid particle is in the first conformation.
23. The method of clause 22, wherein, in the first conformation, the structured nucleic acid particle comprises a second strand and the second strand is hybridized to a third strand.
24. The method of clause 23, wherein the second strand comprises a nucleotide sequence that is identical to a nucleotide sequence of the first strand.
25. The method of clause 24, wherein the converting of the structured nucleic acid particle to the second conformation comprises removing the third strand from the second strand, thereby hybridizing the linker to the second strand.
26. The method of clause 25, wherein the third strand comprises a single-stranded toehold sequence, and wherein the removing the third strand from the second strand comprises hybridizing the third strand to a fourth strand by toehold-mediated strand displacement.
27. The method of clause 26, wherein the second strand is located on a surface of the structured nucleic acid particle.
28. The method of clause 27, wherein, in the second conformation, the first strand comprises a toehold sequence.
29. The method of any one of clauses 22 through 28, further comprising:
   (c) after step (b), converting the structured nucleic acid particle to the first conformation by hybridizing a fifth strand to the second strand by toehold-mediated strand displacement; and
   (d) binding the analyte to a second affinity reagent while the structured nucleic acid particle is in the first conformation.
30. The method of clause 29, wherein, in the first conformation, (i) the first strand comprises a single stranded nucleotide sequence, and (ii) the structured nucleic acid particle comprises a second strand.
31. The method of clause 30, wherein the converting of the structured nucleic acid particle to the second conformation comprises hybridizing a splint strand to the first strand and the second strand.
32. The method of clause 30 or 31, wherein the splint strand comprises a single-stranded toehold sequence when hybridized to the first strand and the second strand.
33. The method of clause 32, further comprising:
   (c) after step (b), converting the structured nucleic acid particle to the first conformation by hybridizing a reset strand to the splint strand by toehold-mediated strand displacement; and
   (d) binding the analyte to a second affinity reagent while the structured nucleic acid particle is in the first conformation.
34. The method of any one of clauses 11 through 33, wherein the linker comprises a first moiety connected to the structured nucleic acid particle and a second moiety connected to the analyte.
35. The method of clause 34, wherein the first moiety comprises a backbone region in a nucleotide strand.
36. The method of clause 35, wherein the nucleotide strand passes through the linker and through a region of the structured nucleic acid particle other than the linker.
37. The method of clause 35 or 36, wherein the first moiety comprises at least two, non-contiguous backbone regions in the nucleotide sequence of the nucleotide strand.
38. The method of any one of clauses 34 through 37, wherein the linker comprises a length of at least 10 nm between the first moiety and the second moiety.
39. The method of any one of clauses 34 through 37, wherein the linker comprises a continuous chain of covalent bonds attaching the structured nucleic acid particle and the analyte.
40. The method of any one of clauses 34 through 39, wherein the linker comprises a non-covalent bond which is requisite for attaching the structured nucleic acid particle to the analyte.
41. The method of clause 40, wherein the non-covalent bond comprises a receptor bound to a ligand.
42. The method of any one of clauses 1 through 41, wherein the structured nucleic acid particle comprises nucleic acid origami.
43. The method of clause 42, wherein the nucleic acid origami comprises a scaffold strand passing through the linker and through the surface of the structured nucleic acid particle.
44. The method of clause 1, wherein the analyte is attached to a surface of the structured nucleic acid particle in the first conformation,
   wherein the surface is exposed to the exterior of the structured nucleic acid particle in the first conformation, and
   wherein the surface is sequestered within the structured nucleic acid particle in the second conformation.
45. The method of clause 44, wherein the surface is substantially planar in the first conformation.
46. The method of clause 45, wherein the surface is inwardly curved or inwardly folded in the second conformation.
47. The method of clause 44, 45 or 46, wherein the converting of the structured nucleic acid particle to the second conformation comprises contacting the structured nucleic acid particle with divalent cations.
48. A structured nucleic acid particle, comprising
   (a) a scaffold strand comprising a nucleotide sequence;
   (b) a plurality of different staple strands each comprising a different nucleotide sequence, wherein the staple strands are hybridized to the scaffold strand;
   (c) an analyte handle comprising the nucleotide sequence of APT 40.2 (CACTCACCTCCATCTCCACTCCTACCCATCCAACTCCCAC) hybridized to the nucleotide sequence of TCOv40.2 (GTGGGAGTTGGATGGGTAGGAGTGGAGATGGAGGTGAGTG);
   (d) a plurality of label handles comprising the nucleotide sequence of DYE40.22.9 (CGTTTCTTTGCTTGTGTCTTGTCTTCGTCGTGCTTGTTCC) hybridized to the nucleotide sequence of DYE647v40.22.9 (GGAACAAGCACGACGAAGACAAGACACAAGCAAAGAAACG); and
   (e) a plurality of attachment handles comprising the nucleotide sequence of JF20.22.9 (ACAGTTATTAGCCCGCATTT) hybridized to the nucleotide sequence of JFDBCOv20.22.9 (AAATGCGGGCTAATAACTGT).
49. The structured nucleic acid particle of clause 48, wherein the nucleotide sequence of APT 40.2 is present in a nucleic acid strand that is hybridized to the scaffold strand or to a staple strand of the plurality of different staple strands.
50. The structured nucleic acid particle of clause 48 or 49, wherein the nucleotide sequence of TCOv40.2 is present in a nucleic acid strand functionalized with an analyte of interest.
51. The structured nucleic acid particle of any one of clauses 48 through 50, wherein the nucleotide sequence of DYE40.22.9 is present in a nucleic acid strand that is hybridized to the scaffold strand or to a staple strand of the plurality of different staple strands.
52. The structured nucleic acid particle of clause 48 or 51, wherein the nucleotide sequence of DYE647v40.22.9 is present in a nucleic acid strand functionalized with an exogenous label.
53. The structured nucleic acid particle of any one of clauses 48 through 52, wherein the nucleotide sequence of JF20.22.9 is present in a nucleic acid strand that is hybridized to the scaffold strand or to a staple strand of the plurality of different staple strands.
54. The structured nucleic acid particle of clause 48 or 53, wherein the nucleotide sequence of JFDBCOv20.22.9 is present in a nucleic acid strand attached to a solid support.
55. The structured nucleic acid particle of clause 48, wherein the nucleotide sequence of TCOv40.2 is present in a nucleic acid strand that is hybridized to the scaffold strand or to a staple strand of the plurality of different staple strand strands.
56. The structured nucleic acid particle of clause 48 or 55, wherein the nucleotide sequence of APT 40.2 is present in a nucleic acid strand functionalized with an analyte of interest.
57. The structured nucleic acid particle of any one of clauses 48, 55 or 56, wherein the nucleotide sequence of DYE647v40.22.9 is present in a nucleic acid strand that is hybridized to the scaffold strand or to a staple strand of the plurality of different staple strands.
58. The structured nucleic acid particle of clause 48 or 57, wherein the nucleotide sequence of DYE40.22.9 is present in a nucleic acid strand functionalized with an exogenous label.
59. The structured nucleic acid particle of any one of clauses 48 or 55 through 58, wherein the nucleotide sequence of JFDBCOv20.22.9 is present in a nucleic acid strand that is hybridized to the scaffold strand or to a staple strand of the plurality of different staple strands.
60. The structured nucleic acid particle of clause 48 or 59, wherein the nucleotide sequence of JF20.22.9 is present in a nucleic acid strand attached to a solid support.
61. The structured nucleic acid particle of any one of clauses 48 through 60, comprising one and only one analyte handle comprising the nucleotide sequence of APT 40.2 hybridized to the nucleotide sequence of TCOv40.2.
62. The structured nucleic acid particle of clause 61, wherein the analyte handle is attached to a protein.
63. The structured nucleic acid particle of clause 61, wherein the analyte handle is attached to a bioorthogonal reactant or click reactant.
64. The structured nucleic acid particle of clause 61, wherein the analyte handle is attached to streptavidin, biotin, Spycatcher, or SpyTag.
65. The structured nucleic acid particle of any one of clauses 48 through 61, comprising a plurality of at least four label handles each comprising the nucleotide sequence of DYE40.22.9 hybridized to the nucleotide sequence of DYE647v40.22.9.
66. The structured nucleic acid particle of clause 65, wherein at least one of the label handles is attached to a luminescent label.
67. The structured nucleic acid particle of clause 65, wherein a first label handle is attached to a first luminescent label and a second label handle is attached to a second luminescent label, the first luminescent label having a different structure from the second luminescent label.
68. The structured nucleic acid particle of clause 67, wherein the first luminescent label and the second luminescent label emit luminescence at an overlapping region of the electromagnetic spectrum.
69. The structured nucleic acid particle of clause 67 or 68, wherein the first luminescent label and the second luminescent label emit luminescence at non overlapping regions of the electromagnetic spectrum.
70. The structured nucleic acid particle of any one of clauses 48 through 65, comprising a plurality of at least four attachment handles comprising the nucleotide sequence of JF20.22.9 hybridized to the nucleotide sequence of JFDBCOv20.22.9.
71. The structured nucleic acid particle of clause 70, wherein at least one of the attachment handles is attached to a solid support.
72. The structured nucleic acid particle of any one of clauses 48 through 71, wherein each of the staple strands is hybridized to two non-contiguous regions in the nucleotide sequence of the scaffold nucleic acid.
73. The structured nucleic acid particle of any one of clauses 48 through 71, wherein the structured nucleic acid particle is stored in a frozen medium.
74. A structured nucleic acid particle, comprising
   (a) a scaffold strand comprising a nucleotide sequence;
   (b) a plurality of different staple strands comprising different nucleotide sequences hybridized to the scaffold strand, wherein each of the staple strands is hybridized to two non-contiguous regions in the nucleotide sequence of the scaffold strand; and
   (c) a handle strand comprising a nucleotide sequence hybridized to the scaffold strand or to a staple strand, wherein the handle strand is attached to the scaffold strand or to the staple strand by an artificial cross-link.
75. The structured nucleic acid particle of clause 74, wherein the artificial cross-link comprises a cyclobutane thymidine dimer.
76. The structured nucleic acid particle of clause 74, wherein the artificial cross-link comprises a psoralen cross-link.
77. The structured nucleic acid particle of any one of clauses 74 through 76, wherein the handle strand is attached to the scaffold strand by the artificial cross-link.
78. The structured nucleic acid particle of any one of clauses 74 through 76, wherein the handle strand is attached to a staple strand by the artificial cross-link.
79. The structured nucleic acid particle of any one of clauses 74 through 76, wherein a staple strand of the plurality of staple strands is attached to the scaffold strand by a second artificial cross-link.
80. The structured nucleic acid particle of clause 79, wherein at least two staple strands of the plurality of staple strands are each attached to the scaffold strand by a second artificial cross-link.
81. The structured nucleic acid particle of clause 79 or 80, wherein the second artificial cross-link comprises a cyclobutane thymidine dimer or psoralen cross-link.
82. The structured nucleic acid particle of any one of clauses 74 through 81, wherein the structured nucleic acid particle is stored in a frozen medium.
83. The structured nucleic acid particle of any one of clauses 74 through 82, wherein the artificial cross-link comprises an artificial covalent bond between to nucleic acid strands.
84. The structured nucleic acid particle of any one of clauses 74 through 82, wherein the artificial cross-link comprises a transitional metal.
85. The structured nucleic acid particle of any one of clauses 74 through 82, wherein the artificial cross-link comprises a peptide nucleic acid or locked nucleic acid.
86. A structured nucleic acid particle comprising
   (a) a nucleic acid scaffold hybridized to a plurality of nucleic acid staples to form a nucleic acid origami having an isotropic shape; and
   (b) an affinity moiety attached to the nucleic acid origami.
87. The structured nucleic acid particle of clause 86, wherein the isotropic shape is a cube.
88. The structured nucleic acid particle of clause 87, wherein the nucleic acid origami comprises up to or at least 96 helices arranged in a hexagonal lattice.
89. The structured nucleic acid particle of clause 88, wherein the dimensions of the cube are from 15-30 nm on a side, and particularly approximately 24 nm on a side+/−5 nm.
90. The structured nucleic acid particle of any one of clauses 86 through 89, further comprising a plurality of labels, wherein the nucleic acid origami comprises a plurality of planar faces that are attached to a label of the plurality of labels.
91. The structured nucleic acid particle of any one of clauses 86 through 90, further comprising a plurality of affinity moieties, wherein the nucleic acid origami comprises a plurality of planar faces that are attached to an affinity moiety of the plurality of affinity moieties.
92. The structured nucleic acid moiety of clause 91, wherein at least one tether is attached to each of the planar faces in the plurality of planar faces that is attached to an affinity moiety.
93. The structured nucleic acid moiety of clause 92, wherein an affinity moiety that is attached to at least one of the planar faces is bound to an epitope of an analyte, and wherein a tether that is attached to the at least one of the planar faces is bound to a docker of the analyte.
94. A method for purifying a structured nucleic acid particle having nucleic acid origami comprising steps of (a) providing a mixture comprising a nucleic acid origami and excess reactants for assembly or modification of the nucleic acid origami; and (b) separating the structured nucleic acid particles from the excess reactants.
95. The method of clause 94, wherein the excess reactants are selected from the group consisting of nucleic acid scaffolds, nucleic acid staples, nucleic acid handles, labels, affinity reagents, analyte of interest, tethers and dockers.
96. The method of clause 94, wherein the separating comprises contacting the mixture with ion exchange chromatography media.

1. A structured nucleic acid particle, comprising:
a single stranded nucleic acid scaffold; and
a plurality of single stranded nucleic acid staple strands hybridized to the scaffold;
wherein the plurality of staple strands hybridized to the scaffold form the scaffold into a structured nucleic acid particle having a first planar structure coupled to a first protruding structure extending from a face of the first planar structure.

2. The structured nucleic acid particle of claim 1, wherein the plurality of staple strands comprises different nucleotide sequences.

3. The structured nucleic acid particle of claim 1, wherein each of the staple strands is hybridized to two non-contiguous regions in the scaffold.

4. The structured nucleic acid particle of claim 1, further comprising a plurality of handles selected from the group consisting of one or more analyte handles, one or more dockers, one or more label handles, one or more attachment handles, and a combination thereof.

5. The structured nucleic acid particle of claim 4, wherein each of the plurality of handles attaches to the scaffold or a staple strand of the plurality of single stranded nucleic acid staple strands.

6. The structured nucleic acid particle of claim 4, wherein the one or more label handles are functionalized with an exogenous label.

7. The structured nucleic acid particle of claim 4, wherein the one or more analyte handles are functionalized with an analyte.

8. The structured nucleic acid particle of claim 4, wherein the one or more analyte handles are attached to a bioorthogonal reactant or click reactant.

9. The structured nucleic acid particle of claim 4, wherein the one or more analyte handles are attached to streptavidin, biotin, Spycatcher, or Spytag.

10. The structured nucleic acid particle of claim 4, wherein the one or more attachment handles are attached to a solid support.

11. The structured nucleic acid particle of claim 1, wherein a staple strand of the plurality of staple strands is attached to the scaffold by a first artificial cross-link.

12. The structured nucleic acid particle of claim 11, wherein any of the plurality of handles comprises a nucleotide sequence hybridized to the scaffold strand or to the staple strand of the plurality of single stranded nucleic acid staple strands, and wherein the any of the plurality of handles is attached to the scaffold strand or to the staple strand by a second artificial cross-link.

13. The structured nucleic acid particle of claim 12, wherein the first artificial cross-link, the second artificial cross-link or both comprises a cyclobutane thymidine dimer or psoralen cross-link.

14. The structured nucleic acid particle of claim 12, wherein the first artificial cross-link, the second artificial cross-link or both comprises an artificial covalent bond between to nucleic acid strands.

15. The structured nucleic acid particle of claim 12, wherein the first artificial cross-link, the second artificial cross-link or both comprises a transitional metal.

16. The structured nucleic acid particle of claim 12, wherein the first artificial cross-link, the second artificial cross-link or both comprises a peptide nucleic acid or locked nucleic acid.

17. The structured nucleic acid particle of claim 1, wherein an analyte is attached to the first protruding structure at a face opposite from the first planar structure.

18. The structured nucleic acid particle of claim 1, wherein the first protruding structure ranges from about 10 nm-40 nm, about 15 nm-40 nm or about 15 nm-35 nm in length.

19. The structured nucleic acid particle of claim 1, wherein the structured nucleic acid particle further comprises a second planar structure extending in parallel from the first planar structure.

20. The structured nucleic acid particle of claim 1, wherein the structured nucleic acid particle is a nucleic acid origami comprising helices arranged in a hexagonal lattice.

21. The structured nucleic acid particle of claim 1, wherein the single stranded nucleic acid scaffold ranges from about 7 kb-22 kb in length.

22.-40. (canceled)
